(19) United States
(12) Patent Application Publication
(10) Pub. No.: US 2024/0417434 A1
(43) Pub. Date: Dec. 19, 2024

(54) NUCLEIC ACID CONSTRUCTS, VIRAL VECTORS AND VIRAL PARTICLES

(71) Applicant: UCB BIOPHARMA SRL, Brussels (BE)

(72) Inventors: Natalia RODRIGUEZ ALVAREZ, Slough, Berkshire (GB); Csilla SIPEKY, Slough, Berkshire (GB); Brittany Nicole VALLETTE, Slough, Berkshire (GB); Christian Gilbert Joseph WOLFF, Slough, Berkshire (GB); Meiyu XU, Slough, Berkshire (GB)

(21) Appl. No.: 18/704,867

(22) PCT Filed: Oct. 26, 2022
(86) PCT No.: PCT/EP2022/080020

§ 371 Date: Apr. 25, 2024

Related U.S. Application Data
(60) Provisional application No. 63/263,175, filed on Oct. 28, 2021.

(51) Int. Cl. C07K 14/47; A61K 48/00; A61P 25/08; C12N 15/86

(52) U.S. Cl. CPC C07K 14/4702; A61K 48/0025; A61K 48/0058; A61P 25/08; C12N 15/86; C12N 2750/14142; C12N 2750/14143; C12N 2830/50

(57) ABSTRACT

The present invention relates to a nucleic acid construct comprising a transgene encoding syntaxin binding protein 1 (STXBP1, Munc-18), a viral vector for packaging said nucleic acid in a viral particle; and use of such viral particle for treating disease associated with a loss of STXBP1 functional activity.

### FIELD OF THE INVENTION

The present invention relates to nucleic acid constructs, viral vectors and viral particles for use in the treatment and/or prevention of disease associated with a loss of syntaxin binding protein 1 (STXBP1) functional activity, identified for example in neurodevelopmental disorders associated with epilepsy such as Ohtahara syndrome, West syndrome and Dravet syndrome.

### BACKGROUND OF THE INVENTION

Many neurodevelopmental disorders have been associated with genetic alterations leading to the manifestation of severe clinical symptoms early in life. Genetic alteration of STXBP1 is associated with severe early onset epileptic encephalopathies (EOEE) such as Ohtahara syndrome, West syndrome and Dravet syndrome (Saitsu et al. 2008, Stamberger et al. 2016). STXBP1 encephalopathies (STXBP1-E) are characterized by a large spectrum of symptoms but it is now established that all patients have profound intellectual disability and up to 85% of patients develop seizures (Abramov et al. 2020). STXBP1-E may be caused by dominant, heterozygous, de novo mutations in the STXBP1 (Munc-18) gene. Multiple genetic variants have been reported including missense, nonsense, frameshift, deletions, duplication and splice site variants. Most cases are caused by heterozygous loss of function (LoF) mutations, typically de novo but in rare cases inherited from heterozygous or mosaic parents. A recent variant has been described that leads to a homozygous mutation of STXBP1. Genotype-phenotype correlation studies have failed to identify a clear association between mutation type and the different expressions of STXBP1-E to date.

STXBP1 (Munc-18; Syntaxin binding protein 1) is an essential component of the molecular machinery that controls SNARE-mediated (N-ethylmaleimide-sensitive factor attachment protein receptor) membrane fusion in neurons and neuroendocrine cells. STXBP1 regulates the formation of the SNARE complex by binding to the closed conformation of syntaxin-1, a process that drives the fusion of synaptic vesicles and the neurotransmitter release at the synapse.

FIG. 1 shows a schematic drawing of STXBP1 impact on synaptic transmission under normal (A) and disease conditions (B). STXBP1 (Munc18) is a major component of the synaptic machinery and its interaction with Syntaxin-1 at the presynaptic membrane is a critical step to trigger neurotransmitter release. Under normal conditions (A), STXBP1 is abundantly expressed at the presynaptic membrane and the complex formation with Syntaxin-1 ensures efficient synaptic vesicle fusion which results in neurotransmitter release and the generation of post synaptic currents. Under disease conditions (B), mutant STXBP1 does not express or is unable to directly bind to Syntaxin-1 and the remaining normal STXBP1 levels are not enough to maintain efficient neurotransmitter release resulting into reduced post synaptic currents [Patzke et al. 2015].

STXBP1 knock-out (KO) studies have demonstrated that absence of the protein in neurons leads to a complete loss of neurotransmitter secretion from synaptic vesicles throughout development (Verhage et al. 2000). The characterization of heterozygous KO models for STXBP1 (HET) indicated that a reduction of about 50% of the STXBP1 protein levels results in a strong seizure phenotype characterized by myoclonic jerks and spike-wave discharges (Kovacevic et al. 2018, Orock et al. 2018, Chen et al. 2020). The extended phenotyping of such HET mice also showed impaired cognitive performance, hyperactivity and anxiety-like behavior. The generation of mice with a heterozygous expression in only gabaergic neurons provided further insights into the mechanism of STXBP1 mutations by highlighting differences in synaptic transmission from gabaergic interneurons to glutamatergic pyramidal neurons (Chen et al. 2020).

The STXBP1 HET mouse neurons show normal synaptic transmission although more detailed analysis indicated that reduced levels of STXBP1 result in increased synaptic depression during intense stimulation at glutamatergic, GABAergic, and neuromuscular synapses (Toonen et al. 2006). Experiments with stem cell derived human neurons indicated that a 20-30% reduction in STXBP1 levels results in a dramatic decrease of the normal synaptic function (Patzke et al. 2015) further highlighting that the effects of STXBP1 mutations may vary between neuronal subtypes and species background. On the contrary, overexpression of STXBP1 in normal mouse neurons results in increased synaptic function (Toonen et al. 2006) and phenotypic analysis of a transgenic mouse strain that overexpresses the protein isoform munc18-1a in the brain displayed several schizophrenia-related behaviors (Uriguen et al. 2013). Additional non-synaptic roles have been described for STXBP1 and suggested to regulate the mechanism of radial migration of cortical neurons. STXBP1 may thus also modulate vesicle fusion at the plasma membrane to distribute various proteins on the cell surface and the vesicle transport from Golgi to the plasma membrane (Hamada et al. 2016).

Mutations in STXBP1 that result in loss of functional activity have been characterized in vitro and in model systems to establish the impact on neuronal function. Mutations that lead to a truncation of the STXBP1 protein, in general linked to nonsense, frameshift or deletions, are not detected in neuronal systems and it is hypothesized that such mutant proteins are rapidly downregulated by a nonsense mediated decay mechanism of their RNA messengers. About 40-50% of mutations in STXBP1 are missense mutations (Abramov et al. 2020) and in vitro experiments have demonstrated that such point mutations result in a decreased stability of the STXBP1 protein and leading to a reduced expression levels in neuronal systems (Kovacevik et al. 2018, Zhu et al. 2020). The study of stem cell derived neurons from Othahara patients carrying STXBP1 missense mutations also indicated a reduction of STXBP1 protein levels (Yamashita et al. 2016). More recently, a homozygous STXBP1 mutation was identified and in vitro studies indicated that the homozygous L446F mutation causes a gain-of-function phenotype while having less impact on protein levels than previously reported for the heterozygous mutations (Lammertse et al. 2020).

Overall, STXBP1 genetic disorder is linked to a loss of function of the STXBP1 protein and multiple therapeutic approaches have been proposed including, small molecule chaperons to prevent aggregation of mutant forms and antisense oligonucleotides to downregulate specific miRNA that negatively regulate STXBP1 expression (Abramov et al. 2020). The complexity for developing a disease modifying therapy for STXBP1 lies in the development of new specific tools able to restore normal STXBP1 functional activity and that can be translated to the clinic. No approved drug therapy addressing the underlying disease mechanism is available at this point.

There is a clear unmet medical need for effective treatment of STXBP1 genetic disorders. The present disclosure provides a disease modifying gene therapy overexpressing STXBP1 to restore normal STXBP1 functional activity with the potential to cure.

### SUMMARY OF THE INVENTION

The present invention provides by means of gene therapy, a healthy copy of the STXBP1 gene, that is capable of compensating for the effects of STXBP1 mutation and restoring normal STXBP1 functional activity.

The present invention provides:

A nucleic acid construct comprising a transgene encoding:
i. a syntaxin binding protein 1 (STXBP1) comprising isoform a, b, c, d, e, f, g or h, having the sequence given in SEQ ID NO: 9, 10, 11, 12, 13, 14, 15 or 16 respectively; or
ii. a sequence having at least 95% or 96% or 97% or 98% or 99% or 99.5% sequence identity to SEQ ID NO: 9, 10, 11, 12, 13, 14, 15 or 16 and retaining functionality as STXBP1; or
iii. a naturally-occurring variant comprising, with reference to SEQ ID NO:9, one or more mutations as shown in Table 7.

The invention further provides:
A viral vector comprising the nucleic acid construct.
A viral particle comprising the viral vector.
The medical use of the viral particle for the treatment and/or prevention of an STXBP1 genetic disorder.
A method of treating and/or preventing a disease characterised by STXBP1 mutation(s), comprising administering the viral particle to a subject in need thereof.

### BRIEF DESCRIPTION OF THE DRAWINGS

FIG. 1: Schematic drawing of STXBP1 impact on synaptic transmission under normal (A) and disease conditions (B). STXBP1 (Munc18) is a major component of the synaptic machinery and its interaction with Syntaxin-1 at the presynaptic membrane is a critical step to trigger neurotransmitter release. Under normal conditions (A), STXBP1 is abundantly expressed at the presynaptic membrane and the complex formation with Syntaxin-1 ensures efficient synaptic vesicle fusion which results in neurotransmitter release and the generation of post synaptic currents. Under disease conditions (B), mutant STXBP1 does not express or is unable to directly bind to Syntaxin-1 and the remaining normal STXBP1 levels are not enough to maintain efficient neurotransmitter release resulting in reduced post synaptic currents [Patzke et al. 2015].

FIG. 2: Protein sequence alignment of the human, monkey and mouse STXBP1 sequences (human isoform a according to SEQ ID NO: 9). The alignment shows the high sequence homology across the species. The monkey and mouse amino acid sequences are identical to the human amino acid sequence.

FIG. 3: Schematic cartoon of the designed constructs. In the figure, “prom” means promoter; “INT” means intron, “h” means human, SV40 means polyadenylation sequence SV40; “tag” means an HA or Myc tag, located either at the N or at the C terminus of a construct.

FIG. 4: (A) Immunofluorescence imaging of AD-HEK293 cells transfected with hSTXBP1 plasmids driven by various promoters (CAG, MECP2 and MECP2-intron) detected with anti-STXBP1 antibody. (B) The magnification section shows that STXBP1 is localized to the cell membrane. AD=adherent, NC=negative control.

FIG. 5: (A) Immunofluorescence imaging of Neuro-2A cells transfected with hSTXBP1 plasmids driven by various promoters (CAG, MECP2 and MECP2-intron) detected with anti-STXBP1 antibody. (B) Magnification showing that STXBP1 is localized to the cell membrane. NC=negative control.

FIG. 6: Western blot analysis of Neuro-2A cells transfected with hSTXBP1 driven by various promoters (CAG, MECP2 and MECP2-intron). Two technical replicates of each condition are shown. NC=negative control, 1=MECP2-intron-hSTXBP1, 2=CAG-hSTXBP1, 3=MECP2-hSTXBP1.

FIG. 7: Western blot analysis of (A) Myc-tagged hSTXBP1 driven by CAG promoter in AD-HEK293 cells detected with anti-Myc antibody and (B) HA-tagged hSTXBP1 driven by hSYN promoter in AD-HEK293 cells, SH-SY5Y cells and Neuro-2a cells detected with anti-HA antibody. Two technical replicates of each condition are shown. (C) Epitope tagged proteins were also detected in AD-HEK293 cells using anti-STXBP1 antibodies. NC=negative control, 1=CAG-hSTXBP1-Myc, 2=CAG-Myc-hSTXBP1, 3=hSYN-HA-hSTXBP1. The background protein band in the NC lane in (A) is due to detection of endogenous Myc by the anti-Myc antibody.

FIG. 8: Lentiviral vector transduction of SXTBP1 cassettes in iPSCs derived glutamatergic neurons. Images show representative pictures of STXBP1 expression under control conditions (non transduced) and following transduction of cassettes under the control of the hSyn or MECP2 promoters.

FIG. 9: AAV9 transduction of STXBP1 in mouse primary neurons. (A) Representative images of STXBP1 staining in primary mouse cortical neurons transduced with AAV9 viral vector at MOI 5.0E+5 GC/cell. Pictures show control conditions (non transduced) and STXBP1 expression under control of hSyn, MECP2 or MECP2-intron promoters. (B) Comparison of the HA staining (right) with the STXBP1 staining (left) in the same primary mouse cortical neurons.

FIG. 10: Co-localization of STXBP1 over-expression in MAP2 positive neurons. Images are representative pictures of anti-HA tag staining (left panels) and anti-MAP2 staining (right panels) in mouse primary neurons following transduction of the AAV9 viral vectors. Arrows indicate examples of cells that express STXBP1 (HA) and the neuronal marker (MAP2).

FIG. 11: Viral vector DNA copies analysis. qPCR data of SV40 pA (polyA signal of simian virus 40) normalized by the number of diploid mouse genomes from the left hippocampus and left frontal cortex of 5 weeks old mice following AAV treatment. Data is shown for vehicle and the four AAV9 transduced groups (control virus, hSyn, MECP2, MECP2-intron). Results are shown as mean±SD.

FIG. 12: STXBP1 mRNA expression analysis. Data are expressed as relative expression normalized to two reference genes and scaled to the average expression of all groups (mean±SD). Analysis was performed from tissues of the left hippocampus and left frontal cortex of 5 weeks old mice following AAV treatment. Data is shown for vehicle and the four AAV9 transduced groups (control virus, hSyn, MECP2, MECP2-intron).

FIG. 13: Protein analysis by Western Blot. (A) Western blot showing HA-tag expression for the different cassettes in the cortex (n=5-7 per group). GAPDH was used as a loading control. (B) Quantification of the HA-tag band intensities, each sample is normalized to the GAPDH loading control. Results are shown as the mean±SD.

FIG. 14: Distribution of infected cells in the mouse brain using GFP reporter from AAV9-hSyn-NLS-eGFP-NLS virus. (A) Sagittal section of a mouse brain that received AAV9-hSyn1-NLS-GFP-NLS icv, sacrificed 1 month later, and immunostained to label GFP. The distribution of cells expressing GFP was observed from front to back of the entire brain. Some of the main brain regions exhibiting GFP+ cells are highlighted with rectangles. (B-G): High magnification of the brain regions showing GFP+ cells from A (arrows point to GFP+ cells).

FIG. 15: Characterization of cells expressing GFP reporter from AAV9-hSyn-NLS-eGFP-NLS virus. Double immunofluorescent labeling was performed to detect (A-F) GFP and the neuronal marker NeuN, (G-L) GFP and the astrocytic marker GFAP. Cell positives both for (A-C) GFP and (D-F) NeuN were observed in all brain regions (arrows point to double-labeled cells) indicating that neurons were transduced and expressed the reporter gene. To the opposite, no GFP (G-I) signal was detected in GFAP positive cells (J-L), suggesting that astrocytes were not expressing the reporter gene.

FIG. 16: Distribution of HA-STXBP1 fusion protein from different promoters in the mouse brain following AAV9 administration. The distribution of HA-tagged STXBP1 overexpressed from different promoters was studied in the brain of mice by immunohistochemistry against HA. As negative control conditions, no HA signal was observed in animals that received (A) PBS only or (B) AAV9-hSyn-GFP virus icv. (C) As a negative control (NC) of antibody selectivity, no HA signal was observed in animals that received AAV9-MECP2-intron-HA-STXBP1 virus but for which the primary HA antibody was omitted during the immunohistochemistry procedure. (D-F) HA signal was observed in the brain of all the animals injected with the different viruses expressing HA-STXBP1 from the different promoters. The 3 promoters led to a common pattern of HA distribution across the whole brain with main expression observed in the cerebral cortex, hippocampus, striatum, olfactory bulbs, substantia nigra and fiber tracts in the forebrain. Noticeable differences in HA distribution between promoters are reported in table 16.

FIG. 17: Distribution of HA-STXBP1 fusion protein from different promoters in the hippocampus following AAV9 administration. Double immunofluorescent labeling was performed to detect (A-C) HA and (D-F) the neuronal marker NeuN which was used to identify the different parts of the hippocampus. All 3 promoters led to HA expression in the entire hippocampus, mainly in neuronal projections (Mol, LMol, Or, MF) and occasionally in cell bodies. (F) MECP2-intron promoter led to a better coverage and higher HA signal intensity compared to the other 2 promoters (D, E). LMol: lacunosum molecular layer the hippocampus; MF: mossy fibers; Mol: molecular layer of the dentate gyrus; Or: stratum oriens.

FIG. 18: Characterization of cells expressing HA-STXBP1 from different promoters. Double immunofluorescent labeling was performed to detect (A-C) HA and (D-F) the neuronal marker NeuN. The cell bodies that were positive for HA and observed occasionally in different regions of the brain were also positive for NeuN supporting that all 3 promoters drive transgene expression in neurons. Arrows point to double labeled cells.

FIG. 19: Analysis of STXBP1 variant mRNA levels in mouse brain by qPCR. mRNA analysis of brain tissue samples from caudal cortex (right hemisphere) of WT (wild-type) littermates and the HET (heterozygotic) mice (n=11-13 per group). (A): mRNA expression analysis of total endogenous STXBP1 (common probe that recognizes all STXBP1 transcripts). (B) and (C): mRNA expression analysis of STXBP1 variants, using two distinct probes that specific recognize the long isoform (B) or the short protein isoform (C). Data are shown as mRNA expression level, by calculating the 2^{-ΔCt} value, where the expression was normalized to the average of the two reference genes. Results are shown as Mean±SD.

FIG. 20: Analysis of STXBP1 protein levels in mouse brain by Western Blot. Tissue samples from the right frontal (medial) cortex of WT (wild-type) littermates and the HET (heterozygotic) mice (n=11-13 per group) have been analyzed. (A): Western blots representing the total STXBP1 protein expression. (B): quantification data of the respective Western blots in (A). B-Actin was used as a loading control, for the normalization. The “WT” group was used as the scaling group. Results are shown as the mean±SD.

FIG. 21: Analysis of STXBP1 variant protein levels in mouse brain by LC-MS. Tissue samples from the lateral half of the frontal cortex of WT (wild-type) littermates and HET (heterozygotic) mice (n=11-13 per group) have been analyzed. (A): Quantification of total STXBP1 peptide vs STXBP1 long isoform vs STXBP1 short isoform. Results are shown as Mean±SD. (B): Western blots representing the STXBP1 short and long isoforms. (C): combined quantification data of the respective Western blots in (B). B-Actin was used as a loading control. Data shown as the ratio between band intensity of each STXBP1 isoform and the respective B-Actin band. Results are shown as the mean±SD.

FIG. 22: Analysis of Syntaxin-1A (STX1A) protein levels in mouse brain by Western Blot. Quantification of STX1A protein expression in the mouse brain tissue samples (n=11-13 per group). B-Actin was used as a loading control, for the normalization. The “WT” group was used as the scaling group. Results are shown as the mean±SD.

FIG. 23: Analysis of AAV transduction efficiency in mouse brain by qPCR (7 weeks post injection) (A) Absolute quantification by qPCR of viral genome copies in WT mice injected with vehicle-PBS (WT), HET mice injected with vehicle-PBS (HET), HET mice injected with STXBP1 long variant (HET-AAV9(L)) and HET mice injected with STXBP1 short variant (HET-AAV9(S)). Samples were collected from the caudal cortex (right hemisphere) and quantified using SV40 pA normalized to the absolute number of diploid mouse genome. Results are shown as Mean±SD. Per group, n=14-15 animals were analyzed and a non-parametric one-way ANOVA (Kruskal-Wallis test) followed by a Dunn's post hoc multiple comparisons test was applied. No significant difference was observed between the transduced groups. (B) mRNA expression analysis of SV40 PolyA and (C) human-specific STXBP1. Data are shown as mRNA expression level, by calculating the 2^{-ΔCt} value, where the expression was normalized to the average of the two reference genes. Results are shown as mean±SD. Per group, n=14-15 animals were analyzed, and a non-parametric one-way ANOVA (Kruskal-Wallis test) followed by a Dunn's post hoc multiple comparisons test was applied. No significant difference was observed between the transduced groups.

FIG. 24: Analysis of STXBP1 variant expression following AAV treatment in mouse brain by qPCR (7 weeks post injection). Analysis of STXBP1 variant mRNA expression was performed using probes that specifically measure total (mouse and human) levels of the short (A) or the long variant (B). Data are shown as mRNA expression level, by calculating the 2^{-ΔCt} value, where the expression was normalized to the average of the two reference genes. Results are shown as Mean±SD. Per group, n=14-16 animals were analyzed.

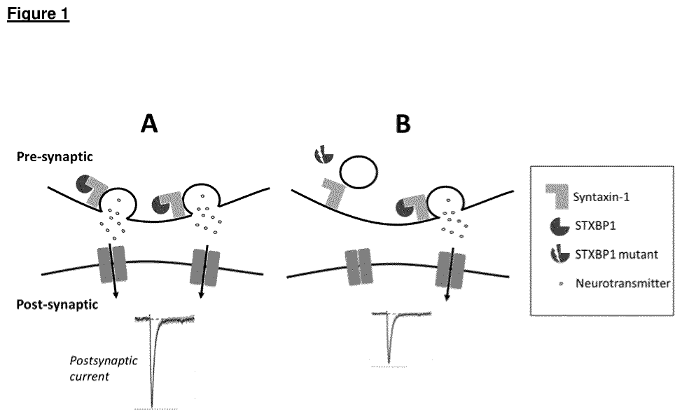

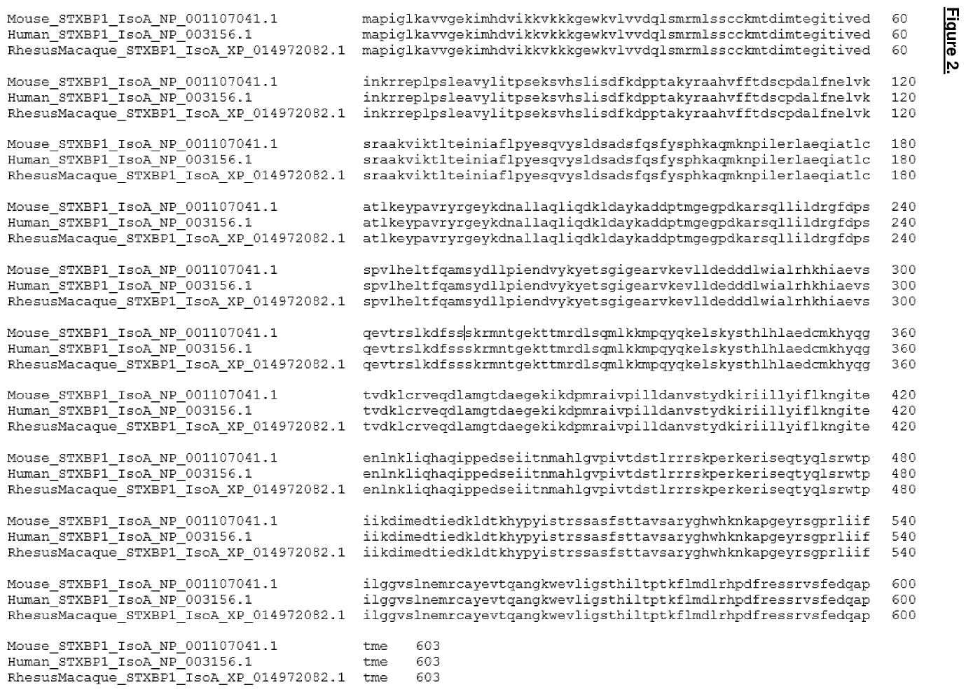

FIG. 25: Analysis of STXBP1 variant expression following AAV treatment in mouse brain by Western blot (7 weeks post injection). Protein analysis by Western blot of samples from right frontal (medial) cortex, in WT mice injected with vehicle-PBS (WT), HET mice injected with vehicle-PBS (HET), HET mice injected with STXBP1 long variant (HET-AAV9(L)) and HET mice injected with STXBP1 short variant (HET-AAV9(S))
(A) Quantification of western blot data for total STXBP1 (long and short variants) protein expression.
(B) Quantification of western blot data for the long STXBP1 variant protein expression.
(C) Quantification of western blot data for the short STXBP1 variant protein expression.
(D) Quantification of western blot data for Syntaxin-1A protein expression
   B-Actin was used as a loading control, for the normalization of each STXBP1 and STX1A band intensity. The vehicle WT group (WT) was used as the scaling group. Results are shown as the mean±SD. The data was analyzed using non-parametric one-way ANOVA (Kruskal-Wallis test) followed by a Dunn's post hoc multiple comparisons test (*<p.0.05; **p<0.01 ***p<0.001; ****p<0.0001).

FIG. 26: Brain distribution of HA− tagged STXBP1 expression following AAV treatment in mouse brain by immunohistochemistry (7 weeks post injection). HA tag staining was performed on saggital sections from HET mice injected with the HA-tagged STXBP1 long variant and compared to vehicle (PBS) treated mice. An example of representative brain section are shown for the AAV treated group (animal 6023) and the vehicle treated group (animal 6009). A strong HA staining is observed in major brain regions in animal 6023 (AAV treated) whereas no HA staining is observed in the PBS treated groups (animal 6009).

FIG. 27: Analysis of spike wave discharges (SWD) following AAV treatment in STXBP1 HET mouse brain by EEG (6-7 weeks) (A,B) and 24 weeks (C,D) post injection. (A) Average number of SWDs in WT mice injected with vehicle-PBS (WT, n=10), HET mice injected with vehicle-PBS (HET, n=19), HET mice injected with STXBP1 long variant (HET-AAV9(L), n=15) and HET mice injected with STXBP1 short variant (HET-AAV9(S), n=16). SWDs were analyzed 6-7 weeks after injection over a period of 24 h for 7 consecutive days. (B) Analysis of the number of animals that are “seizure free” (without any SWD detected during recordings) and animals “with seizures” (SWD detected during recordings). (C) Average number of SWDs in WT mice injected with vehicle-PBS (WT, n=5), HET mice injected with vehicle-PBS (HET, n=12), HET mice injected with STXBP1 long variant (HET-AAV9(L), n=9) and HET mice injected with STXBP1 short variant (HET-AAV9(S), n=11). SWDs were analyzed 24 weeks after injection over a period of 24 h for 7 consecutive days. (D) Analysis of the number of animals that are “seizure free” (without any SWD detected during recordings) and animals “with seizures” (SWD detected during recordings) 24 weeks after injection. The difference between groups was analyzed by non-parametric one-way ANOVA (Kruskal-Wallis test) followed by a Dunn's post hoc multiple comparisons test (****p<0.0001), (***p<0.001) and for Seizure free analysis a chi-square contingency test was used.

FIG. 28: Analysis of body weight following AAV treatment in STXBP1 HET mice (1-22 weeks post injection) (A) Average body weights as a function of age in WT (n=17) and HET mice (n=16) injected with vehicle-PBS. The difference between groups was analyzed by two-way repeated measures ANOVA followed by an uncorrected Fisher's LSD post hoc multiple comparisons test (*<p.0.05; **p<0.01; ***p<0.001; ****p<0.0001). (B) Average body weight measured at 22 weeks old in WT mice injected with vehicle-PBS (WT, n=17) and HET mice injected with vehicle-PBS (HET, n=16), HET mice injected with STXBP1 long variant (HET-AAV9(L), n=10) and HET mice injected with STXBP1 short variant (HET-AAV9(S), n=13). The difference between groups was analyzed by parametric one-way ANOVA followed by an uncorrected Fisher's LSD post hoc multiple comparisons test **p<0.01; ****p<0.0001; ns, nonsignificant). Bar graphs are mean±SEM.

FIG. 29: Analysis of hindlimb clasping following AAV treatment in STXBP1 HET mice (4-22 weeks post injection) (A) Average hindlimb clasping score as function of age in WT mice injected with vehicle-PBS (WT, n=17) and HET mice injected with vehicle-PBS (HET, n=16), HET mice injected with STXBP1 long variant (HET-AAV9(L), n=10) and HET mice injected with STXBP1 short variant (HET-AAV9(S), n=13). (B) Average hindlimb clasping score recorded at 22 weeks old in WT mice injected with vehicle-PBS (n=17) and HET mice injected with vehicle-PBS (n=16), AAV9/MECP2-int-STXBP1-L (n=10) and AAV9/MECP2-int-STXBP1-S(n=13). The difference between groups was analyzed by non-parametric one-way ANOVA (Kruskal-Wallis test) followed by an uncorrected Dunn's post hoc multiple comparisons test (*<p.0.05; **p<0.01; ****p<0.001; ns, nonsignificant). Bar graphs are mean±SEM.

FIG. 30: Analysis of STXBP1 HET mice in the wire hanging test following AAV treatment (8 weeks post injection). Latency to fall measured in the four limbs wire hanging test at 8 weeks old in WT mice injected with vehicle-PBS (WT, n=17) and HET mice injected with vehicle-PBS (HET, n=16), HET mice injected with STXBP1 long variant (HET-AAV9(L), n=10) and HET mice injected with STXBP1 short variant (HET-AAV9(S), n=13). The difference between groups was analyzed by parametric one-way ANOVA followed by an uncorrected Fisher's LSD post hoc multiple comparisons test (****p<0.0001; ns, nonsignificant). Bar graphs are mean±SEM.

FIG. 31: Analysis of STXBP1 HET mice in the fear conditioning test following AAV treatment (10 weeks post injection) (A) Average freezing behavior during contextual fear memory test performed at 10 weeks old 24 h after the fear conditioning training phase in WT mice injected with vehicle-PBS (WT, n=17) and HET mice injected with vehicle-PBS (HET, n=16), HET mice injected with STXBP1 long variant (HET-AAV9(L), n=10) and HET mice injected with STXBP1 short variant (HET-AAV9(S), n=13). The difference between groups was analyzed by parametric one-way ANOVA followed by an uncorrected Fisher's LSD post hoc multiple comparisons test (*<p.0.05; ****p<0.0001). (B) Average freezing behavior during cued fear memory test performed in the same animals as in (A) 1 h after the contextual fear memory test. The difference between groups was analyzed by parametric one-way ANOVA followed by an uncorrected Fisher's LSD post hoc multiple comparisons test (*p<0.05; ***p<0.001; ****p<0.0001). Bar graphs are mean±SEM.

### BRIEF DESCRIPTION OF THE SEQUENCES

TABLE 1
Sequences summary

| Sequence | |
|---|---|
| identifier | Sequence name |
| SEQ ID NO: 1 | CAG 1.6kb promoter |
| SEQ ID NO: 2 | hSYN promoter |
| SEQ ID NO: 3 | MECP2 promoter |
| SEQ ID NO: 4 | hNSE promoter |
| SEQ ID NO: 5 | CamKII promoter |
| SEQ ID NO: 6 | Endogenous hSTXBP1 promoter |
| SEQ ID NO: 7 | Human STXBP1 cDNA sequence encoding isoform a |
| SEQ ID NO: 8 | SV40 poly A |
| SEQ ID NO: 9 | Human STXBP1 isoform a (603 amino acids) |
| SEQ ID NO: 10 | Human STXBP1 isoform b (594 amino acids) |
| SEQ ID NO: 11 | Human STXBP1 isoform c |
| SEQ ID NO: 12 | Human STXBP1 isoform d |
| SEQ ID NO: 13 | Human STXBP1 isoform e |
| SEQ ID NO: 14 | Human STXBP1 isoform f |
| SEQ ID NO: 15 | Human STXBP1 isoform g |
| SEQ ID NO: 16 | Human STXBP1 isoform h |
| SEQ ID NO: 17 | AAV9.hu14 DNA sequence |
| SEQ ID NO: 18 | 3′ ITR |
| SEQ ID NO: 19 | 5′ ITR |
| SEQ ID NO: 20 | AAVtt (amino acid sequence) |
| SEQ ID NO: 21 | AAV9 (amino acid sequence) |
| SEQ ID NO: 22 | Human STXBP1 (transcript variant 1) |
| | encoding isoform a |
| SEQ ID NO: 23 | Human STXBP1 (transcript variant 2) |
| | encoding isoform b |
| SEQ ID NO: 24 | Human STXBP1 (transcript variant 3) |
| | encoding isoform c |
| SEQ ID NO: 25 | Human STXBP1 (transcript variant 4) |
| | encoding isoform d |
| SEQ ID NO: 26 | Human STXBP1 (transcript variant 5) |
| | encoding isoform d |
| SEQ ID NO: 27 | Human STXBP1 (transcript variant 6) |
| | encoding isoform e |
| SEQ ID NO: 28 | Human STXBP1 (transcript variant 7) |
| | encoding isoform e |
| SEQ ID NO: 29 | Human STXBP1 (transcript variant 8) |
| | encoding isoform e |
| SEQ ID NO: 30 | Human STXBP1 (transcript variant 9) |
| | encoding isoform e |
| SEQ ID NO: 31 | Human STXBP1 (transcript variant 10) |
| | encoding isoform f |
| SEQ ID NO: 32 | Human STXBP1 (transcript variant 11) |
| | encoding isoform g |
| SEQ ID NO: 33 | Human STXBP1 (transcript variant 12) |
| | encoding isoform h |
| SEQ ID NO: 34 | AAVtt DNA sequence |
| SEQ ID NO: 35 | MYC TAG |
| SEQ ID NO: 36 | HA TAG |
| SEQ ID NO: 37 | MECP2 intron |
| SEQ ID NO: 38 | Munc18-1a (aa 568-603) |
| SEQ ID NO: 39 | Munc18-1b (aa 568-594) |
| SEQ ID NO: 40 | Forward primer used in Example 4. |
| SEQ ID NO: 41 | Reverse primer used in Example 4. |
| SEQ ID NO: 42 | Probe 6-Fam/Zen/3′IB FQ used in Example 4. |
| SEQ ID NO: 43 | STXBP1 peptide (Example 9). |
| SEQ ID NO: 44 | STXBP1 peptide (Example 9). |
| SEQ ID NO: 45 | STXBP1 peptide (Example 9). |
| SEQ ID NO: 46 | STXBP1 peptide long isoform specific (Example 9). |
| SEQ ID NO: 47 | STXBP1 peptide short isoform specific (Example 9). |

### DETAILED DESCRIPTION OF THE INVENTION

The present invention will now be described with respect to particular non-limiting aspects and embodiments thereof and with reference to certain figures and examples.

Technical terms are used by their common sense unless indicated otherwise. If a specific meaning is conveyed to certain terms, definitions of terms will be given in the context of which the terms are used.

Where an indefinite or definite article is used when referring to a singular noun, e.g. “a”, “an” or “the”, this includes a plural of that noun unless something else is specifically stated.

As used here, the term “comprising” does not exclude other elements. For the purposes of the present disclosure, the term “consisting of” is considered to be a preferred embodiment of the term “comprising”.

As used herein, the terms “treatment”, “treating” and the like, refer to obtaining a desired pharmacologic and/or physiological effect. The effect may be prophylactic in terms of completely or partially preventing a disease or symptom thereof and/or may be therapeutic in terms of a partial or complete cure for a disease and/or adverse symptoms attributable to the disease. Treatment thus covers any treatment of a disease in a mammal, particularly in a human, and includes: (a) preventing the disease symptoms from occurring in a subject, i.e. a human, which may be predisposed to the disease but has not yet been diagnosed as having it; (b) inhibiting the disease, i.e., arresting its development; and (c) relieving the disease, i.e., causing regression of the disease.

### Syntaxin Binding Protein 1

12 transcript variants of human STXBP1 have been identified, encoding 8 protein isoforms. The amino acid sequences are highly conserved between rodents and humans. In the central nervous system, STXBP1 is specifically expressed in neurons and broadly distributed across major brain areas including cortex, cerebellum, hippocampus and basal ganglia (Kalidas et al. 2000). Two major splice variants have been described, including a short and a long version:

Munc18-1a (aa 568-603):
(SEQ ID NO: 38)
GSTHILTPTKFLMDLRHPDFRESSRVSFEDQAPTME.
Munc18-1b (aa 568-594):
(SEQ ID NO: 39)
GSTHILTPQKLLDTLKKLNKTDEEISS

The longer splice version (M18L, Munc18-1a, 603 amino acids) shows a difference in the last 25 C-terminal amino acids and is reported to be expressed to a major part at the synaptic level and in gabaergic neurons in the rat brain (Ramos et al. 2015). The smaller splice version (M18S, Munc18-1b, 594 amino acids) has been localized in different cellular compartments and is more ubiquitously expressed in gabaergic and glutamatergic neurons. Functional studies indicated that STXBP1 splice variants could play different roles in synaptic plasticity (Meijer et al. 2015).

STXBP1 gene is located on the chromosome 9q34.11 (GRCh38 genomic coordinates: chr9:127,579,370-127,696,029) and the human encoded protein has a high level of identity with both rat and murine STXBP1 (Swanson et al. 1998). STXBP1 gene contains 25 exons. Alternative splicing of the final exon in the STXBP1 primary transcript may include or skip a sequence of 110 bp containing a stop codon, resulting in two different C-terminal amino acid sequences for STXBP1. The STXBP1-202 transcript (ENST00000373302.8) (SEQ ID NO: 22) is the longest, encoding for a 603 amino acid protein (SEQ ID NO: 9). STXBP1-201 (ENST00000373299.5) (SEQ ID NO: 23) encodes for a 594 amino acid protein (SEQ ID NO: 10). Both of these variants are detected in the central nervous system although their expression pattern may vary between brain tissues and cell types (Ramos-Miguel et al. 2015).

The 12 transcript variants and 8 protein isoforms of human STXBP1 are summarised in Table 2.

TABLE 2
STXBP1 transcript variants and protein isoforms

| Human | Protein | | DNA |
|---|---|---|---|
| STXBP1 | Sequence | | sequence |
| isoform | identifier | Transcript variant | identifier |
| isoform a | SEQ ID NO: 9 | transcript variant 1 encoding isoform a | SEQ ID NO: 22 |
| isoform b | SEQ ID NO: 10 | transcript variant 2 encoding isoform b | SEQ ID NO: 23 |
| isoform c | SEQ ID NO: 11 | transcript variant 3 encoding isoform c | SEQ ID NO: 24 |
| isoform d | SEQ ID NO: 12 | transcript variant 4 encoding isoform d | SEQ ID NO: 25 |
| | | transcript variant 5 encoding isoform d | SEQ ID NO: 26 |
| isoform e | SEQ ID NO: 13 | transcript variant 6 encoding isoform e | SEQ ID NO: 27 |
| | | transcript variant 7 encoding isoform e | SEQ ID NO: 28 |
| | | transcript variant 8 encoding isoform e | SEQ ID NO: 29 |
| | | transcript variant 9 encoding isoform e | SEQ ID NO: 30 |
| isoform f | SEQ ID NO: 14 | transcript variant 10 encoding isoform f | SEQ ID NO: 31 |
| isoform g | SEQ ID NO: 15 | transcript variant 11 encoding isoform g | SEQ ID NO: 32 |
| isoform h | SEQ ID NO: 16 | transcript variant 12 encoding isoform h | SEQ ID NO: 33 |

Syntaxin binding protein 1 or STXBP1 is sometimes referred to in the art by the alternative names listed in Table 3. The most common ones are “Munc18-1” and to a lesser extent “Sec1”. The accepted gene name is STXBP1.

TABLE 3
Syntaxin binding protein 1 alternative names
Syntaxin Binding Protein 1
Syntaxin-Binding Protein 1
MUNC18-1
Protein Unc-18 Homolog 1
Protein Unc-18 Homolog A
Unc-18A
Unc18-1
N-Sec1
UNC18
P67
Neuronal SEC1
RBSEC1
STXBP1
HUNC18
RbSec1
UNC18A
NSEC1
M18

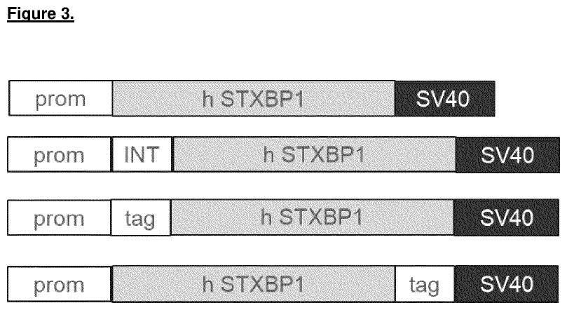

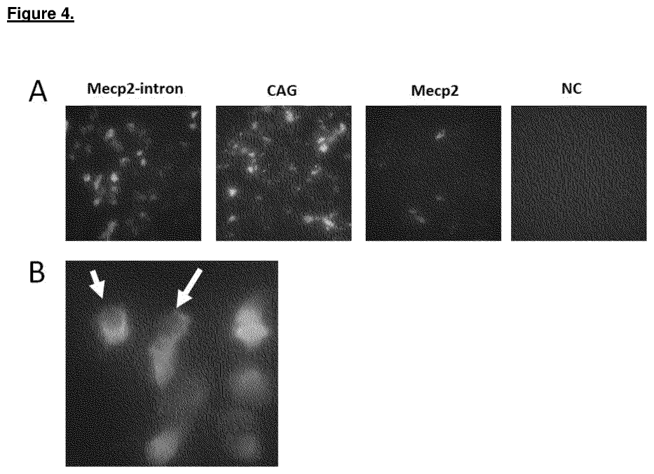

Protein sequence alignment of the human, monkey and mouse STXBP1 sequences (human isoform a according to SEQ ID NO: 9) is shown in FIG. 2. The alignment shows the high sequence homology across the species. The monkey and mouse amino acid sequences are identical to the human amino acid sequence.

### Transgene

The present invention provides a nucleic acid construct comprising a transgene encoding:
i. a syntaxin binding protein 1 (STXBP1) comprising isoform a, b, c, d, e, f, g or h, having the sequence given in SEQ ID NO: 9, 10, 11, 12, 13, 14, 15 or 16 respectively; or
ii. a sequence having at least 95% or 96% or 97% or 98% or 99% or 99.5% sequence identity to SEQ ID NO:9, 10, 11, 12, 13, 14, 15 or 16 and retaining functionality as STXBP1; or
iii. a naturally-occurring variant comprising, with reference to SEQ ID NO:9, one or more mutations as shown in Table 7.

The term “transgene” refers to a nucleic acid molecule (“nucleic acid molecule” and “nucleic acid” are used interchangeably), DNA or cDNA encoding a gene product for use as the active principle in gene therapy. The gene product may be one or more peptides or proteins.

In one embodiment the transgene encodes STXBP1 isoform a having the sequence given in SEQ ID NO: 9; or a sequence having at least 95% or 96% or 97% or 98% or 99% or 99.5% sequence identity to SEQ ID NO: 9.

In one embodiment the transgene encodes STXBP1 isoform b having the sequence given in SEQ ID NO: 10; or a sequence having at least 95% or 96% or 97% or 98% or 99% or 99.5% sequence identity to SEQ ID NO: 10.

In one embodiment the transgene encodes STXBP1 isoform c having the sequence given in SEQ ID NO: 11; or a sequence having at least 95% or 96% or 97% or 98% or 99% or 99.5% sequence identity to SEQ ID NO:11.

In one embodiment the transgene encodes STXBP1 isoform d having the sequence given in SEQ ID NO: 12; or a sequence having at least 95% or 96% or 97% or 98% or 99% or 99.5% sequence identity to SEQ ID NO:12.

In one embodiment the transgene encodes STXBP1 isoform e having the sequence given in SEQ ID NO: 13; or a sequence having at least 95% or 96% or 97% or 98% or 99% or 99.5% sequence identity to SEQ ID NO: 13.

In one embodiment the transgene encodes STXBP1 isoform f having the sequence given in SEQ ID NO: 14; or a sequence having at least 95% or 96% or 97% or 98% or 99% or 99.5% sequence identity to SEQ ID NO: 14.

In one embodiment the transgene encodes STXBP1 isoform g having the sequence given in SEQ ID NO: 15; or a sequence having at least 95% or 96% or 97% or 98% or 99% or 99.5% sequence identity to SEQ ID NO: 15.

In one embodiment the transgene encodes STXBP1 isoform h having the sequence given in SEQ ID NO: 16; or a sequence having at least 95% or 96% or 97% or 98% or 99% or 99.5% sequence identity to SEQ ID NO: 16.

In one embodiment the transgene encodes:
i. STXBP1 transcript variant 1, 2, 3, 4, 5, 6, 7, 8, 9, 10, 11, or 12, having the sequence given in SEQ ID NO: 22, 23, 24, 25, 26, 27, 28, 29, 30, 31, 32 or 33 respectively; or
ii. a sequence having at least 95% or 96% or 97% or 98% or 99% or 99.5% sequence identity to SEQ ID NO: 22, 23, 24, 25, 26, 27, 28, 29, 30, 31, 32 or 33.

As is conventional practice in the art, the mRNA sequences of STXBP1 transcript variants 1 to 12 are reported as DNA sequences for consistency with the reference genome sequence. (National Center for Biotechnology Information, www.ncbi.nlm.nih.gov). The goal of this is to more directly perform a genomic alignment with fewer mismatches reported. In order to express STXBP1 isoform a, b, or c, for example, a person skilled in the art would express a cDNA from transcript variant 1, 2, or 3.

In one embodiment the transgene encodes STXBP1 isoform a and comprises a cDNA sequence of SEQ ID NO: 7; or a sequence having at least 95% or 96% or 97% or 98% or 99% or 99.5% sequence identity to SEQ ID NO: 7.

The terms “nucleic acid” and “polynucleotide” or “nucleotide sequence” may be used interchangeably to refer to any molecule composed of or comprising monomeric nucleotides. A nucleic acid may be an oligonucleotide or a polynucleotide. A nucleotide sequence may be a DNA or RNA. A nucleotide sequence may be chemically modified or artificial. Nucleotide sequences include peptide nucleic acids (PNA), morpholinos and locked nucleic acids (LNA), as well as glycol nucleic acids (GNA) and threose nucleic acid (TNA). Each of these sequences is distinguished from naturally-occurring DNA or RNA by changes to the backbone of the molecule. Phosphorothioate nucleotides may be used. Other deoxynucleotide analogs include methylphosphonates, phosphoramidates, phosphorodithioates, N3′P5′-phosphoramidates and oligoribonucleotide phosphorothioates and their 2′-0-allyl analogs and 2′-0-methylribonucleotide methylphosphonates.

The term “nucleic acid construct” refers to a non-naturally occurring nucleic acid resulting from the use of recombinant DNA technology. Especially, a nucleic acid construct is a nucleic acid molecule which has been modified to contain segments of nucleic acid sequences, which are combined or juxtaposed in a manner which does not exist in nature.

In specific embodiments, said nucleic acid construct comprises all or a fragment of a coding nucleic acid sequence having at least 70%, 80%, 90%; 95%, 99% or 100% identity to the coding sequence of a naturally-occurring or recombinant functional variant of STXBP1.

The term “fragment” as used herein refers to a contiguous portion of a reference sequence. For example, a fragment of a sequence having 1000 nucleotides in length may refer to 5, 50, 500 contiguous nucleotides of said sequence.

The term “pathological variant” as used herein refers to a nucleic acid or amino acid sequence which is modified relative to a reference sequence and which has impaired function compared to said reference sequence. Pathological variants and likely pathological variants of STXBP1 are shown in Tables 5 and 6 respectively.

The term “functional variant” as used herein refers to a nucleic acid or amino acid sequence which is modified relative to a reference sequence but which retains the function of said reference sequence. Functional variants of STXBP1 are shown in Table 7.

The term “sequence identity” or “identity” refers to the number of matches (identical nucleic acid or amino acid residues) in positions from an alignment of two polynucleotide or polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970, J Mol Biol.; 48(3):443-53) which aligns the sequences optimally over the entire length, while sequences of substantially different lengths are preferably aligned using a local alignment algorithm (e.g. Smith and Waterman algorithm (Smith and Waterman, 1981, J Theor Biol.; 91(2):379-80) or Altschul algorithm (Altschul S F et al., 1997, Nucleic Acids Res.; 25(17):3389-402.; Altschul S F et al., 2005, Bioinformatics.; 21(8):1451-6). Alignment for purposes of determining percent nucleic acid or amino acid sequence identity can be achieved in various ways that are within the skill in the art, for instance, using publicly available computer software available on internet web sites such as http://blast.ncbi.nlm.nih.gov/or http://www.ebi.ac.uk/Tools/emboss/. Those skilled in the art can determine appropriate parameters for measuring alignment, including any algorithms needed to achieve maximal alignment over the full length of the sequences being compared. For purposes herein, % nucleic acid or amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

The nucleic acid construct according to the present disclosure comprises a transgene and at least a suitable nucleic acid element for its expression in a host, such as in a host cell.

For example, said nucleic acid construct comprises a transgene encoding STXBP1 and one or more control sequences required for expression of STXBP1 in the relevant host. Generally, the nucleic acid construct comprises a transgene and regulatory sequences preceding (5′ non-coding sequences) and following (3′ non-coding sequences) the transgene that are required for expression of STXBP1.

### Promoter

In one embodiment, the nucleic acid construct comprises a transgene encoding STXBP1 and a promoter operably-linked to said transgene. Preferably, the transgene is under the control of the promoter.

The term “promoter” refers to a regulatory element that directs the transcription of a nucleic acid to which it is operably linked. A promoter can regulate both rate and efficiency of transcription of an operably-linked nucleic acid. A promoter may also be operably-linked to other regulatory elements which enhance (“enhancers”) or repress (“repressors”) promoter-dependent transcription of a nucleic acid. These regulatory elements include, without limitation, transcription factor binding sites, repressor and activator protein binding sites, and any other sequences of nucleotides known by one of skill in the art to act directly or indirectly to regulate the amount of transcription from the promoter, e.g. attenuators, enhancers, and silencers. The promoter is located near the transcription start site of the gene or coding sequence to which is operably-linked, on the same strand and upstream of the DNA sequence (towards the 5′ region of the sense strand). A promoter can be about 100-1000 base pairs long. Positions in a promoter are designated relative to the transcriptional start site for a particular gene (i.e., positions upstream are negative numbers counting back from −1, for example −100 is a position 100 base pairs upstream).

The term “operably-linked in a 5′ to 3′ orientation” or simply “operably-linked” refers to a linkage of two or more nucleotide sequences in a functional relationship which allows each of said two or more sequences to perform their normal function. Typically, the term operably-linked is used to refer to the juxtaposition of a regulatory element such as promoter and a transgene encoding a protein of interest. For example, an operable linkage between a promoter and a transgene permits the promoter to function to drive the 5′ expression of the transgene in a suitable expression system, such as in a cell.

The promoter may be a tissue or cell type specific promoter, or an organ-specific promoter, or a promoter specific to multiple organs, or a systemic or ubiquitous promoter.

The term “ubiquitous promoter” more specifically relates to a promoter that is active in a variety of distinct cells or tissues, for example in both the neurons and astrocytes.

Examples of promoter suitable for expression of the transgene across the central nervous system include chicken beta actin (CBA) promoter (Miyazaki 1989, Gene 79:269-277), the CAG promoter (Niwa 1991, Gene 108:193-199), the Elongation factor 1 alpha promoter (EF1α) (Nakai 1998, Blood 91:4600-4607), the human synapsin 1 gene promoter (hSyn) (Kugler S. et al. Gene Ther. 2003. 10(4):337-47) or the phosphoglycerate kinase 1 promoter (PGK1) (Hannan 1993, Gene 130:233-239), the methyl CPG Binding Protein 2 (MECP2) promoter (Adachi et al., Hum. Mol. Genetics. 2005; 14(23): 3709-3722), the human neuron-specific enolase (NSE) promoter (Twyman, R. M. and E. A. Jones (1997). J Mol Neurosci 8(1): 63-73)), the calcium/calmodulin dependent protein-kinase II (CAMKII) promoter (Nathanson, J. L., et al. (2009). Neuroscience 161(2): 441-450) and the human ubiquitin C (UBC) promoter (Schorpp, M., et al. (1996). Nucleic Acids Res 24(9): 1787-1788).

In one embodiment, the promoter is a CAG 1.6 kb promoter of SEQ ID NO: 1.

In one embodiment, the promoter is a hSYN promoter of SEQ ID NO: 2.

In one embodiment, the promoter is a MECP2 promoter of SEQ ID NO: 3.

In one embodiment, the promoter is a hNSE promoter of SEQ ID NO: 4.

In one embodiment, the promoter is a CamKII promoter of SEQ ID NO: 5.

In one embodiment, the promoter is an endogenous hSTXBP1 promoter of SEQ ID NO: 6.

In one embodiment, the promoter is a MECP2 promoter of SEQ ID NO: 3, operably-linked in a 5′ to 3′ orientation to a MECP2 intron of SEQ ID NO: 37.

In alternative embodiments, the nucleic acid construct comprises a transgene encoding STXBP1 and a promoter operably-linked to said transgene, wherein the promoter is at least 90%, 91%, 92%, 93%, 94%, 95%, 96%, 97%, 98%, or 99% identical to:
(a) CAG 1.6 kb promoter (SEQ ID NO: 1).
(b) hSYN promoter (SEQ ID NO: 2).
(c) MECP2 promoter (SEQ ID NO: 3).
(d) hNSE promoter (SEQ ID NO: 4).
(e) CamKII promoter (SEQ ID NO: 5).
(f) endogenous hSTXBP1 promoter (SEQ ID NO: 6).
(g) MECP2 promoter (SEQ ID NO: 3) operably-linked in a 5′ to 3′ orientation to MECP2 intron (SEQ ID NO: 37).

The promoter may be a functional variant or fragment of the promoters described herein. A functional variant or fragment of a promoter may be functional in the sense that it retains the characteristics of the corresponding non-variant or full-length promoter. Thus, a functional variant or fragment of a promoter retains the capacity to drive the transcription of a transgene to which it is operably linked, thereby driving the expression of STXBP1 encoded by said transgene. A functional variant or fragment of a promoter may retain specificity for a particular tissue type. For example, a functional variant or fragment of a promoter may be specific for cells of the CNS. A functional variant or fragment of a promoter may specifically drive expression of STXBP1 in neurons.

The promoter may comprise a “minimal sequence”, which means a nucleotide sequence of the promoter having sufficient length and containing the required elements to function as a promoter, i.e. capable of driving the transcription of the transgene to which said promoter is operably linked, thereby driving the expression of STXBP1.

The minimal promoter used in the nucleic acid constructs of the present invention may be for example the CAG promoter comprising SEQ ID NO: 1, or the hSYN promoter comprising SEQ ID NO: 2, or the MECP2 promoter comprising SEQ ID NO: 3.

The promoter may comprise one or more introns. The term “intron” refers to an intragenic non-coding nucleotide sequence. Typically, introns are transcribed from DNA into messenger RNA (mRNA) during transcription of a gene but are excised from the mRNA transcript by splicing prior to its translation.

The promoter may comprise a functional variant or fragment of an intron described herein. A functional variant or fragment of an intron may be functional in the sense that it retains the characteristics of the corresponding non-variant or full-length intron. Thus, functional variants or fragments of an intron described herein are non-coding. Functional variants or fragments of an intron described herein may also retain the capacity to be transcribed from DNA to mRNA and/or the capacity to be excised from mRNA by splicing.

Introns that may be incorporated in the promoters used in the present invention may be from naturally non-coding regions or may be engineered.

In one embodiment, the intron is a MECP2 intron comprising or consisting of SEQ ID NO: 37; or a functional variant or fragment thereof having at least 90%, 91%, 92%, 93%, 94%, 95%, 96%, 97%, 98%, 99%, 99.5%, or 99.9% identity to SEQ ID NO: 37.

A promoter and/or intron may be combined with one or more non-expressing exonic sequence(s). Non-expressing exonic sequences are not capable of producing a transcript, rather they may flank an intronic sequence to provide splice sites.

Alternatively, a promoter may be a chemically-inducible promoter. A chemically-inducible promoter is a promoter that is regulated by the in vivo administration of a chemical inducer to a subject in need thereof. Examples of suitable chemically-inducible promoters include without limitation tetracycline/minocycline inducible promoters (Chtarto 2003, Neurosci Lett. 352:155-158) or rapamycin inducible promoters (Sanftner 2006, Mol Ther. 13:167-174).

### Polyadenylation Signal Sequence

The nucleic acid construct may comprise a 3′ untranslated region comprising a polyadenylation signal sequence and/or transcription terminator.

The term “polyadenylation signal sequence”, (or “polyadenylation site or “poly(A) signal” which are all used interchangeably) refers to a specific recognition sequence within the 3′ untranslated region (3′ UTR) of a gene, which is transcribed into precursor mRNA and guides the termination of gene transcription. The polyadenylation signal sequence acts as a signal for the endonucleolytic cleavage of the newly formed precursor mRNA at its 3′-end, and for the addition to this 3′-end of a stretch of RNA consisting only of adenine bases (polyadenylation process; poly(A) tail). The polyadenylation signal sequence is important for the nuclear export, translation, and stability of mRNA. In the context of the invention, the polyadenylation signal sequence is a recognition sequence that can direct polyadenylation of mammalian genes and/or viral genes, in mammalian cells.

The polyadenylation signal sequence typically consists of (a) a consensus sequence AAUAAA, which has been shown to be required for both 3′-end cleavage and polyadenylation of pre-messenger RNA (pre-mRNA) as well as to promote downstream transcriptional termination; and (b) additional elements upstream and downstream of AAUAAA that control the efficiency of utilization of AAUAAA as a poly(A) signal. There is considerable variability in these motifs in mammalian genes.

In one embodiment, optionally in combination with one or more features of the various embodiments described herein, the polyadenylation signal sequence of the nucleic acid construct of the invention is a polyadenylation signal sequence of a mammalian gene or a viral gene. Suitable polyadenylation signals include, among others, a SV40 early polyadenylation signal, a SV40 late polyadenylation signal, a HSV thymidine kinase polyadenylation signal, a protamine gene polyadenylation signal, an adenovirus 5 Elb polyadenylation signal, a growth hormone polyadenylation signal, a PBGD polyadenylation signal, or an in silico designed synthetic polyadenylation signal.

In one embodiment, the polyadenylation signal sequence is a SV40 polyadenylation signal sequence comprising SEQ ID NO: 8.

### Other Regulatory Elements

The nucleic acid construct may comprise additional regulatory elements, for example enhancer sequence, intron, microRNA targeted sequence, a polylinker sequence facilitating the insertion of a DNA fragment within a vector and/or splicing signal sequence.

### Viral Vector

The present invention further provides a viral vector comprising the nucleic acid construct as described herein.

The term “viral vector” refers to the nucleic acid part of the viral particle as disclosed herein, which may be packaged in a capsid.

Viral vectors typically comprise at least (i) a nucleic acid construct including a transgene and suitable nucleic acid elements for its expression in a host, and (ii) all or a portion of a viral genome, for example inverted terminal repeats of a viral genome.

The term “inverted terminal repeat” (ITR) refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome, for the rescue from the host genome and for the encapsidation of viral nucleic acid into mature virions. The ITRs are required in cis for the vector genome replication and its packaging into the viral particles.

In one embodiment, the viral vector comprises a 5′ITR, and a 3′ITR of a virus.

In one embodiment, the viral vector comprises a 5′ITR and a 3′ITR of a virus independently selected from the group consisting of parvoviruses (in particular adeno-associated viruses), adenoviruses, alphaviruses, retroviruses (in particular gamma retroviruses and lentiviruses), herpesviruses, and SV40.

In one embodiment the virus is an adeno-associated virus (AAV), an adenovirus (Ad), or a lentivirus.

In one embodiment the virus is an AAV.

In one embodiment, the viral vector comprises a 5′ITR and a 3′ITR of an AAV.

AAV has generated considerable interest as a potential vector for human gene therapy. Among the favourable properties of the virus are its lack of association with any human disease, its ability to infect both dividing and non-dividing cells, and the wide range of cell lines derived from different tissues that can be infected. The AAV genome is composed of a linear, single-stranded DNA molecule which contains 4681 bases (Berns and Bohenzky, 1987, Advances in Virus Research (Academic Press, Inc.) 32:243-307). The genome includes inverted terminal repeats (ITRs) at each end, which function in cis as origins of DNA replication and as packaging signals for the virus. The ITRs are typically about 100-150 bp in length.

AAV ITRs may have a wild-type nucleotide sequence or may be altered by the insertion, deletion or substitution of one or more nucleotides, typically, no more than 5, 4, 3, 2 or 1 nucleotide insertion, deletion or substitution as compared to known AAV ITRs. The serotype of the inverted terminal repeats (ITRs) of the AAV vector may be selected from any known human or non-human AAV serotype.

In specific embodiments, the viral vector may comprise ITRs of any AAV serotype. Known AAV ITRs include without limitation, AAV1, AAV2, AAV3 (including types 3A and 3B), AAV-LK03, AAV4, AAV5, AAV6, AAV7, AAV8, AAV9, AAV10 (AAVrh10), AAV11, AAV12, avian AAV, bovine AAV, canine AAV, equine AAV, ovine AAV. Recombinant serotypes such as Rec2 and Rec3 identified from primate brain are also included.

Alternatively, the viral vector may comprise a synthetic 5′ITR and/or 3′ITR.

In one embodiment, the nucleic acid construct of the present invention is comprised in a viral vector which further comprises a 5′ITR and/or a 3′ITR of an AAV of a serotype AAV2.

In one embodiment, the viral vector comprises a 3′ITR and/or 5′ITR of an AAV of a serotype AAV2, having the sequence given in SEQ ID NO: 18 and/or 19 respectively; or a sequence having at least 80% or at least 90% identity with SEQ ID NO: 18 and/or 19 respectively.

### Viral Particle

The present invention further provides a viral particle comprising the nucleic acid construct or the viral vector as described herein.

The term “viral particle” refers to an infectious and typically replication-defective virus particle comprising (i) a viral vector packaged within (optionally comprising a nucleic acid construct) and (ii) a capsid.

In one embodiment, the capsid is formed of capsid proteins of an adeno-associated virus.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

AAV-based gene therapy targeting the CNS is reviewed in Pignataro D, Sucunza D, Rico A J et al., J Neural Transm 2018; 125:575-589. Viral particles may be selected and/or engineered to target at least neuronal cells in various area of the brain and CNS.

AAV viruses are commonly referred to in terms of their serotype. A serotype corresponds to a variant subspecies of AAV which, owing to its profile of expression of capsid surface antigens, has a distinctive reactivity which can be used to distinguish it from other variant subspecies. AAV serotypes include AAV1, AAV2, AAV3 (including A and B) AAV-LK03, AAV4, AAV5, AAV6, AAV7, AAV8, AAV9, AAV10 (AAVrh10) or AAV11, or combinations thereof. The AAV may be a recombinant serotype, such as Rec2 or Rec3 identified from primate brain; and AAV2-true-type (AAVtt). AAVtt is described in detail in Tordo et al., Brain. 2018; 141(7): 2014-2031 and WO 2015/121501. Reviews of AAV serotypes may be found in Choi et al (Curr Gene Ther. 2005; 5(3); 299-310) and Wu et al (Molecular Therapy. 2006; 14(3), 316-327).

In the viral particle of the invention, the capsid may be derived from any AAV serotype or from a combination of serotypes (such as VP1 from one AAV serotype and VP2 and/or VP3 from a different serotype).

In specific embodiments, the capsid proteins may be derived from AAV2, AAV5, AAV8, AAV9, AAV2-retro or AAVtt.

In one embodiment, the viral particle comprises at least a VP1 capsid protein from an AAV, wherein said capsid protein is derived from AAV2, AAV5, AAV6, AAV8, AAV9 (for example AAV9.hu14 as shown in SEQ ID NO: 21), AAV10, AAV-true type (AAVtt as shown in SEQ ID NO: 20) or combinations thereof.

In one embodiment, the viral particle comprises the capsid protein from AAVtt as shown in SEQ ID NO: 20. In one embodiment the capsid protein is at least 98.5%, 99% or 99.5% identical to SEQ ID NO: 20.

In one embodiment, the viral particle comprises the capsid protein from AAV9 as shown in SEQ ID NO: 21. In one embodiment, the capsid protein is at least 98.5%, 99% or 99.5% identical to SEQ ID NO: 21.

AAV genomes or elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065,5AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV viruses may also be referred to in terms of clades or clones. This refers to the phylogenetic relationship of naturally derived AAV viruses, and typically to a phylogenetic group of AAV viruses which can be traced back to a common ancestor, and includes all descendants thereof. Additionally, AAV viruses may be referred to in terms of a specific isolate, i.e. a genetic isolate of a specific AAV virus found in nature.

The term “genetic isolate” describes a population of AAV viruses which has undergone limited genetic mixing with other naturally occurring AAV viruses, thereby defining a recognizably distinct population at a genetic level. Examples of clades and isolates of AAV that may be used in the invention include:
Clade A: AAV1 NC_002077, AF063497, AAV6 NC_001862, Hu. 48 AY530611, Hu 43 AY530606, Hu 44 AY530607, Hu 46 AY530609;
Clade B: Hu. 19 AY530584, Hu. 20 AY530586, Hu 23 AY530589, Hu22 AY530588, Hu24 AY530590, Hu21 AY530587, Hu27 AY530592, Hu28 AY530593, Hu 29 AY530594, Hu63 AYS30624, Hu64 AY530625, Hul3 AY530578, Hu56 AY530618, Hu57 AY530619, Hu49 AY530612, Hu58 25 AY530620, Hu34 AY530598, Hu35 AY530599, AAV2 NC_001401, Hu45 AY530608, Hu47 AY530610, Hu51 AY530613, Hu52 AY530614, Hu T41 AY695378, Hu S17 AY695376, Hu T88 AY695375, Hu T71 AY695374, HuT70 AY695373, Hu T40 AY695372, Hu T32 AY695371, Hu T17 AY695370, Hu LG15 AY695377;
Clade C: Hu9 AY530629, HulO AY530576, Hull AY530577, Hu53 AY530615, Hu55 AY530617, Hu54 AY530616, Hu7 AY530628, Hu18 AY530583, Hul5 AY530580, Hul6 AY530581, Hu25 AY530591, Hu60 AY530622, Ch5 AY243021, Hu3 AY530595, Hul AY530575, Hu4 AY530602 Hu2, AY530585, Hu61 AY530623;
Clade D: Rh62 AY530573, Rh48 AY530561, Rh54 AY530567, Rh55 AY530568, C5 y2 AY243020, AAV7 AF513851, Rh35 AY243000, Rh37 AY242998, Rh36 AY242999, Cy6 AY243016, Cy4 AY243018, Cy3 AY243019, Cy5 AY243017, Rhl3 AY243013;
Clade E: Rh38 AY530558, Hu66 AY530626, Hu42 AY530605, Hu67 AY530627, Hu40 AY530603, Hu41 AY530604, Hu37 AY530600, Rh40 10 AY530559, Rh2 AY243007, Bbl AY243023, Bb2 AY243022, RhlO AY243015, Hul7 AY530582, Hub AY530621, Rh25 AY530557, Pi2 AY530554, Pil AY530553, Pi3 AY530555, Rh57 AY530569, Rh50 AY530563, Rh49 AY530562, Hu39 AY530601, Rh58 AY530570, Rhbl AY530572, Rh52AY530565, Rh53 AY530566, Rh51 AY530564, Rh64 AY530574, Rh43 15 AY530560, AAV8 AF513852, Rh8 AY242997, Rhl AY530556; and
Clade F: Hu 14 (AAV9) AY530579, Hu31 AY530596, Hu32 AY530597; Clonal Isolate AAV5 Y18065, AF085716, AAV 3 NC_001729, AAV 3B NC_001863, AAV4 15 NC_001829, Rh34 AY243001, Rh33 AY243002, Rh32 AY243003.

The invention encompasses the use of capsid protein sequences from different serotypes, clades, clones, or isolates of AAV within the same vector. The invention also encompasses the packaging of the genome of one serotype into the capsid of another serotype i.e. pseudotyping. Chimeric, shuffled or capsid-modified derivatives may be selected to provide one or more desired functionalities. Thus, these derivatives may display increased efficiency of gene delivery, decreased immunogenicity (humoral or cellular), an altered tropism range and/or improved targeting of a particular cell type compared to an AAV viral vector comprising a naturally occurring AAV capsid. Increased efficiency of gene delivery may be achieved by improved receptor or co-receptor binding at the cell surface, improved internalization, improved trafficking within the cell and into the nucleus, improved uncoating of the viral particle or improved conversion of a single-stranded genome to double-stranded form. Increased efficiency may also relate to an altered tropism range or targeting of a specific cell population, such that the vector dose is not diluted by delivery to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins include those generated by engineering of capsid protein sequences to transfer specific capsid protein domains, surface loops or specific amino acid residues between two or more capsid proteins, for example between two or more capsid proteins of different serotypes. Shuffled or chimeric capsid proteins may be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may be genetically modified to introduce specific deletions, substitutions or insertions with respect to the native wild-type sequence. For example, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the viral particle to a particular cell population. The unrelated protein may be one which assists purification of the viral particle as part of the production process i.e. an epitope or affinity tag. The site of insertion will typically be selected so as not to interfere with other functions of the viral particle such as internalization or trafficking of the viral particle. Suitable insertion sites are disclosed in Choi et al (Curr Gene Ther. 2005; 5(3); 299-310).

In one embodiment, a viral particle may be prepared by encapsulating an AAV viral vector derived from a particular AAV serotype in a viral particle formed by natural Cap proteins corresponding to an AAV of the same serotype.

Nevertheless, several techniques have been developed to modify and improve the structural and functional properties of naturally occurring viral particles (Bunning H et al. J Gene Med 2008; 10: 717-733).

Thus, in another embodiment, a viral particle may include a nucleic acid construct comprising a transgene encoding STXBP1, flanked by ITR(s) of a given AAV serotype, packaged into:
(a) a viral particle comprising capsid proteins derived from a different AAV serotype, for example AAV2 ITRs and AAV9 capsid proteins; or AAV2 ITRs and AAVtt capsid proteins; or
(b) a mosaic viral particle comprising a mixture of capsid proteins from different AAV serotypes or mutants, for example AAV2 ITRs with a capsid formed by proteins of two or more AAV serotypes; or
(c) a chimeric viral particle comprising capsid proteins that have been truncated by domain swapping between different AAV serotypes or variants, for example AAV2 ITRs with AAV5 capsid proteins comprising AAV3 domains; or
(d) a viral particle engineered to display selective binding domains, enabling stringent interaction with target cell specific receptors.

AAVtt capsid also named AAV2 true-type capsid is described in WO2015/121501. In one embodiment, AAVtt VP1 capsid protein comprises at least one amino acid substitution with respect to the wild-type VP1 capsid protein at a position corresponding to one or more of the following positions in an AAV2 protein sequence (NCBI Reference sequence: YP_680426.1): 125, 151, 162, 205, 312, 457, 492, 499, 533, 546, 548, 585, 588 and/or 593.

In one embodiment, AAVtt comprises one or more of the following amino acid substitutions with respect to a wild-type AAV2 VP1 capsid protein (NCBI Reference sequence: YP_680426.1): V1251, V151A, A162S, T205S, N312S, Q457M, S492A, E499D, F533Y, G546D, E548G, R585S, R588T and/or A593S.

In one embodiment, AAVtt comprises four or more mutations with respect to the wild type AAV2 VP1 capsid protein at the positions 457, 492, 499 and 533.

The construction of recombinant AAV viral particles is generally known in the art and has been described for instance in U.S. Pat. Nos. 5,173,414; 5,139,941; WO 92/01070; WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801.

### Production of Viral Particles

Production of viral particles carrying the viral vector and nucleic acid construct as described herein can be performed by means of conventional methods and protocols, which are selected by taking into account the structural features of the viral particles to be produced.

Briefly, viral particles can be produced in a host cell, more particularly in a specific virus-producing cell (packaging cell), which is transfected with the nucleic acid construct or viral vector in the presence of a helper vector or virus or other DNA construct(s).

The term “packaging cell” refers to a cell or cell line which may be transfected with a nucleic acid construct or viral vector and provides in trans all the missing functions that are required for the complete replication and packaging of a viral vector. Packaging cells may express such missing viral functions in a constitutive or inducible manner. Packaging cells may be adherent or suspension cells.

Typically, a process of producing viral particles comprises the following steps:
(a) culturing a packaging cell comprising a nucleic acid construct or viral vector in a culture medium; and
(b) harvesting the viral particles from the cell culture supernatant and/or inside the cells.

Conventional methods can be used to produce viral particles, which involve transient cell co-transfection with a nucleic acid construct or expression vector (e.g. a plasmid) carrying the transgene encoding STXBP1; a second nucleic acid construct (e.g. an AAV helper plasmid) that encodes rep and cap genes, but does not carry ITR sequences; and a third nucleic acid construct (e.g. a plasmid) providing the adenoviral functions necessary for AAV replication.

Viral genes necessary for AAV replication are referred to as viral helper genes. Typically, said genes necessary for AAV replication are adenoviral helper genes, such as E1A, E1B, E2a, E4, or VA RNAs. In one embodiment, the adenoviral helper genes are of the Ad5 or Ad2 serotype.

Production of AAV particles may alternatively be carried out by infection of insect cells with a combination of recombinant baculoviruses (Urabe et al. Hum. Gene Ther. 2002; 13: 1935-1943). SF9 cells are co-infected with two or three baculovirus vectors respectively expressing AAV rep, AAV cap and the AAV vector to be packaged. The recombinant baculovirus vectors provide the viral helper gene functions required for virus replication and/or packaging. Smith et al 2009 (Molecular Therapy, vol. 17, no. 11, pp 1888-1896) describes a dual baculovirus expression system for large-scale production of AAV particles in insect cells.

Suitable culture media are known to a person skilled in the art. The ingredients that make up a culture medium may vary depending on the type of cell to be cultured. In addition to nutrient composition, osmolarity and pH are considered important parameters of culture media. The cell growth medium comprises a number of ingredients well known by the person skilled in the art, including amino acids, vitamins, organic and inorganic salts, sources of carbohydrate, lipids, trace elements (to name a few, CuS04, FeS04, Fe(N03)3, ZnS04), each ingredient being present in an amount which supports the cultivation of a cell in vitro (i.e., survival and growth of cells). Ingredients may also include auxiliary substances, such as buffer substances (for example sodium bicarbonate, Hepes, Tris or similarly performing buffers), oxidation stabilisers, stabilisers to counteract mechanical stress, protease inhibitors, animal growth factors, plant hydrolysates, anti-clumping agents, anti-foaming agents. Characteristics and compositions of cell growth media vary depending on the particular cellular requirements. Examples of commercially available cell growth media include: MEM (Minimum Essential Medium), BME (Basal Medium Eagle), DMEM (Dulbecco's modified Eagle's Medium), Iscoves DMEM (Iscove's modification of Dulbecco's Medium), GMEM, RPMI 1640, Leibovitz L-15, McCoy's, Medium 199, Ham (Ham's Media) F10 and derivatives, Ham F12, DMEM/F12.

Further guidance for the construction and production of viral vectors for use according to the disclosure can be found in Viral Vectors for Gene Therapy, Methods and Protocols. Series: Methods in Molecular Biology, Vol. 737. Merten and Al-Rubeai (Eds.); 2011 Humana Press (Springer); Gene Therapy. M. Giacca. 2010 Springer-Verlag; Heilbronn R. and Weger S. Viral Vectors for Gene Transfer: Current Status of Gene Therapeutics. In: Drug Delivery, Handbook of Experimental Pharmacology 197; M. Schäfer-Korting (Ed.). 2010 Springer-Verlag; pp. 143-170; Adeno-Associated Virus: Methods and Protocols. R. O. Snyder and P. Moulllier (Eds). 2011 Humana Press (Springer); Bünning H. et al. Recent developments in adeno-associated virus technology. J. Gene Med. 2008; 10:717-733; Adenovirus: Methods and Protocols. M. Chillón and A. Bosch (Eds.); Third Edition. 2014 Humana Press (Springer).

### Host Cell

The disclosure further provides a host cell comprising a nucleic acid construct or a viral vector encoding STXBP1 as described herein. The host cell according to the disclosure is a virus-producing cell, also named packaging cell which is transfected with the nucleic acid construct or viral vector in the presence of a helper vector or virus or other DNA constructs; and provides in trans all the missing functions which are required for the complete replication and packaging of a viral particle. Said packaging cells can be adherent or suspension cells.

The packaging cell may be a eukaryotic cell such as a mammalian cell, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HeLa cells (ATCC CCL2) and fetal rhesus lung cells (ATCC CL-160). Examples of non-human primate cells are Vero cells (ATCC CCL81), COS-1 cells (ATCC CRL-1650) or COS-7 cells (ATCC CRL-1651). Examples of dog cells are MDCK cells (ATCC CCL-34). Examples of rodent cells are hamster cells, such as BHK21-F, HKCC cells, or CHO cells.

As an alternative to mammalian sources, the packaging cell for producing the viral particles may be derived from an avian source such as chicken, duck, goose, quail or pheasant. Examples of avian cell lines include avian embryonic stem cells (WO01/85938; WO03/076601), immortalized duck retina cells (WO2005/042728), and avian embryonic stem cell derived cells including chicken cells (WO2006/108846) or duck cells, such as EB66 cell line (WO2008/129058; WO2008/142124).

In another embodiment, the host cell can be any packaging cell permissive for baculovirus infection and replication. In one example said cells are insect cells, such as SF9 cells (ATCC CRL-1711), Sf21 cells (IPLB-Sf21), MG1 cells (BTI-TN-MG1) or High Five™ cells (BTI-TN-5B1-4).

In one embodiment the host cell comprises:
(a) a first nucleic acid construct or viral vector comprising a transgene encoding human STXBP1;
(b) a second nucleic acid construct, for example a plasmid, encoding AAV rep and/or cap genes, wherein said second nucleic acid construct does not carry the ITR sequences; and, optionally,
(c) a third nucleic acid construct, for example a plasmid or virus, comprising viral helper genes.

The disclosure further provides a host cell transduced with a viral particle of the disclosure and the term “host cell” as used herein refers to any cell line that is susceptible to infection by a virus of interest, and amenable to culture in vitro.

### Pharmaceutical Composition

The present disclosure further provides a pharmaceutical composition comprising a nucleic acid construct, a viral vector, a viral particle of the disclosure in combination with a pharmaceutical acceptable excipient, diluent or carrier.

The term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

Any suitable pharmaceutically acceptable carrier, diluent or excipient can be used in the preparation of a pharmaceutical composition (See e.g., Remington: The Science and Practice of Pharmacy, Alfonso R. Gennaro (Editor) Mack Publishing Company, April 1997). Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol or sorbitol, or salts such as sodium chloride in the composition.

In one embodiment, the pharmaceutical composition is formulated as a solution, for example a buffered saline solution.

Supplementary active compounds may be incorporated into the pharmaceutical compositions of the disclosure. Guidance on co-administration of additional therapeutics can be found in the Compendium of Pharmaceutical and Specialties (CPS) of the Canadian Pharmacists Association.

In one embodiment, the pharmaceutical composition is a composition suitable for intraparenchymal, intracerebral, intravenous, or intrathecal administration. These pharmaceutical compositions are exemplary only and do not limit the pharmaceutical compositions suitable for other parenteral and non-parenteral administration routes. The pharmaceutical compositions described herein can be packaged in single unit dosage or in multi-dosage forms.

### Medical Use

Pharmaceutical compositions, nucleic acid constructs, viral vectors and viral particles of the present disclosure may be used in treating or preventing any condition that is associated with a loss of STXBP1 functional activity; for example any condition associated with STXBP1 mutation.

Such conditions include Dravet syndrome, Lennox-Gastaut syndrome, infantile spasms, myoclonic epilepsy, epileptic encephalopathy, early myoclonic encephalopathy, non-syndromic epilepsy, Ohtahara syndrome, early onset epileptic encephalopathy, West syndrome, development delay, autism spectrum disorders, ataxia-tremor-retardation syndrome, Rett syndrome and intellectual disability without epilepsy.

Pharmaceutical compositions, nucleic acid constructs, viral vectors and viral particles of the present disclosure may be especially useful for treating or preventing neurodevelopmental and/or epileptic disorders associated with genetic mutations in the STXBP1 gene, for example mutations that contribute to the development of syndromes such as Ohtahara, Dravet and West syndrome.

Thus in one embodiment, the pharmaceutical composition, nucleic acid construct, viral vector or viral particle is provided for use in therapy.

In one embodiment, the pharmaceutical composition, nucleic acid construct, viral vector or viral particle is provided for use in the treatment of an STXBP1 genetic disorder.

In one embodiment, the STXBP1 genetic disorder is Dravet syndrome, Lennox-Gastaut syndrome, infantile spasms, myoclonic epilepsy, epileptic encephalopathy, early myoclonic encephalopathy, non-syndromic epilepsy, Ohtahara syndrome, early onset epileptic encephalopathy, West syndrome, development delay, autism spectrum disorders, ataxia-tremor-retardation syndrome, Rett syndrome or intellectual disability without epilepsy.

In one embodiment, the STXBP1 genetic disorder is Dravet syndrome, Ohtahara syndrome or West syndrome.

In one embodiment, use of the nucleic acid construct, viral vector or viral particle is provided for the manufacture of a medicament for the treatment of an STXBP1 genetic disorder.

In one embodiment, the present disclosure provides a method of treating an STXBP1 genetic disorder, comprising administering a therapeutically effective amount of a pharmaceutical composition or viral particle to a patient in need thereof.

The term “therapeutically effective amount” refers to a number of viral particles or an amount of a pharmaceutical formulation comprising such viral particles, which, when administered to a patient or subject, achieves a desired therapeutic result. Desired therapeutic results include:
a significant reduction in frequency or duration of different seizure types, for example atonic seizures (drop attacks), myoclonic seizures, generalised seizures, partial seizures, febrile seizures, infantile spasms;
a significant achievement of sustained seizure freedom;
a significant impact on the progression of neurodevelopmental symptoms such as developmental delay, intellectual disability, language impairment, cognitive impairment, involuntary movements, gait disturbance, autistic features.

The term “patient” or “subject” as interchangeably used, refers to mammals. Any mammalian species may benefit from the methods of treatment. Typically, the patient is human. The patient may be a neonate, an infant, a child or an adolescent.

STXBP1 genetic disorder may be identified by known genetic mutations.

In one embodiment, the STXBP1 genetic disorder is associated with a pathological STXBP1 variant comprising a mutation or combination of mutations.

The term “pathological STXBP1 variant” means a variant of STXBP1 found in patient samples and identified through clinical testing or research, which is reported as being associated with a pathological phenotype. Pathological and likely pathological STXBP1 variants are described in Example 3 and illustrated in Tables 5 and 6 respectively.

In one embodiment the pathological STXBP1 variant comprises one or more mutation(s) selected from the group listed in Table 5.

In one embodiment the pathological STXBP1 variant comprises one or more mutation(s) selected from the group listed in Table 6.

The STXBP1 gene therapy described herein may be administered in combination with anti-epileptic drugs or other neuromodulatory treatments.

The pharmaceutical compositions, nucleic acid constructs, viral vectors or viral particles may be administered to the brain and/or the cerebrospinal fluid (CSF) of the patient. For example, they may be administered by injection or by the use of a purpose-specific administration device. Delivery to the brain may be selected from intracerebral delivery, intraparenchymal delivery, intracortical delivery, intrahippocampal delivery, intraputaminal delivery, intracerebellar delivery, and combinations thereof. Delivery to the CSF may be selected from intra-cisterna magna delivery, intrathecal delivery, intracerebroventricular (ICV) delivery, and combinations thereof.

The treatment may be provided as a single dose, but repeat doses may be considered, for example in cases where the treatment may not have targeted the correct region, or in future years and/or with different AAV serotypes.

### Sequences

The sequences included in the present invention are shown in Table 4.

TABLE 4
Sequences

| Sequence | |
|---|---|
| identifier | |
| and name | Sequence |
| SEQ ID NO: 1 | CGTTACATAACTTACGGTAAATGGCCCGCCTGGCTGACCGCCCAACGACCCCCGC |
| CAG 1.6 kb | CCATTGACGTCAATAATGACGTATGTTCCCATAGTAACGCCAATAGGGACTTTCC |
| promoter | ATTGACGTCAATGGGTGGAGTATTTACGGTAAACTGCCCACTTGGCAGTACATCA |
| | AGTGTATCATATGCCAAGTACGCCCCCTATTGACGTCAATGACGGTAAATGGCCC |
| | GCCTGGCATTATGCCCAGTACATGACCTTATGGGACTTTCCTACTTGGCAGTACA |
| | TCTACGTATTAGTCATCGCTATTACCATGCGTCGAGGTGAGCCCCACGTTCTGCT |
| | TCACTCTCCCCATCTCCCCCCCCTCCCCACCCCCAATTTTGTATTTATTTATTTT |
| | TTAATTATTTTATGCAGCGATGGGGGCGGGGGGGGGGGGGGCGCGCGCCAGGCGG |
| | GGCGGGGCGGGGCGAGGGGCGGGGCGGGGCGAGGCGGAGAGGTGCGGCGGCAGCC |
| | AATCAGAGCGGCGCGCTCCGAAAGTTTCCTTTTATGGCGAGGCGGCGGCGGCGGC |
| | GGCCCTATAAAAAGCGAAGCGCGCGGCGGGCG |
| SEQ ID NO: 2 | AGTGCAAGTGGGTTTTAGGACCAGGATGAGGCGGGGTGGGGGTGCCTACCTGACG |
| hSYN | ACCGACCCCGACCCACTGGACAAGCACCCAACCCCCATTCCCCAAATTGCGCATC |
| promoter | CCCTATCAGAGAGGGGGAGGGGAAACAGGATGCGGCGAGGCGCGTGCGCACTGCC |
| | AGCTTCAGCACCGCGGACAGTGCCTTCGCCCCCGCCTGGCGGCGCGCGCCACCGC |
| | CGCCTCAGCACTGAAGGCGCGCTGACGTCACTCGCCGGTCCCCCGCAAACTCCCC |
| | TTCCCGGCCACCTTGGTCGCGTCCGCGCCGCCGCCGGCCCAGCCGGACCGCACCA |
| | CGCGAGGCGCGAGATAGGGGGGCACGGGCGCGACCATCTGCGCTGCGGCGCCGGC |
| | GACTCAGCGCTGCCTCAGTCTGCGGTGGGCAGCGGAGGAGTCGTGTCGTGCCTGA |
| | GAGCGCAG |
| SEQ ID NO: 3 | AGCTGAATGGGGTCCGCCTCTTTTCCCTGCCTAAACAGACAGGAACTCCTGCCAA |
| MECP2 | TTGAGGGCGTCACCGCTAAGGCTCCGCCCCAGCCTGGGCTCCACAACCAATGAAG |
| promoter | GGTAATCTCGACAAAGAGCAAGGGGTGGGGCGCGGGCGCGCAGGTGCAGCAGCAC |
| | ACAGGCTGGTCGGGAGGGCGGGGCGCGACGTCTGCCGTGCGGGGTCCCGGCATCG |
| | GTTGCGCGC |
| SEQ ID NO: 4 | ATGCAGCTGGACCTAGGAGAGAAGCAGGAGAGGAAGATCCAGCACAAAAAATCCG |
| hNSE | AAGCTAAAAACAGGACACAGAGATGGGGGAAGAAAAGAGGGCAGAGTGAGGCAAA |
| promoter | AAGAGACTGAAGAGATGAGGGTGGCCGCCAGGCACTTTAGATAGGGGAGAGGCTT |
| | TATTTACCTCTGTTTGTTTTTTTTTTTTTTTTTTTTTTTTTTTTTTTGCGAGGTA |
| | GTCTTGCTTAGTCTCCAGGCTGGAGTGCAGTGGCACAATCTCAGCTCACTGCAAC |
| | TTCCACCTCCTGGGTTCAAGCAATTCTCCTGCCTCAGCCTCCCGAGTAGCTGGGA |
| | CTACAGGCGCATGCAACCGCGCCTGGCTAATTTTTGTATTTTTAGTAGAAACGGG |
| | GTTTCACCACGTTAGCCAGGATGGTCTGGATCTCCTGACCTCGTGATCTGCCCGC |
| | CTCCGCCTTCCAAAGTGCTGGGATTACAGGGGTGAGCCACAGCGCCTGGTCCCTA |
| | TTTACTTCTGTCTTCTACCTCCAGGAGATCAAAGACGCTGGCCTTCAGACCTGAT |
| | CAGACTCCCAGGGGCAGCCACCACATGTATGACAGAGAACAGAGGATGCCTGTTT |
| | TTGCCCCAAAGCTGGAAATTCATCACAACCTGAGGCCCAGGATCTGCTCTGTGCC |
| | GGTCCTCTGGGCAGTGTGGGGTGCAGAATGGGGTGCCTAGGCCTGAGCGTTGCCT |
| | GGAGCCTAGGCCGGGGGCCGCCCTCGGGCAGGCGTGGGTGAGAGCCAAGACCGCG |
| | TGGGCCGCGGGGTGCTGGTAGGAGTGGTTGGAGAGACTTGCGAAGGCGGCTGGGG |
| | TGTTCGGATTTCCAATAAAGAAACAGAGTGATGCTCCTGTGTCTGACCGGGTTTG |
| | TGAGACATTGAGGCTGTCTTGGGCTTCACTGGCAGTGTGGGCCTTCGTACCCGGG |
| | CTACAGGGGTGCGGCTCTGCCTGTTACTGTCGAGTGGGTCGGGCCGTGGGTATGA |
| | GCGCTTGTGTGCGCTGGGGCCAGGTCGTGGGTGCCCCCACCCTTCCCCCATCCTC |
| | CTCCCTTCCCCACTCCACCCTCGTCGGTCCCCCACCCGCGCTCGTACGTGCGCCT |
| | CCGCCGGCAGCTCCTGACTCATCGGGGGCTCCGGGTCACATGCGCCCGCGCGGCC |
| | CTATAGGCGCCTCCTCCGCCCGCCGCCCGGGAGCCGCAGCCGCCGCCGCCACTGC |
| | CACTCCCGCTCTCTCAGCGCCGCCGTCGCCACCGCCACCGCCACCGCCACTACCA |
| | CCGAGATCTGCGATCTAAGTAAGCTTGGCATTCCGGTACTGTTGGTAAAGCC |
| SEQ ID NO: 5 | CATTATGGCCTTAGGTCACTTCATCTCCATGGGGTTCTTCTTCTGATTTTCTAGA |
| CamKIl | AAATGAGATGGGGGTGCAGAGAGCTTCCTCAGTGACCTGCCCAGGGTCACATCAG |
| promoter | AAATGTCAGAGCTAGAACTTGAACTCAGATTACTAATCTTAAATTCCATGCCTTG |
| | GGGGCATGCAAGTACGATATACAGAAGGAGTGAACTCATTAGGGCAGATGACCAA |
| | TGAGTTTAGGAAAGAAGAGTCCAGGGCAGGGTACATCTACACCACCCGCCCAGCC |
| | CTGGGTGAGTCCAGCCACGTTCACCTCATTATAGTTGCCTCTCTCCAGTCCTACC |
| | TTGACGGGAAGCACAAGCAGAAACTGGGACAGGAGCCCCAGGAGACCAAATCTTC |
| | ATGGTCCCTCTGGGAGGATGGGTGGGGAGAGCTGTGGCAGAGGCCTCAGGAGGGG |
| | CCCTGCTGCTCAGTGGTGACAGATAGGGGTGAGAAAGCAGACAGAGTCATTCCGT |
| | CAGCATTCTGGGTCTGTTTGGTACTTCTTCTCACGCTAAGGTGGCGGTGTGATAT |
| | GCACAATGGCTAAAAAGCAGGGAGAGCTGGAAAGAAACAAGGACAGAGACAGAGG |
| | CCAAGTCAACCAGACCAATTCCCAGAGGAAGCAAAGAAACCATTACAGAGACTAC |
| | AAGGGGGAAGGGAAGGAGAGATGAATTAGCTTCCCCTGTAAACCTTAGAACCCAG |
| | CTGTTGCCAGGGCAACGGGGCAATACCTGTCTCTTCAGAGGAGATGAAGTTGCCA |
| | GGGTAACTACATCCTGTCTTTCTCAAGGACCATCCCAGAATGTGGCACCCACTAG |
| | CCGTTACCATAGCAACTGCCTCTTTGCCCCACTTAATCCCATCCCGTCTGTTAAA |
| | AGGGCCCTATAGTTGGAGGTGGGGGAGGTAGGAAGAGCGATGATCACTTGTGGAC |
| | TAAGTTTGTTCGCATCCCCTTCTCCAACCCCCTCAGTACATCACCCTGGGGGAAC |
| | AGGGTCCACTTGCTCCTGGGCCCACACAGTCCTGCAGTATTGTGTATATAAGGCC |
| | AGGGCAAAGAGGAGCAGGTTTTAAAGTGAAAGGCAGGCAGGTGTTGGGGAGGCAG |
| | TTACCGGGGCAACGGGAACAGGGCGTTTCGGAGGTGGTTGCCATGGGGACCTGGA |
| | TGCTGACGAAGGCTCGCGAGGCTGTGAGCAGCCACAGTGCCCTGCTCAGAAGCCC |
| | CAAGCTCGTCAGTCAAGCCGGTTCTCCGTTTGCACTCAGGAGCACGGGCAGGCGA |
| | GTGGCCCCTAGTTCTGGGGGCAGC |
| SEQ ID NO: 6 | TAAAAAGCAATGCCCAGTGATTGGAGGATTTGATGAGATGATGCCCGCGAGGTGC |
| Endogenous | TTGGCACGGAGTTTGACACAAGAACTCAGTGTTGGTGAATGCACGAATGCAGGTA |
| hSTXBP1 | CCCAGCGACAGGGAGGTGCTGTGGGTGGATTCACCCTGGCTTCGCTGCGGCTGGG |
| promoter | AGTGGGCGCTGCTAGTAAGAGATCTCGCCTCCAAGCTCCTTCCGTGGGCAGGAAA |
| | AAACGGAAGCTCCCAGAAAGAGGAAAGACAGGACCCGAGCGGGGTTTCAGGCAGA |
| | TGGAGCGCGTCGGTAGCCTGTGGCCAGGGATCCCAGCACCGACGGGAAAGAGGAG |
| | GCCTGGGTACCCTGCGCCCCGGGCGCGCGCGGCGCGTGAGGTGAGGGGGAGGGCG |
| | CGGCTCCGCACCAGCCAGCGGCCGCCTGGCGCCCAGCCGCATCTCGGGGGGCGGG |
| | GTTGAGCCTCCGGGCCGCAGTGCGATTGGCGGAGGCGAGTGGGTGACGCCAACGG |
| | CCGGCGCGAGGCCCCGCCCCCGGCTTGCCCCGCCCCCGCGCGCGCCGGCGGCGGG |
| | GCAGCCTCGCTCTGGCTCGCGCCGCGCCCCCGCGCCCAGTCCGCGCGTCAGTCGG |
| | TCCCTAGCGCGGCTGCGGGGCGGAGAGCTGCGGCTGGCCCAGCGCGCCCACCTGA |
| | GGAGGCGGCGGGGTCCGCAGGCGTCGCGGGACGAGGAGATCGGAGCCGGGAGACT |
| | CGCGCAGCGCC |
| SEQ ID NO: 7 | ATGGCCCCCATTGGCCTCAAAGCTGTTGTCGGAGAGAAGATTATGCATGATGTGA |
| Human | TAAAGAAGGTCAAGAAGAAGGGGGAATGGAAGGTGCTGGTGGTGGATCAGTTAAG |
| STXBP1 | CATGAGGATGCTGTCCTCCTGCTGCAAGATGACAGACATCATGACCGAGGGCATA |
| | ACGATTGTGGAAGATATCAATAAGCGCAGAGAGCCGCTCCCCAGCCTGGAGGCTG |
| | TGTATCTCATCACTCCATCCGAGAAGTCCGTCCACTCTCTCATCAGTGACTTTAA |
| | GGACCCGCCGACTGCTAAATACCGGGCTGCACACGTCTTCTTCACTGACTCTTGT |
| | CCAGATGCCCTGTTTAATGAACTGGTAAAATCCCGAGCAGCCAAAGTCATCAAAA |
| | CTCTGACGGAAATCAATATTGCATTTCTCCCGTATGAATCCCAGGTCTATTCCTT |
| | GGACTCTGCTGACTCTTTCCAAAGCTTCTACAGTCCCCACAAGGCTCAGATGAAG |
| | AATCCTATACTGGAGCGCCTGGCAGAGCAGATCGCGACCCTTTGTGCCACCCTGA |
| | AGGAGTACCCGGCTGTGCGGTATCGGGGGGAATACAAGGACAATGCCCTGCTGGC |
| | TCAGCTAATCCAGGACAAGCTCGATGCCTATAAAGCTGATGATCCAACAATGGGG |
| | GAGGGCCCAGACAAGGCACGCTCCCAGCTCCTGATCCTGGATCGAGGCTTTGACC |
| | CCAGCTCCCCTGTGCTCCATGAATTGACTTTTCAGGCTATGAGTTATGATCTGCT |
| | GCCTATCGAAAATGATGTATACAAGTATGAGACCAGCGGCATCGGGGAGGCACGG |
| | GTGAAGGAGGTGCTCCTGGACGAGGACGACGACCTGTGGATAGCACTGCGCCACA |
| | AGCACATCGCAGAGGTGTCCCAGGAAGTCACCCGGTCTCTGAAAGATTTTTCTTC |
| | TAGCAAGAGAATGAATACTGGAGAGAAGACCACCATGCGGGACCTGTCCCAGATG |
| | CTGAAGAAGATGCCTCAGTACCAGAAAGAGCTCAGCAAGTACTCCACCCACCTGC |
| | ACCTTGCTGAGGACTGTATGAAGCATTACCAAGGCACCGTAGACAAACTCTGCCG |
| | AGTGGAGCAGGACCTGGCCATGGGCACAGATGCTGAGGGAGAGAAGATCAAGGAC |
| | CCTATGCGAGCCATCGTCCCCATTCTGCTGGATGCCAATGTCAGCACTTATGACA |
| | AAATCCGCATCATCCTTCTCTACATCTTTTTGAAGAATGGCATCACGGAGGAAAA |
| | CCTGAACAAACTGATCCAGCACGCCCAGATACCCCCGGAGGATAGTGAGATCATC |
| | ACCAACATGGCTCACCTCGGCGTGCCCATCGTCACCGATTCCACGCTGCGTCGCC |
| | GGAGCAAGCCGGAGCGGAAGGAACGCATCAGCGAGCAGACCTACCAGCTCTCACG |
| | GTGGACTCCGATTATCAAGGACATCATGGAGGACACTATTGAGGACAAACTTGAC |
| | ACCAAACACTACCCTTATATCTCTACCCGTTCCTCTGCCTCCTTCAGCACCACCG |
| | CCGTCAGCGCCCGCTATGGGCACTGGCATAAGAACAAGGCCCCAGGCGAGTACCG |
| | CAGTGGCCCCCGCCTCATCATTTTCATCCTTGGGGGTGTGAGCCTGAATGAGATG |
| | CGCTGCGCCTACGAGGTGACCCAGGCCAACGGAAAGTGGGAGGTGCTGATAGGTT |
| | CTACTCACATTCTTACTCCCACCAAATTTCTCATGGACCTGAGACACCCCGACTT |
| | CAGGGAGTCCTCTAGGGTATCTTTTGAGGATCAGGCTCCAACAATGGAGTGA |
| SEQ ID NO: 8 | GATCCAGACATGATAAGATACATTGATGAGTTTGGACAAACCACAACTAGAATGC |
| SV40 poly A | AGTGAAAAAAATGCTTTATTTGTGAAATTTGTGATGCTATTGCTTTATTTGTAAC |
| | CATTATAAGCTGCAATAAACAAGTT |
| SEQ ID NO: 9 | MAPIGLKAVVGEKIMHDVIKKVKKKGEWKVLVVDQLSMRMLSSCCKMTDIMTEGI |
| Human | TIVEDINKRREPLPSLEAVYLITPSEKSVHSLISDEKDPPTAKYRAAHVFFTDSC |
| STXBP1 | PDALFNELVKSRAAKVIKTLTEINIAFLPYESQVYSLDSADSFQSFYSPHKAQMK |
| isoform a | NPILERLAEQIATLCATLKEYPAVRYRGEYKDNALLAQLIQDKLDAYKADDPTMG |
| | EGPDKARSQLLILDRGEDPSSPVLHELTFQAMSYDLLPIENDVYKYETSGIGEAR |
| | VKEVLLDEDDDLWIALRHKHIAEVSQEVTRSLKDESSSKRMNTGEKTTMRDLSQM |
| | LKKMPQYQKELSKYSTHLHLAEDCMKHYQGTVDKLCRVEQDLAMGTDAEGEKIKD |
| | PMRAIVPILLDANVSTYDKIRIILLYIFLKNGITEENLNKLIQHAQIPPEDSEII |
| | TNMAHLGVPIVTDSTLRRRSKPERKERISEQTYQLSRWTPIIKDIMEDTIEDKLD |
| | TKHYPYISTRSSASFSTTAVSARYGHWHKNKAPGEYRSGPRLIIFILGGVSLNEM |
| | RCAYEVTQANGKWEVLIGSTHILTPTKELMDLRHPDFRESSRVSFEDQAPTME |
| SEQ ID NO: | MAPIGLKAVVGEKIMHDVIKKVKKKGEWKVLVVDQLSMRMLSSCCKMTDIMTEGI |
| 10 | TIVEDINKRREPLPSLEAVYLITPSEKSVHSLISDEKDPPTAKYRAAHVFFTDSC |
| Human | PDALFNELVKSRAAKVIKTLTEINIAFLPYESQVYSLDSADSFQSFYSPHKAQMK |
| STXBP1 | NPILERLAEQIATLCATLKEYPAVRYRGEYKDNALLAQLIQDKLDAYKADDPTMG |
| isoform b | EGPDKARSQLLILDRGFDPSSPVLHELTFQAMSYDLLPIENDVYKYETSGIGEAR |
| | VKEVLLDEDDDLWIALRHKHIAEVSQEVTRSLKDESSSKRMNTGEKTTMRDLSQM |
| | LKKMPQYQKELSKYSTHLHLAEDCMKHYQGTVDKLCRVEQDLAMGTDAEGEKIKD |
| | PMRAIVPILLDANVSTYDKIRIILLYIFLKNGITEENLNKLIQHAQIPPEDSEII |
| | TNMAHLGVPIVTDSTLRRRSKPERKERISEQTYQLSRWTPIIKDIMEDTIEDKLD |
| | TKHYPYISTRSSASFSTTAVSARYGHWHKNKAPGEYRSGPRLIIFILGGVSLNEM |
| | RCAYEVTQANGKWEVLIGSTHILTPQKLLDTLKKLNKTDEEISS |
| SEQ ID NO: | MAPIGLKAVVGEKIMHDVIKKVKKKGEWKVLVVDQLSMRMLSSCCKMTDIMTEGI |
| 11 | TIVEDINKRREPLPSLEAVYLITPSEKSVHSLISDEKDPPTAKYRAAHVFFTDYA |
| Human | LFNELVKSRAAKVIKTLTEINIAFLPYESQVYSLDSADSFQSFYSPHKAQMKNPI |
| STXBP1 | LERLAEQIATLCATLKEYPAVRYRGEYKDNALLAQLIQDKLDAYKADDPTMGEGP |
| isoform c | DKARSQLLILDRGFDPSSPVLHELTFQAMSYDLLPIENDVYKYETSGIGEARVKE |
| | VLLDEDDDLWIALRHKHIAEVSQEVTRSLKDESSSKRMNTGEKTTMRDLSQMLKK |
| | MPQYQKELSKYSTHLHLAEDCMKHYQGTVDKLCRVEQDLAMGTDAEGEKIKDPMR |
| | AIVPILLDANVSTYDKIRIILLYIFLKNGITEENLNKLIQHAQIPPEDSEIITNM |
| | AHLGVPIVTDSTLRRRSKPERKERISEQTYQLSRWTPIIKDIMEDTIEDKLDTKH |
| | YPYISTRSSASFSTTAVSARYGHWHKNKAPGEYRSGPRLIIFILGGVSLNEMRCA |
| | YEVTQANGKWEVLIGSTHILTPQKLLDTLKKLNKTDEEISS |
| SEQ ID NO: | MHDVIKKVKKKGEWKVLVVDQLSMRMLSSCCKMTDIMTEGITIVEDINKRREPLP |
| 12 | SLEAVYLITPSEKSVHSLISDFKDPPTAKYRAAHVFFTDSCPDALFNELVKSRAA |
| Human | KVIKTLTEINIAFLPYESQVYSLDSADSFQSFYSPHKAQMKNPILERLAEQIATL |
| STXBP1 | CATLKEYPAVRYRGEYKDNALLAQLIQDKLDAYKADDPTMGEGPDKARSQLLILD |
| isoform d | RGFDPSSPVLHELTFQAMSYDLLPIENDVYKYETSGIGEARVKEVLLDEDDDLWI |
| | ALRHKHIAEVSQEVTRSLKDESSSKRMNTGEKTTMRDLSQMLKKMPQYQKELSKY |
| | STHLHLAEDCMKHYQGTVDKLCRVEQDLAMGTDAEGEKIKDPMRAIVPILLDANV |
| | STYDKIRIILLYIFLKNGITEENLNKLIQHAQIPPEDSEIITNMAHLGVPIVTDS |
| | TLRRRSKPERKERISEQTYQLSRWTPIIKDIMEDTIEDKLDTKHYPYISTRSSAS |
| | FSTTAVSARYGHWHKNKAPGEYRSGPRLIIFILGGVSLNEMRCAYEVTQANGKWE |
| | VLIGSTHILTPTKFLMDLRHPDFRESSRVSFEDQAPTME |
| SEQ ID NO: | MHDVIKKVKKKGEWKVLVVDQLSMRMLSSCCKMTDIMTEGITIVEDINKRREPLP |
| 13 | SLEAVYLITPSEKSVHSLISDEKDPPTAKYRAAHVFFTDSCPDALFNELVKSRAA |
| Human | KVIKTLTEINIAFLPYESQVYSLDSADSFQSFYSPHKAQMKNPILERLAEQIATL |
| STXBP1 | CATLKEYPAVRYRGEYKDNALLAQLIQDKLDAYKADDPTMGEGPDKARSQLLILD |
| isoform e | RGFDPSSPVLHELTFQAMSYDLLPIENDVYKYETSGIGEARVKEVLLDEDDDLWI |
| | ALRHKHIAEVSQEVTRSLKDESSSKRMNTGEKTTMRDLSQMLKKMPQYQKELSKY |
| | STHLHLAEDCMKHYQGTVDKLCRVEQDLAMGTDAEGEKIKDPMRAIVPILLDANV |
| | STYDKIRIILLYIFLKNGITEENLNKLIQHAQIPPEDSEIITNMAHLGVPIVTDS |
| | TLRRRSKPERKERISEQTYQLSRWTPIIKDIMEDTIEDKLDTKHYPYISTRSSAS |
| | FSTTAVSARYGHWHKNKAPGEYRSGPRLIIFILGGVSLNEMRCAYEVTQANGKWE |
| | VLIGSTHILTPQKLLDTLKKLNKTDEEISS |
| SEQ ID NO: | MAPIGLKAVVGEKIMHDVIKKVKKKGEWKVLVVDQLSMRMLSSCCKMTDIMTEGI |
| 14 | TIVEDINKRREPLPSLEAVYLITPSEKSVHSLISDEKDPPTAKYRAAHVFFTDSC |
| Human | PDALFNELVKSRAAKVIKTLTEINIAFLPYESQVYSLDSADSFQSFYSPHKAQMK |
| STXBP1 | NPILERLAEQIATLCATLKEYPAVRYRGEYKDNALLAQLIQDKLDAYKADDPTMG |
| isoform f | EGPDKARSQLLILDRGFDPSSPVLHELTFQAMSYDLLPIENDVYKYETSGIGEAR |
| | VKEVLLDEDDDLWIALRHKHIAEVSQEVTRSLKDESSSKRMNTGEKTTMRDLSQM |
| | LKKMPQYQKELSKYSTHLHLAEDCMKHYQGTVDKLCRVEQDLAMGTDAEGEKIKD |
| | PMRAIVPILLDANVSTYDKIRIILLYIFLKNGITEENLNKLIQHAQIPPEDSEII |
| | TNMAHLGVPIVTDSTLRRRSKPERKERISEQTYQLSRWTPIIKDIMEDTIEDKLD |
| | TKHYPYISTRSSASFSTTAVSARYGHWHKNKAPGEYRSGPRLIIFILGGVSLNEM |
| | RCAYEVTQANGKWEVLIGEWPERTLAR |
| SEQ ID NO: | MAPIGLKAVVGEKIMHDVIKKVKKKGEWKVLVVDQLSMRMLSSCCKMTDIMTEGI |
| 15 | TIVEDINKRREPLPSLEAVYLITPSEKSVHSLISDEKDPPTAKYRAAHVFFTDSC |
| Human | PDALFNELVKSRAAKVIKTLTEINIAFLPYESQVYSLDSADSFQSFYSPHKAQMK |
| STXBP1 | NPILERLAEQIATLCATLKEYPAVRYRGEYKDNALLAQLIQDKLDAYKADDPTMG |
| isoform g | EGPDKARSQLLILDRGFDPSSPVLHELTFQAMSYDLLPIENDVYKYETSGIGEAR |
| | VKEVLLDEDDDLWIALRHKHIAEVSQEVTRSLKDESSSKRMNTGEKTTMRDLSQM |
| | LKKMPQYQKELSKYSTHLHLAEDCMKHYQGTVDKLCRVEQDLAMGTDAEGEKIKD |
| | PMRAIVPILLDANVSTYDKIRIILLYIFLKNGITEENLNKLIQHAQIPPEDSEII |
| | TNMAHLGVPIVTDSTLRRRSKPERKERISEQTYQLSRWTPIIKDIMEDTIEDKLD |
| | TKHYPYISTRSSASESTTAVSARYGHWHKNKAPGEYRSGPRLIIFILGGVSLNEM |
| | RCAYEVTQANGKWEVLIVPVEAGS |
| SEQ ID NO: | MAPIGLKAVVGEKIMHDVIKKVKKKGEWKVLVVDQLSMRMLSSCCKMTDIMTEGI |
| 16 | TIVEDINKRREPLPSLEAVYLITPSEKSVHSLISDEKDPPTAKYRAAHVFFTDSC |
| Human | PDALFNELVKSRAAKVIKTLTEINIAFLPYESQVYSLDSADSFQSFYSPHKAQMK |
| STXBP1 | NPILERLAEQIATLCATLKEYPAVRYRGEYKDNALLAQLIQDKLDAYKADDPTMG |
| isoform h | EGPDKARSQLLILDRGFDPSSPVLHELTFQAMSYDLLPIENDVYKEVTRSLKDES |
| | SSKRMNTGEKTTMRDLSQMLKKMPQYQKELSKYSTHLHLAEDCMKHYQGTVDKLC |
| | RVEQDLAMGTDAEGEKIKDPMRAIVPILLDANVSTYDKIRIILLYIFLKNGITEE |
| | NLNKLIQHAQIPPEDSEIITNMAHLGVPIVTDSTLRRRSKPERKERISEQTYQLS |
| | RWTPIIKDIMEDTIEDKLDTKHYPYISTRSSASFSTTAVSARYGHWHKNKAPGEY |
| | RSGPRLIIFILGGVSLNEMRCAYEVTQANGKWEVLIGSTHILTPTKFLMDLRHPD |
| | FRESSRVSFEDQAPTME |
| SEQ ID NO: | AGAAAAACTCATCGAGCATCAAATGAAATTGCAATTTATTCATATCAGGATTATC |
| 17 | AATACCATATTTTTGAAAAAGCCGTTTCTGTAATGAAGGAGAAAACTCACCGAGG |
| AAV9.hu14 | CAGTTCCATAGGATGGCAAGATCCTGGTATCGGTCTGCGATTCCGACTCGTCCAA |
| DNA | CATCAATACAACCTATTAATTTCCCCTCGTCAAAAATAAGGTTATCAAGTGAGAA |
| sequence | ATCACCATGAGTGACGACTGAATCCGGTGAGAATGGCAAAAGTTTATGCATTTCT |
| | TTCCAGACTTGTTCAACAGGCCAGCCATTACGCTCGTCATCAAAATCACTCGCAT |
| | CAACCAAACCGTTATTCATTCGTGATTGCGCCTGAGCGAGGCGAAATACGCGATC |
| | GCTGTTAAAAGGACAATTACAAACAGGAATCGAGTGCAACCGGCGCAGGAACACT |
| | GCCAGCGCATCAACAATATTTTCACCTGAATCAGGATATTCTTCTAATACCTGGA |
| | ACGCTGTTTTTCCGGGGATCGCAGTGGTGAGTAACCATGCATCATCAGGAGTACG |
| | GATAAAATGCTTGATGGTCGGAAGTGGCATAAATTCCGTCAGCCAGTTTAGTCTG |
| | ACCATCTCATCTGTAACATCATTGGCAACGCTACCTTTGCCATGTTTCAGAAACA |
| | ACTCTGGCGCATCGGGCTTCCCATACAAGCGATAGATTGTCGCACCTGATTGCCC |
| | GACATTATCGCGAGCCCATTTATACCCATATAAATCAGCATCCATGTTGGAATTT |
| | AATCGCGGCCTCGACGTTTCCCGTTGAATATGGCTCATATTCTTCCTTTTTCAAT |
| | ATTATTGAAGCATTTATCAGGGTTATTGTCTCATGAGCGGATACATATTTGAATG |
| | TATTTAGAAAAATAAACAAATAGGGGTCAGTGTTACAACCAATTAACCAATTCTG |
| | AACATTATCGCGAGCCCATTTATACCTGAATATGGCTCATAACACCCCTTGTTTG |
| | CCTGGCGGCAGTAGCGCGGTGGTCCCACCTGACCCCATGCCGAACTCAGAAGTGA |
| | AACGCCGTAGCGCCGATGGTAGTGTGGGGACTCCCCATGCGAGAGTAGGGAACTG |
| | CCAGGCATCAAATAAAACGAAAGGCTCAGTCGAAAGACTGGGCCTTTCGCCCGGG |
| | CTAATTAGGGGGTGTCGCCCTTCGCTGAAGTCCTGTATTAGAGGTCACGTGAGTG |
| | TTTTGCGACATTTTGCGACACCATGTGGTCACGCTGGGTATTTAAGCCCGAGTGA |
| | GCACGCAGGGTCTCCATTTTGAAGCGGGAGGTTTGAACGCGCAGCCGCCATGCCG |
| | GGGTTTTACGAGATTGTGATTAAGGTCCCCAGCGACCTTGACGAGCATCTGCCCG |
| | GCATTTCTGACAGCTTTGTGAACTGGGTGGCCGAGAAGGAATGGGAGTTGCCGCC |
| | AGATTCTGACATGGATCTGAATCTGATTGAGCAGGCACCCCTGACCGTGGCCGAG |
| | AAGCTGCAGCGCGACTTTCTGACGGAATGGCGCCGTGTGAGTAAGGCCCCGGAGG |
| | CCCTTTTCTTTGTGCAATTTGAGAAGGGAGAGAGCTACTTCCACATGCACGTGCT |
| | CGTGGAAACCACCGGGGTGAAATCCATGGTTTTGGGACGTTTCCTGAGTCAGATT |
| | CGCGAAAAACTGATTCAGAGAATTTACCGCGGGATCGAGCCGACTTTGCCAAACT |
| | GGTTCGCGGTCACAAAGACCAGAAATGGCGCCGGAGGCGGGAACAAGGTGGTGGA |
| | TGAGTGCTACATCCCCAATTACTTGCTCCCCAAAACCCAGCCTGAGCTCCAGTGG |
| | GCGTGGACTAATATGGAACAGTATTTAAGCGCCTGTTTGAATCTCACGGAGCGTA |
| | AACGGTTGGTGGCGCAGCATCTGACGCACGTGTCGCAGACGCAGGAGCAGAACAA |
| | AGAGAATCAGAATCCCAATTCTGATGCGCCGGTGATCAGATCAAAAACTTCAGCC |
| | AGGTACATGGAGCTGGTCGGGTGGCTCGTGGACAAGGGGATTACCTCGGAGAAGC |
| | AGTGGATCCAGGAGGACCAGGCCTCATACATCTCCTTCAATGCGGCCTCCAACTC |
| | GCGGTCCCAAATCAAGGCTGCCTTGGACAATGCGGGAAAGATTATGAGCCTGACT |
| | AAAACCGCCCCCGACTACCTGGTGGGCCAGCAGCCCGTGGAGGACATTTCCAGCA |
| | ATCGGATTTATAAAATTTTGGAACTAAACGGGTACGATCCCCAATATGCGGCTTC |
| | CGTCTTTCTGGGATGGGCCACGAAAAAGTTCGGCAAGAGGAACACCATCTGGCTG |
| | TTTGGGCCTGCAACTACCGGGAAGACCAACATCGCGGAGGCCATAGCCCACACTG |
| | TGCCCTTCTACGGGTGCGTAAACTGGACCAATGAGAACTTTCCCTTCAACGACTG |
| | TGTCGACAAGATGGTGATCTGGTGGGAGGAGGGGAAGATGACCGCCAAGGTCGTG |
| | GAGTCGGCCAAAGCCATTCTCGGAGGAAGCAAGGTGCGCGTGGACCAGAAATGCA |
| | AGTCCTCGGCCCAGATAGACCCGACTCCCGTGATCGTCACCTCCAACACCAACAT |
| | GTGCGCCGTGATTGACGGGAACTCAACGACCTTCGAACACCAGCAGCCGTTGCAA |
| | GACCGGATGTTCAAATTTGAACTCACCCGCCGTCTGGATCATGACTTTGGGAAGG |
| | TCACCAAGCAGGAAGTCAAAGACTTTTTCCGGTGGGCAAAGGATCACGTGGTTGA |
| | GGTGGAGCATGAATTCTACGTCAAAAAGGGTGGAGCCAAGAAAAGACCCGCCCCC |
| | AGTGACGCAGATATAAGTGAGCCCAAACGGGTGCGCGAGTCAGTTGCGCAGCCAT |
| | CGACGTCAGACGCGGAAGCTTCGATCAACTACGCAGACAGGTACCAAAACAAATG |
| | TTCTCGTCACGTGGGCATGAATCTGATGCTGTTTCCCTGCAGACAATGCGAGAGA |
| | CTGAATCAGAATTCAAATATCTGCTTCACTCACGGTGTCAAAGACTGTTTAGAGT |
| | GCTTTCCCGTGTCAGAATCTCAACCCGTTTCTGTCGTCAAAAAGGCGTATCAGAA |
| | ACTGTGCTACATTCATCACATCATGGGAAAGGTGCCAGACGCTTGCACTGCTTGC |
| | GACCTGGTCAATGTGGACTTGGATGACTGTGTTTCTGAACAATAAATGACTTAAA |
| | CCAGGTATGGCTGCCGATGGTTATCTTCCAGATTGGCTCGAGGACAACCTTAGTG |
| | AAGGAATTCGCGAGTGGTGGGCTTTGAAACCTGGAGCCCCTCAACCCAAGGCAAA |
| | TCAACAACATCAAGACAACGCTCGAGGTCTTGTGCTTCCGGGTTACAAATACCTT |
| | GGACCCGGCAACGGACTCGACAAGGGGGAGCCGGTCAACGCAGCAGACGCGGCGG |
| | CCCTCGAGCACGACAAGGCCTACGACCAGCAGCTCAAGGCCGGAGACAACCCGTA |
| | CCTCAAGTACAACCACGCCGACGCCGAGTTCCAGGAGCGGCTCAAAGAAGATACG |
| | TCTTTTGGGGGCAACCTCGGGCGAGCAGTCTTCCAGGCCAAAAAGAGGCTTCTTG |
| | AACCTCTTGGTCTGGTTGAGGAAGCGGCTAAGACGGCTCCTGGAAAGAAGAGGCC |
| | TGTAGAGCAGTCTCCTCAGGAACCGGACTCCTCCGCGGGTATTGGCAAATCGGGT |
| | GCACAGCCCGCTAAAAAGAGACTCAATTTCGGTCAGACTGGCGACACAGAGTCAG |
| | TCCCAGACCCTCAACCAATCGGAGAACCTCCCGCAGCCCCCTCAGGTGTGGGATC |
| | TCTTACAATGGCTTCAGGTGGTGGCGCACCAGTGGCAGACAATAACGAAGGTGCC |
| | GATGGAGTGGGTAGTTCCTCGGGAAATTGGCATTGCGATTCCCAATGGCTGGGGG |
| | ACAGAGTCATCACCACCAGCACCCGAACCTGGGCCCTGCCCACCTACAACAATCA |
| | CCTCTACAAGCAAATCTCCAACAGCACATCTGGAGGATCTTCAAATGACAACGCC |
| | TACTTCGGCTACAGCACCCCCTGGGGGTATTTTGACTTCAACAGATTCCACTGCC |
| | ACTTCTCACCACGTGACTGGCAGCGACTCATCAACAACAACTGGGGATTCCGGCC |
| | TAAGCGACTCAACTTCAAGCTCTTCAACATTCAGGTCAAAGAGGTTACGGACAAC |
| | AATGGAGTCAAGACCATCGCCAATAACCTTACCAGCACGGTCCAGGTCTTCACGG |
| | ACTCAGACTATCAGCTCCCGTACGTGCTCGGGTCGGCTCACGAGGGCTGCCTCCC |
| | GCCGTTCCCAGCGGACGTTTTCATGATTCCTCAGTACGGGTATCTGACGCTTAAT |
| | GATGGAAGCCAGGCCGTGGGTCGTTCGTCCTTTTACTGCCTGGAATATTTCCCGT |
| | CGCAAATGCTAAGAACGGGTAACAACTTCCAGTTCAGCTACGAGTTTGAGAACGT |
| | ACCTTTCCATAGCAGCTACGCTCACAGCCAAAGCCTGGACCGACTAATGAATCCA |
| | CTCATCGACCAATACTTGTACTATCTCTCAAAGACTATTAACGGTTCTGGACAGA |
| | ATCAACAAACGCTAAAATTCAGTGTGGCCGGACCCAGCAACATGGCTGTCCAGGG |
| | AAGAAACTACATACCTGGACCCAGCTACCGACAACAACGTGTCTCAACCACTGTG |
| | ACTCAAAACAACAACAGCGAATTTGCTTGGCCTGGAGCTTCTTCTTGGGCTCTCA |
| | ATGGACGTAATAGCTTGATGAATCCTGGACCTGCTATGGCCAGCCACAAAGAAGG |
| | AGAGGACCGTTTCTTTCCTTTGTCTGGATCTTTAATTTTTGGCAAACAAGGAACT |
| | GGAAGAGACAACGTGGATGCGGACAAAGTCATGATAACCAACGAAGAAGAAATTA |
| | AAACTACTAACCCGGTAGCAACGGAGTCCTATGGACAAGTGGCCACAAACCACCA |
| | GAGTGCCCAAGCACAGGCGCAGACCGGCTGGGTTCAAAACCAAGGAATACTTCCG |
| | GGTATGGTTTGGCAGGACAGAGATGTGTACCTGCAAGGACCCATTTGGGCCAAAA |
| | TTCCTCACACGGACGGCAACTTTCACCCTTCTCCGCTGATGGGAGGGTTTGGAAT |
| | GAAGCACCCGCCTCCTCAGATCCTCATCAAAAACACACCTGTACCTGCGGATCCT |
| | CCAACGGCCTTCAACAAGGACAAGCTGAACTCTTTCATCACCCAGTATTCTACTG |
| | GCCAAGTCAGCGTGGAGATCGAGTGGGAGCTGCAGAAGGAAAACAGCAAGCGCTG |
| | GAACCCGGAGATCCAGTACACTTCCAACTATTACAAGTCTAATAATGTTGAATTT |
| | GCTGTTAATACTGAAGGTGTATATAGTGAACCCCGCCCCATTGGCACCAGATACC |
| | TGACTCGTAATCTGTAATTGCTTGTTAATCAATAAACCGTTTAATTCGTTTCAGT |
| | TGAACTTTGGTCTCTGCGCGTCAAAAGGGCGACACAAAATTTATTCTAAATGCAT |
| | AATAAATACTGATAACATCTTATAGTTTGTATTATATTTTGTATTATCGTTGACA |
| | TGTATAATTTTGATATCAAAAACTGATTTTCCCTTTATTATTTTCGAGATTTATT |
| | TTCTTAATTCTCTTTAACAAACTAGAAATATTGTATATACAAAAAATCATAAATA |
| | ATAGATGAATAGTTTAATTATAGGTGTTCATCAATCGAAAAAGCAACGTATCTTA |
| | TTTAAAGTGCGTTGCTTTTTTCTCATTTATAAGGTTAAATAATTCTCATATATCA |
| | AGCAAAGTGACAGGCGCCCTTAAATATTCTGACAAATGCTCTTTCCCTAAACTCC |
| | CCCCATAAAAAAACCCGCCGAAGCGGGTTTTTACGTTATTTGCGGATTAACGATT |
| | ACTCGTTATCAGAACCGCCCAGGGGGCCCGAGCTTAAGACTGGCCGTCGTTTTAC |
| | AACACAGAAAGAGTTTGTAGAAACGCAAAAAGGCCATCCGTCAGGGGCCTTCTGC |
| | TTAGTTTGATGCCTGGCAGTTCCCTACTCTCGCCTTCCGCTTCCTCGCTCACTGA |
| | CTCGCTGCGCTCGGTCGTTCGGCTGCGGCGAGCGGTATCAGCTCACTCAAAGGCG |
| | GTAATACGGTTATCCACAGAATCAGGGGATAACGCAGGAAAGAACATGTGAGCAA |
| | AAGGCCAGCAAAAGGCCAGGAACCGTAAAAAGGCCGCGTTGCTGGCGTTTTTCCA |
| | TAGGCTCCGCCCCCCTGACGAGCATCACAAAAATCGACGCTCAAGTCAGAGGTGG |
| | CGAAACCCGACAGGACTATAAAGATACCAGGCGTTTCCCCCTGGAAGCTCCCTCG |
| | TGCGCTCTCCTGTTCCGACCCTGCCGCTTACCGGATACCTGTCCGCCTTTCTCCC |
| | TTCGGGAAGCGTGGCGCTTTCTCATAGCTCACGCTGTAGGTATCTCAGTTCGGTG |
| | TAGGTCGTTCGCTCCAAGCTGGGCTGTGTGCACGAACCCCCCGTTCAGCCCGACC |
| | GCTGCGCCTTATCCGGTAACTATCGTCTTGAGTCCAACCCGGTAAGACACGACTT |
| | ATCGCCACTGGCAGCAGCCACTGGTAACAGGATTAGCAGAGCGAGGTATGTAGGC |
| | GGTGCTACAGAGTTCTTGAAGTGGTGGGCTAACTACGGCTACACTAGAAGAACAG |
| | TATTTGGTATCTGCGCTCTGCTGAAGCCAGTTACCTTCGGAAAAAGAGTTGGTAG |
| | CTCTTGATCCGGCAAACAAACCACCGCTGGTAGCGGTGGTTTTTTTGTTTGCAAG |
| | CAGCAGATTACGCGCAGAAAAAAAGGATCTCAAGAAGATCCTTTGATCTTTTCTA |
| | CGGGGTCTGACGCTCAGTGGAACGACGCGCGCGTAACTCACGTTAAGGGATTTTG |
| | GTCATGAGCTTGCGCCGTCCCGTCAAGTCAGCGTAATGCTCTGCTT |
| SEQ ID NO: | AGGAACCCCTAGTGATGGAGTTGGCCACTCCCTCTCTGCGCGCTCGCTCGCTCAC |
| 18 | TGAGGCCGGGCGACCAAAGGTCGCCCGACGCCCGGGCTTTGCCCGGGCGGCCTCA |
| 3′ ITR | GTGAGCGAGCGAGCGCGC |
| SEQ ID NO: | GCGCGCTCGCTCGCTCACTGAGGCCGCCCGGGCAAAGCCCGGGCGTCGGGCGACC |
| 19 | TTTGGTCGCCCGGCCTCAGTGAGCGAGCGAGCGCGCAGAGAGGGAGTGGCCAACT |
| 5′ ITR | CCATCACTAGGGGTTCCT |
| SEQ ID NO: | MAADGYLPDWLEDTLSEGIRQWWKLKPGPPPPKPAERHKDDSRGLVLPGYKYLGP |
| 20 | FNGLDKGEPVNEADAAALEHDKAYDRQLDSGDNPYLKYNHADAEFQERLKEDTSE |
| AAVtt | GGNLGRAVEQAKKRILEPLGLVEEPVKTAPGKKRPVEHSPAEPDSSSGTGKSGQQ |
| | PARKRLNFGQTGDADSVPDPQPLGQPPAAPSGLGTNTMASGSGAPMADNNEGADG |
| | VGNSSGNWHCDSTWMGDRVITTSTRTWALPTYNNHLYKQISSQSGASNDNHYFGY |
| | STPWGYFDENRFHCHESPRDWQRLINNNWGFRPKRLSFKLENIQVKEVTQNDGTT |
| | TIANNLTSTVQVFTDSEYQLPYVLGSAHQGCLPPFPADVEMVPQYGYLTLNNGSQ |
| | AVGRSSFYCLEYFPSQMLRTGNNFTFSYTFEDVPFHSSYAHSQSLDRLMNPLIDQ |
| | YLYYLSRTNTPSGTTTMSRLQFSQAGASDIRDQSRNWLPGPCYRQQRVSKTAADN |
| | NNSDYSWTGATKYHLNGRDSLVNPGPAMASHKDDEEKYFPQSGVLIFGKQDSGKT |
| | NVDIEKVMITDEEEIRTTNPVATEQYGSVSTNLQSGNTQAATSDVNTQGVLPGMV |
| | WQDRDVYLQGPIWAKIPHTDGHFHPSPLMGGFGLKHPPPQILIKNTPVPANPSTT |
| | FSAAKFASFITQYSTGQVSVEIEWELQKENSKRWNPEIQYTSNYNKSVNVDFTVD |
| | TNGVYSEPRPIGTRYLTRNL |
| SEQ ID NO: | MAADGYLPDWLEDNLSEGIREWWALKPGAPQPKANQQHQDNARGLVLPGYKYLGP |
| 21 | GNGLDKGEPVNAADAAALEHDKAYDQQLKAGDNPYLKYNHADAEFQERLKEDTSE |
| AAV9 | GGNLGRAVFQAKKRLLEPLGLVEEAAKTAPGKKRPVEQSPQEPDSSAGIGKSGAQ |
| | PAKKRLNFGQTGDTESVPDPQPIGEPPAAPSGVGSLTMASGGGAPVADNNEGADG |
| | VGSSSGNWHCDSQWLGDRVITTSTRTWALPTYNNHLYKQISNSTSGGSSNDNAYF |
| | GYSTPWGYFDENRFHCHESPRDWQRLINNNWGFRPKRLNFKLFNIQVKEVTDNNG |
| | VKTIANNLTSTVQVFTDSDYQLPYVLGSAHEGCLPPFPADVEMIPQYGYLTLNDG |
| | SQAVGRSSFYCLEYFPSQMLRTGNNFQFSYEFENVPFHSSYAHSQSLDRLMNPLI |
| | DQYLYYLSKTINGSGQNQQTLKESVAGPSNMAVQGRNYIPGPSYRQQRVSTTVTQ |
| | NNNSEFAWPGASSWALNGRNSLMNPGPAMASHKEGEDRFFPLSGSLIFGKQGTGR |
| | DNVDADKVMITNEEEIKTTNPVATESYGQVATNHQSAQAQAQTGWVQNQGILPGM |
| | VWQDRDVYLQGPIWAKIPHTDGNFHPSPLMGGFGMKHPPPQILIKNTPVPADPPT |
| | AFNKDKLNSFITQYSTGQVSVEIEWELQKENSKRWNPEIQYTSNYYKSNNVEFAV |
| | NTEGVYSEPRPIGTRYLTRNL |
| SEQ ID NO: | AGTCGGTCCCTAGCGCGGCTGCGGGGCGGAGAGCTGCGGCTGGCCCAGCGCGCCC |
| 22 | ACCTGAGGAGGCGGCGGGGTCCGCAGGCGTCGCGGGACGAGGAGATCGGAGCCGG |
| Human | GAGACTCGCGCAGCGCCATGGCCCCCATTGGCCTCAAAGCTGTTGTCGGAGAGAA |
| STXBP1 | GATTATGCATGATGTGATAAAGAAGGTCAAGAAGAAGGGGGAATGGAAGGTGCTG |
| (transcript | GTGGTGGATCAGTTAAGCATGAGGATGCTGTCCTCCTGCTGCAAGATGACAGACA |
| variant 1) | TCATGACCGAGGGCATAACGATTGTGGAAGATATCAATAAGCGCAGAGAGCCGCT |
| encoding | CCCCAGCCTGGAGGCTGTGTATCTCATCACTCCATCCGAGAAGTCCGTCCACTCT |
| isoform a | CTCATCAGTGACTTTAAGGACCCGCCGACTGCTAAATACCGGGCTGCACACGTCT |
| | TCTTCACTGACTCTTGTCCAGATGCCCTGTTTAATGAACTGGTAAAATCCCGAGC |
| | AGCCAAAGTCATCAAAACTCTGACGGAAATCAATATTGCATTTCTCCCGTATGAA |
| | TCCCAGGTCTATTCCTTGGACTCTGCTGACTCTTTCCAAAGCTTCTACAGTCCCC |
| | ACAAGGCTCAGATGAAGAATCCTATACTGGAGCGCCTGGCAGAGCAGATCGCGAC |
| | CCTTTGTGCCACCCTGAAGGAGTACCCGGCTGTGCGGTATCGGGGGGAATACAAG |
| | GACAATGCCCTGCTGGCTCAGCTAATCCAGGACAAGCTCGATGCCTATAAAGCTG |
| | ATGATCCAACAATGGGGGAGGGCCCAGACAAGGCACGCTCCCAGCTCCTGATCCT |
| | GGATCGAGGCTTTGACCCCAGCTCCCCTGTGCTCCATGAATTGACTTTTCAGGCT |
| | ATGAGTTATGATCTGCTGCCTATCGAAAATGATGTATACAAGTATGAGACCAGCG |
| | GCATCGGGGAGGCACGGGTGAAGGAGGTGCTCCTGGACGAGGACGACGACCTGTG |
| | GATAGCACTGCGCCACAAGCACATCGCAGAGGTGTCCCAGGAAGTCACCCGGTCT |
| | CTGAAAGATTTTTCTTCTAGCAAGAGAATGAATACTGGAGAGAAGACCACCATGC |
| | GGGACCTGTCCCAGATGCTGAAGAAGATGCCTCAGTACCAGAAAGAGCTCAGCAA |
| | GTACTCCACCCACCTGCACCTTGCTGAGGACTGTATGAAGCATTACCAAGGCACC |
| | GTAGACAAACTCTGCCGAGTGGAGCAGGACCTGGCCATGGGCACAGATGCTGAGG |
| | GAGAGAAGATCAAGGACCCTATGCGAGCCATCGTCCCCATTCTGCTGGATGCCAA |
| | TGTCAGCACTTATGACAAAATCCGCATCATCCTTCTCTACATCTTTTTGAAGAAT |
| | GGCATCACGGAGGAAAACCTGAACAAACTGATCCAGCACGCCCAGATACCCCCGG |
| | AGGATAGTGAGATCATCACCAACATGGCTCACCTCGGCGTGCCCATCGTCACCGA |
| | TTCCACGCTGCGTCGCCGGAGCAAGCCGGAGCGGAAGGAACGCATCAGCGAGCAG |
| | ACCTACCAGCTCTCACGGTGGACTCCGATTATCAAGGACATCATGGAGGACACTA |
| | TTGAGGACAAACTTGACACCAAACACTACCCTTATATCTCTACCCGTTCCTCTGC |
| | CTCCTTCAGCACCACCGCCGTCAGCGCCCGCTATGGGCACTGGCATAAGAACAAG |
| | GCCCCAGGCGAGTACCGCAGTGGCCCCCGCCTCATCATTTTCATCCTTGGGGGTG |
| | TGAGCCTGAATGAGATGCGCTGCGCCTACGAGGTGACCCAGGCCAACGGAAAGTG |
| | GGAGGTGCTGATAGGTTCTACTCACATTCTTACTCCCACCAAATTTCTCATGGAC |
| | CTGAGACACCCCGACTTCAGGGAGTCCTCTAGGGTATCTTTTGAGGATCAGGCTC |
| | CAACAATGGAGTGAGAGCCAAAGAAACAAAGATCCACACACATCCTCACCCCACA |
| | GAAACTGCTGGACACACTGAAGAAACTGAATAAAACAGATGAAGAAATAAGCAGT |
| | TAAAAAAATAAGTCGCCCCTCCAAAACACGCCCCCATCCCACAGCGCTCCGCAGC |
| | TTCCCACCACCGCCCGCCTCAGTTCCTTTGCGTCTGTTGCCTCCCCAGCCCTGCA |
| | CGCCCTGGCTGGCACTGTTGCCGCTGCATTCTCGTGTTCAGTGATGCCCTCTTCT |
| | TGTTTGAAACAAAAGAAAATAATGCATTGTGTTTTTTAAAAAGAGTATCTTATAC |
| | ATGTATCCTAAAAAGAGAAGCTCATGTGCAATTGGTGCACAGCAGGAGAAATTTC |
| | TGGACTGTTAGGATGAATGGACGCCTTCTCCCCGTTATTTAAGATTTGTGACCTT |
| | GTACATAACCCTGGGTGACGTGCACATTGCTTGGGTATGGAACGGTAGAAATTTG |
| | GGTGTTTTTAAAACCTTGTTTGGGGTTGTTCCTGTCCTTGTTGAGAATCATAGAG |
| | ATGTCTGTGTTCTTGGAGTATTTCACACTGAGGACTAATCTGCTATCTTCATTCC |
| | AGTCCCTACCCCTCAGTGCCTGCTCTCATCCAAATAACCTGGGAGGTGACAATCA |
| | GGATATCTCAGGAGGTCCAAGGTGGAACAGACCTCTTTGCCTTTCCCAGCGTCTC |
| | ATACCCCCGGTAGTGCAGCTGTGGGTGGAGGCTGGGGTGTCTGCACGAAGTCAGG |
| | CCAGCGTCCTCCTCCACAGCCTGTCACTGCCCCCTCCCCAGCCTGTGTCCACAGT |
| | GCTGTGATCCCGAGGGAAGTCCTCCAGTCTAAGTCACAGTGCCCTGACAGGTGAG |
| | AAGCAAACTCCCGCTGGAAGCCTCCATCTCTTTGGAAAAACAGTTAGTCTGGAGC |
| | CTGTGGCCCAGGCCCTTCTGTCCCCAGGCATCATCCCAACAGCTCATTTTCCCTA |
| | GTCCGCCTTCGTTCAAGGGTCAGGAATGGACCAGAACAGATGGGTTCTGGAGGCC |
| | CCTGAACAGAGGGCTATGGCTGTGGAGAAGGTTCTTGGCCCGTTGGACTCACACA |
| | GACCCTGTACCCTCTCGGCAAGCATCTTCAGTCAGATTATCCTCAGTTTCAGATA |
| | CTTCATAATACCTTGTGTTGTGTGGGGTCATACATCATCGTGTTTGTAAGAGAAG |
| | ATGGTCATTTTATTCTCTGTATAAAACTTAGCTCTAAAGCAGAAACTAAAGCAGC |
| | AAATGCAGGAAGGCTGTCTCGCCATCCTCAAGACTCAGCAGCTCTCATTCTCCAG |
| | TGGTGAGCACACCATTTGTGCTGCTGCTGTTGTCGTGAAATATAATAACAGTGGA |
| | AGTCACAAAAATGTCCCCTGCCCAGCCCCCTCGCCGCCCTTGACCTCCTGCAGGC |
| | CATGTGTGTATTACTTGTCTAGTGATGTCCTCTCAAAGTGCTGTACGCGAGCTCG |
| | GCGCCACCTCCGCCTCCCTTTCAGAGCCTGCTCCCCGCCCTCTCTGCTCGCTGCA |
| | TTGTGGTGTTCTCTTCTCAAGGCTTTGAAATCTCCCCTTGCACTGAGATTAGTCG |
| | TCAGATCTCTCCCCGTCTCCCTCCCAACTTATACGACCTGATTTCCTTAGGACGG |
| | AACCGCAGGCACCTGCGCCGGGCGTCTTACTCCCGCTGCTTGTTCTGTCCCCTCC |
| | CTCGGACCAAACAGTGCTCATGCTTCAGGACCTTGTTTGTCGAAGATGTTGGTTT |
| | CCCTTTCTCTGTTATTTATATAAAAATAATTTATCAAAAGGATATTTTAAAAAAG |
| | CTAGTCTGTCTTGAAACTTGTTTACCTTAAAATTATCAGAATCTCAGTGTTTGAA |
| | AGTACTGAAGCACAAACATATATCATCTCTGTACCATTCTGTACTAAAGCACTTG |
| | AGTCTAATAAATAAAGAAATCAGCACCCCT |
| SEQ ID NO: | AGTCGGTCCCTAGCGCGGCTGCGGGGCGGAGAGCTGCGGCTGGCCCAGCGCGCCC |
| 23 | ACCTGAGGAGGCGGCGGGGTCCGCAGGCGTCGCGGGACGAGGAGATCGGAGCCGG |
| Human | GAGACTCGCGCAGCGCCATGGCCCCCATTGGCCTCAAAGCTGTTGTCGGAGAGAA |
| STXBP1 | GATTATGCATGATGTGATAAAGAAGGTCAAGAAGAAGGGGGAATGGAAGGTGCTG |
| (transcript | GTGGTGGATCAGTTAAGCATGAGGATGCTGTCCTCCTGCTGCAAGATGACAGACA |
| variant 2) | TCATGACCGAGGGCATAACGATTGTGGAAGATATCAATAAGCGCAGAGAGCCGCT |
| encoding | CCCCAGCCTGGAGGCTGTGTATCTCATCACTCCATCCGAGAAGTCCGTCCACTCT |
| isoform b | CTCATCAGTGACTTTAAGGACCCGCCGACTGCTAAATACCGGGCTGCACACGTCT |
| | TCTTCACTGACTCTTGTCCAGATGCCCTGTTTAATGAACTGGTAAAATCCCGAGC |
| | AGCCAAAGTCATCAAAACTCTGACGGAAATCAATATTGCATTTCTCCCGTATGAA |
| | TCCCAGGTCTATTCCTTGGACTCTGCTGACTCTTTCCAAAGCTTCTACAGTCCCC |
| | ACAAGGCTCAGATGAAGAATCCTATACTGGAGCGCCTGGCAGAGCAGATCGCGAC |
| | CCTTTGTGCCACCCTGAAGGAGTACCCGGCTGTGCGGTATCGGGGGGAATACAAG |
| | GACAATGCCCTGCTGGCTCAGCTAATCCAGGACAAGCTCGATGCCTATAAAGCTG |
| | ATGATCCAACAATGGGGGAGGGCCCAGACAAGGCACGCTCCCAGCTCCTGATCCT |
| | GGATCGAGGCTTTGACCCCAGCTCCCCTGTGCTCCATGAATTGACTTTTCAGGCT |
| | ATGAGTTATGATCTGCTGCCTATCGAAAATGATGTATACAAGTATGAGACCAGCG |
| | GCATCGGGGAGGCACGGGTGAAGGAGGTGCTCCTGGACGAGGACGACGACCTGTG |
| | GATAGCACTGCGCCACAAGCACATCGCAGAGGTGTCCCAGGAAGTCACCCGGTCT |
| | CTGAAAGATTTTTCTTCTAGCAAGAGAATGAATACTGGAGAGAAGACCACCATGC |
| | GGGACCTGTCCCAGATGCTGAAGAAGATGCCTCAGTACCAGAAAGAGCTCAGCAA |
| | GTACTCCACCCACCTGCACCTTGCTGAGGACTGTATGAAGCATTACCAAGGCACC |
| | GTAGACAAACTCTGCCGAGTGGAGCAGGACCTGGCCATGGGCACAGATGCTGAGG |
| | GAGAGAAGATCAAGGACCCTATGCGAGCCATCGTCCCCATTCTGCTGGATGCCAA |
| | TGTCAGCACTTATGACAAAATCCGCATCATCCTTCTCTACATCTTTTTGAAGAAT |
| | GGCATCACGGAGGAAAACCTGAACAAACTGATCCAGCACGCCCAGATACCCCCGG |
| | AGGATAGTGAGATCATCACCAACATGGCTCACCTCGGCGTGCCCATCGTCACCGA |
| | TTCCACGCTGCGTCGCCGGAGCAAGCCGGAGCGGAAGGAACGCATCAGCGAGCAG |
| | ACCTACCAGCTCTCACGGTGGACTCCGATTATCAAGGACATCATGGAGGACACTA |
| | TTGAGGACAAACTTGACACCAAACACTACCCTTATATCTCTACCCGTTCCTCTGC |
| | CTCCTTCAGCACCACCGCCGTCAGCGCCCGCTATGGGCACTGGCATAAGAACAAG |
| | GCCCCAGGCGAGTACCGCAGTGGCCCCCGCCTCATCATTTTCATCCTTGGGGGTG |
| | TGAGCCTGAATGAGATGCGCTGCGCCTACGAGGTGACCCAGGCCAACGGAAAGTG |
| | GGAGGTGCTGATAGGATCCACACACATCCTCACCCCACAGAAACTGCTGGACACA |
| | CTGAAGAAACTGAATAAAACAGATGAAGAAATAAGCAGTTAAAAAAATAAGTCGC |
| | CCCTCCAAAACACGCCCCCATCCCACAGCGCTCCGCAGCTTCCCACCACCGCCCG |
| | CCTCAGTTCCTTTGCGTCTGTTGCCTCCCCAGCCCTGCACGCCCTGGCTGGCACT |
| | GTTGCCGCTGCATTCTCGTGTTCAGTGATGCCCTCTTCTTGTTTGAAACAAAAGA |
| | AAATAATGCATTGTGTTTTTTAAAAAGAGTATCTTATACATGTATCCTAAAAAGA |
| | GAAGCTCATGTGCAATTGGTGCACAGCAGGAGAAATTTCTGGACTGTTAGGATGA |
| | ATGGACGCCTTCTCCCCGTTATTTAAGATTTGTGACCTTGTACATAACCCTGGGT |
| | GACGTGCACATTGCTTGGGTATGGAACGGTAGAAATTTGGGTGTTTTTAAAACCT |
| | TGTTTGGGGTTGTTCCTGTCCTTGTTGAGAATCATAGAGATGTCTGTGTTCTTGG |
| | AGTATTTCACACTGAGGACTAATCTGCTATCTTCATTCCAGTCCCTACCCCTCAG |
| | TGCCTGCTCTCATCCAAATAACCTGGGAGGTGACAATCAGGATATCTCAGGAGGT |
| | CCAAGGTGGAACAGACCTCTTTGCCTTTCCCAGCGTCTCATACCCCCGGTAGTGC |
| | AGCTGTGGGTGGAGGCTGGGGTGTCTGCACGAAGTCAGGCCAGCGTCCTCCTCCA |
| | CAGCCTGTCACTGCCCCCTCCCCAGCCTGTGTCCACAGTGCTGTGATCCCGAGGG |
| | AAGTCCTCCAGTCTAAGTCACAGTGCCCTGACAGGTGAGAAGCAAACTCCCGCTG |
| | GAAGCCTCCATCTCTTTGGAAAAACAGTTAGTCTGGAGCCTGTGGCCCAGGCCCT |
| | TCTGTCCCCAGGCATCATCCCAACAGCTCATTTTCCCTAGTCCGCCTTCGTTCAA |
| | GGGTCAGGAATGGACCAGAACAGATGGGTTCTGGAGGCCCCTGAACAGAGGGCTA |
| | TGGCTGTGGAGAAGGTTCTTGGCCCGTTGGACTCACACAGACCCTGTACCCTCTC |
| | GGCAAGCATCTTCAGTCAGATTATCCTCAGTTTCAGATACTTCATAATACCTTGT |
| | GTTGTGTGGGGTCATACATCATCGTGTTTGTAAGAGAAGATGGTCATTTTATTCT |
| | CTGTATAAAACTTAGCTCTAAAGCAGAAACTAAAGCAGCAAATGCAGGAAGGCTG |
| | TCTCGCCATCCTCAAGACTCAGCAGCTCTCATTCTCCAGTGGTGAGCACACCATT |
| | TGTGCTGCTGCTGTTGTCGTGAAATATAATAACAGTGGAAGTCACAAAAATGTCC |
| | CCTGCCCAGCCCCCTCGCCGCCCTTGACCTCCTGCAGGCCATGTGTGTATTACTT |
| | GTCTAGTGATGTCCTCTCAAAGTGCTGTACGCGAGCTCGGCGCCACCTCCGCCTC |
| | CCTTTCAGAGCCTGCTCCCCGCCCTCTCTGCTCGCTGCATTGTGGTGTTCTCTTC |
| | TCAAGGCTTTGAAATCTCCCCTTGCACTGAGATTAGTCGTCAGATCTCTCCCCGT |
| | CTCCCTCCCAACTTATACGACCTGATTTCCTTAGGACGGAACCGCAGGCACCTGC |
| | GCCGGGCGTCTTACTCCCGCTGCTTGTTCTGTCCCCTCCCTCGGACCAAACAGTG |
| | CTCATGCTTCAGGACCTTGTTTGTCGAAGATGTTGGTTTCCCTTTCTCTGTTATT |
| | TATATAAAAATAATTTATCAAAAGGATATTTTAAAAAAGCTAGTCTGTCTTGAAA |
| | CTTGTTTACCTTAAAATTATCAGAATCTCAGTGTTTGAAAGTACTGAAGCACAAA |
| | CATATATCATCTCTGTACCATTCTGTACTAAAGCACTTGAGTCTAATAAATAAAG |
| | AAATCAGCACCCCT |
| SEQ ID NO: | AGTCGGTCCCTAGCGCGGCTGCGGGGCGGAGAGCTGCGGCTGGCCCAGCGCGCCC |
| 24 | ACCTGAGGAGGCGGCGGGGTCCGCAGGCGTCGCGGGACGAGGAGATCGGAGCCGG |
| Human | GAGACTCGCGCAGCGCCATGGCCCCCATTGGCCTCAAAGCTGTTGTCGGAGAGAA |
| STXBP1 | GATTATGCATGATGTGATAAAGAAGGTCAAGAAGAAGGGGGAATGGAAGGTGCTG |
| (transcript | GTGGTGGATCAGTTAAGCATGAGGATGCTGTCCTCCTGCTGCAAGATGACAGACA |
| variant 3) | TCATGACCGAGGGCATAACGATTGTGGAAGATATCAATAAGCGCAGAGAGCCGCT |
| encoding | CCCCAGCCTGGAGGCTGTGTATCTCATCACTCCATCCGAGAAGTCCGTCCACTCT |
| isoform c | CTCATCAGTGACTTTAAGGACCCGCCGACTGCTAAATACCGGGCTGCACACGTCT |
| | TCTTCACTGACTATGCCCTGTTTAATGAACTGGTAAAATCCCGAGCAGCCAAAGT |
| | CATCAAAACTCTGACGGAAATCAATATTGCATTTCTCCCGTATGAATCCCAGGTC |
| | TATTCCTTGGACTCTGCTGACTCTTTCCAAAGCTTCTACAGTCCCCACAAGGCTC |
| | AGATGAAGAATCCTATACTGGAGCGCCTGGCAGAGCAGATCGCGACCCTTTGTGC |
| | CACCCTGAAGGAGTACCCGGCTGTGCGGTATCGGGGGGAATACAAGGACAATGCC |
| | CTGCTGGCTCAGCTAATCCAGGACAAGCTCGATGCCTATAAAGCTGATGATCCAA |
| | CAATGGGGGAGGGCCCAGACAAGGCACGCTCCCAGCTCCTGATCCTGGATCGAGG |
| | CTTTGACCCCAGCTCCCCTGTGCTCCATGAATTGACTTTTCAGGCTATGAGTTAT |
| | GATCTGCTGCCTATCGAAAATGATGTATACAAGTATGAGACCAGCGGCATCGGGG |
| | AGGCACGGGTGAAGGAGGTGCTCCTGGACGAGGACGACGACCTGTGGATAGCACT |
| | GCGCCACAAGCACATCGCAGAGGTGTCCCAGGAAGTCACCCGGTCTCTGAAAGAT |
| | TTTTCTTCTAGCAAGAGAATGAATACTGGAGAGAAGACCACCATGCGGGACCTGT |
| | CCCAGATGCTGAAGAAGATGCCTCAGTACCAGAAAGAGCTCAGCAAGTACTCCAC |
| | CCACCTGCACCTTGCTGAGGACTGTATGAAGCATTACCAAGGCACCGTAGACAAA |
| | CTCTGCCGAGTGGAGCAGGACCTGGCCATGGGCACAGATGCTGAGGGAGAGAAGA |
| | TCAAGGACCCTATGCGAGCCATCGTCCCCATTCTGCTGGATGCCAATGTCAGCAC |
| | TTATGACAAAATCCGCATCATCCTTCTCTACATCTTTTTGAAGAATGGCATCACG |
| | GAGGAAAACCTGAACAAACTGATCCAGCACGCCCAGATACCCCCGGAGGATAGTG |
| | AGATCATCACCAACATGGCTCACCTCGGCGTGCCCATCGTCACCGATTCCACGCT |
| | GCGTCGCCGGAGCAAGCCGGAGCGGAAGGAACGCATCAGCGAGCAGACCTACCAG |
| | CTCTCACGGTGGACTCCGATTATCAAGGACATCATGGAGGACACTATTGAGGACA |
| | AACTTGACACCAAACACTACCCTTATATCTCTACCCGTTCCTCTGCCTCCTTCAG |
| | CACCACCGCCGTCAGCGCCCGCTATGGGCACTGGCATAAGAACAAGGCCCCAGGC |
| | GAGTACCGCAGTGGCCCCCGCCTCATCATTTTCATCCTTGGGGGTGTGAGCCTGA |
| | ATGAGATGCGCTGCGCCTACGAGGTGACCCAGGCCAACGGAAAGTGGGAGGTGCT |
| | GATAGGATCCACACACATCCTCACCCCACAGAAACTGCTGGACACACTGAAGAAA |
| | CTGAATAAAACAGATGAAGAAATAAGCAGTTAAAAAAATAAGTCGCCCCTCCAAA |
| | ACACGCCCCCATCCCACAGCGCTCCGCAGCTTCCCACCACCGCCCGCCTCAGTTC |
| | CTTTGCGTCTGTTGCCTCCCCAGCCCTGCACGCCCTGGCTGGCACTGTTGCCGCT |
| | GCATTCTCGTGTTCAGTGATGCCCTCTTCTTGTTTGAAACAAAAGAAAATAATGC |
| | ATTGTGTTTTTTAAAAAGAGTATCTTATACATGTATCCTAAAAAGAGAAGCTCAT |
| | GTGCAATTGGTGCACAGCAGGAGAAATTTCTGGACTGTTAGGATGAATGGACGCC |
| | TTCTCCCCGTTATTTAAGATTTGTGACCTTGTACATAACCCTGGGTGACGTGCAC |
| | ATTGCTTGGGTATGGAACGGTAGAAATTTGGGTGTTTTTAAAACCTTGTTTGGGG |
| | TTGTTCCTGTCCTTGTTGAGAATCATAGAGATGTCTGTGTTCTTGGAGTATTTCA |
| | CACTGAGGACTAATCTGCTATCTTCATTCCAGTCCCTACCCCTCAGTGCCTGCTC |
| | TCATCCAAATAACCTGGGAGGTGACAATCAGGATATCTCAGGAGGTCCAAGGTGG |
| | AACAGACCTCTTTGCCTTTCCCAGCGTCTCATACCCCCGGTAGTGCAGCTGTGGG |
| | TGGAGGCTGGGGTGTCTGCACGAAGTCAGGCCAGCGTCCTCCTCCACAGCCTGTC |
| | ACTGCCCCCTCCCCAGCCTGTGTCCACAGTGCTGTGATCCCGAGGGAAGTCCTCC |
| | AGTCTAAGTCACAGTGCCCTGACAGGTGAGAAGCAAACTCCCGCTGGAAGCCTCC |
| | ATCTCTTTGGAAAAACAGTTAGTCTGGAGCCTGTGGCCCAGGCCCTTCTGTCCCC |
| | AGGCATCATCCCAACAGCTCATTTTCCCTAGTCCGCCTTCGTTCAAGGGTCAGGA |
| | ATGGACCAGAACAGATGGGTTCTGGAGGCCCCTGAACAGAGGGCTATGGCTGTGG |
| | AGAAGGTTCTTGGCCCGTTGGACTCACACAGACCCTGTACCCTCTCGGCAAGCAT |
| | CTTCAGTCAGATTATCCTCAGTTTCAGATACTTCATAATACCTTGTGTTGTGTGG |
| | GGTCATACATCATCGTGTTTGTAAGAGAAGATGGTCATTTTATTCTCTGTATAAA |
| | ACTTAGCTCTAAAGCAGAAACTAAAGCAGCAAATGCAGGAAGGCTGTCTCGCCAT |
| | CCTCAAGACTCAGCAGCTCTCATTCTCCAGTGGTGAGCACACCATTTGTGCTGCT |
| | GCTGTTGTCGTGAAATATAATAACAGTGGAAGTCACAAAAATGTCCCCTGCCCAG |
| | CCCCCTCGCCGCCCTTGACCTCCTGCAGGCCATGTGTGTATTACTTGTCTAGTGA |
| | TGTCCTCTCAAAGTGCTGTACGCGAGCTCGGCGCCACCTCCGCCTCCCTTTCAGA |
| | GCCTGCTCCCCGCCCTCTCTGCTCGCTGCATTGTGGTGTTCTCTTCTCAAGGCTT |
| | TGAAATCTCCCCTTGCACTGAGATTAGTCGTCAGATCTCTCCCCGTCTCCCTCCC |
| | AACTTATACGACCTGATTTCCTTAGGACGGAACCGCAGGCACCTGCGCCGGGCGT |
| | CTTACTCCCGCTGCTTGTTCTGTCCCCTCCCTCGGACCAAACAGTGCTCATGCTT |
| | CAGGACCTTGTTTGTCGAAGATGTTGGTTTCCCTTTCTCTGTTATTTATATAAAA |
| | ATAATTTATCAAAAGGATATTTTAAAAAAGCTAGTCTGTCTTGAAACTTGTTTAC |
| | CTTAAAATTATCAGAATCTCAGTGTTTGAAAGTACTGAAGCACAAACATATATCA |
| | TCTCTGTACCATTCTGTACTAAAGCACTTGAGTCTAATAAATAAAGAAATCAGCA |
| | CCCCT |
| SEQ ID NO: | AGTCGGTCCCTAGCGCGGCTGCGGGGCGGAGAGCTGCGGCTGGCCCAGCGCGCCC |
| 25 | ACCTGAGGAGGCGGCGGGGTCCGCAGGCGTCGCGGGACGAGGAGATCGGAGCCGG |
| Human | GAGACTCGCGCAGCGCCATGGCCCCCATTGGCCTCAAAGCTGTTGTCGGAGAGAG |
| STXBP1 | AAAGAGGAAGACTACCTAGACATGTTGCTCCAGAGCAGAATTTCCAGCAGTAGAT |
| (transcript | TGAACCTTCGGAGGCCGAGTCCCAGCCCAGTGTAAACTGAGATTATGCATGATGT |
| variant 4) | GATAAAGAAGGTCAAGAAGAAGGGGGAATGGAAGGTGCTGGTGGTGGATCAGTTA |
| encoding | AGCATGAGGATGCTGTCCTCCTGCTGCAAGATGACAGACATCATGACCGAGGGCA |
| isoform d | TAACGATTGTGGAAGATATCAATAAGCGCAGAGAGCCGCTCCCCAGCCTGGAGGC |
| | TGTGTATCTCATCACTCCATCCGAGAAGTCCGTCCACTCTCTCATCAGTGACTTT |
| | AAGGACCCGCCGACTGCTAAATACCGGGCTGCACACGTCTTCTTCACTGACTCTT |
| | GTCCAGATGCCCTGTTTAATGAACTGGTAAAATCCCGAGCAGCCAAAGTCATCAA |
| | AACTCTGACGGAAATCAATATTGCATTTCTCCCGTATGAATCCCAGGTCTATTCC |
| | TTGGACTCTGCTGACTCTTTCCAAAGCTTCTACAGTCCCCACAAGGCTCAGATGA |
| | AGAATCCTATACTGGAGCGCCTGGCAGAGCAGATCGCGACCCTTTGTGCCACCCT |
| | GAAGGAGTACCCGGCTGTGCGGTATCGGGGGGAATACAAGGACAATGCCCTGCTG |
| | GCTCAGCTAATCCAGGACAAGCTCGATGCCTATAAAGCTGATGATCCAACAATGG |
| | GGGAGGGCCCAGACAAGGCACGCTCCCAGCTCCTGATCCTGGATCGAGGCTTTGA |
| | CCCCAGCTCCCCTGTGCTCCATGAATTGACTTTTCAGGCTATGAGTTATGATCTG |
| | CTGCCTATCGAAAATGATGTATACAAGTATGAGACCAGCGGCATCGGGGAGGCAC |
| | GGGTGAAGGAGGTGCTCCTGGACGAGGACGACGACCTGTGGATAGCACTGCGCCA |
| | CAAGCACATCGCAGAGGTGTCCCAGGAAGTCACCCGGTCTCTGAAAGATTTTTCT |
| | TCTAGCAAGAGAATGAATACTGGAGAGAAGACCACCATGCGGGACCTGTCCCAGA |
| | TGCTGAAGAAGATGCCTCAGTACCAGAAAGAGCTCAGCAAGTACTCCACCCACCT |
| | GCACCTTGCTGAGGACTGTATGAAGCATTACCAAGGCACCGTAGACAAACTCTGC |
| | CGAGTGGAGCAGGACCTGGCCATGGGCACAGATGCTGAGGGAGAGAAGATCAAGG |
| | ACCCTATGCGAGCCATCGTCCCCATTCTGCTGGATGCCAATGTCAGCACTTATGA |
| | CAAAATCCGCATCATCCTTCTCTACATCTTTTTGAAGAATGGCATCACGGAGGAA |
| | AACCTGAACAAACTGATCCAGCACGCCCAGATACCCCCGGAGGATAGTGAGATCA |
| | TCACCAACATGGCTCACCTCGGCGTGCCCATCGTCACCGATTCCACGCTGCGTCG |
| | CCGGAGCAAGCCGGAGCGGAAGGAACGCATCAGCGAGCAGACCTACCAGCTCTCA |
| | CGGTGGACTCCGATTATCAAGGACATCATGGAGGACACTATTGAGGACAAACTTG |
| | ACACCAAACACTACCCTTATATCTCTACCCGTTCCTCTGCCTCCTTCAGCACCAC |
| | CGCCGTCAGCGCCCGCTATGGGCACTGGCATAAGAACAAGGCCCCAGGCGAGTAC |
| | CGCAGTGGCCCCCGCCTCATCATTTTCATCCTTGGGGGTGTGAGCCTGAATGAGA |
| | TGCGCTGCGCCTACGAGGTGACCCAGGCCAACGGAAAGTGGGAGGTGCTGATAGG |
| | TTCTACTCACATTCTTACTCCCACCAAATTTCTCATGGACCTGAGACACCCCGAC |
| | TTCAGGGAGTCCTCTAGGGTATCTTTTGAGGATCAGGCTCCAACAATGGAGTGAG |
| | AGCCAAAGAAACAAAGATCCACACACATCCTCACCCCACAGAAACTGCTGGACAC |
| | ACTGAAGAAACTGAATAAAACAGATGAAGAAATAAGCAGTTAAAAAAATAAGTCG |
| | CCCCTCCAAAACACGCCCCCATCCCACAGCGCTCCGCAGCTTCCCACCACCGCCC |
| | GCCTCAGTTCCTTTGCGTCTGTTGCCTCCCCAGCCCTGCACGCCCTGGCTGGCAC |
| | TGTTGCCGCTGCATTCTCGTGTTCAGTGATGCCCTCTTCTTGTTTGAAACAAAAG |
| | AAAATAATGCATTGTGTTTTTTAAAAAGAGTATCTTATACATGTATCCTAAAAAG |
| | AGAAGCTCATGTGCAATTGGTGCACAGCAGGAGAAATTTCTGGACTGTTAGGATG |
| | AATGGACGCCTTCTCCCCGTTATTTAAGATTTGTGACCTTGTACATAACCCTGGG |
| | TGACGTGCACATTGCTTGGGTATGGAACGGTAGAAATTTGGGTGTTTTTAAAACC |
| | TTGTTTGGGGTTGTTCCTGTCCTTGTTGAGAATCATAGAGATGTCTGTGTTCTTG |
| | GAGTATTTCACACTGAGGACTAATCTGCTATCTTCATTCCAGTCCCTACCCCTCA |
| | GTGCCTGCTCTCATCCAAATAACCTGGGAGGTGACAATCAGGATATCTCAGGAGG |
| | TCCAAGGTGGAACAGACCTCTTTGCCTTTCCCAGCGTCTCATACCCCCGGTAGTG |
| | CAGCTGTGGGTGGAGGCTGGGGTGTCTGCACGAAGTCAGGCCAGCGTCCTCCTCC |
| | ACAGCCTGTCACTGCCCCCTCCCCAGCCTGTGTCCACAGTGCTGTGATCCCGAGG |
| | GAAGTCCTCCAGTCTAAGTCACAGTGCCCTGACAGGTGAGAAGCAAACTCCCGCT |
| | GGAAGCCTCCATCTCTTTGGAAAAACAGTTAGTCTGGAGCCTGTGGCCCAGGCCC |
| | TTCTGTCCCCAGGCATCATCCCAACAGCTCATTTTCCCTAGTCCGCCTTCGTTCA |
| | AGGGTCAGGAATGGACCAGAACAGATGGGTTCTGGAGGCCCCTGAACAGAGGGCT |
| | ATGGCTGTGGAGAAGGTTCTTGGCCCGTTGGACTCACACAGACCCTGTACCCTCT |
| | CGGCAAGCATCTTCAGTCAGATTATCCTCAGTTTCAGATACTTCATAATACCTTG |
| | TGTTGTGTGGGGTCATACATCATCGTGTTTGTAAGAGAAGATGGTCATTTTATTC |
| | TCTGTATAAAACTTAGCTCTAAAGCAGAAACTAAAGCAGCAAATGCAGGAAGGCT |
| | GTCTCGCCATCCTCAAGACTCAGCAGCTCTCATTCTCCAGTGGTGAGCACACCAT |
| | TTGTGCTGCTGCTGTTGTCGTGAAATATAATAACAGTGGAAGTCACAAAAATGTC |
| | CCCTGCCCAGCCCCCTCGCCGCCCTTGACCTCCTGCAGGCCATGTGTGTATTACT |
| | TGTCTAGTGATGTCCTCTCAAAGTGCTGTACGCGAGCTCGGCGCCACCTCCGCCT |
| | CCCTTTCAGAGCCTGCTCCCCGCCCTCTCTGCTCGCTGCATTGTGGTGTTCTCTT |
| | CTCAAGGCTTTGAAATCTCCCCTTGCACTGAGATTAGTCGTCAGATCTCTCCCCG |
| | TCTCCCTCCCAACTTATACGACCTGATTTCCTTAGGACGGAACCGCAGGCACCTG |
| | CGCCGGGCGTCTTACTCCCGCTGCTTGTTCTGTCCCCTCCCTCGGACCAAACAGT |
| | GCTCATGCTTCAGGACCTTGTTTGTCGAAGATGTTGGTTTCCCTTTCTCTGTTAT |
| | TTATATAAAAATAATTTATCAAAAGGATATTTTAAAAAAGCTAGTCTGTCTTGAA |
| | ACTTGTTTACCTTAAAATTATCAGAATCTCAGTGTTTGAAAGTACTGAAGCACAA |
| | ACATATATCATCTCTGTACCATTCTGTACTAAAGCACTTGAGTCTAATAAATAAA |
| | GAAATCAGCACCCCT |
| SEQ ID NO: | AGTCGGTCCCTAGCGCGGCTGCGGGGCGGAGAGCTGCGGCTGGCCCAGCGCGCCC |
| 26 | ACCTGAGGAGGCGGCGGGGTCCGCAGGCGTCGCGGGACGAGGAGATCGGAGCCGG |
| Human | GAGACTCGCGCAGCGCCATGGCCCCCATTGGCCTCAAAGCTGTTGTCGGAGAGAG |
| STXBP1 | ATATATACCTAGGAATGGAGTTGCTGGATCTCTATGTTTGGTTATTTGAGGAGCT |
| (transcript | GCCGGCCTGTTTTCCACATCAGCTGTACCATTTTACATTCTCACCAGCAGTGCAT |
| variant 5) | GACGGTCCAGTGCCTTTTTATCTTACTGCGCTTAAGGTGTAATTCCTTCTTTCTC |
| encoding | AGACTGGACTGTAAATTCTTTGAGAGATTATGCATGATGTGATAAAGAAGGTCAA |
| isoform d | GAAGAAGGGGGAATGGAAGGTGCTGGTGGTGGATCAGTTAAGCATGAGGATGCTG |
| | TCCTCCTGCTGCAAGATGACAGACATCATGACCGAGGGCATAACGATTGTGGAAG |
| | ATATCAATAAGCGCAGAGAGCCGCTCCCCAGCCTGGAGGCTGTGTATCTCATCAC |
| | TCCATCCGAGAAGTCCGTCCACTCTCTCATCAGTGACTTTAAGGACCCGCCGACT |
| | GCTAAATACCGGGCTGCACACGTCTTCTTCACTGACTCTTGTCCAGATGCCCTGT |
| | TTAATGAACTGGTAAAATCCCGAGCAGCCAAAGTCATCAAAACTCTGACGGAAAT |
| | CAATATTGCATTTCTCCCGTATGAATCCCAGGTCTATTCCTTGGACTCTGCTGAC |
| | TCTTTCCAAAGCTTCTACAGTCCCCACAAGGCTCAGATGAAGAATCCTATACTGG |
| | AGCGCCTGGCAGAGCAGATCGCGACCCTTTGTGCCACCCTGAAGGAGTACCCGGC |
| | TGTGCGGTATCGGGGGGAATACAAGGACAATGCCCTGCTGGCTCAGCTAATCCAG |
| | GACAAGCTCGATGCCTATAAAGCTGATGATCCAACAATGGGGGAGGGCCCAGACA |
| | AGGCACGCTCCCAGCTCCTGATCCTGGATCGAGGCTTTGACCCCAGCTCCCCTGT |
| | GCTCCATGAATTGACTTTTCAGGCTATGAGTTATGATCTGCTGCCTATCGAAAAT |
| | GATGTATACAAGTATGAGACCAGCGGCATCGGGGAGGCACGGGTGAAGGAGGTGC |
| | TCCTGGACGAGGACGACGACCTGTGGATAGCACTGCGCCACAAGCACATCGCAGA |
| | GGTGTCCCAGGAAGTCACCCGGTCTCTGAAAGATTTTTCTTCTAGCAAGAGAATG |
| | AATACTGGAGAGAAGACCACCATGCGGGACCTGTCCCAGATGCTGAAGAAGATGC |
| | CTCAGTACCAGAAAGAGCTCAGCAAGTACTCCACCCACCTGCACCTTGCTGAGGA |
| | CTGTATGAAGCATTACCAAGGCACCGTAGACAAACTCTGCCGAGTGGAGCAGGAC |
| | CTGGCCATGGGCACAGATGCTGAGGGAGAGAAGATCAAGGACCCTATGCGAGCCA |
| | TCGTCCCCATTCTGCTGGATGCCAATGTCAGCACTTATGACAAAATCCGCATCAT |
| | CCTTCTCTACATCTTTTTGAAGAATGGCATCACGGAGGAAAACCTGAACAAACTG |
| | ATCCAGCACGCCCAGATACCCCCGGAGGATAGTGAGATCATCACCAACATGGCTC |
| | ACCTCGGCGTGCCCATCGTCACCGATTCCACGCTGCGTCGCCGGAGCAAGCCGGA |
| | GCGGAAGGAACGCATCAGCGAGCAGACCTACCAGCTCTCACGGTGGACTCCGATT |
| | ATCAAGGACATCATGGAGGACACTATTGAGGACAAACTTGACACCAAACACTACC |
| | CTTATATCTCTACCCGTTCCTCTGCCTCCTTCAGCACCACCGCCGTCAGCGCCCG |
| | CTATGGGCACTGGCATAAGAACAAGGCCCCAGGCGAGTACCGCAGTGGCCCCCGC |
| | CTCATCATTTTCATCCTTGGGGGTGTGAGCCTGAATGAGATGCGCTGCGCCTACG |
| | AGGTGACCCAGGCCAACGGAAAGTGGGAGGTGCTGATAGGTTCTACTCACATTCT |
| | TACTCCCACCAAATTTCTCATGGACCTGAGACACCCCGACTTCAGGGAGTCCTCT |
| | AGGGTATCTTTTGAGGATCAGGCTCCAACAATGGAGTGAGAGCCAAAGAAACAAA |
| | GATCCACACACATCCTCACCCCACAGAAACTGCTGGACACACTGAAGAAACTGAA |
| | TAAAACAGATGAAGAAATAAGCAGTTAAAAAAATAAGTCGCCCCTCCAAAACACG |
| | CCCCCATCCCACAGCGCTCCGCAGCTTCCCACCACCGCCCGCCTCAGTTCCTTTG |
| | CGTCTGTTGCCTCCCCAGCCCTGCACGCCCTGGCTGGCACTGTTGCCGCTGCATT |
| | CTCGTGTTCAGTGATGCCCTCTTCTTGTTTGAAACAAAAGAAAATAATGCATTGT |
| | GTTTTTTAAAAAGAGTATCTTATACATGTATCCTAAAAAGAGAAGCTCATGTGCA |
| | ATTGGTGCACAGCAGGAGAAATTTCTGGACTGTTAGGATGAATGGACGCCTTCTC |
| | CCCGTTATTTAAGATTTGTGACCTTGTACATAACCCTGGGTGACGTGCACATTGC |
| | TTGGGTATGGAACGGTAGAAATTTGGGTGTTTTTAAAACCTTGTTTGGGGTTGTT |
| | CCTGTCCTTGTTGAGAATCATAGAGATGTCTGTGTTCTTGGAGTATTTCACACTG |
| | AGGACTAATCTGCTATCTTCATTCCAGTCCCTACCCCTCAGTGCCTGCTCTCATC |
| | CAAATAACCTGGGAGGTGACAATCAGGATATCTCAGGAGGTCCAAGGTGGAACAG |
| | ACCTCTTTGCCTTTCCCAGCGTCTCATACCCCCGGTAGTGCAGCTGTGGGTGGAG |
| | GCTGGGGTGTCTGCACGAAGTCAGGCCAGCGTCCTCCTCCACAGCCTGTCACTGC |
| | CCCCTCCCCAGCCTGTGTCCACAGTGCTGTGATCCCGAGGGAAGTCCTCCAGTCT |
| | AAGTCACAGTGCCCTGACAGGTGAGAAGCAAACTCCCGCTGGAAGCCTCCATCTC |
| | TTTGGAAAAACAGTTAGTCTGGAGCCTGTGGCCCAGGCCCTTCTGTCCCCAGGCA |
| | TCATCCCAACAGCTCATTTTCCCTAGTCCGCCTTCGTTCAAGGGTCAGGAATGGA |
| | CCAGAACAGATGGGTTCTGGAGGCCCCTGAACAGAGGGCTATGGCTGTGGAGAAG |
| | GTTCTTGGCCCGTTGGACTCACACAGACCCTGTACCCTCTCGGCAAGCATCTTCA |
| | GTCAGATTATCCTCAGTTTCAGATACTTCATAATACCTTGTGTTGTGTGGGGTCA |
| | TACATCATCGTGTTTGTAAGAGAAGATGGTCATTTTATTCTCTGTATAAAACTTA |
| | GCTCTAAAGCAGAAACTAAAGCAGCAAATGCAGGAAGGCTGTCTCGCCATCCTCA |
| | AGACTCAGCAGCTCTCATTCTCCAGTGGTGAGCACACCATTTGTGCTGCTGCTGT |
| | TGTCGTGAAATATAATAACAGTGGAAGTCACAAAAATGTCCCCTGCCCAGCCCCC |
| | TCGCCGCCCTTGACCTCCTGCAGGCCATGTGTGTATTACTTGTCTAGTGATGTCC |
| | TCTCAAAGTGCTGTACGCGAGCTCGGCGCCACCTCCGCCTCCCTTTCAGAGCCTG |
| | CTCCCCGCCCTCTCTGCTCGCTGCATTGTGGTGTTCTCTTCTCAAGGCTTTGAAA |
| | TCTCCCCTTGCACTGAGATTAGTCGTCAGATCTCTCCCCGTCTCCCTCCCAACTT |
| | ATACGACCTGATTTCCTTAGGACGGAACCGCAGGCACCTGCGCCGGGCGTCTTAC |
| | TCCCGCTGCTTGTTCTGTCCCCTCCCTCGGACCAAACAGTGCTCATGCTTCAGGA |
| | CCTTGTTTGTCGAAGATGTTGGTTTCCCTTTCTCTGTTATTTATATAAAAATAAT |
| | TTATCAAAAGGATATTTTAAAAAAGCTAGTCTGTCTTGAAACTTGTTTACCTTAA |
| | AATTATCAGAATCTCAGTGTTTGAAAGTACTGAAGCACAAACATATATCATCTCT |
| | GTACCATTCTGTACTAAAGCACTTGAGTCTAATAAATAAAGAAATCAGCACCCCT |
| SEQ ID NO: | AGAAAGAGGAAAGACAGGACCCGAGCGGGGTTTCAGGCAGATGGAGCGCGTCGGT |
| 27 | AGCCTGTGGCCAGGGATCCCAGCACCGACGGGAAAGAGGAGGCCTGGGTACCCTG |
| Human | CGCCCCGGGCGCGCGCGGCGCGTGAGAGATTATGCATGATGTGATAAAGAAGGTC |
| STXBP1 | AAGAAGAAGGGGGAATGGAAGGTGCTGGTGGTGGATCAGTTAAGCATGAGGATGC |
| (transcript | TGTCCTCCTGCTGCAAGATGACAGACATCATGACCGAGGGCATAACGATTGTGGA |
| variant 6) | AGATATCAATAAGCGCAGAGAGCCGCTCCCCAGCCTGGAGGCTGTGTATCTCATC |
| encoding | ACTCCATCCGAGAAGTCCGTCCACTCTCTCATCAGTGACTTTAAGGACCCGCCGA |
| isoform e | CTGCTAAATACCGGGCTGCACACGTCTTCTTCACTGACTCTTGTCCAGATGCCCT |
| | GTTTAATGAACTGGTAAAATCCCGAGCAGCCAAAGTCATCAAAACTCTGACGGAA |
| | ATCAATATTGCATTTCTCCCGTATGAATCCCAGGTCTATTCCTTGGACTCTGCTG |
| | ACTCTTTCCAAAGCTTCTACAGTCCCCACAAGGCTCAGATGAAGAATCCTATACT |
| | GGAGCGCCTGGCAGAGCAGATCGCGACCCTTTGTGCCACCCTGAAGGAGTACCCG |
| | GCTGTGCGGTATCGGGGGGAATACAAGGACAATGCCCTGCTGGCTCAGCTAATCC |
| | AGGACAAGCTCGATGCCTATAAAGCTGATGATCCAACAATGGGGGAGGGCCCAGA |
| | CAAGGCACGCTCCCAGCTCCTGATCCTGGATCGAGGCTTTGACCCCAGCTCCCCT |
| | GTGCTCCATGAATTGACTTTTCAGGCTATGAGTTATGATCTGCTGCCTATCGAAA |
| | ATGATGTATACAAGTATGAGACCAGCGGCATCGGGGAGGCACGGGTGAAGGAGGT |
| | GCTCCTGGACGAGGACGACGACCTGTGGATAGCACTGCGCCACAAGCACATCGCA |
| | GAGGTGTCCCAGGAAGTCACCCGGTCTCTGAAAGATTTTTCTTCTAGCAAGAGAA |
| | TGAATACTGGAGAGAAGACCACCATGCGGGACCTGTCCCAGATGCTGAAGAAGAT |
| | GCCTCAGTACCAGAAAGAGCTCAGCAAGTACTCCACCCACCTGCACCTTGCTGAG |
| | GACTGTATGAAGCATTACCAAGGCACCGTAGACAAACTCTGCCGAGTGGAGCAGG |
| | ACCTGGCCATGGGCACAGATGCTGAGGGAGAGAAGATCAAGGACCCTATGCGAGC |
| | CATCGTCCCCATTCTGCTGGATGCCAATGTCAGCACTTATGACAAAATCCGCATC |
| | ATCCTTCTCTACATCTTTTTGAAGAATGGCATCACGGAGGAAAACCTGAACAAAC |
| | TGATCCAGCACGCCCAGATACCCCCGGAGGATAGTGAGATCATCACCAACATGGC |
| | TCACCTCGGCGTGCCCATCGTCACCGATTCCACGCTGCGTCGCCGGAGCAAGCCG |
| | GAGCGGAAGGAACGCATCAGCGAGCAGACCTACCAGCTCTCACGGTGGACTCCGA |
| | TTATCAAGGACATCATGGAGGACACTATTGAGGACAAACTTGACACCAAACACTA |
| | CCCTTATATCTCTACCCGTTCCTCTGCCTCCTTCAGCACCACCGCCGTCAGCGCC |
| | CGCTATGGGCACTGGCATAAGAACAAGGCCCCAGGCGAGTACCGCAGTGGCCCCC |
| | GCCTCATCATTTTCATCCTTGGGGGTGTGAGCCTGAATGAGATGCGCTGCGCCTA |
| | CGAGGTGACCCAGGCCAACGGAAAGTGGGAGGTGCTGATAGGATCCACACACATC |
| | CTCACCCCACAGAAACTGCTGGACACACTGAAGAAACTGAATAAAACAGATGAAG |
| | AAATAAGCAGTTAAAAAAATAAGTCGCCCCTCCAAAACACGCCCCCATCCCACAG |
| | CGCTCCGCAGCTTCCCACCACCGCCCGCCTCAGTTCCTTTGCGTCTGTTGCCTCC |
| | CCAGCCCTGCACGCCCTGGCTGGCACTGTTGCCGCTGCATTCTCGTGTTCAGTGA |
| | TGCCCTCTTCTTGTTTGAAACAAAAGAAAATAATGCATTGTGTTTTTTAAAAAGA |
| | GTATCTTATACATGTATCCTAAAAAGAGAAGCTCATGTGCAATTGGTGCACAGCA |
| | GGAGAAATTTCTGGACTGTTAGGATGAATGGACGCCTTCTCCCCGTTATTTAAGA |
| | TTTGTGACCTTGTACATAACCCTGGGTGACGTGCACATTGCTTGGGTATGGAACG |
| | GTAGAAATTTGGGTGTTTTTAAAACCTTGTTTGGGGTTGTTCCTGTCCTTGTTGA |
| | GAATCATAGAGATGTCTGTGTTCTTGGAGTATTTCACACTGAGGACTAATCTGCT |
| | ATCTTCATTCCAGTCCCTACCCCTCAGTGCCTGCTCTCATCCAAATAACCTGGGA |
| | GGTGACAATCAGGATATCTCAGGAGGTCCAAGGTGGAACAGACCTCTTTGCCTTT |
| | CCCAGCGTCTCATACCCCCGGTAGTGCAGCTGTGGGTGGAGGCTGGGGTGTCTGC |
| | ACGAAGTCAGGCCAGCGTCCTCCTCCACAGCCTGTCACTGCCCCCTCCCCAGCCT |
| | GTGTCCACAGTGCTGTGATCCCGAGGGAAGTCCTCCAGTCTAAGTCACAGTGCCC |
| | TGACAGGTGAGAAGCAAACTCCCGCTGGAAGCCTCCATCTCTTTGGAAAAACAGT |
| | TAGTCTGGAGCCTGTGGCCCAGGCCCTTCTGTCCCCAGGCATCATCCCAACAGCT |
| | CATTTTCCCTAGTCCGCCTTCGTTCAAGGGTCAGGAATGGACCAGAACAGATGGG |
| | TTCTGGAGGCCCCTGAACAGAGGGCTATGGCTGTGGAGAAGGTTCTTGGCCCGTT |
| | GGACTCACACAGACCCTGTACCCTCTCGGCAAGCATCTTCAGTCAGATTATCCTC |
| | AGTTTCAGATACTTCATAATACCTTGTGTTGTGTGGGGTCATACATCATCGTGTT |
| | TGTAAGAGAAGATGGTCATTTTATTCTCTGTATAAAACTTAGCTCTAAAGCAGAA |
| | ACTAAAGCAGCAAATGCAGGAAGGCTGTCTCGCCATCCTCAAGACTCAGCAGCTC |
| | TCATTCTCCAGTGGTGAGCACACCATTTGTGCTGCTGCTGTTGTCGTGAAATATA |
| | ATAACAGTGGAAGTCACAAAAATGTCCCCTGCCCAGCCCCCTCGCCGCCCTTGAC |
| | CTCCTGCAGGCCATGTGTGTATTACTTGTCTAGTGATGTCCTCTCAAAGTGCTGT |
| | ACGCGAGCTCGGCGCCACCTCCGCCTCCCTTTCAGAGCCTGCTCCCCGCCCTCTC |
| | TGCTCGCTGCATTGTGGTGTTCTCTTCTCAAGGCTTTGAAATCTCCCCTTGCACT |
| | GAGATTAGTCGTCAGATCTCTCCCCGTCTCCCTCCCAACTTATACGACCTGATTT |
| | CCTTAGGACGGAACCGCAGGCACCTGCGCCGGGCGTCTTACTCCCGCTGCTTGTT |
| | CTGTCCCCTCCCTCGGACCAAACAGTGCTCATGCTTCAGGACCTTGTTTGTCGAA |
| | GATGTTGGTTTCCCTTTCTCTGTTATTTATATAAAAATAATTTATCAAAAGGATA |
| | TTTTAAAAAAGCTAGTCTGTCTTGAAACTTGTTTACCTTAAAATTATCAGAATCT |
| | CAGTGTTTGAAAGTACTGAAGCACAAACATATATCATCTCTGTACCATTCTGTAC |
| | TAAAGCACTTGAGTCTAATAAATAAAGAAATCAGCACCCCT |
| SEQ ID NO: | AGTCGGTCCCTAGCGCGGCTGCGGGGCGGAGAGCTGCGGCTGGCCCAGCGCGCCC |
| 28 | ACCTGAGGAGGCGGCGGGGTCCGCAGGCGTCGCGGGACGAGGAGATCGGAGCCGG |
| Human | GAGACTCGCGCAGCGCCATGGCCCCCATTGGCCTCAAAGCTGTTGTCGGAGAGAG |
| STXBP1 | ATATATACCTAGGAATGGAGTTGCTGGATCTCTATGTTTGGTTATTTGAGGAGCT |
| (transcript | GCCGGCCTGTTTTCCACATCAGCTGTACCATTTTACATTCTCACCAGCAGTGCAT |
| variant 7) | GACGGTCCAGTGCCTTTTTATCTTACTGCGCTTAAGGTGTAATTCCTTCTTTCTC |
| encoding | AGACTGGACTGTAAATTCTTTGAGAGATTATGCATGATGTGATAAAGAAGGTCAA |
| isoform e | GAAGAAGGGGGAATGGAAGGTGCTGGTGGTGGATCAGTTAAGCATGAGGATGCTG |
| | TCCTCCTGCTGCAAGATGACAGACATCATGACCGAGGGCATAACGATTGTGGAAG |
| | ATATCAATAAGCGCAGAGAGCCGCTCCCCAGCCTGGAGGCTGTGTATCTCATCAC |
| | TCCATCCGAGAAGTCCGTCCACTCTCTCATCAGTGACTTTAAGGACCCGCCGACT |
| | GCTAAATACCGGGCTGCACACGTCTTCTTCACTGACTCTTGTCCAGATGCCCTGT |
| | TTAATGAACTGGTAAAATCCCGAGCAGCCAAAGTCATCAAAACTCTGACGGAAAT |
| | CAATATTGCATTTCTCCCGTATGAATCCCAGGTCTATTCCTTGGACTCTGCTGAC |
| | TCTTTCCAAAGCTTCTACAGTCCCCACAAGGCTCAGATGAAGAATCCTATACTGG |
| | AGCGCCTGGCAGAGCAGATCGCGACCCTTTGTGCCACCCTGAAGGAGTACCCGGC |
| | TGTGCGGTATCGGGGGGAATACAAGGACAATGCCCTGCTGGCTCAGCTAATCCAG |
| | GACAAGCTCGATGCCTATAAAGCTGATGATCCAACAATGGGGGAGGGCCCAGACA |
| | AGGCACGCTCCCAGCTCCTGATCCTGGATCGAGGCTTTGACCCCAGCTCCCCTGT |
| | GCTCCATGAATTGACTTTTCAGGCTATGAGTTATGATCTGCTGCCTATCGAAAAT |
| | GATGTATACAAGTATGAGACCAGCGGCATCGGGGAGGCACGGGTGAAGGAGGTGC |
| | TCCTGGACGAGGACGACGACCTGTGGATAGCACTGCGCCACAAGCACATCGCAGA |
| | GGTGTCCCAGGAAGTCACCCGGTCTCTGAAAGATTTTTCTTCTAGCAAGAGAATG |
| | AATACTGGAGAGAAGACCACCATGCGGGACCTGTCCCAGATGCTGAAGAAGATGC |
| | CTCAGTACCAGAAAGAGCTCAGCAAGTACTCCACCCACCTGCACCTTGCTGAGGA |
| | CTGTATGAAGCATTACCAAGGCACCGTAGACAAACTCTGCCGAGTGGAGCAGGAC |
| | CTGGCCATGGGCACAGATGCTGAGGGAGAGAAGATCAAGGACCCTATGCGAGCCA |
| | TCGTCCCCATTCTGCTGGATGCCAATGTCAGCACTTATGACAAAATCCGCATCAT |
| | CCTTCTCTACATCTTTTTGAAGAATGGCATCACGGAGGAAAACCTGAACAAACTG |
| | ATCCAGCACGCCCAGATACCCCCGGAGGATAGTGAGATCATCACCAACATGGCTC |
| | ACCTCGGCGTGCCCATCGTCACCGATTCCACGCTGCGTCGCCGGAGCAAGCCGGA |
| | GCGGAAGGAACGCATCAGCGAGCAGACCTACCAGCTCTCACGGTGGACTCCGATT |
| | ATCAAGGACATCATGGAGGACACTATTGAGGACAAACTTGACACCAAACACTACC |
| | CTTATATCTCTACCCGTTCCTCTGCCTCCTTCAGCACCACCGCCGTCAGCGCCCG |
| | CTATGGGCACTGGCATAAGAACAAGGCCCCAGGCGAGTACCGCAGTGGCCCCCGC |
| | CTCATCATTTTCATCCTTGGGGGTGTGAGCCTGAATGAGATGCGCTGCGCCTACG |
| | AGGTGACCCAGGCCAACGGAAAGTGGGAGGTGCTGATAGGATCCACACACATCCT |
| | CACCCCACAGAAACTGCTGGACACACTGAAGAAACTGAATAAAACAGATGAAGAA |
| | ATAAGCAGTTAAAAAAATAAGTCGCCCCTCCAAAACACGCCCCCATCCCACAGCG |
| | CTCCGCAGCTTCCCACCACCGCCCGCCTCAGTTCCTTTGCGTCTGTTGCCTCCCC |
| | AGCCCTGCACGCCCTGGCTGGCACTGTTGCCGCTGCATTCTCGTGTTCAGTGATG |
| | CCCTCTTCTTGTTTGAAACAAAAGAAAATAATGCATTGTGTTTTTTAAAAAGAGT |
| | ATCTTATACATGTATCCTAAAAAGAGAAGCTCATGTGCAATTGGTGCACAGCAGG |
| | AGAAATTTCTGGACTGTTAGGATGAATGGACGCCTTCTCCCCGTTATTTAAGATT |
| | TGTGACCTTGTACATAACCCTGGGTGACGTGCACATTGCTTGGGTATGGAACGGT |
| | AGAAATTTGGGTGTTTTTAAAACCTTGTTTGGGGTTGTTCCTGTCCTTGTTGAGA |
| | ATCATAGAGATGTCTGTGTTCTTGGAGTATTTCACACTGAGGACTAATCTGCTAT |
| | CTTCATTCCAGTCCCTACCCCTCAGTGCCTGCTCTCATCCAAATAACCTGGGAGG |
| | TGACAATCAGGATATCTCAGGAGGTCCAAGGTGGAACAGACCTCTTTGCCTTTCC |
| | CAGCGTCTCATACCCCCGGTAGTGCAGCTGTGGGTGGAGGCTGGGGTGTCTGCAC |
| | GAAGTCAGGCCAGCGTCCTCCTCCACAGCCTGTCACTGCCCCCTCCCCAGCCTGT |
| | GTCCACAGTGCTGTGATCCCGAGGGAAGTCCTCCAGTCTAAGTCACAGTGCCCTG |
| | ACAGGTGAGAAGCAAACTCCCGCTGGAAGCCTCCATCTCTTTGGAAAAACAGTTA |
| | GTCTGGAGCCTGTGGCCCAGGCCCTTCTGTCCCCAGGCATCATCCCAACAGCTCA |
| | TTTTCCCTAGTCCGCCTTCGTTCAAGGGTCAGGAATGGACCAGAACAGATGGGTT |
| | CTGGAGGCCCCTGAACAGAGGGCTATGGCTGTGGAGAAGGTTCTTGGCCCGTTGG |
| | ACTCACACAGACCCTGTACCCTCTCGGCAAGCATCTTCAGTCAGATTATCCTCAG |
| | TTTCAGATACTTCATAATACCTTGTGTTGTGTGGGGTCATACATCATCGTGTTTG |
| | TAAGAGAAGATGGTCATTTTATTCTCTGTATAAAACTTAGCTCTAAAGCAGAAAC |
| | TAAAGCAGCAAATGCAGGAAGGCTGTCTCGCCATCCTCAAGACTCAGCAGCTCTC |
| | ATTCTCCAGTGGTGAGCACACCATTTGTGCTGCTGCTGTTGTCGTGAAATATAAT |
| | AACAGTGGAAGTCACAAAAATGTCCCCTGCCCAGCCCCCTCGCCGCCCTTGACCT |
| | CCTGCAGGCCATGTGTGTATTACTTGTCTAGTGATGTCCTCTCAAAGTGCTGTAC |
| | GCGAGCTCGGCGCCACCTCCGCCTCCCTTTCAGAGCCTGCTCCCCGCCCTCTCTG |
| | CTCGCTGCATTGTGGTGTTCTCTTCTCAAGGCTTTGAAATCTCCCCTTGCACTGA |
| | GATTAGTCGTCAGATCTCTCCCCGTCTCCCTCCCAACTTATACGACCTGATTTCC |
| | TTAGGACGGAACCGCAGGCACCTGCGCCGGGCGTCTTACTCCCGCTGCTTGTTCT |
| | GTCCCCTCCCTCGGACCAAACAGTGCTCATGCTTCAGGACCTTGTTTGTCGAAGA |
| | TGTTGGTTTCCCTTTCTCTGTTATTTATATAAAAATAATTTATCAAAAGGATATT |
| | TTAAAAAAGCTAGTCTGTCTTGAAACTTGTTTACCTTAAAATTATCAGAATCTCA |
| | GTGTTTGAAAGTACTGAAGCACAAACATATATCATCTCTGTACCATTCTGTACTA |
| | AAGCACTTGAGTCTAATAAATAAAGAAATCAGCACCCCT |
| SEQ ID NO: | AGTCGGTCCCTAGCGCGGCTGCGGGGCGGAGAGCTGCGGCTGGCCCAGCGCGCCC |
| 29 | ACCTGAGGAGGCGGCGGGGTCCGCAGGCGTCGCGGGACGAGGAGATCGGAGCCGG |
| Human | GAGACTCGCGCAGCGCCATGGCCCCCATTGGCCTCAAAGCTGTTGTCGGAGAGAG |
| STXBP1 | AAAGAGGAAGACTACCTAGACATGTTGCTCCAGAGCAGAATTTCCAGCAGTAGAT |
| (transcript | TGAACCTTCGGAGGCCGAGTCCCAGCCCAGTGTAAACTGAGATTATGCATGATGT |
| variant 8) | GATAAAGAAGGTCAAGAAGAAGGGGGAATGGAAGGTGCTGGTGGTGGATCAGTTA |
| encoding | AGCATGAGGATGCTGTCCTCCTGCTGCAAGATGACAGACATCATGACCGAGGGCA |
| isoform e | TAACGATTGTGGAAGATATCAATAAGCGCAGAGAGCCGCTCCCCAGCCTGGAGGC |
| | TGTGTATCTCATCACTCCATCCGAGAAGTCCGTCCACTCTCTCATCAGTGACTTT |
| | AAGGACCCGCCGACTGCTAAATACCGGGCTGCACACGTCTTCTTCACTGACTCTT |
| | GTCCAGATGCCCTGTTTAATGAACTGGTAAAATCCCGAGCAGCCAAAGTCATCAA |
| | AACTCTGACGGAAATCAATATTGCATTTCTCCCGTATGAATCCCAGGTCTATTCC |
| | TTGGACTCTGCTGACTCTTTCCAAAGCTTCTACAGTCCCCACAAGGCTCAGATGA |
| | AGAATCCTATACTGGAGCGCCTGGCAGAGCAGATCGCGACCCTTTGTGCCACCCT |
| | GAAGGAGTACCCGGCTGTGCGGTATCGGGGGGAATACAAGGACAATGCCCTGCTG |
| | GCTCAGCTAATCCAGGACAAGCTCGATGCCTATAAAGCTGATGATCCAACAATGG |
| | GGGAGGGCCCAGACAAGGCACGCTCCCAGCTCCTGATCCTGGATCGAGGCTTTGA |
| | CCCCAGCTCCCCTGTGCTCCATGAATTGACTTTTCAGGCTATGAGTTATGATCTG |
| | CTGCCTATCGAAAATGATGTATACAAGTATGAGACCAGCGGCATCGGGGAGGCAC |
| | GGGTGAAGGAGGTGCTCCTGGACGAGGACGACGACCTGTGGATAGCACTGCGCCA |
| | CAAGCACATCGCAGAGGTGTCCCAGGAAGTCACCCGGTCTCTGAAAGATTTTTCT |
| | TCTAGCAAGAGAATGAATACTGGAGAGAAGACCACCATGCGGGACCTGTCCCAGA |
| | TGCTGAAGAAGATGCCTCAGTACCAGAAAGAGCTCAGCAAGTACTCCACCCACCT |
| | GCACCTTGCTGAGGACTGTATGAAGCATTACCAAGGCACCGTAGACAAACTCTGC |
| | CGAGTGGAGCAGGACCTGGCCATGGGCACAGATGCTGAGGGAGAGAAGATCAAGG |
| | ACCCTATGCGAGCCATCGTCCCCATTCTGCTGGATGCCAATGTCAGCACTTATGA |
| | CAAAATCCGCATCATCCTTCTCTACATCTTTTTGAAGAATGGCATCACGGAGGAA |
| | AACCTGAACAAACTGATCCAGCACGCCCAGATACCCCCGGAGGATAGTGAGATCA |
| | TCACCAACATGGCTCACCTCGGCGTGCCCATCGTCACCGATTCCACGCTGCGTCG |
| | CCGGAGCAAGCCGGAGCGGAAGGAACGCATCAGCGAGCAGACCTACCAGCTCTCA |
| | CGGTGGACTCCGATTATCAAGGACATCATGGAGGACACTATTGAGGACAAACTTG |
| | ACACCAAACACTACCCTTATATCTCTACCCGTTCCTCTGCCTCCTTCAGCACCAC |
| | CGCCGTCAGCGCCCGCTATGGGCACTGGCATAAGAACAAGGCCCCAGGCGAGTAC |
| | CGCAGTGGCCCCCGCCTCATCATTTTCATCCTTGGGGGTGTGAGCCTGAATGAGA |
| | TGCGCTGCGCCTACGAGGTGACCCAGGCCAACGGAAAGTGGGAGGTGCTGATAGG |
| | ATCCACACACATCCTCACCCCACAGAAACTGCTGGACACACTGAAGAAACTGAAT |
| | AAAACAGATGAAGAAATAAGCAGTTAAAAAAATAAGTCGCCCCTCCAAAACACGC |
| | CCCCATCCCACAGCGCTCCGCAGCTTCCCACCACCGCCCGCCTCAGTTCCTTTGC |
| | GTCTGTTGCCTCCCCAGCCCTGCACGCCCTGGCTGGCACTGTTGCCGCTGCATTC |
| | TCGTGTTCAGTGATGCCCTCTTCTTGTTTGAAACAAAAGAAAATAATGCATTGTG |
| | TTTTTTAAAAAGAGTATCTTATACATGTATCCTAAAAAGAGAAGCTCATGTGCAA |
| | TTGGTGCACAGCAGGAGAAATTTCTGGACTGTTAGGATGAATGGACGCCTTCTCC |
| | CCGTTATTTAAGATTTGTGACCTTGTACATAACCCTGGGTGACGTGCACATTGCT |
| | TGGGTATGGAACGGTAGAAATTTGGGTGTTTTTAAAACCTTGTTTGGGGTTGTTC |
| | CTGTCCTTGTTGAGAATCATAGAGATGTCTGTGTTCTTGGAGTATTTCACACTGA |
| | GGACTAATCTGCTATCTTCATTCCAGTCCCTACCCCTCAGTGCCTGCTCTCATCC |
| | AAATAACCTGGGAGGTGACAATCAGGATATCTCAGGAGGTCCAAGGTGGAACAGA |
| | CCTCTTTGCCTTTCCCAGCGTCTCATACCCCCGGTAGTGCAGCTGTGGGTGGAGG |
| | CTGGGGTGTCTGCACGAAGTCAGGCCAGCGTCCTCCTCCACAGCCTGTCACTGCC |
| | CCCTCCCCAGCCTGTGTCCACAGTGCTGTGATCCCGAGGGAAGTCCTCCAGTCTA |
| | AGTCACAGTGCCCTGACAGGTGAGAAGCAAACTCCCGCTGGAAGCCTCCATCTCT |
| | TTGGAAAAACAGTTAGTCTGGAGCCTGTGGCCCAGGCCCTTCTGTCCCCAGGCAT |
| | CATCCCAACAGCTCATTTTCCCTAGTCCGCCTTCGTTCAAGGGTCAGGAATGGAC |
| | CAGAACAGATGGGTTCTGGAGGCCCCTGAACAGAGGGCTATGGCTGTGGAGAAGG |
| | TTCTTGGCCCGTTGGACTCACACAGACCCTGTACCCTCTCGGCAAGCATCTTCAG |
| | TCAGATTATCCTCAGTTTCAGATACTTCATAATACCTTGTGTTGTGTGGGGTCAT |
| | ACATCATCGTGTTTGTAAGAGAAGATGGTCATTTTATTCTCTGTATAAAACTTAG |
| | CTCTAAAGCAGAAACTAAAGCAGCAAATGCAGGAAGGCTGTCTCGCCATCCTCAA |
| | GACTCAGCAGCTCTCATTCTCCAGTGGTGAGCACACCATTTGTGCTGCTGCTGTT |
| | GTCGTGAAATATAATAACAGTGGAAGTCACAAAAATGTCCCCTGCCCAGCCCCCT |
| | CGCCGCCCTTGACCTCCTGCAGGCCATGTGTGTATTACTTGTCTAGTGATGTCCT |
| | CTCAAAGTGCTGTACGCGAGCTCGGCGCCACCTCCGCCTCCCTTTCAGAGCCTGC |
| | TCCCCGCCCTCTCTGCTCGCTGCATTGTGGTGTTCTCTTCTCAAGGCTTTGAAAT |
| | CTCCCCTTGCACTGAGATTAGTCGTCAGATCTCTCCCCGTCTCCCTCCCAACTTA |
| | TACGACCTGATTTCCTTAGGACGGAACCGCAGGCACCTGCGCCGGGCGTCTTACT |
| | CCCGCTGCTTGTTCTGTCCCCTCCCTCGGACCAAACAGTGCTCATGCTTCAGGAC |
| | CTTGTTTGTCGAAGATGTTGGTTTCCCTTTCTCTGTTATTTATATAAAAATAATT |
| | TATCAAAAGGATATTTTAAAAAAGCTAGTCTGTCTTGAAACTTGTTTACCTTAAA |
| | ATTATCAGAATCTCAGTGTTTGAAAGTACTGAAGCACAAACATATATCATCTCTG |
| | TACCATTCTGTACTAAAGCACTTGAGTCTAATAAATAAAGAAATCAGCACCCCT |
| SEQ ID NO: | AGTCGGTCCCTAGCGCGGCTGCGGGGCGGAGAGCTGCGGCTGGCCCAGCGCGCCC |
| 30 | ACCTGAGGAGGCGGCGGGGTCCGCAGGCGTCGCGGGACGAGGAGATCGGAGCCGG |
| Human | GAGACTCGCGCAGCGCCATGGCCCCCATTGGCCTCAAAGCTGTTGTCGGAGAGAG |
| STXBP1 | AAAGAGGAAGACTACCTAGACATGTTGCTCCAGAGCAGAATTTCCAGCAGTAGAT |
| (transcript | TGAACCTTCGGAGGCCGAGTCCCAGCCCAGTGTAAACTGGTAAAGAAGATTATGC |
| variant 9) | ATGATGTGATAAAGAAGGTCAAGAAGAAGGGGGAATGGAAGGTGCTGGTGGTGGA |
| encoding | TCAGTTAAGCATGAGGATGCTGTCCTCCTGCTGCAAGATGACAGACATCATGACC |
| isoform e | GAGGGCATAACGATTGTGGAAGATATCAATAAGCGCAGAGAGCCGCTCCCCAGCC |
| | TGGAGGCTGTGTATCTCATCACTCCATCCGAGAAGTCCGTCCACTCTCTCATCAG |
| | TGACTTTAAGGACCCGCCGACTGCTAAATACCGGGCTGCACACGTCTTCTTCACT |
| | GACTCTTGTCCAGATGCCCTGTTTAATGAACTGGTAAAATCCCGAGCAGCCAAAG |
| | TCATCAAAACTCTGACGGAAATCAATATTGCATTTCTCCCGTATGAATCCCAGGT |
| | CTATTCCTTGGACTCTGCTGACTCTTTCCAAAGCTTCTACAGTCCCCACAAGGCT |
| | CAGATGAAGAATCCTATACTGGAGCGCCTGGCAGAGCAGATCGCGACCCTTTGTG |
| | CCACCCTGAAGGAGTACCCGGCTGTGCGGTATCGGGGGGAATACAAGGACAATGC |
| | CCTGCTGGCTCAGCTAATCCAGGACAAGCTCGATGCCTATAAAGCTGATGATCCA |
| | ACAATGGGGGAGGGCCCAGACAAGGCACGCTCCCAGCTCCTGATCCTGGATCGAG |
| | GCTTTGACCCCAGCTCCCCTGTGCTCCATGAATTGACTTTTCAGGCTATGAGTTA |
| | TGATCTGCTGCCTATCGAAAATGATGTATACAAGTATGAGACCAGCGGCATCGGG |
| | GAGGCACGGGTGAAGGAGGTGCTCCTGGACGAGGACGACGACCTGTGGATAGCAC |
| | TGCGCCACAAGCACATCGCAGAGGTGTCCCAGGAAGTCACCCGGTCTCTGAAAGA |
| | TTTTTCTTCTAGCAAGAGAATGAATACTGGAGAGAAGACCACCATGCGGGACCTG |
| | TCCCAGATGCTGAAGAAGATGCCTCAGTACCAGAAAGAGCTCAGCAAGTACTCCA |
| | CCCACCTGCACCTTGCTGAGGACTGTATGAAGCATTACCAAGGCACCGTAGACAA |
| | ACTCTGCCGAGTGGAGCAGGACCTGGCCATGGGCACAGATGCTGAGGGAGAGAAG |
| | ATCAAGGACCCTATGCGAGCCATCGTCCCCATTCTGCTGGATGCCAATGTCAGCA |
| | CTTATGACAAAATCCGCATCATCCTTCTCTACATCTTTTTGAAGAATGGCATCAC |
| | GGAGGAAAACCTGAACAAACTGATCCAGCACGCCCAGATACCCCCGGAGGATAGT |
| | GAGATCATCACCAACATGGCTCACCTCGGCGTGCCCATCGTCACCGATTCCACGC |
| | TGCGTCGCCGGAGCAAGCCGGAGCGGAAGGAACGCATCAGCGAGCAGACCTACCA |
| | GCTCTCACGGTGGACTCCGATTATCAAGGACATCATGGAGGACACTATTGAGGAC |
| | AAACTTGACACCAAACACTACCCTTATATCTCTACCCGTTCCTCTGCCTCCTTCA |
| | GCACCACCGCCGTCAGCGCCCGCTATGGGCACTGGCATAAGAACAAGGCCCCAGG |
| | CGAGTACCGCAGTGGCCCCCGCCTCATCATTTTCATCCTTGGGGGTGTGAGCCTG |
| | AATGAGATGCGCTGCGCCTACGAGGTGACCCAGGCCAACGGAAAGTGGGAGGTGC |
| | TGATAGGATCCACACACATCCTCACCCCACAGAAACTGCTGGACACACTGAAGAA |
| | ACTGAATAAAACAGATGAAGAAATAAGCAGTTAAAAAAATAAGTCGCCCCTCCAA |
| | AACACGCCCCCATCCCACAGCGCTCCGCAGCTTCCCACCACCGCCCGCCTCAGTT |
| | CCTTTGCGTCTGTTGCCTCCCCAGCCCTGCACGCCCTGGCTGGCACTGTTGCCGC |
| | TGCATTCTCGTGTTCAGTGATGCCCTCTTCTTGTTTGAAACAAAAGAAAATAATG |
| | CATTGTGTTTTTTAAAAAGAGTATCTTATACATGTATCCTAAAAAGAGAAGCTCA |
| | TGTGCAATTGGTGCACAGCAGGAGAAATTTCTGGACTGTTAGGATGAATGGACGC |
| | CTTCTCCCCGTTATTTAAGATTTGTGACCTTGTACATAACCCTGGGTGACGTGCA |
| | CATTGCTTGGGTATGGAACGGTAGAAATTTGGGTGTTTTTAAAACCTTGTTTGGG |
| | GTTGTTCCTGTCCTTGTTGAGAATCATAGAGATGTCTGTGTTCTTGGAGTATTTC |
| | ACACTGAGGACTAATCTGCTATCTTCATTCCAGTCCCTACCCCTCAGTGCCTGCT |
| | CTCATCCAAATAACCTGGGAGGTGACAATCAGGATATCTCAGGAGGTCCAAGGTG |
| | GAACAGACCTCTTTGCCTTTCCCAGCGTCTCATACCCCCGGTAGTGCAGCTGTGG |
| | GTGGAGGCTGGGGTGTCTGCACGAAGTCAGGCCAGCGTCCTCCTCCACAGCCTGT |
| | CACTGCCCCCTCCCCAGCCTGTGTCCACAGTGCTGTGATCCCGAGGGAAGTCCTC |
| | CAGTCTAAGTCACAGTGCCCTGACAGGTGAGAAGCAAACTCCCGCTGGAAGCCTC |
| | CATCTCTTTGGAAAAACAGTTAGTCTGGAGCCTGTGGCCCAGGCCCTTCTGTCCC |
| | CAGGCATCATCCCAACAGCTCATTTTCCCTAGTCCGCCTTCGTTCAAGGGTCAGG |
| | AATGGACCAGAACAGATGGGTTCTGGAGGCCCCTGAACAGAGGGCTATGGCTGTG |
| | GAGAAGGTTCTTGGCCCGTTGGACTCACACAGACCCTGTACCCTCTCGGCAAGCA |
| | TCTTCAGTCAGATTATCCTCAGTTTCAGATACTTCATAATACCTTGTGTTGTGTG |
| | GGGTCATACATCATCGTGTTTGTAAGAGAAGATGGTCATTTTATTCTCTGTATAA |
| | AACTTAGCTCTAAAGCAGAAACTAAAGCAGCAAATGCAGGAAGGCTGTCTCGCCA |
| | TCCTCAAGACTCAGCAGCTCTCATTCTCCAGTGGTGAGCACACCATTIGTGCTGC |
| | TGCTGTTGTCGTGAAATATAATAACAGTGGAAGTCACAAAAATGTCCCCTGCCCA |
| | GCCCCCTCGCCGCCCTTGACCTCCTGCAGGCCATGTGTGTATTACTTGTCTAGTG |
| | ATGTCCTCTCAAAGTGCTGTACGCGAGCTCGGCGCCACCTCCGCCTCCCTTTCAG |
| | AGCCTGCTCCCCGCCCTCTCTGCTCGCTGCATTGTGGTGTTCTCTTCTCAAGGCT |
| | TTGAAATCTCCCCTTGCACTGAGATTAGTCGTCAGATCTCTCCCCGTCTCCCTCC |
| | CAACTTATACGACCTGATTTCCTTAGGACGGAACCGCAGGCACCTGCGCCGGGCG |
| | TCTTACTCCCGCTGCTTGTTCTGTCCCCTCCCTCGGACCAAACAGTGCTCATGCT |
| | TCAGGACCTTGTTTGTCGAAGATGTTGGTTTCCCTTTCTCTGTTATTTATATAAA |
| | AATAATTTATCAAAAGGATATTTTAAAAAAGCTAGTCTGTCTTGAAACTTGTTTA |
| | CCTTAAAATTATCAGAATCTCAGTGTTTGAAAGTACTGAAGCACAAACATATATC |
| | ATCTCTGTACCATTCTGTACTAAAGCACTTGAGTCTAATAAATAAAGAAATCAGC |
| | ACCCCT |
| SEQ ID NO: | AGTCGGTCCCTAGCGCGGCTGCGGGGCGGAGAGCTGCGGCTGGCCCAGCGCGCCC |
| 31 | ACCTGAGGAGGCGGCGGGGTCCGCAGGCGTCGCGGGACGAGGAGATCGGAGCCGG |
| Human | GAGACTCGCGCAGCGCCATGGCCCCCATTGGCCTCAAAGCTGTTGTCGGAGAGAA |
| STXBP1 | GATTATGCATGATGTGATAAAGAAGGTCAAGAAGAAGGGGGAATGGAAGGTGCTG |
| (transcript | GTGGTGGATCAGTTAAGCATGAGGATGCTGTCCTCCTGCTGCAAGATGACAGACA |
| variant 10) | TCATGACCGAGGGCATAACGATTGTGGAAGATATCAATAAGCGCAGAGAGCCGCT |
| encoding | CCCCAGCCTGGAGGCTGTGTATCTCATCACTCCATCCGAGAAGTCCGTCCACTCT |
| isoform f | CTCATCAGTGACTTTAAGGACCCGCCGACTGCTAAATACCGGGCTGCACACGTCT |
| | TCTTCACTGACTCTTGTCCAGATGCCCTGTTTAATGAACTGGTAAAATCCCGAGC |
| | AGCCAAAGTCATCAAAACTCTGACGGAAATCAATATTGCATTTCTCCCGTATGAA |
| | TCCCAGGTCTATTCCTTGGACTCTGCTGACTCTTTCCAAAGCTTCTACAGTCCCC |
| | ACAAGGCTCAGATGAAGAATCCTATACTGGAGCGCCTGGCAGAGCAGATCGCGAC |
| | CCTTTGTGCCACCCTGAAGGAGTACCCGGCTGTGCGGTATCGGGGGGAATACAAG |
| | GACAATGCCCTGCTGGCTCAGCTAATCCAGGACAAGCTCGATGCCTATAAAGCTG |
| | ATGATCCAACAATGGGGGAGGGCCCAGACAAGGCACGCTCCCAGCTCCTGATCCT |
| | GGATCGAGGCTTTGACCCCAGCTCCCCTGTGCTCCATGAATTGACTTTTCAGGCT |
| | ATGAGTTATGATCTGCTGCCTATCGAAAATGATGTATACAAGTATGAGACCAGCG |
| | GCATCGGGGAGGCACGGGTGAAGGAGGTGCTCCTGGACGAGGACGACGACCTGTG |
| | GATAGCACTGCGCCACAAGCACATCGCAGAGGTGTCCCAGGAAGTCACCCGGTCT |
| | CTGAAAGATTTTTCTTCTAGCAAGAGAATGAATACTGGAGAGAAGACCACCATGC |
| | GGGACCTGTCCCAGATGCTGAAGAAGATGCCTCAGTACCAGAAAGAGCTCAGCAA |
| | GTACTCCACCCACCTGCACCTTGCTGAGGACTGTATGAAGCATTACCAAGGCACC |
| | GTAGACAAACTCTGCCGAGTGGAGCAGGACCTGGCCATGGGCACAGATGCTGAGG |
| | GAGAGAAGATCAAGGACCCTATGCGAGCCATCGTCCCCATTCTGCTGGATGCCAA |
| | TGTCAGCACTTATGACAAAATCCGCATCATCCTTCTCTACATCTTTTTGAAGAAT |
| | GGCATCACGGAGGAAAACCTGAACAAACTGATCCAGCACGCCCAGATACCCCCGG |
| | AGGATAGTGAGATCATCACCAACATGGCTCACCTCGGCGTGCCCATCGTCACCGA |
| | TTCCACGCTGCGTCGCCGGAGCAAGCCGGAGCGGAAGGAACGCATCAGCGAGCAG |
| | ACCTACCAGCTCTCACGGTGGACTCCGATTATCAAGGACATCATGGAGGACACTA |
| | TTGAGGACAAACTTGACACCAAACACTACCCTTATATCTCTACCCGTTCCTCTGC |
| | CTCCTTCAGCACCACCGCCGTCAGCGCCCGCTATGGGCACTGGCATAAGAACAAG |
| | GCCCCAGGCGAGTACCGCAGTGGCCCCCGCCTCATCATTTTCATCCTTGGGGGTG |
| | TGAGCCTGAATGAGATGCGCTGCGCCTACGAGGTGACCCAGGCCAACGGAAAGTG |
| | GGAGGTGCTGATAGGGGAATGGCCCTTCAGAACCTTGGCTAGGTGACATCAGAAG |
| | CTGCTTGTGTATGTAAGGAAAATGGGGCTTCCTCCTAAGGATCCACACACATCCT |
| | CACCCCACAGAAACTGCTGGACACACTGAAGAAACTGAATAAAACAGATGAAGAA |
| | ATAAGCAGTTAAAAAAATAAGTCGCCCCTCCAAAACACGCCCCCATCCCACAGCG |
| | CTCCGCAGCTTCCCACCACCGCCCGCCTCAGTTCCTTTGCGTCTGTTGCCTCCCC |
| | AGCCCTGCACGCCCTGGCTGGCACTGTTGCCGCTGCATTCTCGTGTTCAGTGATG |
| | CCCTCTTCTTGTTTGAAACAAAAGAAAATAATGCATTGTGTTTTTTAAAAAGAGT |
| | ATCTTATACATGTATCCTAAAAAGAGAAGCTCATGTGCAATTGGTGCACAGCAGG |
| | AGAAATTTCTGGACTGTTAGGATGAATGGACGCCTTCTCCCCGTTATTTAAGATT |
| | TGTGACCTTGTACATAACCCTGGGTGACGTGCACATTGCTTGGGTATGGAACGGT |
| | AGAAATTTGGGTGTTTTTAAAACCTTGTTTGGGGTTGTTCCTGTCCTTGTTGAGA |
| | ATCATAGAGATGTCTGTGTTCTTGGAGTATTTCACACTGAGGACTAATCTGCTAT |
| | CTTCATTCCAGTCCCTACCCCTCAGTGCCTGCTCTCATCCAAATAACCTGGGAGG |
| | TGACAATCAGGATATCTCAGGAGGTCCAAGGTGGAACAGACCTCTTTGCCTTTCC |
| | CAGCGTCTCATACCCCCGGTAGTGCAGCTGTGGGTGGAGGCTGGGGTGTCTGCAC |
| | GAAGTCAGGCCAGCGTCCTCCTCCACAGCCTGTCACTGCCCCCTCCCCAGCCTGT |
| | GTCCACAGTGCTGTGATCCCGAGGGAAGTCCTCCAGTCTAAGTCACAGTGCCCTG |
| | ACAGGTGAGAAGCAAACTCCCGCTGGAAGCCTCCATCTCTTTGGAAAAACAGTTA |
| | GTCTGGAGCCTGTGGCCCAGGCCCTTCTGTCCCCAGGCATCATCCCAACAGCTCA |
| | TTTTCCCTAGTCCGCCTTCGTTCAAGGGTCAGGAATGGACCAGAACAGATGGGTT |
| | CTGGAGGCCCCTGAACAGAGGGCTATGGCTGTGGAGAAGGTTCTTGGCCCGTTGG |
| | ACTCACACAGACCCTGTACCCTCTCGGCAAGCATCTTCAGTCAGATTATCCTCAG |
| | TTTCAGATACTTCATAATACCTTGTGTTGTGTGGGGTCATACATCATCGTGTTTG |
| | TAAGAGAAGATGGTCATTTTATTCTCTGTATAAAACTTAGCTCTAAAGCAGAAAC |
| | TAAAGCAGCAAATGCAGGAAGGCTGTCTCGCCATCCTCAAGACTCAGCAGCTCTC |
| | ATTCTCCAGTGGTGAGCACACCATTTGTGCTGCTGCTGTTGTCGTGAAATATAAT |
| | AACAGTGGAAGTCACAAAAATGTCCCCTGCCCAGCCCCCTCGCCGCCCTTGACCT |
| | CCTGCAGGCCATGTGTGTATTACTTGTCTAGTGATGTCCTCTCAAAGTGCTGTAC |
| | GCGAGCTCGGCGCCACCTCCGCCTCCCTTTCAGAGCCTGCTCCCCGCCCTCTCTG |
| | CTCGCTGCATTGTGGTGTTCTCTTCTCAAGGCTTTGAAATCTCCCCTTGCACTGA |
| | GATTAGTCGTCAGATCTCTCCCCGTCTCCCTCCCAACTTATACGACCTGATTTCC |
| | TTAGGACGGAACCGCAGGCACCTGCGCCGGGCGTCTTACTCCCGCTGCTTGTTCT |
| | GTCCCCTCCCTCGGACCAAACAGTGCTCATGCTTCAGGACCTTGTTTGTCGAAGA |
| | TGTTGGTTTCCCTTTCTCTGTTATTTATATAAAAATAATTTATCAAAAGGATATT |
| | TTAAAAAAGCTAGTCTGTCTTGAAACTTGTTTACCTTAAAATTATCAGAATCTCA |
| | GTGTTTGAAAGTACTGAAGCACAAACATATATCATCTCTGTACCATTCTGTACTA |
| | AAGCACTTGAGTCTAATAAATAAAGAAATCAGCACCCCT |
| SEQ ID NO: | AGTCGGTCCCTAGCGCGGCTGCGGGGCGGAGAGCTGCGGCTGGCCCAGCGCGCCC |
| 32 | ACCTGAGGAGGCGGCGGGGTCCGCAGGCGTCGCGGGACGAGGAGATCGGAGCCGG |
| Human | GAGACTCGCGCAGCGCCATGGCCCCCATTGGCCTCAAAGCTGTTGTCGGAGAGAA |
| STXBP1 | GATTATGCATGATGTGATAAAGAAGGTCAAGAAGAAGGGGGAATGGAAGGTGCTG |
| (transcript | GTGGTGGATCAGTTAAGCATGAGGATGCTGTCCTCCTGCTGCAAGATGACAGACA |
| variant 11) | TCATGACCGAGGGCATAACGATTGTGGAAGATATCAATAAGCGCAGAGAGCCGCT |
| encoding | CCCCAGCCTGGAGGCTGTGTATCTCATCACTCCATCCGAGAAGTCCGTCCACTCT |
| isoform g | CTCATCAGTGACTTTAAGGACCCGCCGACTGCTAAATACCGGGCTGCACACGTCT |
| | TCTTCACTGACTCTTGTCCAGATGCCCTGTTTAATGAACTGGTAAAATCCCGAGC |
| | AGCCAAAGTCATCAAAACTCTGACGGAAATCAATATTGCATTTCTCCCGTATGAA |
| | TCCCAGGTCTATTCCTTGGACTCTGCTGACTCTTTCCAAAGCTTCTACAGTCCCC |
| | ACAAGGCTCAGATGAAGAATCCTATACTGGAGCGCCTGGCAGAGCAGATCGCGAC |
| | CCTTTGTGCCACCCTGAAGGAGTACCCGGCTGTGCGGTATCGGGGGGAATACAAG |
| | GACAATGCCCTGCTGGCTCAGCTAATCCAGGACAAGCTCGATGCCTATAAAGCTG |
| | ATGATCCAACAATGGGGGAGGGCCCAGACAAGGCACGCTCCCAGCTCCTGATCCT |
| | GGATCGAGGCTTTGACCCCAGCTCCCCTGTGCTCCATGAATTGACTTTTCAGGCT |
| | ATGAGTTATGATCTGCTGCCTATCGAAAATGATGTATACAAGTATGAGACCAGCG |
| | GCATCGGGGAGGCACGGGTGAAGGAGGTGCTCCTGGACGAGGACGACGACCTGTG |
| | GATAGCACTGCGCCACAAGCACATCGCAGAGGTGTCCCAGGAAGTCACCCGGTCT |
| | CTGAAAGATTTTTCTTCTAGCAAGAGAATGAATACTGGAGAGAAGACCACCATGC |
| | GGGACCTGTCCCAGATGCTGAAGAAGATGCCTCAGTACCAGAAAGAGCTCAGCAA |
| | GTACTCCACCCACCTGCACCTTGCTGAGGACTGTATGAAGCATTACCAAGGCACC |
| | GTAGACAAACTCTGCCGAGTGGAGCAGGACCTGGCCATGGGCACAGATGCTGAGG |
| | GAGAGAAGATCAAGGACCCTATGCGAGCCATCGTCCCCATTCTGCTGGATGCCAA |
| | TGTCAGCACTTATGACAAAATCCGCATCATCCTTCTCTACATCTTTTTGAAGAAT |
| | GGCATCACGGAGGAAAACCTGAACAAACTGATCCAGCACGCCCAGATACCCCCGG |
| | AGGATAGTGAGATCATCACCAACATGGCTCACCTCGGCGTGCCCATCGTCACCGA |
| | TTCCACGCTGCGTCGCCGGAGCAAGCCGGAGCGGAAGGAACGCATCAGCGAGCAG |
| | ACCTACCAGCTCTCACGGTGGACTCCGATTATCAAGGACATCATGGAGGACACTA |
| | TTGAGGACAAACTTGACACCAAACACTACCCTTATATCTCTACCCGTTCCTCTGC |
| | CTCCTTCAGCACCACCGCCGTCAGCGCCCGCTATGGGCACTGGCATAAGAACAAG |
| | GCCCCAGGCGAGTACCGCAGTGGCCCCCGCCTCATCATTTTCATCCTTGGGGGTG |
| | TGAGCCTGAATGAGATGCGCTGCGCCTACGAGGTGACCCAGGCCAACGGAAAGTG |
| | GGAGGTGCTGATAGTGCCCGTGGAGGCGGGAAGCTGAGCCCGGCTCCCAGGAAGC |
| | ACAGTGAGGGCTCAGCCATTGATGATGATGATGATGATGATGATGATGATGATGA |
| | TGATGGTGATGATGATGATGCTGCTGCTGTTGACGATGAGTCCAAGCCAAGCCTG |
| | GGGAGAAAAGAATAAATAGTCTCATAAATTTAAAAAAGCCCAATGCTCATCAAGA |
| | AGAGAACAGATAAACTGTGTATTCACCCAATGGTGAGTTTAAAGCAGTTAAAAAT |
| | GCATGACACACAGTTGCATATGTCAACATGAATAGATCTCAGACACTTAACAATG |
| | AGTGAAAAGAGCACATTATGGGAGGACACAGACAGGATAGTGCATTTATATAAAG |
| | CTTCAATGTTGCAGGCTGAAAGAGTGAGGGTCGTGATCAACTCAGTATCCTGGAG |
| | GCTACATGGGTAAACAGCAAACTGTTCTCATAAATGCAGAATGTTGGCAAACTGA |
| | CAAACTGCATCTGCCGCCCAGAAGGAATGCGGAGGGCAGCCACTCCCTAAGCGCA |
| | GTTTTCTTGTGATTAGGTACATCTGAAGCCTGTTAGCAATAATGTGAACCTGTGA |
| | TCAATTAAGCAGCTGACCAATCATTACCTCCTCTTCCCTGCTCTTTCTACCCAGT |
| | AAATACAAAGGGCTGTAGAAGCTCAGAGCTGCTGCTTTTGCTCAGTAGAAGCAGG |
| | GAGTCCTCTTCTTCTTCCCCCGACCCCTTCTTTTAAAACAGTTTCTTTTAAGTTT |
| | TCATTTCTGCGTTCATCCTCCTTCATTCAGTCCCGTAGTAACCGTGGCAAACCAC |
| | GGCACTTCAAAACCTCATAAAAAGCACCACATATTGCTTATTTGGATACATATGT |
| | TGTAAATGTGTGAAAACATGCATGGGAATGACAAATACCAAAGTCAGGATAGTGA |
| | CTGGCCATGGGGAAGGGATGGTTATTATTGTCGTCTTATATCTTCAATTCCAAAT |
| | GAGCCACGGGCTTAAAAGTACTTCATTGAATAAAAAAAAACCAACAATGTATTAA |
| | TGCAA |
| SEQ ID NO: | AGTCGGTCCCTAGCGCGGCTGCGGGGCGGAGAGCTGCGGCTGGCCCAGCGCGCCC |
| 33 | ACCTGAGGAGGCGGCGGGGTCCGCAGGCGTCGCGGGACGAGGAGATCGGAGCCGG |
| Human | GAGACTCGCGCAGCGCCATGGCCCCCATTGGCCTCAAAGCTGTTGTCGGAGAGAA |
| STXBP1 | GATTATGCATGATGTGATAAAGAAGGTCAAGAAGAAGGGGGAATGGAAGGTGCTG |
| (transcript | GTGGTGGATCAGTTAAGCATGAGGATGCTGTCCTCCTGCTGCAAGATGACAGACA |
| variant 12) | TCATGACCGAGGGCATAACGATTGTGGAAGATATCAATAAGCGCAGAGAGCCGCT |
| encoding | CCCCAGCCTGGAGGCTGTGTATCTCATCACTCCATCCGAGAAGTCCGTCCACTCT |
| isoform h | CTCATCAGTGACTTTAAGGACCCGCCGACTGCTAAATACCGGGCTGCACACGTCT |
| | TCTTCACTGACTCTTGTCCAGATGCCCTGTTTAATGAACTGGTAAAATCCCGAGC |
| | AGCCAAAGTCATCAAAACTCTGACGGAAATCAATATTGCATTTCTCCCGTATGAA |
| | TCCCAGGTCTATTCCTTGGACTCTGCTGACTCTTTCCAAAGCTTCTACAGTCCCC |
| | ACAAGGCTCAGATGAAGAATCCTATACTGGAGCGCCTGGCAGAGCAGATCGCGAC |
| | CCTTTGTGCCACCCTGAAGGAGTACCCGGCTGTGCGGTATCGGGGGGAATACAAG |
| | GACAATGCCCTGCTGGCTCAGCTAATCCAGGACAAGCTCGATGCCTATAAAGCTG |
| | ATGATCCAACAATGGGGGAGGGCCCAGACAAGGCACGCTCCCAGCTCCTGATCCT |
| | GGATCGAGGCTTTGACCCCAGCTCCCCTGTGCTCCATGAATTGACTTTTCAGGCT |
| | ATGAGTTATGATCTGCTGCCTATCGAAAATGATGTATACAAGGAAGTCACCCGGT |
| | CTCTGAAAGATTTTTCTTCTAGCAAGAGAATGAATACTGGAGAGAAGACCACCAT |
| | GCGGGACCTGTCCCAGATGCTGAAGAAGATGCCTCAGTACCAGAAAGAGCTCAGC |
| | AAGTACTCCACCCACCTGCACCTTGCTGAGGACTGTATGAAGCATTACCAAGGCA |
| | CCGTAGACAAACTCTGCCGAGTGGAGCAGGACCTGGCCATGGGCACAGATGCTGA |
| | GGGAGAGAAGATCAAGGACCCTATGCGAGCCATCGTCCCCATTCTGCTGGATGCC |
| | AATGTCAGCACTTATGACAAAATCCGCATCATCCTTCTCTACATCTTTTTGAAGA |
| | ATGGCATCACGGAGGAAAACCTGAACAAACTGATCCAGCACGCCCAGATACCCCC |
| | GGAGGATAGTGAGATCATCACCAACATGGCTCACCTCGGCGTGCCCATCGTCACC |
| | GATTCCACGCTGCGTCGCCGGAGCAAGCCGGAGCGGAAGGAACGCATCAGCGAGC |
| | AGACCTACCAGCTCTCACGGTGGACTCCGATTATCAAGGACATCATGGAGGACAC |
| | TATTGAGGACAAACTTGACACCAAACACTACCCTTATATCTCTACCCGTTCCTCT |
| | GCCTCCTTCAGCACCACCGCCGTCAGCGCCCGCTATGGGCACTGGCATAAGAACA |
| | AGGCCCCAGGCGAGTACCGCAGTGGCCCCCGCCTCATCATTTTCATCCTTGGGGG |
| | TGTGAGCCTGAATGAGATGCGCTGCGCCTACGAGGTGACCCAGGCCAACGGAAAG |
| | TGGGAGGTGCTGATAGGTTCTACTCACATTCTTACTCCCACCAAATTTCTCATGG |
| | ACCTGAGACACCCCGACTTCAGGGAGTCCTCTAGGGTATCTTTTGAGGATCAGGC |
| | TCCAACAATGGAGTGAGAGCCAAAGAAACAAAGATCCACACACATCCTCACCCCA |
| | CAGAAACTGCTGGACACACTGAAGAAACTGAATAAAACAGATGAAGAAATAAGCA |
| | GTTAAAAAAATAAGTCGCCCCTCCAAAACACGCCCCCATCCCACAGCGCTCCGCA |
| | GCTTCCCACCACCGCCCGCCTCAGTTCCTTTGCGTCTGTTGCCTCCCCAGCCCTG |
| | CACGCCCTGGCTGGCACTGTTGCCGCTGCATTCTCGTGTTCAGTGATGCCCTCTT |
| | CTTGTTTGAAACAAAAGAAAATAATGCATTGTGTTTTTTAAAAAGAGTATCTTAT |
| | ACATGTATCCTAAAAAGAGAAGCTCATGTGCAATTGGTGCACAGCAGGAGAAATT |
| | TCTGGACTGTTAGGATGAATGGACGCCTTCTCCCCGTTATTTAAGATTTGTGACC |
| | TTGTACATAACCCTGGGTGACGTGCACATTGCTTGGGTATGGAACGGTAGAAATT |
| | TGGGTGTTTTTAAAACCTTGTTTGGGGTTGTTCCTGTCCTTGTTGAGAATCATAG |
| | AGATGTCTGTGTTCTTGGAGTATTTCACACTGAGGACTAATCTGCTATCTTCATT |
| | CCAGTCCCTACCCCTCAGTGCCTGCTCTCATCCAAATAACCTGGGAGGTGACAAT |
| | CAGGATATCTCAGGAGGTCCAAGGTGGAACAGACCTCTTTGCCTTTCCCAGCGTC |
| | TCATACCCCCGGTAGTGCAGCTGTGGGTGGAGGCTGGGGTGTCTGCACGAAGTCA |
| | GGCCAGCGTCCTCCTCCACAGCCTGTCACTGCCCCCTCCCCAGCCTGTGTCCACA |
| | GTGCTGTGATCCCGAGGGAAGTCCTCCAGTCTAAGTCACAGTGCCCTGACAGGTG |
| | AGAAGCAAACTCCCGCTGGAAGCCTCCATCTCTTTGGAAAAACAGTTAGTCTGGA |
| | GCCTGTGGCCCAGGCCCTTCTGTCCCCAGGCATCATCCCAACAGCTCATTTTCCC |
| | TAGTCCGCCTTCGTTCAAGGGTCAGGAATGGACCAGAACAGATGGGTTCTGGAGG |
| | CCCCTGAACAGAGGGCTATGGCTGTGGAGAAGGTTCTTGGCCCGTTGGACTCACA |
| | CAGACCCTGTACCCTCTCGGCAAGCATCTTCAGTCAGATTATCCTCAGTTTCAGA |
| | TACTTCATAATACCTTGTGTTGTGTGGGGTCATACATCATCGTGTTTGTAAGAGA |
| | AGATGGTCATTTTATTCTCTGTATAAAACTTAGCTCTAAAGCAGAAACTAAAGCA |
| | GCAAATGCAGGAAGGCTGTCTCGCCATCCTCAAGACTCAGCAGCTCTCATTCTCC |
| | AGTGGTGAGCACACCATTTGTGCTGCTGCTGTTGTCGTGAAATATAATAACAGTG |
| | GAAGTCACAAAAATGTCCCCTGCCCAGCCCCCTCGCCGCCCTTGACCTCCTGCAG |
| | GCCATGTGTGTATTACTTGTCTAGTGATGTCCTCTCAAAGTGCTGTACGCGAGCT |
| | CGGCGCCACCTCCGCCTCCCTTTCAGAGCCTGCTCCCCGCCCTCTCTGCTCGCTG |
| | CATTGTGGTGTTCTCTTCTCAAGGCTTTGAAATCTCCCCTTGCACTGAGATTAGT |
| | CGTCAGATCTCTCCCCGTCTCCCTCCCAACTTATACGACCTGATTTCCTTAGGAC |
| | GGAACCGCAGGCACCTGCGCCGGGCGTCTTACTCCCGCTGCTTGTTCTGTCCCCT |
| | CCCTCGGACCAAACAGTGCTCATGCTTCAGGACCTTGTTTGTCGAAGATGTTGGT |
| | TTCCCTTTCTCTGTTATTTATATAAAAATAATTTATCAAAAGGATATTTTAAAAA |
| | AGCTAGTCTGTCTTGAAACTTGTTTACCTTAAAATTATCAGAATCTCAGTGTTTG |
| | AAAGTACTGAAGCACAAACATATATCATCTCTGTACCATTCTGTACTAAAGCACT |
| | TGAGTCTAATAAATAAAGAAATCAGCACCCCT |
| SEQ ID NO: | TTTCAATATTATTGAAGCATTTATCAGGGTTATTGTCTCATGAGCGGATACATAT |
| 34 | TTGAATGTATTTAGAAAAATAAACAAATAGGGGTCAGTGTTACAACCAATTAACC |
| AAVtt DNA | AATTCTGAACATTATCGCGAGCCCATTTATACCTGAATATGGCTCATAACACCCC |
| sequence | TTGTTTGCCTGGCGGCAGTAGCGCGGTGGTCCCACCTGACCCCATGCCGAACTCA |
| | GAAGTGAAACGCCGTAGCGCCGATGGTAGTGTGGGGACTCCCCATGCGAGAGTAG |
| | GGAACTGCCAGGCATCAAATAAAACGAAAGGCTCAGTCGAAAGACTGGGCCTTTC |
| | GCCCGGGCTAATTAGGGGGTGTCGCCCTTCGCTGAAGTCCTGTATTAGAGGTCAC |
| | GTGAGTGTTTTGCGACATTTTGCGACACCATGTGGTCACGCTGGGTATTTAAGCC |
| | CGAGTGAGCACGCAGGGTCTCCATTTTGAAGCGGGAGGTTTGAACGCGCAGCCGC |
| | CATGCCGGGGTTTTACGAGATTGTGATTAAGGTCCCCAGCGACCTTGACGAGCAT |
| | CTGCCCGGCATTTCTGACAGCTTTGTGAACTGGGTGGCCGAGAAGGAATGGGAGT |
| | TGCCGCCAGATTCTGACATGGATCTGAATCTGATTGAGCAGGCACCCCTGACCGT |
| | GGCCGAGAAGCTGCAGCGCGACTTTCTGACGGAATGGCGCCGTGTGAGTAAGGCC |
| | CCGGAGGCCCTTTTCTTTGTGCAATTTGAGAAGGGAGAGAGCTACTTCCACATGC |
| | ACGTGCTCGTGGAAACCACCGGGGTGAAATCCATGGTTTTGGGACGTTTCCTGAG |
| | TCAGATTCGCGAAAAACTGATTCAGAGAATTTACCGCGGGATCGAGCCGACTTTG |
| | CCAAACTGGTTCGCGGTCACAAAGACCAGAAATGGCGCCGGAGGCGGGAACAAGG |
| | TGGTGGATGAGTGCTACATCCCCAATTACTTGCTCCCCAAAACCCAGCCTGAGCT |
| | CCAGTGGGCGTGGACTAATATGGAACAGTATTTAAGCGCCTGTTTGAATCTCACG |
| | GAGCGTAAACGGTTGGTGGCGCAGCATCTGACGCACGTGTCGCAGACGCAGGAGC |
| | AGAACAAAGAGAATCAGAATCCCAATTCTGATGCGCCGGTGATCAGATCAAAAAC |
| | TTCAGCCAGGTACATGGAGCTGGTCGGGTGGCTCGTGGACAAGGGGATTACCTCG |
| | GAGAAGCAGTGGATCCAGGAGGACCAGGCCTCATACATCTCCTTCAATGCGGCCT |
| | CCAACTCGCGGTCCCAAATCAAGGCTGCCTTGGACAATGCGGGAAAGATTATGAG |
| | CCTGACTAAAACCGCCCCCGACTACCTGGTGGGCCAGCAGCCCGTGGAGGACATT |
| | TCCAGCAATCGGATTTATAAAATTTTGGAACTAAACGGGTACGATCCCCAATATG |
| | CGGCTTCCGTCTTTCTGGGATGGGCCACGAAAAAGTTCGGCAAGAGGAACACCAT |
| | CTGGCTGTTTGGGCCTGCAACTACCGGGAAGACCAACATCGCGGAGGCCATAGCC |
| | CACACTGTGCCCTTCTACGGGTGCGTAAACTGGACCAATGAGAACTTTCCCTTCA |
| | ACGACTGTGTCGACAAGATGGTGATCTGGTGGGAGGAGGGGAAGATGACCGCCAA |
| | GGTCGTGGAGTCGGCCAAAGCCATTCTCGGAGGAAGCAAGGTGCGCGTGGACCAG |
| | AAATGCAAGTCCTCGGCCCAGATAGACCCGACTCCCGTGATCGTCACCTCCAACA |
| | CCAACATGTGCGCCGTGATTGACGGGAACTCAACGACCTTCGAACACCAGCAGCC |
| | GTTGCAAGACCGGATGTTCAAATTTGAACTCACCCGCCGTCTGGATCATGACTTT |
| | GGGAAGGTCACCAAGCAGGAAGTCAAAGACTTTTTCCGGTGGGCAAAGGATCACG |
| | TGGTTGAGGTGGAGCATGAATTCTACGTCAAAAAGGGTGGAGCCAAGAAAAGACC |
| | CGCCCCCAGTGACGCAGATATAAGTGAGCCCAAACGGGTGCGCGAGTCAGTTGCG |
| | CAGCCATCGACGTCAGACGCGGAAGCTTCGATCAACTACGCAGACAGGTACCAAA |
| | ACAAATGTTCTCGTCACGTGGGCATGAATCTGATGCTGTTTCCCTGCAGACAATG |
| | CGAGAGAATGAATCAGAATTCAAATATCTGCTTCACTCACGGACAGAAAGACTGT |
| | TTAGAGTGCTTTCCCGTGTCAGAATCTCAACCCGTTTCTGTCGTCAAAAAGGCGT |
| | ATCAGAAACTGTGCTACATTCATCATATCATGGGAAAGGTGCCAGACGCTTGCAC |
| | TGCCTGCGATCTGGTCAATGTGGATTTGGATGACTGCATCTTTGAACAATAAATG |
| | ATTTAAATCAGGTATGGCTGCCGATGGTTATCTTCCAGATTGGCTCGAGGACACT |
| | CTCTCTGAAGGAATAAGACAGTGGTGGAAGCTCAAACCTGGCCCACCACCACCAA |
| | AGCCCGCAGAGCGGCATAAGGACGACAGCAGGGGTCTTGTGCTTCCTGGGTACAA |
| | GTACCTCGGACCCTTCAACGGACTCGACAAGGGAGAGCCGGTCAACGAGGCAGAC |
| | GCCGCGGCCCTCGAGCACGACAAAGCCTACGACCGGCAGCTCGACAGCGGAGACA |
| | ACCCGTACCTCAAGTACAACCACGCCGACGCGGAGTTTCAGGAGCGCCTTAAAGA |
| | AGATACGTCTTTTGGGGGCAACCTCGGACGAGCAGTCTTCCAGGCGAAAAAGAGG |
| | ATCCTTGAACCTCTGGGCCTGGTTGAGGAACCTGTTAAGACGGCTCCGGGAAAAA |
| | AGAGGCCGGTAGAGCACTCTCCTGCCGAGCCAGACTCCTCCTCGGGAACCGGAAA |
| | GAGCGGCCAGCAGCCTGCAAGAAAAAGATTGAATTTTGGTCAGACTGGAGACGCA |
| | GACTCAGTACCTGACCCCCAGCCTCTCGGACAGCCACCAGCAGCCCCCTCTGGTC |
| | TGGGAACTAATACGATGGCTAGCGGCAGTGGCGCACCAATGGCAGACAATAACGA |
| | GGGCGCCGACGGAGTGGGTAATTCCTCGGGAAATTGGCATTGCGATTCCACATGG |
| | ATGGGCGACAGAGTCATCACCACCAGCACCCGAACCTGGGCCCTGCCCACCTACA |
| | ACAACCACCTCTACAAACAAATTTCCAGCCAATCAGGAGCCTCGAACGACAATCA |
| | CTACTTTGGCTACAGCACCCCTTGGGGGTATTTTGACTTCAACAGATTCCACTGC |
| | CACTTTTCACCACGTGACTGGCAAAGACTCATCAACAACAACTGGGGATTCCGAC |
| | CCAAGAGACTCAGCTTCAAGCTCTTTAACATTCAAGTCAAAGAGGTCACGCAGAA |
| | TGACGGTACGACGACGATTGCCAATAACCTTACCAGCACGGTTCAGGTGTTTACT |
| | GACTCGGAGTACCAGCTCCCGTACGTCCTCGGCTCGGCGCATCAAGGATGCCTCC |
| | CGCCGTTCCCAGCAGACGTCTTCATGGTGCCACAGTATGGATACCTCACCCTGAA |
| | CAACGGGAGTCAGGCAGTAGGACGCTCTTCATTTTACTGCCTGGAGTACTTTCCT |
| | TCTCAGATGCTGCGTACCGGAAACAACTTTACCTTCAGCTACACTTTTGAGGACG |
| | TTCCTTTCCACAGCAGCTACGCTCACAGCCAGAGTCTGGACCGTCTCATGAATCC |
| | TCTCATCGACCAGTACCTGTATTACTTGAGCAGAACAAACACTCCAAGTGGAACC |
| | ACCACGATGTCAAGGCTTCAGTTTTCTCAGGCCGGAGCGAGTGACATTCGGGACC |
| | AGTCTAGGAACTGGCTTCCTGGACCCTGTTACCGCCAGCAGCGAGTATCAAAGAC |
| | AGCCGCGGATAACAACAACAGTGACTACTCGTGGACTGGAGCTACCAAGTACCAC |
| | CTCAATGGCAGAGACTCTCTGGTGAATCCGGGCCCGGCCATGGCAAGCCACAAGG |
| | ACGATGAAGAAAAGTACTTTCCTCAGAGCGGGGTTCTCATCTTTGGGAAGCAAGA |
| | CTCAGGCAAAACAAATGTGGACATTGAAAAGGTCATGATTACAGACGAAGAGGAA |
| | ATCAGGACAACCAATCCCGTGGCTACGGAGCAGTATGGTTCTGTATCTACCAACC |
| | TCCAGAGCGGCAACACCCAAGCAGCTACCAGCGATGTCAACACACAAGGCGTTCT |
| | TCCAGGCATGGTCTGGCAGGACAGAGATGTGTACCTTCAGGGGCCCATCTGGGCA |
| | AAGATTCCACACACGGACGGACATTTTCACCCCTCTCCCCTCATGGGTGGATTCG |
| | GACTTAAACACCCTCCTCCACAGATTCTCATCAAGAACACCCCGGTACCTGCGAA |
| | TCCTTCGACCACCTTCAGTGCGGCAAAGTTTGCTTCCTTCATCACACAGTACTCC |
| | ACGGGACAGGTCAGCGTGGAGATCGAGTGGGAGCTGCAGAAGGAAAACAGCAAAC |
| | GCTGGAATCCCGAAATTCAGTACACTTCCAACTACAACAAGTCTGTTAATGTGGA |
| | CTTTACTGTGGACACTAATGGCGTGTATTCAGAGCCTCGCCCCATTGGCACCAGA |
| | TACCTGACTCGTAATCTGTAATTGCTTGTTAATCAATAAACCGTTTAATTCGTTT |
| | CAGTTGAACTTTGGTCTCTGCGCGTCAAAAGGGCGACACAAAATTTATTCTAAAT |
| | GCATAATAAATACTGATAACATCTTATAGTTTGTATTATATTTTGTATTATCGTT |
| | GACATGTATAATTTTTCTAGAGCGGCCGCAGATCTCAGCTGGATATCAAAAACTG |
| | ATTTTCCCTTTATTATTTTCGAGATTTATTTTCTTAATTCTCTTTAACAAACTAG |
| | AAATATTGTATATACAAAAAATCATAAATAATAGATGAATAGTTTAATTATAGGT |
| | GTTCATCAATCGAAAAAGCAACGTATCTTATTTAAAGTGCGTTGCTTTTTTCTCA |
| | TTTATAAGGTTAAATAATTCTCATATATCAAGCAAAGTGACAGGCGCCCTTAAAT |
| | ATTCTGACAAATGCTCTTTCCCTAAACTCCCCCCATAAAAAAACCCGCCGAAGCG |
| | GGTTTTTACGTTATTTGCGGATTAACGATTACTCGTTATCAGAACCGCCCAGGGG |
| | GCCCGAGCTTAAGACTGGCCGTCGTTTTACAACACAGAAAGAGTTTGTAGAAACG |
| | CAAAAAGGCCATCCGTCAGGGGCCTTCTGCTTAGTTTGATGCCTGGCAGTTCCCT |
| | ACTCTCGCCTTCCGCTTCCTCGCTCACTGACTCGCTGCGCTCGGTCGTTCGGCTG |
| | CGGCGAGCGGTATCAGCTCACTCAAAGGCGGTAATACGGTTATCCACAGAATCAG |
| | GGGATAACGCAGGAAAGAACATGTGAGCAAAAGGCCAGCAAAAGGCCAGGAACCG |
| | TAAAAAGGCCGCGTTGCTGGCGTTTTTCCATAGGCTCCGCCCCCCTGACGAGCAT |
| | CACAAAAATCGACGCTCAAGTCAGAGGTGGCGAAACCCGACAGGACTATAAAGAT |
| | ACCAGGCGTTTCCCCCTGGAAGCTCCCTCGTGCGCTCTCCTGTTCCGACCCTGCC |
| | GCTTACCGGATACCTGTCCGCCTTTCTCCCTTCGGGAAGCGTGGCGCTTTCTCAT |
| | AGCTCACGCTGTAGGTATCTCAGTTCGGTGTAGGTCGTTCGCTCCAAGCTGGGCT |
| | GTGTGCACGAACCCCCCGTTCAGCCCGACCGCTGCGCCTTATCCGGTAACTATCG |
| | TCTTGAGTCCAACCCGGTAAGACACGACTTATCGCCACTGGCAGCAGCCACTGGT |
| | AACAGGATTAGCAGAGCGAGGTATGTAGGCGGTGCTACAGAGTTCTTGAAGTGGT |
| | GGGCTAACTACGGCTACACTAGAAGAACAGTATTTGGTATCTGCGCTCTGCTGAA |
| | GCCAGTTACCTTCGGAAAAAGAGTTGGTAGCTCTTGATCCGGCAAACAAACCACC |
| | GCTGGTAGCGGTGGTTTTTTTGTTTGCAAGCAGCAGATTACGCGCAGAAAAAAAG |
| | GATCTCAAGAAGATCCTTTGATCTTTTCTACGGGGTCTGACGCTCAGTGGAACGA |
| | CGCGCGCGTAACTCACGTTAAGGGATTTTGGTCATGAGCTTGCGCCGTCCCGTCA |
| | AGTCAGCGTAATGCTCTGCTTTTAGAAAAACTCATCGAGCATCAAATGAAACTGC |
| | AATTTATTCATATCAGGATTATCAATACCATATTTTTGAAAAAGCCGTTTCTGTA |
| | ATGAAGGAGAAAACTCACCGAGGCAGTTCCATAGGATGGCAAGATCCTGGTATCG |
| | GTCTGCGATTCCGACTCGTCCAACATCAATACAACCTATTAATTTCCCCTCGTCA |
| | AAAATAAGGTTATCAAGTGAGAAATCACCATGAGTGACGACTGAATCCGGTGAGA |
| | ATGGCAAAAGTTTATGCATTTCTTTCCAGACTTGTTCAACAGGCCAGCCATTACG |
| | CTCGTCATCAAAATCACTCGCATCAACCAAACCGTTATTCATTCGTGATTGCGCC |
| | TGAGCGAGGCGAAATACGCGATCGCTGTTAAAAGGACAATTACAAACAGGAATCG |
| | AGTGCAACCGGCGCAGGAACACTGCCAGCGCATCAACAATATTTTCACCTGAATC |
| | AGGATATTCTTCTAATACCTGGAACGCTGTTTTTCCGGGGATCGCAGTGGTGAGT |
| | AACCATGCATCATCAGGAGTACGGATAAAATGCTTGATGGTCGGAAGTGGCATAA |
| | ATTCCGTCAGCCAGTTTAGTCTGACCATCTCATCTGTAACATCATTGGCAACGCT |
| | ACCTTTGCCATGTTTCAGAAACAACTCTGGCGCATCGGGCTTCCCATACAAGCGA |
| | TAGATTGTCGCACCTGATTGCCCGACATTATCGCGAGCCCATTTATACCCATATA |
| | AATCAGCATCCATGTTGGAATTTAATCGCGGCCTCGACGTTTCCCGTTGAATATG |
| | GCTCATATTCTTCCTT |
| SEQ ID NO: | GAGCAGAAACTCATCTCAGAAGAGGATCTG |
| 35 | |
| MYC TAG | |
| SEQ ID NO: | TACCCTTACGATGTACCGGATTACGCA |
| 36 | |
| HA TAG | |
| SEQ ID NO: | GCGCTCCCTCCTCTCGGAGAGAGGGCTGTGGTAAAACCCGTCCGGAAATTGGCCG |
| 37 | CCGCTGCCGCCACCGCCGCCGCCGCCGCCGCGCCGAGCGGAGGAGGAGGAGGAGG |
| MECP2 | CGAGGAGGAGAGACTGTGAGTGGGACCGCCAAGGCCGCGGGCGGGGACCCTTGCT |
| intron | GGGGGGCGGGTAGGGGGGGGACGTGGCGCGGGAGGGGCCCGCGGGGTCGGGCGAC |
| | ACGGCTGGCGGTTGGCGTCCCTCCTCTCTACCCTCCCCCTCCCTCTGCCGCCGGT |
| | GGTGGCTTTCTCCACTCGTCTCCCGCAATCGCGAGCGACGGTTCTCAGCGCGATC |
| | TCCCTGGAGCCACCTTCGATTGACGCCCTCCCGCTGCCCGCCCCATCTGTGCGCA |
| | TCCTAGGCCCCAGCTGTGCAAGCGCCCTTGTCGTCTGGGCTTCGCCAGTTGGGGC |
| | TGCGCGCGCTCCTGCCCTTCTTGGGGCTTTGGGCCTCGGCACTGTCGCGCGCCCG |
| | CGGTCCCGGCCTCTCCCTGGATCGCGCTGTCCCCTTCTCCCTCGCGCGCCCCCAC |
| | TCCCGTTACTTGCTCCCCCCTCACACACACAGACTGGCGCGCGTGCGCAGTCCAT |
| | CTCCCGTTGGGAGAGTGCGCCACAAGGGCTCCTGAGCTCTTACCCCCATCTCTGG |
| | GTTTTGCTCCCTCCTCCTCCTCTCCCATTCCGTGACTTTTTGCCCCCACTGCAAG |
| | CGAGTCGGTCCATCAGCTCCATTCCCCACTTGGCAGGAACAAGTTGAGGGTTATT |
| | GTCCACCCACAAAAAGGACTAGACATTTTGTTCCTAGGTCCCACAACTCATCATA |
| | AAGAGTTGGTTGTAGTTCTCATCAGGAACCGTGGGCAAGGGACTGTGCGTTCCTC |
| | AGCACTCGAAGCTCTTCCGTGAGACCTTGCCCGCAGGGTGCTCTGGTTCTTTGGG |
| | GTTGCTGTGCTGTGGCTTCGGAATTTGAGCGTCTTCCCACCCTCCCTCCCCTCCC |
| | TTCGCCAGCGTTCTGTCTACAAGAAAGAATAGGCAGGTGTCCTTGGATATCGTAG |
| | TTGCTAATCGCCTATACACTGTTCTATTACACCTTTCTGCTAAGGATAGGGTTTT |
| | TGGTTTTGGTTTTGGTTTTGTTCCCCACCCTCCAGTTTGGTTTAGTTTTGGTTTT |
| | GGCATTTAGGGTTTTTTGGGGGGGAGTAATATCTTGTGGTAAAGACCCATCTGAC |
| | CCAAGATACCTTTTTTCTCATACTGGAACCCTAGGCAGCAGTTGCTATTTCCCTG |
| | AGTTAGCAATAGTTTTACAGTATTTTGAGGCCTTTTGTCCATAATTCTCACGGAA |
| | TCCCTCAGGGATCAGATTAGCTGCTGTTGGGATCAGGAAATTGGGTTACACCGCT |
| | GAAATCTCTTGCTGGGGCCCTTGTTTTGAATTGGAAAGTCAGGAGGCTGGAACGA |
| | AGGCTCACAAGTTAACAGTGCCAGCTGCTCTTCCAGAAGCCCTGGATTCAGTCCC |
| | ACCAATCCATCGCGGGTCACAACCATCTGTAACTTCAGTCCCAAGGGGTCCGAAG |
| | CCCTCTTCTGGCTTTGCCCTATTATTTTATTTATCTTATCTGTTTTTGTCTTGTC |
| | ATCTGGCAAGCCCAGGGGGCCATTGGGTGCAACTTATAAACTGACTTCTGTATCT |
| | TAAGAAGCCAACCATACAGTGCTTACATTCCAGAAAAAAAATCTGCCACTTTAAC |
| | AGCACTAGAACTAGGGTTTAGAGAAGTATCATAAAGGTCAAATATCTTTGACCAA |
| | TATCACCAGCAACCTAAAGCTGTTAAGAAATCTTTGGGCCCCAGCTTGACCCAAG |
| | GATACAGTATCCTAGGGAAGTTACCAAAATCAGAGATAGTATGCAGCAGCCAGGG |
| | GTCTCATGTGTGGCACTCAAGCTCACCTATACTCACTACTGTGCAGACAGCTGTG |
| | TTCTCTGTAATACTTACATATTTGTTTAATACTTCAGGGAGGAAAAGTCAGAAGA |
| | CCAGGATCTCCAGGGCCTCA |
| SEQ ID NO: | GSTHILTPTKELMDLRHPDFRESSRVSFEDQAPTME |
| 38 | |
| Munc18-1a | |
| (aa 568-603) | |
| SEQ ID NO: | GSTHILTPQKLLDTLKKLNKTDEEISS |
| 39 | |
| Munc18-1b | |
| (aa 568-594) | |
| SEQ ID NO: | GATCCAGACATGATAAGATACATTG |
| 40 | |
| forward | |
| primer | |
| SEQ ID NO: | GCAATAGCATCACAAATTTCAC |
| 41 | |
| reverse | |
| primer | |
| SEQ ID NO: | TGGACAAACCACAACTAGAATGCA |
| 42 | |
| Probe 6- | |
| Fam/Zen/3′ | |
| IB FQ | |
| SEQ ID NO: | DNALLAQLIQDK |
| 43 | |
| STXBP1 | |
| peptide | |
| SEQ ID NO: | YETSGIGEAR |
| 44 | |
| STXBP1 | |
| peptide | |
| SEQ ID NO: | ISEQTYQLSR |
| 45 | |
| STXBP1 | |
| peptide | |
| SEQ ID NO: | WEVLIGSTHILTPTK |
| 46 | |
| STXBP1 | |
| peptide (long | |
| isoform | |
| specific) | |
| SEQ ID NO: | WEVLIGSTHILTPQK |
| 47 | |
| STXBP1 | |
| peptide | |
| (short | |
| isoform | |
| specific). | |

### EXAMPLES

The following Examples illustrate the invention.

### Example 1: Construct Design, Generation and Cloning

Plasmids used in this study were constructed by recombinant DNA techniques. AAV Cis backbone plasmids were synthesized de-novo and contained two AAV inverted terminal repeats (ITRs), a kanamycin resistance cassette, a prokaryotic origin of replication, and an SV40 polyadenylation sequence. DNA sequences coding isoform variant X1 of the human STXBP1 (comprising SEQ ID NO: 7) were synthesized de-novo with convenient cloning restriction sites. Individual promoters were synthesized de-novo with convenient restriction sites. The Human influenza hemagglutinin (HA) or Myc tags (according to SEQ ID NO: 33 and 32, respectively) were synthesized as oligonucleotides from Integrated DNA Technologies™ (Coralville, IA, USA) and inserted at the amino or carboxy terminal. Seven different promoters (MECP2-intron, MECP2, hNSE, CamKII, hSyn, hSTXBP1p, CAG) were tested for human STXBP1 gene.

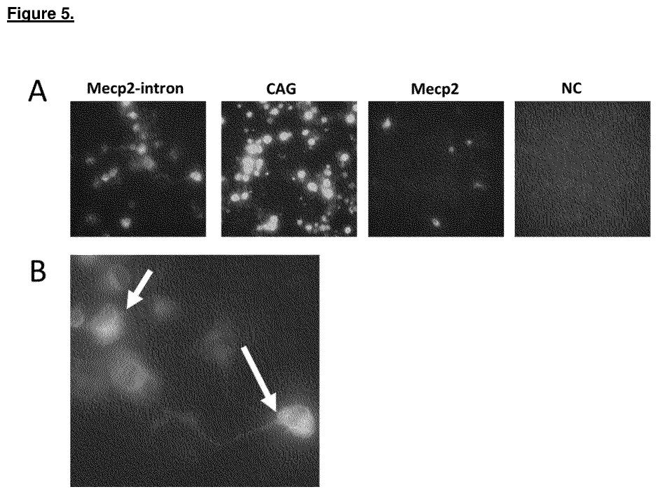

A schematic cartoon of the designed constructs is shown in FIG. 3. In the figure, “prom” means promoter; “INT” means intron, “h” means human, SV40 means polyadenylation sequence SV40; “tag” means an HA or Myc tag, located either at the N or at the C terminus of a construct.

### Example 2: Evaluation of STXBP1 Expression Under Different Promoters

### Cell Culture

The human-derived AD-HEK293 (Agilent Technologies™, Santa Clara, CA, USA) and mouse-derived Neuro-2A (ATCC™, Manassas, VA) cell lines were passaged in DMEM+10% FBS+1% Penicillin/Streptomycin (all from Thermo Fisher Scientific™, Waltham, MA, USA). Neuro-2A cells were differentiated by supplementing the growth media with 10 μM Retinoic Acid (MilliporeSigma™, Burlington, MA, USA) for 72 hours as previously described (Tremblay, R. G. et al. 2010). Cells were transfected using X-tremeGene 360 Transfection reagent (Roche, Mannheim, Germany) according to the manufacturer's protocol. A control transfection, with control plasmid was also included.

### Immunofluorescence and Microscopy

Imaging experiments were performed on a Zeiss Axio Observer 7 epifluorescent microscope (Carl Zeiss AG™, Oberkochen, Germany) equipped with a 40× objective lens, and a Hamamatsu Orca 4 flash cooled monochrome camera (Hamamatsu Photonics KK™ Hamamatsu City, Japan). Transfected AD-HEK293 and Neuro-2A cells were fixed with 4% paraformaldehyde (Electron Microscopy Sciences, Hatfield, PA, 19440), and stained with the rabbit polyclonal anti-STXBP1 (MilliporeSigma™, Burlington, MA, USA) at1:500. Cells were then stained with donkey anti-rabbit secondary antibodies conjugated to Alexa Fluor 488 at 1:1,000 prior to imaging.

FIG. 4: (A) Immunofluorescence imaging of AD-HEK293 cells transfected with hSTXBP1 plasmids driven by various promoters (CAG, MECP2 and MECP2-intron) detected with anti-STXBP1 antibody. (B) The magnification section shows that STXBP1 is localized to the cell membrane. AD=adherent, NC=negative control.

As shown in FIG. 4, transfected cells demonstrated different level expression of the human STXBP1 transgene under the drive of ubiquitous CAG promoter or neuro-specific promoters (MECP2 and MECP2-intron).

Neuro-2A transfected cells transfected with the STXBP1 plasmids driven by ubiquitous CAG promoter and neuro-specific promoters (MECP2 and MECP2-intron) were also analyzed, as shown in FIG. 5.

FIG. 5: (A) Immunofluorescence imaging of Neuro-2A cells transfected with hSTXBP1 plasmids driven by various promoters (CAG, MECP2 and MECP2-intron) detected with anti-STXBP1 antibody. (B) Magnification showing that STXBP1 is localized to the cell membrane. NC=negative control.

STXBP1 is a cytosolic protein interacting with a set of membrane associated proteins. Enlarged images of transfected AD-HEK293 and Neuro-2A show that STXBP1 expressed from these plasmids localizes to the plasma membrane (FIG. 4(B)) or both the neuronal processes and plasma membrane as expected (FIG. 5(B)).

### Western-Blot Analysis

Transfected AD-HEK 293 cells were harvested in 1× Cell Lysis Buffer (Cell Signaling Technology™, Danvers, MA, USA) containing 1× Halt Protease and Phosphatase Inhibitor Cocktail (Thermo Fisher Scientific™, Waltham, MA, USA) according to the manufacturer's instructions. Lithium dodecyl sulfate (LDS) Sample Buffer supplemented with 10% reducing agent (both Thermo Fisher Scientific™, Waltham, MA, US) were added to the protein lysates to a final concentration of 1×. Samples were resolved by 1 D SDS-PAGE gel electrophoresis. For each sample, 30 μg of proteins were loaded per lane. Proteins were transferred to nitrocellulose membranes (Li-Cor Biosciences™, Lincoln, NE, USA) using a semi-dry transfer apparatus (Bio-Rad Laboratories™, Hercules CA). Following transfer, membranes were incubated in blocking solution (Li-Cor Biosciences™, Lincoln, NE, USA) for 1 hour at room temperature. Membranes were then incubated with blocking solution containing primary antibodies overnight at 4° C. The following primary antibodies were used for this analysis: rabbit polyclonal anti-STXBP1 (MilliporeSigma™, Burlington, MA, USA) at 1:1,000, goat polyclonal anti-STXBP1 (Abnova, Taoyuan, Taiwan) at 1:1,000, rabbit polyclonal anti-c-myc at 1,1000 (MilliporeSigma™, Burlington, MA, USA), rabbit monoclonal anti-HA at 1:1,000 (Cell Signalling Technology™, Danvers, MA, USA), mouse monoclonal anti-GAPDH at 1:1,000 (MilliporeSigma™, Burlington, MA, USA). Membranes were washed three times with PBST solution, placed in blocking solution containing IRDye 680RD donkey anti-goat or IRDye 680RD donkey anti-rabbit secondary antibodies or 800CW donkey anti-mouse (1:15,000; Li-Cor Biosciences™, Lincoln, NE, USA) suitable for detection on the far-red spectrum for 1 hour at room temperature. Proteins were visualized using a Li-Cor Odyssey CLx far red imager (Li-Cor Biosciences™, Lincoln, NE, USA.

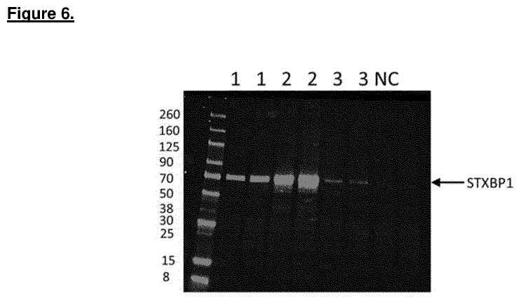

The molecular mass of the STXBP1 monomer under reducing conditions is predicted at ˜70 kDa and the protein was detected by western blot as a monomer. Detection of GAPDH was used as a loading control. These results show that robust expression was achieved by various promoters in differentiated Neuro-2A (FIG. 6).

FIG. 6: Western blot analysis of Neuro-2A cells transfected with hSTXBP1 driven by various promoters (CAG, MECP2 and MECP2-intron). Two technical replicates of each condition are shown. NC=negative control, 1=MECP2-intron-hSTXBP1, 2=CAG-hSTXBP1, 3=MECP2-hSTXBP1.

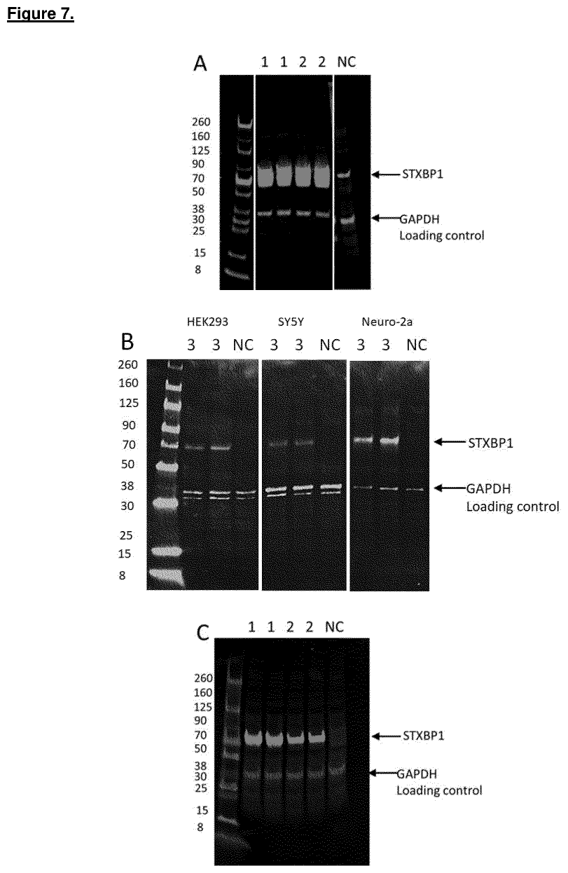

These results also show that robust expression was achieved by both the N- and C-terminal tagged constructs driven by the CAG promoter (FIG. 7).

FIG. 7: Western blot analysis of (A) Myc-tagged hSTXBP1 driven by CAG promoter in AD-HEK293 cells detected with anti-Myc antibody and (B) HA-tagged hSTXBP1 driven by hSYN promoter in AD-HEK293 cells, SH-SY5Y cells and Neuro-2a cells detected with anti-HA antibody. Two technical replicates of each condition are shown. (C) Epitope tagged proteins were also detected in AD-HEK293 cells using anti-STXBP1 antibodies. NC=negative control, 1=CAG-hSTXBP1-Myc, 2=CAG-Myc-hSTXBP1, 3=hSYN-HA-hSTXBP1. The background protein band in the NC lane in (A) is due to detection of endogenous Myc by the anti-Myc antibody.

### Example 3: Naturally Occurring Variants and Pathological Variants Identification and Analysis

The ClinVar database (https://www.ncbi.nlm.nih.gov/clinvar/), a freely accessible, public archive of reports of the relationships among human variations and phenotypes, with supporting evidence, was mined to identify STXBP1 gene variants using search term “STXBP1” and “pathogenic” or “likely pathogenic”. The list of pathogenic variants was 35 complemented with mutations published in scientific peer-reviewed literature and manually curated from a PubMed (https://pubmed.ncbi.nlm.nih.gov/) search using search terms “STXBP1, Munc18, variant, mutation” and defined as pathogenic by the authors to identify additional STXBP1 pathogenic variants not reported in ClinVar.

The pathological variants and likely pathological variants leading to a change in the STXBP1 protein were then identified. (Tables 5 and 6 respectively).

TABLE 5
Pathological variants (with reference to SEQ ID NO: 9)
Protein Change

| | |
|---|---|
| R190Q | Y531* |
| C516*, C549*, C552*, C538* | M294fs, M316fs, M327fs, M330fs |
| D371fs, D385fs, D382fs, D349fs | N489fs, N511fs, N522fs, N525fs |
| D76H, D90H | R278P, R289P, R292P |
| E12fs | R464fs, R428fs, R450fs, R461fs |
| E221fs, E218fs, E207fs | R388* |
| E273* | R100fs |
| E384*, E406*, E417*, E420* | C354Y |
| E39*, E53* | S533fs |
| F115fs | P480L |
| G193V | H245D |
| K340fs, K307fs, K329fs, K343fs | A297T |
| P125Q, P136Q, P139Q | C354R |
| Q203* | C366fs |
| R551H | D224fs |
| S155fs, S141fs, S152fs | E299fs, E302fs, E266fs, E288fs |
| S89fs | E302* |
| W478* | E337fs, E304fs, E326fs, E340fs |
| Y198*, Y209*, Y212* | F237fs |
| Y198*, Y209*, Y212* | G222fs, G233fs, G236fs |
| Y266* | G507E, G540E, G543E, G529E |
| E27* | I393fs |
| E418*, E407*, E421*, E385* | I490fs |
| E527*, E516*, E530*, E494* | I50fs |
| E67*, E81* | I539del |
| G508C, G530C, G541C, G544C | K196* |
| G544D | K328fs, K364fs, K350fs, K361fs |
| H498fs, H462fs, H495fs, H484fs | K447fs, K458fs, K461fs, K425fs |
| I232fs | K98fs, K84fs |
| K111fs, K122fs, K125fs | L130fs |
| M252fs | L36* |
| M443R | L87fs, L73fs |
| Q336* | N134fs |
| S440*, S462*, S473*, S476* | P433fs |
| V84D | Q250* |
| Y330fs, Y344fs, Y308fs, Y341fs | Q338* |
| Y519* | Q359* |
| C180Y | Q558* |
| R122* | Q576fs |
| R292H | R157fs, R168fs, R171fs |
| R190W | R500fs, R522fs, R533fs, R536fs |
| R406C | R522fs, R500fs, R533fs, R536fs |
| R235Q | R551L |
| R235* | R551P |
| G544V | S292fs, S328fs, S314fs, S325fs |
| K461fs | S300fs |
| R551C | S42fs |
| R367*, R331*, R353*, R364* | T504fs |
| E470* | W28* |
| W522* | W563* |
| A214fs | Y140* |
| G319fs | Y344*, Y322*, Y355*, Y358* |
| G544fs | Y402fs |
| R292L | Y551*, Y554*, Y518*, Y540* |
| Y250*, Y261*, Y264* | S476* |
| K120fs | E283K |
| I88fs | H523fs |
| | Q229* |

TABLE 6
Likely Pathological variants (with reference to SEQ ID NO: 9)
Protein change

| | |
|---|---|
| G530R, G541R, G508R, G544R | S28Y, S42Y |
| H311D, H333D, H347D, H344D | Y75C |
| T129P | R406H, R370H, R403H, R392H |
| A102del, A88del | L426P |
| D248Y, D259Y, D262Y | E171G, E182G, E185G |
| H89P, H103P | E207fs, E218fs, E221fs |
| K194fs, K205fs, K208fs | L390R, L412R, L423R, L426R |
| L41Q, L27Q | A517S |
| P65L, P79L | E487D |
| Y145H | G236D |
| W274R, W285R, W288R | H293Y |
| R406L | I232N |
| R551S | I390V |
| P139L | I485N |
| R403G, R392G, R370G, R406G | L256P |
| T361I | L291P |
| K32E, K46E | P335S |
| E455del, E477del, E488del, E491del | T570A |
| P444S, P466S, P477S, P480S | N548D |
| Y358fs | T2481 |

STXBP1 gene variants may include missense mutations, leading to amino acid substitutions. For example, R190Q in Table 5 means that arginine at position 190 with reference to SEQ ID NO: 9 is replaced by glutamine.

Other mutations may also occur. One type of mutation that was identified was a mutation involving the insertion or deletion of nucleotides in which the number of changed base pairs is not divisible by three which leads to the creation of a new amino acid sequence, a frameshift, indicated as “fs”. If the mutation disrupts the correct reading frame, the entire DNA sequence following the mutation will be read incorrectly. For example, E12fs in Table 5 means that glutamic acid at position 12 with reference to SEQ ID NO: 9 is changed due to a frameshift of nucleotides, resulting in an abnormal protein with an incorrect amino acid sequence.

Another type of mutation found in the variants was a mutation at the DNA level which removes one or more amino acid residues in the protein. This type of mutation is indicated as deletion (del) in the Tables. For example, 1539del in Table 5 means that isoleucine at position 539 with reference to SEQ ID NO: 9 is removed.

Other mutations included the introduction of a stop codon, indicated by an asterisk (*), which means that translation of the protein is stopped at this position, resulting in a shortened or truncated protein. For example, Y531* in Table 5 means that the stop mutation occurs in the codon that normally encodes tyrosine 531 with reference to SEQ ID NO: 9, terminating translation of the protein at this position.

Naturally occurring variants in healthy population were derived from gnomAD (The Genome Aggregation Database—https://gnomad.broadinstitute.org/v2.1.1), a publicly available control data-set containing genetic information from 60.146 samples from unrelated individuals using the query term “STXBP1”. The variants extracted from the control dataset include missense, start lost and stop gained variants resulting in amino acid change. The naturally occurring variants resulting in amino acid change are reported in Table 7.

TABLE 7
Naturally occurring variants (with reference to SEQ ID NO: 9)

| | | | | |
|---|---|---|---|---|
| Ala113Thr | Asp207Asn | Ile289Val | Pro67Leu | Val84Ile |
| Ala181Asp | Asp210Asn | Ile450Val | Pro94Leu | Val9Ile |
| Ala389Ser | Asp436Gly | Ile490Thr | Pro95Leu | Ala514dup |
| Ala430Thr | Asp49His | Leu169Met | Ser149Phe | Phe595LeufsTer30 |
| Ala444Val | Asp586Asn | Leu414Ser | Ser328Thr | Val593TyrfsTer32 |
| Ala514Thr | Asp597Asn | Lys161Arg | Ser533Thr | |
| Ala527Thr | Gln154His | Lys25Asn | Ser70Thr | |
| Arg100Gln | Gln338Arg | Lys277Arg | Thr129Ile | |
| Arg100Trp | Glu12Asp | Lys314Asn | Thr401Ile | |
| Arg171His | Gly222Ser | Lys364Arg | Thr419Met | |
| Arg192Gln | Gly520Val | Met15Val | Thr455Met | |
| Arg305GIn | Gly529Ser | Met38Thr | Thr588Ile | |
| Arg305Trp | His16Arg | Met602Val | Tyr519Cys | |
| Arg457Cys | His357Pro | Phe91Leu | Val104Ile | |
| Arg505Cys | Ile168Val | Pro434Leu | Val448Met | |
| Arg583Ser | Ile271Val | Pro462Leu | Val451Ile | |
| Arg64Cys | | | | |
| Asn398Ser | | | | |
| Asn548Ser | | | | |

### Example 4: Production of Viral Particles

### AAV Production

Trans plasmids containing the AAV2 Rep sequences followed by the AAV9.hu14 (hereinafter AAV9) or AAV-true type (hereinafter AAVtt) capsid sequences (according to SEQ ID NO: 17 and 34, respectively) were synthesized de-novo by ATUM™ (Newark, CA, USA). AAV helper plasmid pALD-X80 was purchased from Aldevron, LLC™ (Fargo, ND, USA).

Non-replicating AAV vectors were produced by the triple transfection method. Expi293 cells (Thermo Fisher™, Waltham, MA, USA) were passaged every 3-4 days using Expi293 Expression Media (Thermo Fisher™, Waltham, MA, USA) in shake flasks at a seeding density of 3.0E+05-5.5E+05 cells/mL. The Expi293 cells were cultured on an orbital shaker at 125 rpm in an Eppendorf incubator set at 37° C. with 5% CO₂. To set up the production flasks, a 125 mL shake flask was inoculated the day before transfection at 1.5E+05 cells/mL in a total volume of 30-66 mL per viral preparation. Viable cell density was calculated using a Vi-Cell Blu (Beckman Coulter™, Pasadena, CA, USA).

A transfection complex was created for each flask as follows for the production flask with a 30 mL working volume: 180 μL Polyethylenimine (PEI) MAX at 1 mg/mL (Polysciences Inc™, Warrington, PA, USA) was diluted in 1.5 mL OptiPRO serum free media (Thermo Fisher™, Waltham, MA, USA), vortexed at setting 8 four times and incubated for 5 minutes at room temperature. Separately, 20 μg of the Cis plasmid (as indicated in Table 10), 30 μg of the Rep/Cap plasmid (AAV9 or AAVtt), and 40 μg of the helper plasmid (pALD-X80) were diluted in 1.5 mL OptiPRO serum free media, vortexed at setting 8 four times and incubated for 5 minutes at room temperature. These two mixtures were then combined, vortexed at setting 8 four times, and incubated at room temperature for 15 minutes. Transfection complexes were then added to shake flasks containing cells. Cells were cultured with the transfection mixture at 37° C. with constant agitation at 125 rpm.

After 96 hours, flasks were spiked with the concentrated AAV lysis buffer to a final concentration of 1× (150 mM NaCl, 120 mM Tris-HCl [pH=8.0], 2 mM MgCl2, 0.1% Triton X-100), and Benzonase (MilliporeSigma™, Burlington, MA, USA) to a final concentration of 50 U/mL. This mixture was incubated for 1 hour at 37° C. with constant agitation at 125 rpm. The mixture was clarified by centrifugation at 2,880×g for 10 minutes at 23° C. Samples were stored at −80° C. until further analysis.

### AAV Titer Determination

Each sample was removed from −80° C. and allowed to thaw at room temperature for 15 minutes. Once the sample was thawed, it was briefly vortexed and centrifuged for one minute. After this, 10 μL of sample was added to an individual well of a 96-well PCR plate combined with 10× DNase Buffer, 50 U DNase, and DNase-free water (all from Promega™, Madison, WI, USA) to a total volume of 100 μL in each well.

The plate was then transferred to a Bio-Rad™ (Hercules, CA, USA) thermal cycler and was heated for 30 minutes at 37° C. then cooled to 4° C. Samples were then serially diluted as described in the Table 8.

TABLE 8

| | Inter- | | Inter- | | | |
|---|---|---|---|---|---|---|
| | mediate | Inter- | mediate | Diluent | Total | Total |
| Dilution | Dilution | mediate | Volume | Volume | Volume | Dilution |
| Step | Factor | Sample | (μL) | (μL) | (μL) | factor |
| D0 | 10 | DNase | 100 | NA | 100 | 10 |
| | | treated | | | | |
| | | samples | | | | |
| D1 | 1.5 | D0 | 100 | 50 | 150 | 1.5E+01 |
| D2 | 10 | D1 | 10 | 90 | 100 | 1.5E+02 |
| D3 | 10 | D2 | 10 | 90 | 100 | 1.5E+03 |
| D4 | 10 | D3 | 10 | 90 | 100 | 1.5E+04 |
| D5 | 10 | D4 | 10 | 90 | 100 | 1.5E+05 |

Five (5) μL of dilutions D2, D3, D4, and D5 were mixed with 20 μL of a ddPCR master mix composed of Supermix for Probes (No dUTP; Bio-Rad™, Hercules, CA, USA), forward primer GATCCAGACATGATAAGATACATTG (SEQ ID NO: 40), reverse primer GCAATAGCATCACAAATTTCAC (SEQ ID NO: 41), Probe 6-Fam/Zen/3′IB FQ: TGGACAAACCACAACTAGAATGCA (SEQ ID NO: 42), and DNase-free water to a final concentration of 1×. This primer set targets SV40 polyA region of the transgene. Each sample was run in duplicate in a 96-well PCR plate.

The plate was heat sealed with a foil covering, pulse vortexed, and centrifuged at 1,000×g for 5 minutes. The plate was placed into the Bio-Rad™ QX-200 droplet generator and droplets were generated per the manufacturer's instructions.

After droplet generation, the plate was heat-sealed with a foil covering and placed into a BioRad™ thermocycler programmed to run the cycle described in Table 9.

TABLE 9
PCR amplification Settings (all ramping is set at 2.5° C./Seconds

| Cycle Step | Temperature | Duration | Number of Cycles |
|---|---|---|---|
| Enzyme Activation | 95° C. | 10 minutes | 1 |
| Denaturing | 95° C. | 30 seconds | 39 |
| Annealing/Extension | 56° C. | 1 minute | |
| Enzyme Deactivation | 98° C. | 10 minutes | 1 |
| Hold | 4° C. | Infinite | 1 |

Once complete, the plate was placed into a Bio-Rad™ QX200 droplet for droplet reading per the manufacturer's instruction. The concentration of vector genomes (VG/mL) was quantified using the following formula:

VG/ML:X=[(aY)(1000/b)]D, where:
X is VG/mL;
a is volume of the ddPCR reaction (25 μl);
Y is the ddPCR readout in copies per microliter;
b is the volume of diluter vector in the ddPCR (5 μL);
D is the total dilution applied to the test material.

Assay acceptance criteria were defined as follows:

The % CV between the replicates must be ≤15%; if >15% one outlier may be omitted. If an outlier is omitted and the % CV remains >15%, the assay must be repeated. The inter-dilution % CV must be ≤20% and reported dilutions must be at least two consecutive dilutions. If the % CV is >20%, a dilution can be omitted so long the reported dilutions are at least two consecutive dilutions. If the averaged dilutions are still >20%, the assay must be repeated. Each reaction well must have ≥1,000 accepted droplets. If <10,000 droplets, the well will be excluded from analysis.

### Viral Particle Quantitation by AAV Capsid ELISA

The viral particle titer was determined by ELISA kits (PROGEN™ Biotechnik GmbH, Heidelberg, Germany) according to the manufacturer's instructions. For AAV9, the mouse monoclonal ADK9 antibody was used for both the capture and detection steps. For AAVtt, the A20R monoclonal antibody was used for both capture and detection steps. Washes in the provided 1× Assay Buffer (ASSB) were performed between each step using a Molecular Devices™ (San Jose, CA, USA) AquaMax 4000 microplate washer. Samples were detected with a Molecular Devices™ SpectraMax M5e plate reader. Capsid titers were interpolated from the standard curve and are reported in Table 10.

TABLE 10

| | | Total | | Total | Viral |
|---|---|---|---|---|---|
| | | viral | Capsid | viral | genome |
| | | particles | titers | genomes | titers |
| Cis plasmid | Capsid | (VP) | (VP/mL) | (VG) | (VG/mL) |
| MECP2-hSTXBP1 | AAV9 | 2.15E+13 | 7.17E+11 | 7.77E+11 | 2.59E+10 |
| MECP2-intron- | AAV9 | 1.94E+13 | 6.47E+11 | 6.72E+11 | 2.24E+10 |
| hSTXBP1 | | | | | |
| MECP2-hSTXBP1 | AAVTT | 6.72E+12 | 2.24E+11 | 6.06E+11 | 2.02E+10 |
| MECP2-intron- | AAVTT | 4.38E+12 | 1.46E+11 | 5.04E+11 | 1.68E+10 |
| hSTXBP1 | | | | | |

The viral genome titers obtained by ddPCR and capsid titers obtained by ELISA indicated that both AAV9 and AAVtt viral particles comprising a viral vector with a nucleic acid comprising an indicated promoter operably-linked to a human STXBP1 transgene could be successfully produced.

### Example 5: Lentiviral Expression of STXBP1 Cassettes in NGN2 Differentiated Glutamatergic Neurons

A gene edited iPSC-line (EBiSC, Ref: BIONi010-C-13) carrying a DOX-inducible NGN2 expression cassette was used to generate iPSC derived glutamatergic neurons. In this protocol, the NGN2 transcription factor was induced by doxycycline for 9 days to prime neuronal differentiation. At division (DIV) 21, the iPSC derived NGN2 neurons were transduced with serial dilutions of lentiviral vectors expressing human STXBP1 (SEQ ID NO: 9) under the control of the hSyn or MECP2 promoter. The lentiviral vectors were produced in HEK 293 cells using a third-generation system for improved safety. At DIV28, immunocytochemistry (ICC) analysis was performed as follows: cells were fixed with 2% paraformaldehyde and stained with a primary rabbit polyclonal anti-STXBP1 antibody (Sigma, Ref: HPA008209) at a dilution of 1:250. Cells were then stained with a goat anti-rabbit secondary antibody conjugated to Alexa Fluor 568 at a 1:1000 dilution. Imaging was performed with an InCell analyser 6000 instrument using empirical parameters.

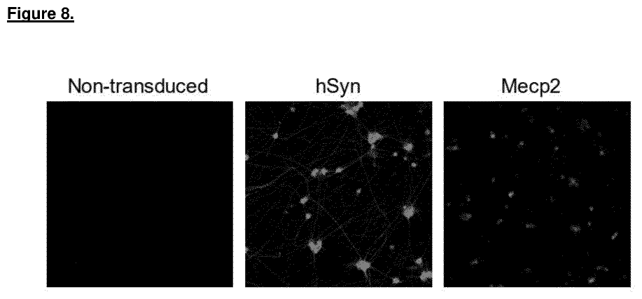

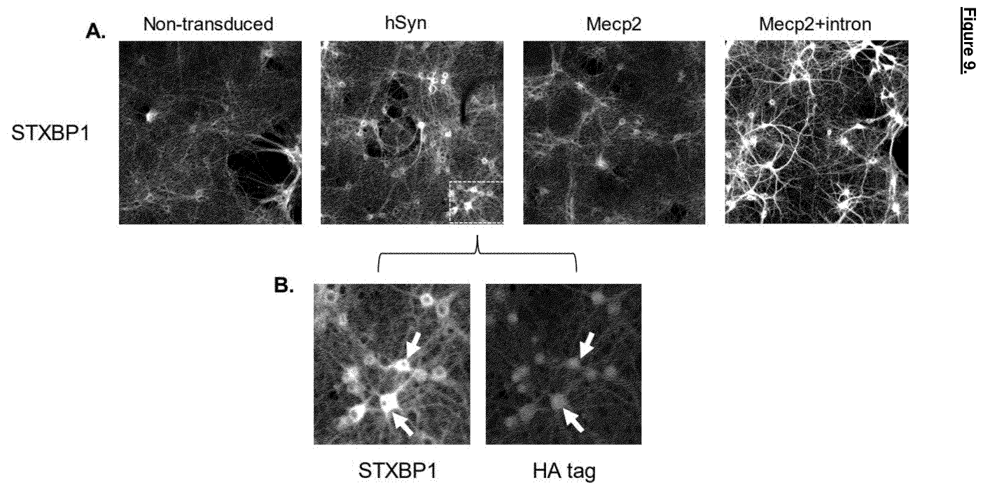

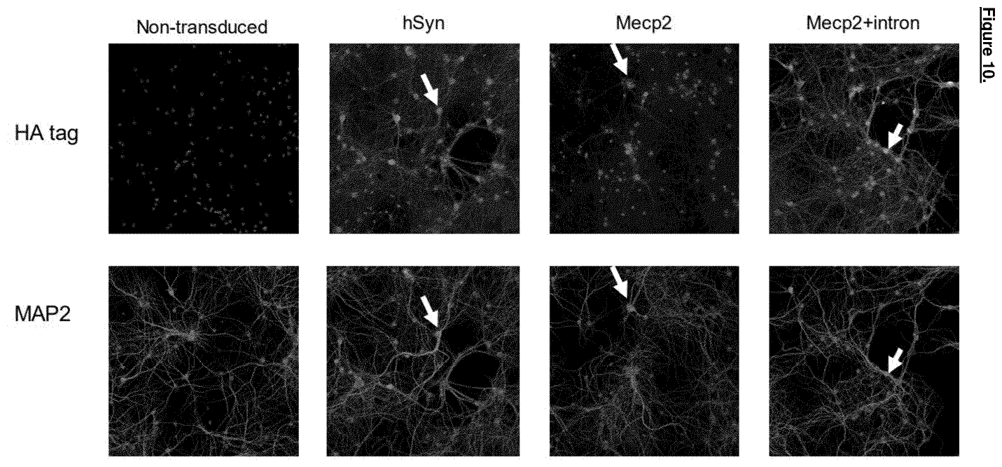

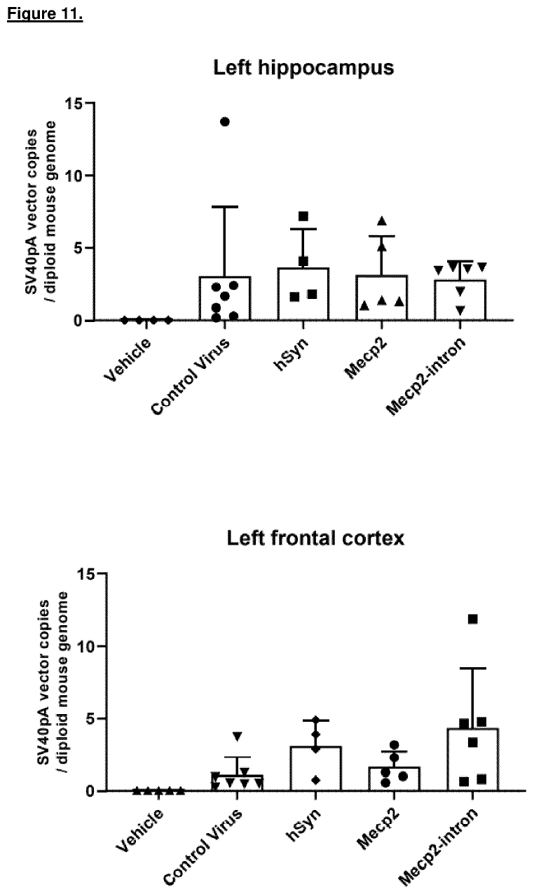

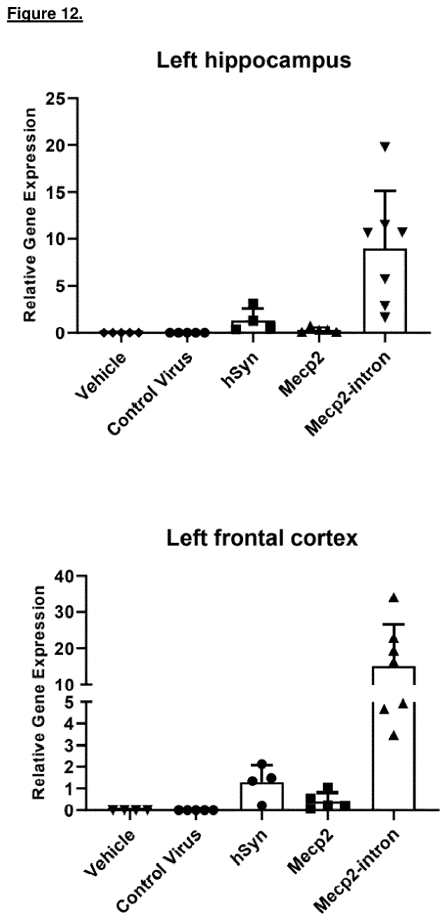

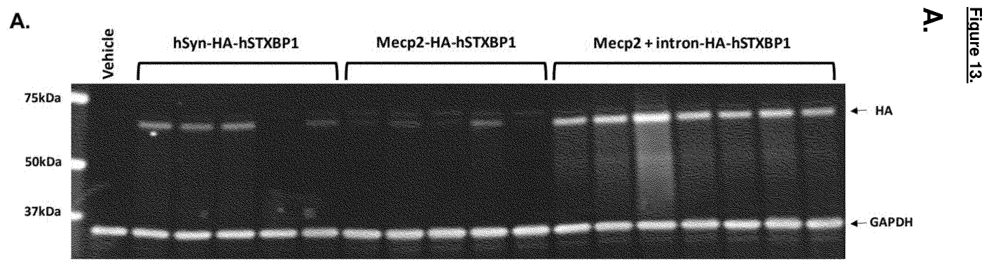

Representative ICC images from cells transduced with lentiviral vectors with a multiplicity of infection (MOI) of 2 are shown in FIG. 8.

FIG. 8: Lentiviral vector transduction of SXTBP1 cassettes in iPSCs derived glutamatergic neurons. Images show representative pictures of STXBP1 expression under control conditions (non transduced) and following transduction of cassettes under the control of the hSyn or MECP2 promoters.

Pictures were taken using the same acquisition setting for all conditions. Comparison of the signal with the non-transduced cells allowed the visualization of the over-expression of STXBP1 in the human iPSCs derived NGN2 neurons. STXBP1 under the control of the hSyn promoter resulted in a higher expression than the MECP2 promoter (summarized in Table 11)

TABLE 11
STXBP1 expression levels following lentiviral transduction
in iPSC derived glutamatergic neurons

| Non transduced | hSYN-STXBP1 | MECP2-STXBP1 |
|---|---|---|
| Not detected | ++ | + |

(+) relative expression levels observed by ICC analysis

### Example 6: AAV-9 Transduction of STXBP1 Cassettes in Primary Mouse Neurons

AAV9 vectors were produced as described in Example 4 and capsid characteristics are listed in Table 12. The transgene expressed STXBP1 protein (SEQ ID NO: 9) fused to a HA tag at the N-terminus and expression was driven by the following promoters: hSyn, MECP2 or MECP2-intron. The HA-tagged protein was used to differentiate transgene expression from endogenous STXBP1 levels. An AAV9 capsid with a CAG-eGFP-NLS cassette was used as a control vector for transduction efficiency. STXBP1 expression was investigated in vitro by transducing mouse primary cortical neurons. Non-transduced cells were used as a control for endogenous STXBP1 expression.

TABLE 12
AAV9 viral vector properties

| | | Viral | |
|---|---|---|---|
| | | genomes | Endotoxin |
| | | titers | (<10 |
| Cassette | Capsid | (vg/mL) | EU/mL) |
| hSyn-eGFP | AAV9 | 1.36E+13 | Pass |
| MECP2-HA- | AAV9 | 1.39E+13 | Pass |
| hSTXBP1 | | | |
| MECP2-intron- | AAV9 | 1.35E+13 | Pass |
| HA-hSTXBP1 | | | |
| hSyn-HA- | AAV9 | 1.28E+13 | Pass |
| hSTXBP1 | | | |
| CAG-HA- | AAV9 | 1.22E+13 | Pass |
| hSTXBP1 | | | |
| CAG-eGFP | AAV9 | 1.30E+13 | Pass |

Mouse primary cortical neuronal cells were prepared from cortical tissue of E17 mouse embryos. Cortical tissues were dissociated using papain for 30 min at 37° C. and maintained in culture in Neurobasal™ Medium supplemented with B27 supplement 2%, GlutaMAX-I 1 mM and Penicillin-Streptomycin 50 units/ml. Half medium change was performed every week.

At division (DIV) 7, cells were transduced with the different AAV9 constructs at two different MOIs (2.5E+6 GC/cell and 5.0E+5 GC/cell). The level of transduction was confirmed by including the hSyn-eGFP-NLS construct which was high in both MOI conditions. At DIV13, cells were fixed with 2% paraformaldehyde and stained with the primary rabbit polyclonal anti-STXBP1 antibody (1:250; Sigma, Ref: HPA008209) and by anti-HA tag staining (1:100; Ref: 2367S, Cell Signaling Technology). Imaging was performed with an InCell analyser 6000 instrument using empirical parameters.

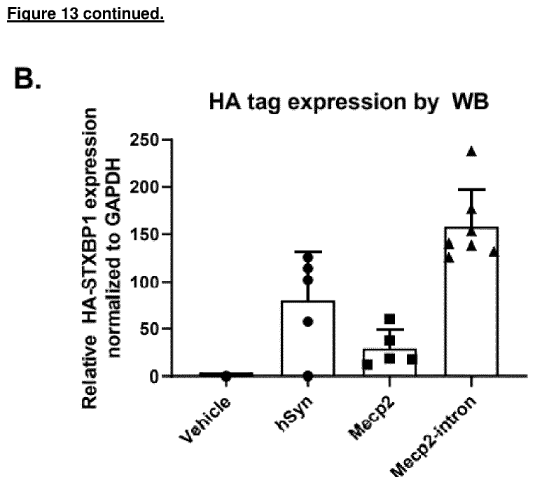

FIG. 9: AAV9 transduction of STXBP1 in mouse primary neurons (A) Representative images of STXBP1 staining in primary mouse cortical neurons transduced with AAV9 viral vector at MOI 5.0E+5 GC/cell. Pictures show control conditions (non transduced) and STXBP1 expression under control of hSyn, MECP2 or MECP2-intron promoters (B) Comparison of the HA staining (right) with the STXBP1 staining (left) in the same primary mouse cortical neurons.

Using similar acquisition parameters we confirmed increased STXBP1 expression levels for all three promoters when compared to non-transduced cells (FIG. 9A) suggesting that AAV9 transduction can achieve STXBP1 expression over baseline levels. The MECP2 with intron promoter showed the highest expression levels, followed by hSyn and MECP2 (Table 13). Moreover, co-localization of anti-HA with STXBP1 staining was observed for all viral vectors studied, a representative image is shown in FIG. 9B (see arrows).

TABLE 13
AAV9 transduction of SXTBP1 in mouse primary neurons
under the control of different neuronal promoters

| | Non- | | | |
|---|---|---|---|---|
| | transduced | hSyn | MECP2 | MECP2-intron |
| STXBP1 | − | ++ | + | +++ |

(+) relative expression levels observed by ICC analysis

To demonstrate that STXBP1 transduction was specific to neuronal cells we counter-stained the mouse primary cultures with an antibody directed against the pan neuronal marker MAP2 (1:5000; Ref: ab5392; Abcam™, Cambridge, MA, USA).

FIG. 10: Co-localization of STXBP1 over-expression in MAP2 positive neurons. Images are representative pictures of anti-HA tag staining (left panels) and anti-MAP2 staining (right panels) in mouse primary neurons following transduction of the AAV9 viral vectors. Arrows indicate examples of cells that express STXBP1 (HA) and the neuronal marker (MAP2).

FIG. 10 shows co-localization of the neuronal marker (MAP2) with the anti-HA staining in the transduced mouse primary cortical neurons (see arrows). This data confirmed the neuronal expression of the HA-tagged STXBP1 transgene product under the control of the different neuronal promoters. The intensity of HA-tag signal (Table 14) correlates with the STXBP1 levels (Table 13) suggesting that promoter strength may be ranked as follows: MECP2-intron>hSyn>MECP2.

TABLE 14
Expression and localization of HA tag in mouse primary neurons

| | Non- | hSyn- | MECP2- | MECP2-intron- |
|---|---|---|---|---|
| | transduced | HA_STXBP1 | HA_STXBP1 | HA_STXBP |
| HA-tag | − | ++ | + | +++ |
| signal | | | | |
| Co- | | yes | yes | yes |
| localization | | | | |

(+) relative expression levels observed by ICC analysis

### Example 7: In Vivo Expression of STXBP1 Following AAV-9 Mediated Transduction in Mouse Brain

AAV9 mediated transduction of STXBP1 in mouse brain was investigated in vivo. Viral vectors were administrated by bilateral intracerebroventricular (ICV) injection into the brain of post-natal 1 day old neonatal mice (PND1). The methodology for ICV neonatal injections has been previously described (Bertrand-Mathon, et al. 2015; Kim, et al. 2014; Hamodi, et al. 2020). Injected animals were monitored for a period of 5 weeks and the expression and distribution of STXBP1 was analyzed by biochemical readouts on brain tissues.

Experiments included 5 groups: Control (vehicle injected), Control virus (AAV9/hSyn_eGFP), AAV9/hSyn-HA-STXBP1, AAV9/MECP2-HA-STXBP1 and AAV9/MECP2-intron-HA-STXBP1. AAV9 vectors were the same as described in Table 12. A summary of the in vivo experimental conditions is shown in Table 15.

TABLE 15
Summary of in vivo experimental conditions

| | | Viral | | In Life | |
|---|---|---|---|---|---|
| Age at | Delivery | Vector | | Assess- | Terminal |
| treatment | Route | Cassette | Titer | ment | Assesment |
| Postnatal | ICV | AAV9/ | 1.28E+13 | Clinical | Brain and |
| day 1 | bilaterally | hSyn- | GC/mL | signs, | organs |
| PND1 | injected | STXBP1 | | adverse | collection 5 |
| | 2 ul/ | AAV9/ | 1.39E+13 | effects, | weeks post- |
| | hemi- | MECP2- | GC/mL | body | injection for |
| | sphere | STXBP1 | | weight, | biochemical |
| | | AAV9/ | 1.35E+13 | mortality | analysis, |
| | | MECP2- | GC/mL | during 5 | histo- |
| | | intron | | weeks | pathology, |
| | | STXBP1 | | post- | immuno- |
| | | AAV9/ | 1.36E+13 | injection | histo- |
| | | hSyn_ | GC/mL | | chemistry, |
| | | EGFP | | | transgene |
| | | Vehicle | (Sterile | | expression. |
| | | | Phosphate- | | |
| | | | buffered | | |
| | | | saline 1X) | | |

Body weight differences were monitored over the course of the study (5 weeks post-injection) to assess the overall health of the mice. There were no significant differences in the body weights of the different cassette groups at the time of the last assessment. None of the groups showed any clinical signs of toxicity. Additionally, there were no obvious signs of morbidity or delays in development in adult wild-type mice treated with AAV9/hSyn-HA-STXBP1, AAV9/MECP2-HA-STXBP1 or AAV9/MECP2-intron-HA-STXBP1. The results of this experiment demonstrated that the viral vector cassettes exhibited long-term tolerance and low toxicity and therefore can be safely used in a pre-clinical setting.

At 5 weeks post-injection, brain tissues were collected, dissected and submitted for biochemical analysis. DNA/RNA was extracted from left frontal cortex and hippocampus, while proteins were extracted from matching right frontal cortex. DNA/RNA extraction was performed using the AllPrep mini kit (Qiagen, 80204) following manufacturer instructions and including a DNAse treatment for the RNA extraction. The tissues were lysed in RLT Plus buffer (supplemented with α-mercaptoethanol) using the Precellys 24 instrument (Bertin Technologies). The DNA concentration was measured and adjusted to 20 ng/μl for all samples. Then, 40 ng were submitted to qPCR using primers/probe specific for the SV40 polyA signal (present in all the AAV cassettes). The amount of mouse genomes was analyzed using the ValidPrime® kit (tataabiocenter, A106P25). The ValidPrime® sequence is specific to a non-transcribed locus of gDNA that is present in exactly one copy per haploid normal genome. For both qPCR, copy numbers were determined using the standard curve method. The RNA concentration was measured, and 500 ng of RNA were submitted to RT using the kit High Capacity cDNA RT Kit+RNase Inhibitor (Applied Biosystems cat n° 4374966). The obtained cDNAs were submitted to the human STXBP1 signal qPCR, as well as two reference genes for normalization of the results obtained. Relative expression was determined and scaled to the average value for all groups. For the protein extraction, tissues were lysed in RIPA buffer (Pierce, 89900) including 2× concentrated Protease and phosphatase inhibitors cocktail (Cell Signaling Technology, #5872) using the Precellys 24 instrument (Bertin Technologies) and cooling system. The samples were left on ice for 30 min, centrifuged and the supernatant was collected as the final protein extract. Protein concentration was determined using the BCA Protein Assay Kit (Pierce, 23227) and 7.5 μg of protein was mixed with Laemli buffer and β-mercaptoethanol and incubated at 90° C. for 10 minutes prior to SDS-Page. Gels were transferred to nitrocellulose membranes and analysed by Western blot. Membranes were incubated in blocking solution (Ref: 927-50000; Li-Cor) for 1 hour at 4° C. followed by incubation with the primary antibodies mouse monoclonal anti-HA (1:2000; Ref: 2367S, Cell Signaling Technology) and mouse monoclonal anti GAPDH (1:10000; Ref: G8795, Sigma). The secondary antibodies used were IRDye® 680RD Donkey anti-Mouse IgG Secondary Antibody (1:20000; Ref: 926-68072, Li-Cor) and IRDye® 800CW Donkey anti-Rabbit IgG Secondary Antibody (1:20000; Ref: 926-32213, Li-Cor).

FIG. 11: Viral vector DNA copies analysis. qPCR data of SV40 pA (polyA signal of simian virus 40) normalized by the number of diploid mouse genomes from the left hippocampus and left frontal cortex of 5 weeks old mice following AAV treatment. Data is shown for vehicle and the four AAV9 transduced groups (control virus, hSyn, MECP2, MECP2-intron). Results are shown as mean±SD.

FIG. 12: STXBP1 mRNA expression analysis. Data are expressed as relative expression normalized to two reference genes and scaled to the average expression of all groups (mean±SD). Analysis was performed from tissues of the left hippocampus and left frontal cortex of 5 weeks old mice following AAV treatment. Data is shown for vehicle and the four AAV9 transduced groups (control virus, hSyn, MECP2, MECP2-intron).

FIG. 13: Protein analysis by Western blot. (A) Western blot showing HA-tag expression for the different cassettes in the cortex (n=5-7 per group). GAPDH was used as a loading control. (B) Quantification of the HA-tag band intensities, each sample is normalized to the GAPDH loading control. Results are shown as the mean±SD.

As illustrated in FIG. 11, significant vector DNA copies per diploid mouse genomes were detected in the DNA extract and demonstrated an efficient AAV9 transduction among the different viral vectors in the hippocampus and cortex (N=5-7 mice). Human STXBP1 transgene expression (mRNA) was observed for all three cassettes and a much stronger expression was observed for the MECP2-intron cassette compared to hSyn and MECP2 (FIG. 12). Western blot analysis of the HA tagged STXBP1 protein confirmed specific transgene product expression in vivo in the prefrontal cortex for all three cassettes studied (FIGS. 13 A and B). All together the data allowed general ranking of promoter strength among the viral vectors. The MECP2-intron showed the highest HA-tag STXBP1 expression followed by hSyn and MECP2. This data was in line with the in vitro data in mouse primary cortical neurons where similar relative ranking was observed.

### Example 8: In Vivo Distribution of STXBP1 Following AAV-9 Mediated Transduction in Mouse Brain

The distribution of STXBP1 expression in the mouse brain following PND1 injection of AAV9 vectors was investigated by immunohistochemistry (IHC). Mouse brain tissues were collected from the same animals as described in Example 7.

Fixed frozen sections (12 μm thickness; sagittal sections) were generated with a cryostat-microtome and stored at −80° C. All of the following incubation steps were carried out at room temperature. The cryosections were rinsed 10 min in PBS 1×, and then incubated with the following primary antibodies: GFP (1:2,000; #1020, Aves), HA (hemagglutinin tag; 1:5,000; #3724, Cell Signaling), NeuN (1:2,000; ab177487, Abcam), GFAP (1:2,000; #173006, Synaptic Systems), parvalbumin (1:500; PV235, Swant), alone or in combination for double immunofluorescence, diluted in PBS containing 0.3% Triton X-100, overnight in a humidified chamber. Following incubation, the sections were washed 3 times with PBS, then incubated for 1 hour with the appropriate Alexa-conjugated secondary antibodies (anti-mouse, rabbit, chicken, conjugated to Alexa 488 or 647). Then, they were counterstained with DAPI (300 nM dilution) to label cell nuclei, and washed 3 times with PBS. The sections were finally mounted with Prolong Gold antifade mounting media (Life Technologies) and a coverslip was applied. Digital images of stained sections were obtained using an AxioScan Z1 slide scanner with a 20× objective (Zeiss) and analyzed using Zen 3 software (Zeiss). To study the distribution of transduced cells in the brain expressing a transgene from a neuronal promoter, mouse pups were injected icv with AAV9/hSyn_eGFP at PND1. The animals were sacrificed 1 month after virus administration and the brains were dissected out and processed for immunohistochemistry to label GFP.

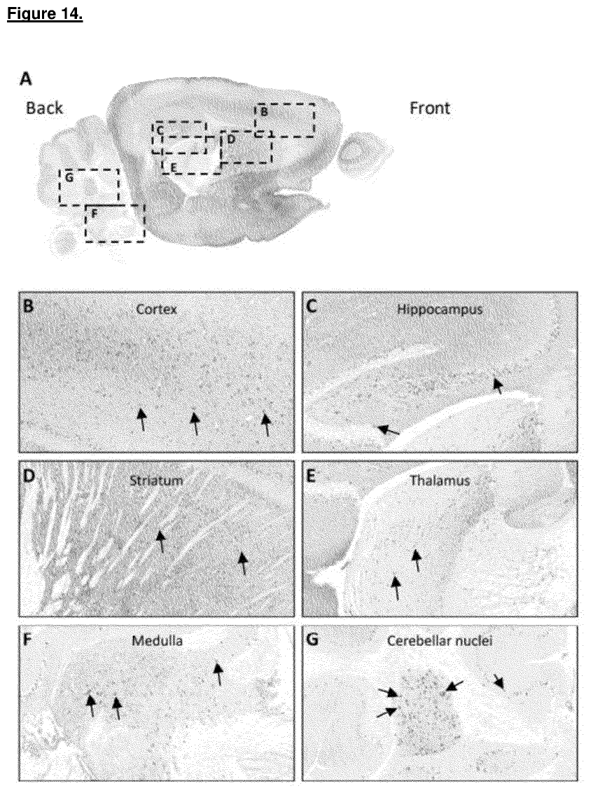

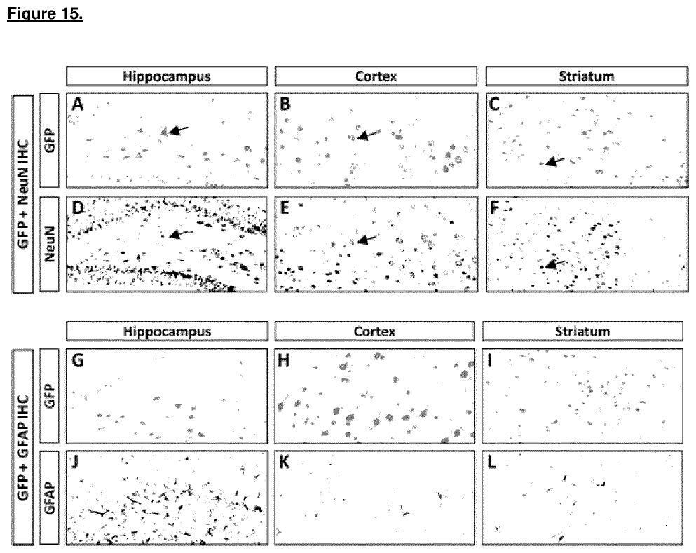

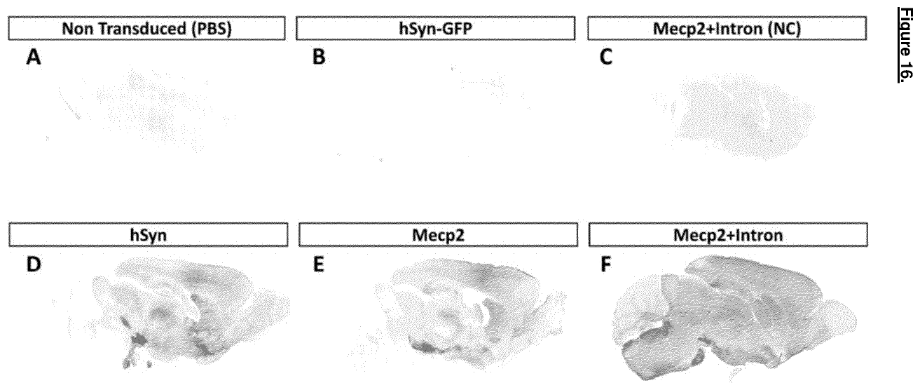

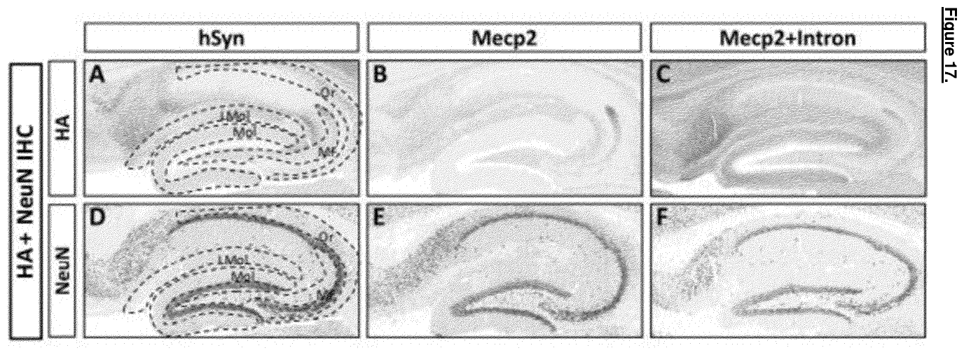

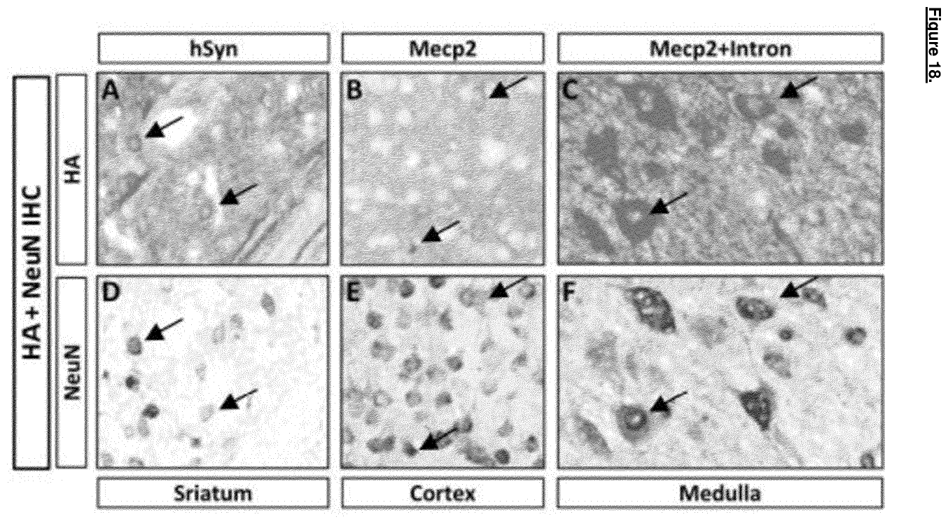

FIG. 14: Distribution of infected cells in the mouse brain using GFP reporter from AAV9-hSyn-NLS-eGFP-NLS virus. (A) Sagittal section of a mouse brain that received AAV9-hSyn1-NLS-GFP-NLS icv, sacrificed 1 month later, and immunostained to label GFP. The distribution of cells expressing GFP was observed from front to back of the entire brain. Some of the main brain regions exhibiting GFP+ cells are highlighted with rectangles. (B-G): High magnification of the brain regions showing GFP+ cells from A (arrows points to GFP+ cells).

FIG. 15: Characterization of cells expressing GFP reporter from AAV9-hSyn-NLS-eGFP-NLS virus. Double immunofluorescent labeling was performed to detect (A-F) GFP and the neuronal marker NeuN, (G-L) GFP and the astrocytic marker GFAP. Cell positives both for (A-C) GFP and (D-F) NeuN were observed in all brain regions (arrows point to double-labeled cells) indicating that neurons were transduced and expressed the reporter gene. To the opposite, no GFP (G-I) signal was detected in GFAP positive cells (J-L), suggesting that astrocytes were not expressing the reporter gene.

FIG. 16: Distribution of HA-STXBP1 fusion protein from different promoters in the mouse brain following AAV9 administration. The distribution of HA-tagged STXBP1 overexpressed from different promoters was studied in the brain of mice by immunohistochemistry against HA. As negative control conditions, no HA signal was observed in animals that received (A) PBS only or (B) AAV9-hSyn-GFP virus icv. (C) As a negative control (NC) of antibody selectivity, no HA signal was observed in animals that received AAV9-MECP2-intron-HA-STXBP1 virus but for which the primary HA antibody was omitted during the immunohistochemistry procedure. (D-F) HA signal was observed in the brain of all the animals injected with the different viruses expressing HA-STXBP1 from the different promoters. The 3 promoters led to a common pattern of HA distribution across the whole brain with main expression observed in the cerebral cortex, hippocampus, striatum, olfactory bulbs, substantia nigra and fiber tracts in the forebrain. Noticeable differences in HA distribution between promoters are reported in table 16.

FIG. 17: Distribution of HA-STXBP1 fusion protein from different promoters in the hippocampus following AAV9 administration. Double immunofluorescent labeling was performed to detect (A-C) HA and (D-F) the neuronal marker NeuN which was used to identify the different parts of the hippocampus. All 3 promoters led to HA expression in the entire hippocampus, mainly in neuronal projections (Mol, LMol, Or, MF) and occasionally in cell bodies. (F) MECP2-intron promoter led to a better coverage and higher HA signal intensity compared to the other 2 promoters (D, E). LMol: lacunosum molecular layer the hippocampus; MF: mossy fibers; Mol: molecular layer of the dentate gyrus; Or: stratum oriens.

FIG. 18: Characterization of cells expressing HA-STXBP1 from different promoters. Double immunofluorescent labeling was performed to detect (A-C) HA and (D-F) the neuronal marker NeuN. The cell bodies that were positive for HA and observed occasionally in different regions of the brain were also positive for NeuN supporting that all 3 promoters drive transgene expression in neurons. Arrows point to double labeled cells.

Overall, GFP+ cells were observed throughout the entire brain from the olfactory bulbs to the cerebellum and brainstem (FIG. 14A-G). A high number of infected cells was notably observed in the striatum (FIG. 14D), cerebral cortex (FIG. 14B), hippocampus (FIG. 14C) and olfactory bulbs. Double immunolabeling confirmed that GFP was expressed exclusively by neurons as attested by colocalization between GFP and the neuronal marker NeuN (FIG. 15A-F) and the absence of colocalization of GFP with the astrocytic marker GFAP (FIG. 15G-L).

The tissue distribution of HA-tagged STXBP1 overexpressed from 3 different neuronal promoters, hSyn, MECP2 or MECP2-intron, was analyzed by performing immunohistochemistry against HA (FIG. 16). The 3 promoters led to a common pattern of HA expression across the whole brain (FIG. 16D-F); the main regions where HA staining was observed were the cerebral cortex, hippocampus, striatum, olfactory bulbs, substantia nigra and fiber tracts in the forebrain. HA signal was detected in the cerebellum only in the animals injected with the AAV including the MECP2-intron promoter (FIG. 16F); the MECP2-intron promoter provided the best HA signal coverage and signal intensity across the brain compared to the 2 other promoters (FIG. 16D-F). A summary of the brain distribution of HA from the 3 promoters is provided in table 16. Of importance for the aim of developing a therapeutic approach to treat epilepsy, HA expression was observed in the hippocampus and cortex, a key region involved in epileptogenesis and seizure generation. All promoters led to HA expression in the entire hippocampus (FIG. 17), mainly in neuronal projections (mossy fibers, molecular layer of the dentate gyrus, lacunosum molecular and stratum oriens layers of the hippocampus). The highest HA signal intensity was observed with the MECP2-intron promoter; hSyn promoter led to an intermediate level of expression while MECP2 promoter provided the weakest signal intensity (FIG. 17A-C). At the cellular level, HA expression was mainly observed in the neuropil and occasionally in cell bodies (FIG. 18A-C) which colocalized with the neuronal marker NeuN (FIG. 18D-F), suggesting that all 3 promoters drive expression in neurons.

TABLE 16
Summary of the distribution of HA-STXBP1 transgene
product for different promoters in the mouse brain

| | | | MECP2- |
|---|---|---|---|
| | hSyn | MECP2 | intron |
| Cortex | x | x | x |
| Hippocampus | x | x | x |
| Olfactory bulbs | x | x | x |
| Striatum | x | x | x |
| Thalamus | x | x | x |
| Fiber tracts | x | x | x |
| (forebrain) | | | |
| Hypothalamus | x | | x |
| Midbrain | x | | x |
| Substantia nigra | x | x | x |
| Cerebellar nuclei | | | x |
| Medulla | | | x |

(x) confirmed tissue expression

### Example 9: Characterization of STXBP1 Variant Expression in WT and Heterozygous STXBP1 (HET) Mouse Brain

To evaluate the expression of STXBP1 protein variants in normal and disease conditions, a transgenic mouse model that recapitulates human STXBP1 haploinsufficiency-mediated epilepsy was generated and that has been described by Kovacevic et al. (2018). This mouse model was acquired through a license from the University of Amsterdam. The heterozygous model was generated with Stxbp1 floxed (Stxbp1fl/fl) mice with loxP sites on either side of exon 2 in the Stxbp1 gene. Stxbp1fl/fl were crossed to Ella-Cre (Jax: 003724) to delete Stxbp1 exon 2 in germ line resulting in Stxbp1fl/− null mutant mice. The floxed allele has been outbred to C57BL/6J generating the Stxbp1+/− KO HET mouse strain. Deletion of exon 2 in one allele leads to a premature stop codon and results in expression of a truncated and non-functional STXBP1 protein. All in vivo experiments were conducted in compliance with guidelines issued by the ethics committee for animal experimentation according to Belgian law. The experiments were performed in accordance with the European Committee Council directive (2010/63/EU). All efforts were made to minimize animal suffering.

To evaluate the endogenous STXBP1 variants expression, heterozygous KO (STXBP1+/−) and wildtype (WT) littermate (STXBP1+/+) male mice were sacrificed 5-7 weeks post-natal and the brain tissues were collected, dissected and analyzed by biochemical readouts. RNA was extracted from caudal cortex (right hemisphere) while protein was extracted from matching right frontal (medial) cortex for Western Blot (WB) analysis and from lateral half of the frontal cortex for Liquid Chromatography Mass Spectrometry (LC-MS) analysis.

### RNA Analysis

For RNA extraction, the samples were transferred into Precellys tubes containing RLT Plus lysis buffer (with 10 μl/ml of β-mercaptoethanol) (Precellys Lysing Kit CK14 −2 ml (VWR, 432-3751)). DNAse treatment was performed for the RNA. The RNA extraction was performed on KingFisher Flex (ThermoFisher), using Mag-Bind Total RNA 96 kit (Omega, M6731). The RNA concentration was measured with Nanodrop, and 500 ng of RNA were submitted to reverse transcription using the kit High Capacity cDNA RT Kit+RNase Inhibitor (catalog n° 4374966, ThermoFisher). Subsequently, the cDNA obtained was analyzed by qPCR, in triplicates, using commercially available and custom-made primers and probes, mouse STXBP1 and mouse and human STXBP1-long and mouse and human STXBP1-short isoforms, as well as two reference genes. mRNA expression level was obtained by calculating the 2^{-ΔCt} value, where the expression of each gene was normalized to the average of the two reference genes.

### Western Blot Analysis

For protein extraction, the tissue was lysed in RIPA buffer (Sigma R0278) containing 2× Protease and phosphatase inhibitors cocktail (Cell Signaling Technology #5872) using the Precellys 24 instrument (Bertin Technologies) and cooling system. The samples were left on ice for 30 min, centrifuged and the supernatant was collected as the final protein extract. Protein concentration was determined using the BCA Protein Assay (Thermo Scientific™) and 10 μg of protein were mixed with Laemmli buffer and β-mercaptoethanol and incubated at 90° C. for 10 minutes prior to SDS-Page. Gels were transferred to nitrocellulose membranes and then submitted to standard Western Blot procedure. First, membranes were incubated in blocking solution (Ref: 927-50000; Li-Cor) for 1 hour at RT. The following primary antibodies were incubated overnight at 4° C.: goat polyclonal anti-STXBP1 (1:1000, Ref:PAB6504, Abnova) rabbit polyclonal anti-STXBP1 (1:1000, Ref:116002, SySy), rabbit polyclonal anti-STXBP1 (1:1000, Ref:HPA008209, Sigma), mouse monoclonal anti-Syntaxin-1A (1:2500, Ref:110111, SySy), mouse monoclonal anti-β-Actin (1:10000, A2228, Sigma) and rabbit monoclonal anti-β-Actin (1:10000, 8457P, Cell Signaling Technology). The secondary antibodies were incubated 1 h at RT, and the following were used: IRDye® 680RD donkey anti-mouse IgG secondary antibody (1:20000; Ref: 926-68072, Li-Cor), IRDye® 800CW donkey anti-rabbit IgG secondary antibody (1:20000; Ref: 926-32213, Li-Cor) and IRDye® 800CW donkey anti-goat IgG secondary antibody (1:20000; Ref: 926-32214, Li-Cor).

### LC-MS Analysis

For LC-MS analysis, the tissue samples were homogenized in 5% SDS/50 mM TEAB/1× protease inhibitor using a Precellys tissue homogenizer (Bertin-Instruments). After, protein concentration was determined by BCA (Pierce, A53227), 100 μg of each sample was reduced and alkylated. Sample clean up and digestion were performed on a 96 well-plate S-Trap per manufacturer instructions (Protifi Llc, Huntington, NY) and using Trypsin/Lys-C (Promega, V5072). Digested samples were eluted from the plate and dried down under vacuum, before resuspension for LC-MS analysis. The resuspension buffer contained heavy labelled AQUA peptides (Thermo, Paisley, UK) at 50 fmol/μl in 0.1% formic acid in water. For the total lysate samples, STXBP1 peptides were measured using a Waters Acquity UPLC M-Class with an IonKey source, connected to a Waters Xevo TQ-XS. Peptides were trapped on a Waters nanoEase M/Z Sym100 C18 column (5 μm, 300 μm×25 mm) and separated on a Waters Peptide BEH C18 iKey (150 μm×100 mm, 130 [symbol] 1.7 μm). A 17 min gradient was applied at a flow rate of 3 μl/min, with mobile phase A (0.1% formic acid/100% H2O) and mobile phase B (0.1% formic acid/100% acetonitrile). The gradient used was: 1.0% B for 0 to 1 min, 1.0-25% B from 1 to 3 min, 25-40% B from 3 to 6 min, 40-99% B from 6 to 9 min, 99-1% B from 12 to 13 min. Column temperature was set at 50° C. A scheduled Multiple Reaction Monitoring (MRM) method was used with the source parameters as follows: capillary voltage—3.8 kV, source temperature—150° C., cone gas—150 L/hr, nebulizer gas—5.3 bar. NanoFlow gas—0.3 bar. For all analyses the peptides monitored were: DNALLAQLIQDK (SEQ ID NO: 43), YETSGIGEAR (SEQ ID NO: 44), ISEQTYQLSR (SEQ ID NO: 45), WEVLIGSTHILTPTK (SEQ ID NO: 46) (long isoform specific), and WEVLIGSTHILTPQK (SEQ ID NO: 47) (short isoform specific). Three transitions per peptide were monitored. Data analysis was performed in Skyline (MacLean et al., 2010). Each analysis included an 8-point standard curve and QC samples (low, mid, high, n=2). These consisted of blank, pooled mouse liver homogenate spiked with purified HA-tagged STXBP1 protein prepared from recombinant expression in E. coli. Endogenous QC samples consisting of pooled mouse brain membrane homogenate (blank and spiked with additional STXBP1) were also included. Quantification of the total protein and short isoform was performed against this standard curve. Relative quantitation of the isoform specific peptides was performed against their respective internal standard.

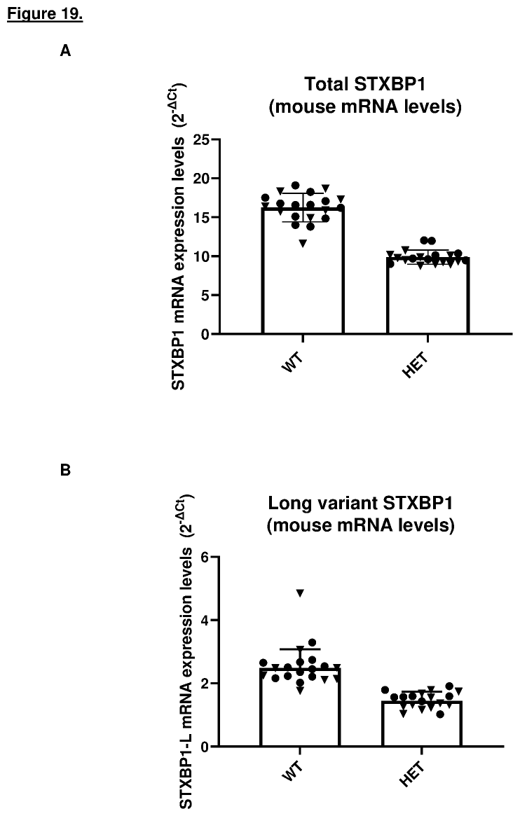

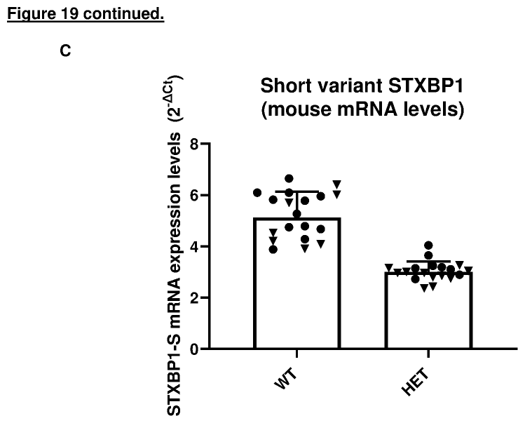

FIG. 19: Analysis of STXBP1 variant mRNA levels in mouse brain by qPCR. mRNA analysis of brain tissue samples from caudal cortex (right hemisphere) of WT (wild-type) littermates and the HET (heterozygotic) mice (n=11-13 per group). (A): mRNA expression analysis of total endogenous STXBP1 (common probe that recognizes all STXBP1 transcripts). (B) and (C): mRNA expression analysis of STXBP1 variants, using two distinct probes that specifically recognize the long isoform (B) or the short protein isoform (C). Data are shown as mRNA expression level, by calculating the 2^{-ΔCt} value, where the expression was normalized to the average of the two reference genes. Results are shown as Mean±SD.

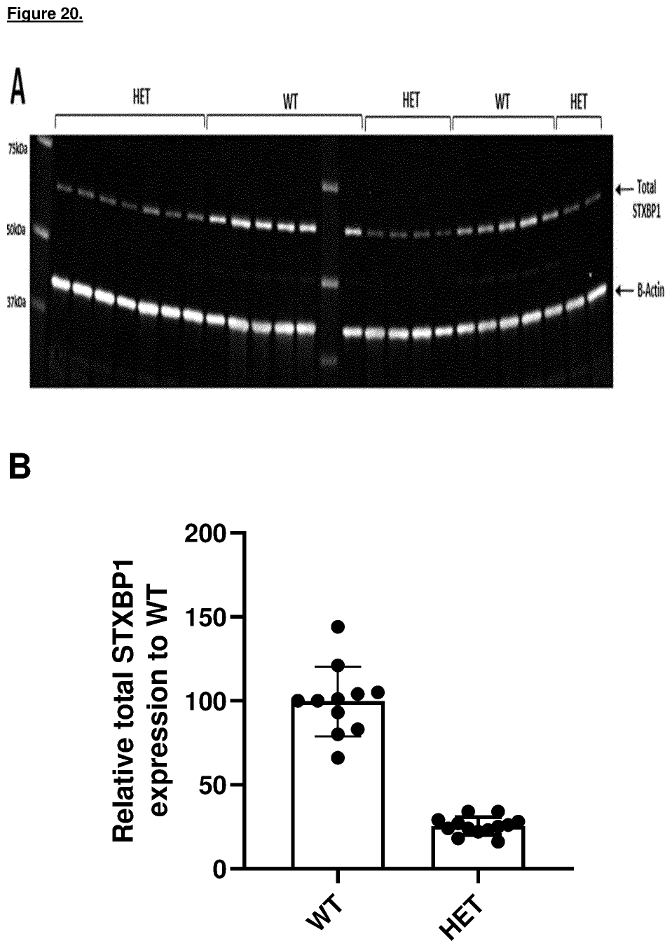

FIG. 20: Analysis of STXBP1 protein levels in mouse brain by Western Blot. Tissue samples from the right frontal (medial) cortex of WT (wild-type) littermates and the HET (heterozygotic) mice (n=11-13 per group) have been analyzed. (A): Western blots representing the total STXBP1 protein expression. (B): quantification data of the respective Western blots in (A). B-Actin was used as a loading control, for the normalization. The “WT” group was used as the scaling group. Results are shown as the mean±SD.

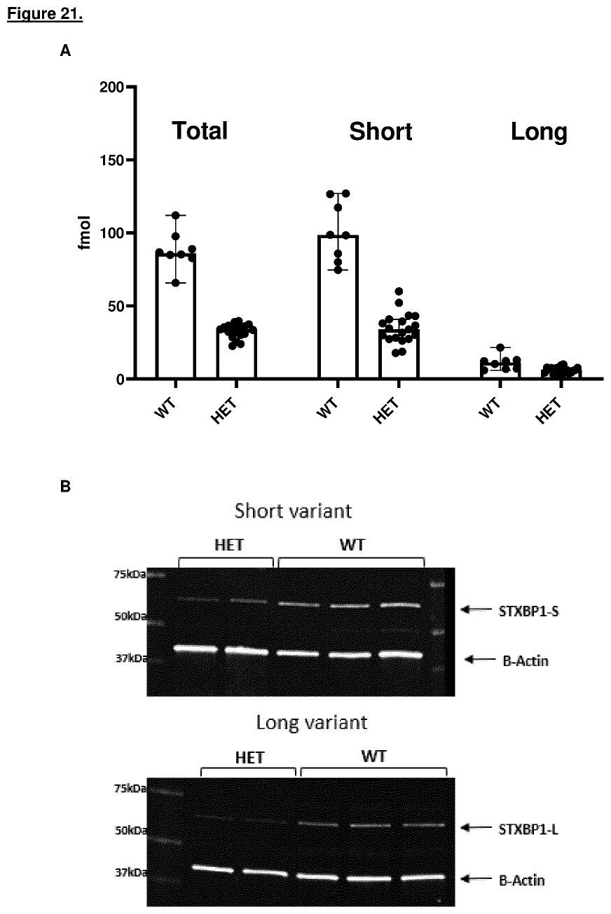

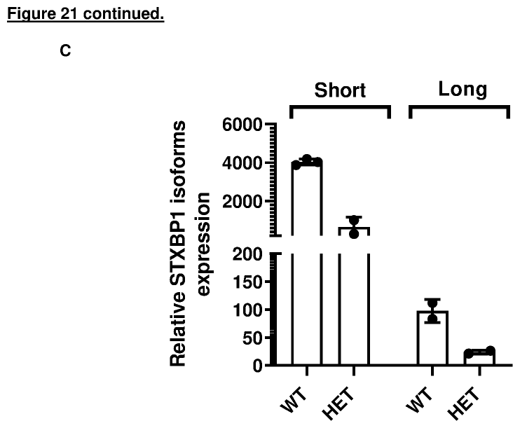

FIG. 21: Analysis of STXBP1 variant protein levels in mouse brain by LC-MS. Tissue samples from the lateral half of the frontal cortex of WT (wild-type) littermates and HET (heterozygotic) mice (n=11-13 per group) have been analyzed. (A): Quantification of total STXBP1 peptide vs STXBP1 long isoform vs STXBP1 short isoform. Results are shown as mean±SD. (B): Western blots representing the STXBP1 short and long isoforms. (C): combined quantification data of the respective Western blots in (B). B-Actin was used as a loading control. Data shown as the ratio between band intensity of each STXBP1 isoform and the respective B-actin band. Results are shown as the mean±SD.

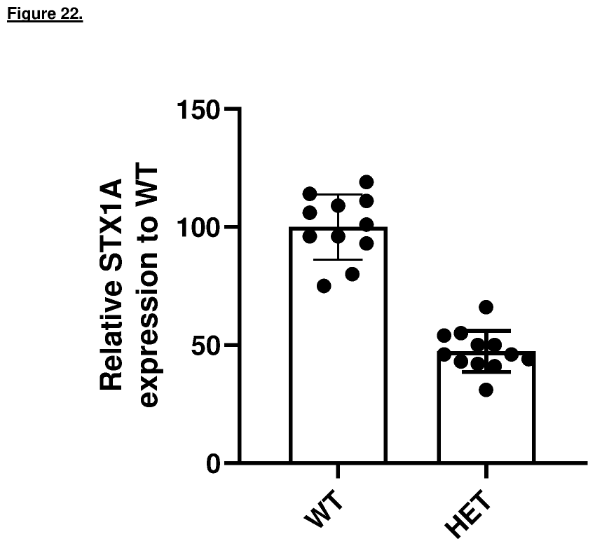

FIG. 22: Analysis of Syntaxin-1A (STX1A) protein levels in mouse brain by Western Blot. Quantification of STX1A protein expression in the mouse brain tissue samples (n=11-13 per group). B-Actin was used as a loading control, for the normalization. The “WT” group was used as the scaling group. Results are shown as the mean±SD.

The results of RNA transcript analysis in WT and heterozygous (+/−) KO mice (referred as HET in the figures) are shown in FIG. 19 (A-C). The endogenous mouse mRNA transcript levels of total STXBP1 are reduced in HET mice (FIG. 19 (A)). We also observed that the short isoform and long isoform of STXBP1 are reduced in HET mice (37-43%) when compared with WT littermates (FIG. 19 (B,C)). Western blot analysis of total STXBP1 confirmed a 60-70% protein reduction in HET mice when compared with WT animals (FIG. 20).

The quantification of STXBP1 protein isoforms by LC-MS (FIG. 21) indicated that the short STXBP1 variant is the most abundant in the mouse brain of WT and HET animals, when compared with the overall levels of the long isoform (FIG. 21 (A)). Quantification of STXBP1 peptides in HET animals indicated that the total, short and long variants of STXBP1 are reduced by ˜60% when compared with the WT littermates. Western blot data also confirmed the same overall reduction in STXBP1 short and long isoforms in HET animals when compared with the WT animals (FIG. 21, Panel B and C).

STXBP1 has been reported to act as a chaperone for the syntaxin-1A protein (STX1A), ensuring the trafficking, docking and release of synaptic vesicles (Dulubova I. et al. 2007, Saitsu H. et al. 2008). As illustrated in FIG. 22, haploinsufficiency of STXBP1 results in a 50-60% reduction of STX1A protein levels in HET mice when compared with WT littermates.

Altogether the data provides for the first time an extensive characterization of the STXBP1 isoforms expression levels in mouse brain and the validation of the reduction of endogenous STXBP1 variant mRNA and protein levels in the transgenic mouse model that recapitulates human STXBP1 haploinsufficiency.

### Example 10: AAV Mediated Overexpression of STXBP1 Variants in a Haploinsufficiency Mouse Model

Viral vectors were administrated by bilateral intracerebroventricular (ICV) injection into the brain of post-natal 1 day old neonatal mice (PND1) as described in Example 7. The methodology for ICV neonatal injections has been previously described (Bertrand-Mathon, et al. 2015; Kim, et al. 2014; Hamodi, et al. 2020). Injected animals were monitored for a period of 7 weeks and the expression and distribution of STXBP1 was analyzed by biochemical readouts on brain tissues. Experiments included the following groups:
Heterozygous KO (STXBP1+/−) (referred as HET)
Wild type littermate (STXBP1+/+) male mice (referred as WT)
HET mice bilaterally injected with one of two viral vectors described in Table 17

TABLE 17
AAV9 viral vector properties

| | | | | Viral | Endo- |
|---|---|---|---|---|---|
| | | | | genomes | toxin |
| Vector | | STXBP1 | | titer | (<10 |
| ID | Promoter | variant | Capsid | (vg/mL) | EU/mL) |
| AAV9(L) | Mecp2_intron | Long variant | AAV9 | 9.43E12 | Pass |
| | (SEQ ID NO: 3 + | (SEQ ID | | | |
| | SEQ ID NO: 37) | NO: 9) | | | |
| AAV9(S) | Mecp2_intron | Short variant | AAV9 | 6.91E12 | Pass |
| | (SEQ ID NO: 3 + | (SEQ ID | | | |
| | SEQ ID NO: 37) | NO: 10) | | | |

One additional group of WT and HET mice were injected with vehicle-PBS to be used as control. A summary of the in vivo experimental conditions is shown in Table 18.

At 7 weeks post-injection, brain tissues were collected, dissected and submitted for biochemical analysis. DNA/RNA were extracted from caudal cortex (right hemisphere) while proteins were extracted from matching right frontal (medial) cortex. The DNA and RNA were both extracted with the same lysis buffer composition, as described in Example 7. Proteinase K and RNase treatment were performed for the DNA. DNA was extracted using Mag-Bind™ HDQ Blood DNA & Tissue 96 Kit (Omega, M6399). The DNA concentration was measured using Qubit™ Flex Fluorometer (ThermoFisher) with Qubit™ dsDNA BR Assay Kit (ThermoFisher, Q32853), and the same total DNA amount was adjusted for all samples, being used 40 ng for qPCR with primers/probe specific for the SV40 20 polyA signal (present in all the AAV cassettes). The amount of mouse genomes was analyzed using the ValidPrime® kit (tataabiocenter, A106P25). The ValidPrime® sequence is specific to a non-transcribed locus of gDNA that is present in exactly one copy per haploid normal genome. For both SV40p and ValidPrime®, absolute copy numbers were determined using the standard curve method.

RNA extraction steps and conversion into cDNA are described in Example 7. The cDNA obtained was analyzed by qPCR, in triplicates, using commercially available and custom-made primers and probes, such as SV40 polyA, human STXBP1, mouse STXBP1, mouse STX1A, mouse and human STXBP1-long isoform and mouse and human STXBP1-short isoform, as well as two reference genes. mRNA expression level was obtained by calculating the 2^{-ΔCt} value, where the expression of each gene was normalized to the average of the two reference genes. The protein extraction and Western Blot analyses were performed as described in Example 7.

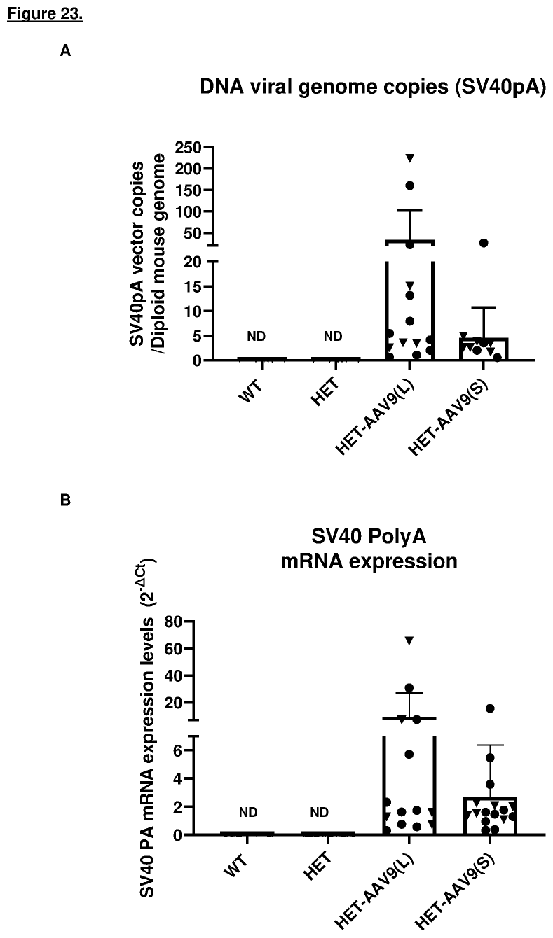

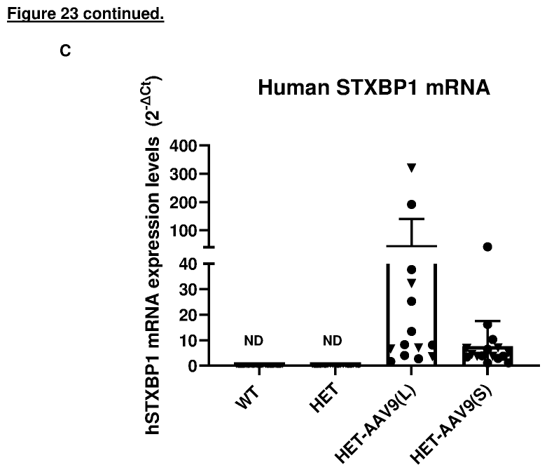

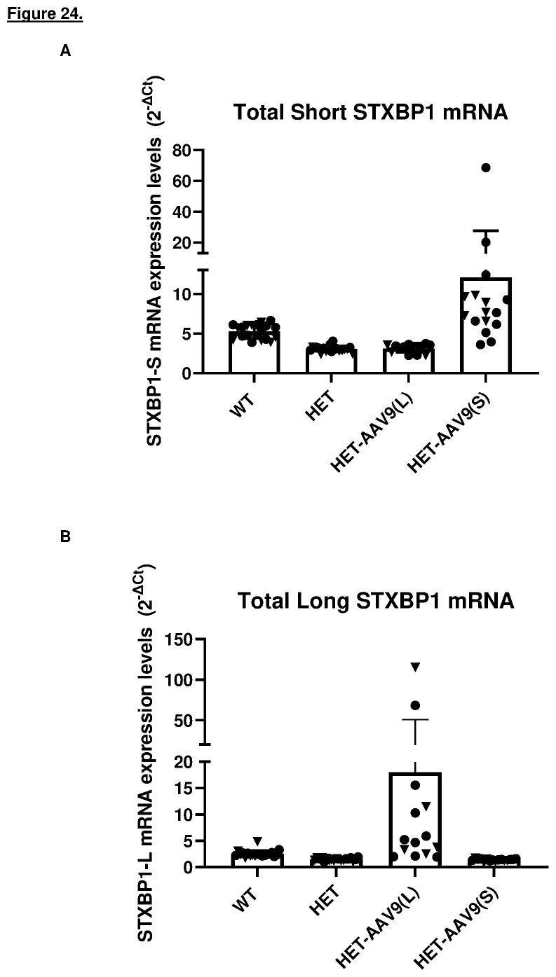

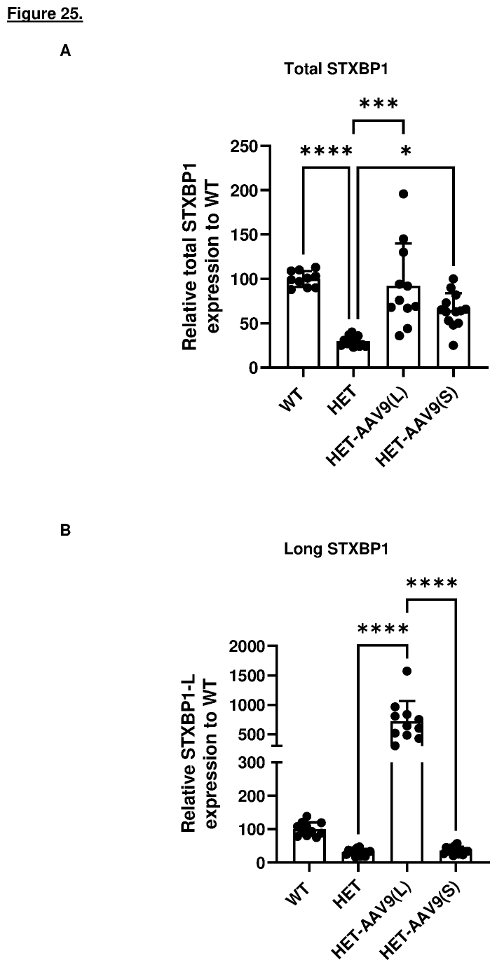

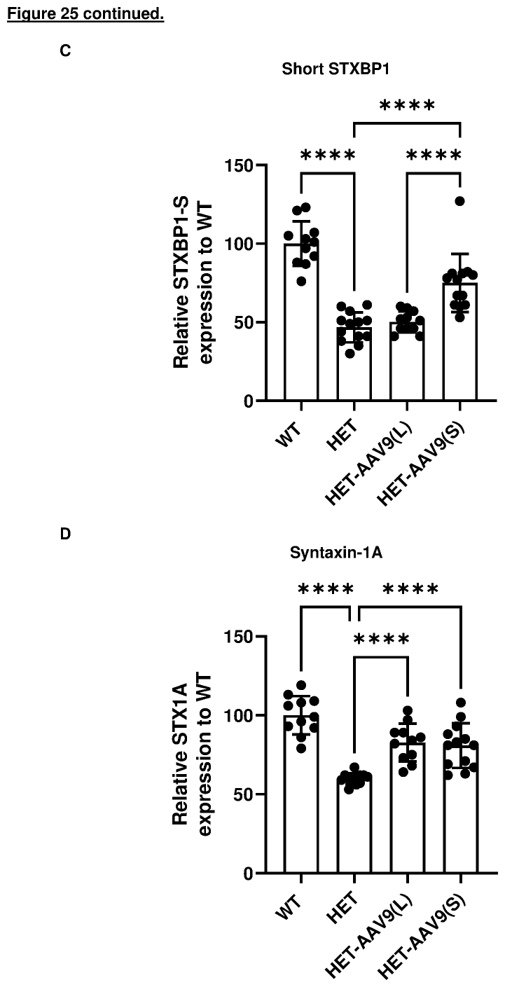

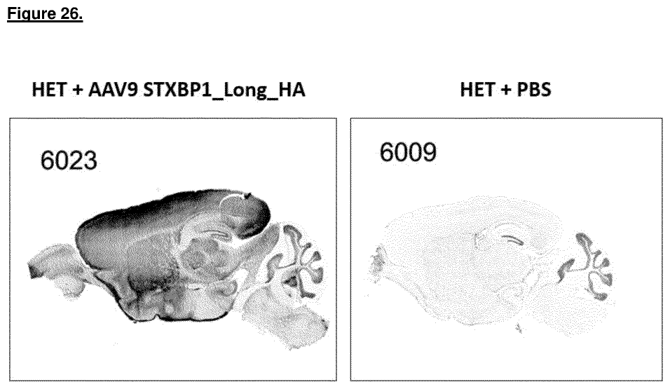

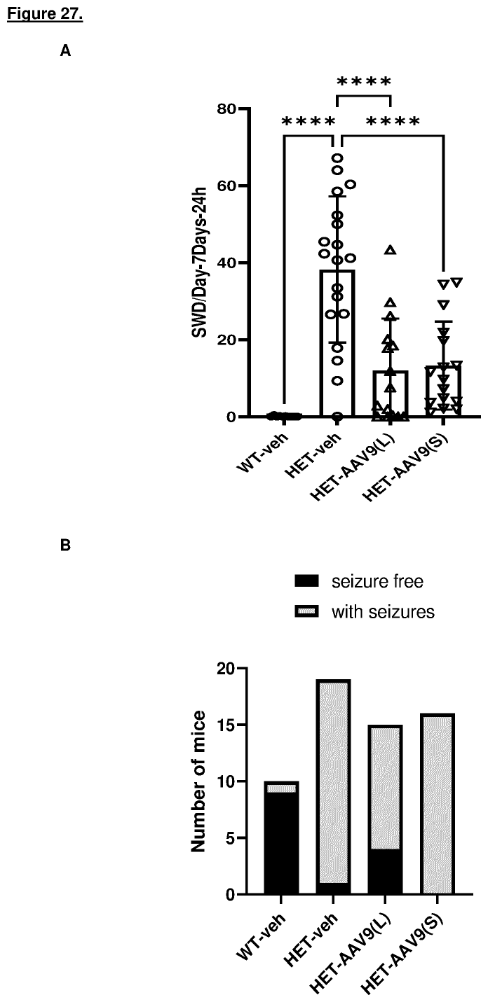

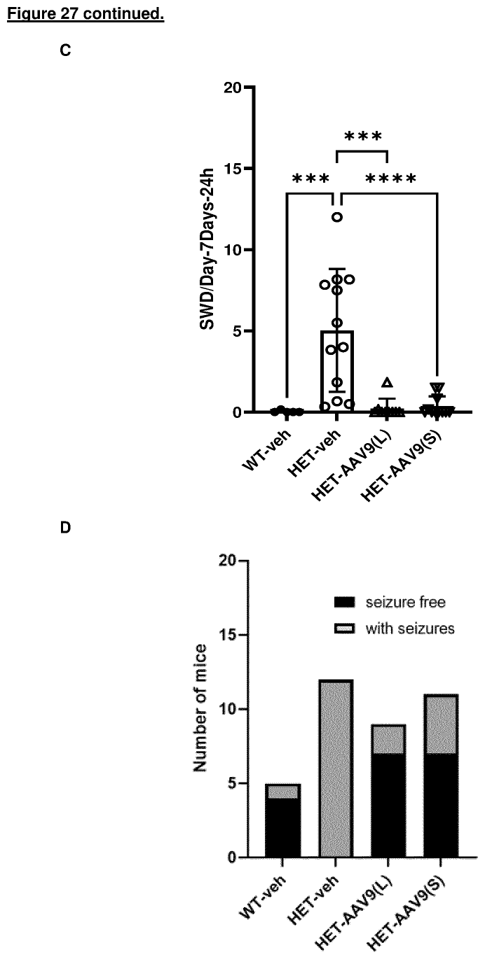

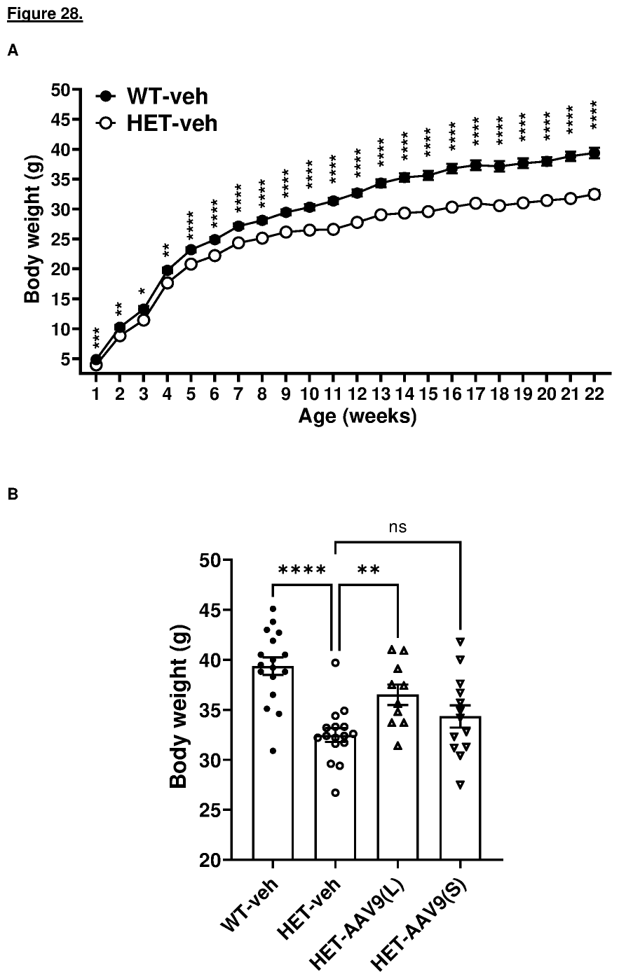

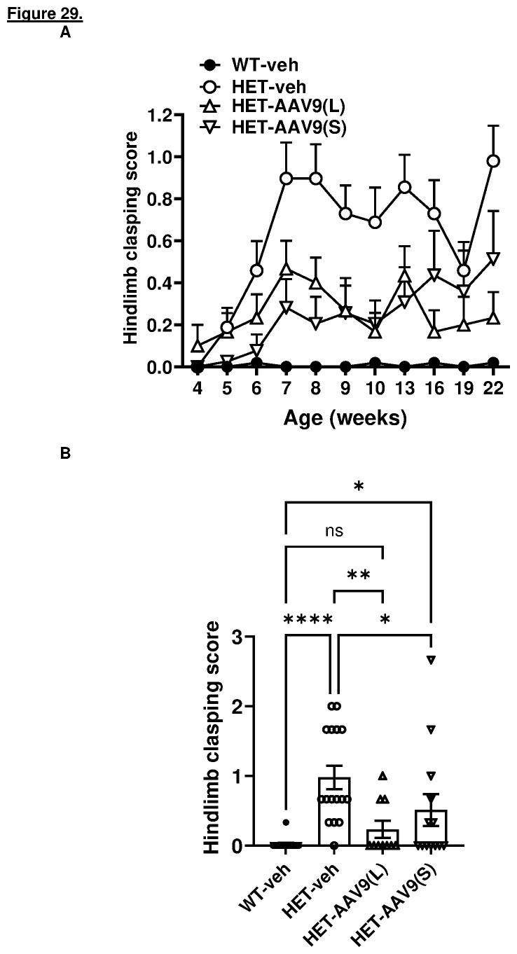

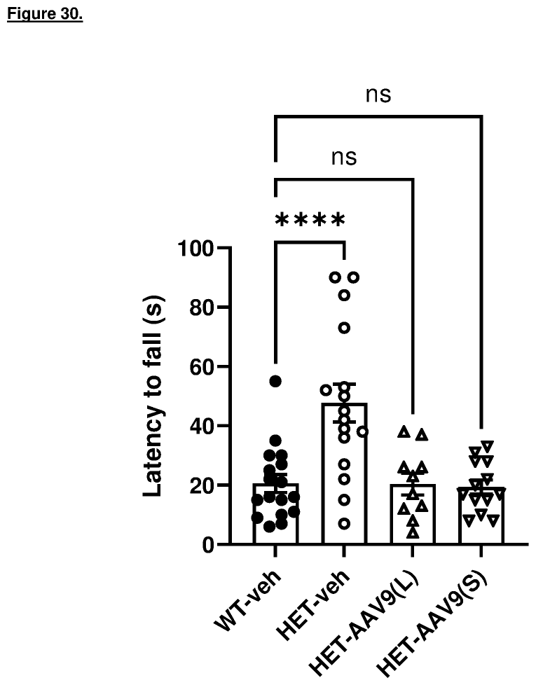

FIG. 23: Analysis of AAV transduction efficiency in mouse brain by qPCR (7 weeks post injection) (A) Absolute quantification by qPCR of viral genome copies in WT mice injected with vehicle-PBS (WT), HET mice injected with vehicle-PBS (HET), HET mice injected with STXBP1 long variant (HET-AAV9(L)) and HET mice injected with STXBP1 short variant (HET-AAV9(S)). Samples were collected from the caudal cortex (right hemisphere) and quantified using SV40 pA normalized to the absolute number of diploid mouse genome. Results are shown as mean±SD. Per group, n=14-15 animals were analyzed and a non-parametric one-way ANOVA (Kruskal-Wallis test) followed by a Dunn's post hoc multiple comparisons test was applied. No significant difference was observed between the transduced groups. (B) mRNA expression analysis of SV40 PolyA and (C) human-specific STXBP1. Data are shown as mRNA expression level, by calculating the 2^{-ΔCt} value, where the expression was normalized to the average of the two reference genes. Results are shown as mean±SD. Per group, n=14-15 animals were analyzed, and a non-parametric one-way ANOVA (Kruskal-Wallis test) followed by a Dunn's post hoc multiple comparisons test was applied. No significant difference was observed between the transduced groups.

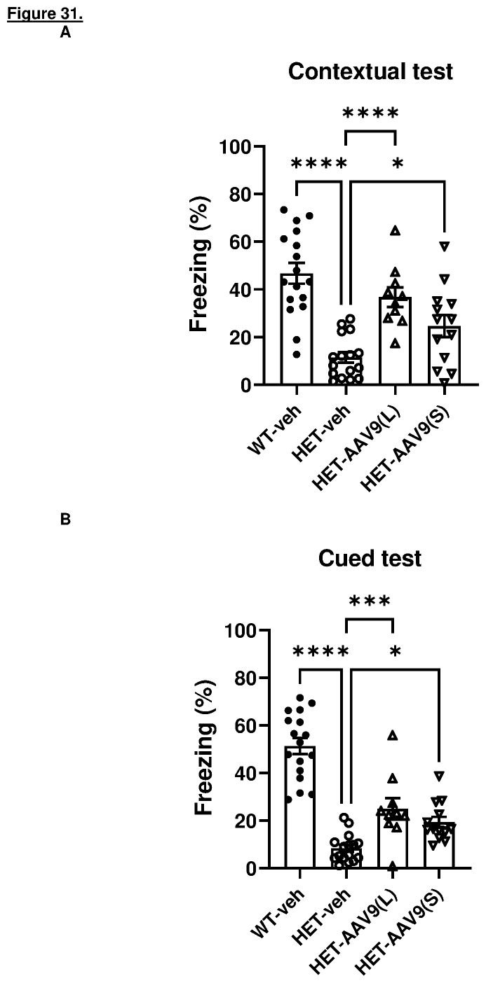

FIG. 24: Analysis of STXBP1 variant expression following AAV treatment in mouse brain by qPCR (7 weeks post injection). Analysis of STXBP1 variant mRNA expression was performed using probes that specifically measure total (mouse and human) levels of the short (A) or the long variant (B). Data are shown as mRNA expression level, by calculating the 2^{-ΔCt} value, where the expression was normalized to the average of the two reference genes. Results are shown as mean±SD. Per group, n=14-16 animals were analyzed.

FIG. 25: Analysis of STXBP1 variant expression following AAV treatment in mouse brain by Western blot (7 weeks post injection). Protein analysis by Western blot of samples from right frontal (medial) cortex, in WT mice injected with vehicle-PBS (WT), HET mice injected with vehicle-PBS (HET), HET mice injected with STXBP1 long variant (HET-AAV9(L)) and HET mice injected with STXBP1 short variant (HET-AAV9(S))
(A) Quantification of western blot data for total STXBP1 (long and short variants) protein expression.
(B) Quantification of western blot data for the long STXBP1 variant protein expression.
(C) Quantification of western blot data for the short STXBP1 variant protein expression.
(D) Quantification of western blot data for Syntaxin-1A protein expression
   B-Actin was used as a loading control, for the normalization of each STXBP1 and STX1A band intensity. The vehicle WT group (WT) was used as the scaling group. Results are shown as the mean±SD. The data was analyzed using non-parametric one-way ANOVA (Kruskal-Wallis test) followed by a Dunn's post hoc multiple comparisons test (*<p.0.05; **p<0.01 ***p<0.001; ****p<0.0001).

FIG. 26: Brain distribution of HA− tagged STXBP1 expression following AAV treatment in mouse brain by immunohistochemistry (7 weeks post injection). HA tag staining was performed on saggital sections from HET mice injected with the HA-tagged STXBP1 long variant and compared to vehicle (PBS) treated mice. An example of representative brain section are shown for the AAV treated group (animal 6023) and the vehicle treated group (animal 6009). A strong HA staining is observed in major brain regions in animal 6023 (AAV treated) whereas no HA staining is observed in the PBS treated groups (animal 6009).

As illustrated in FIG. 23 (A), a significant viral genome copies per diploid mouse genomes were detected in the mouse brain from AAV treated groups, demonstrating an efficient viral transduction with the two cassettes encoding the Long and Short variants of STXBP1. Both human STXBP1 transgene (FIG. 23 (B)) and SV40 pA expression (mRNA) (FIG. 23 (C)) were only detected in groups transduced with the viral vectors, showing a similar expression trend with the viral DNA amounts injected.

AAV9 transduction of HET animals resulted in a robust and selective over-expression of the short and the long STXBP1 variants without affecting endogenous mouse variant expression levels (FIG. 24).

Western Blot analysis confirmed a significant overexpression of total STXBP1 levels in both AAV treated groups, when compared to HET mice injected with vehicle-PBS, as illustrated in FIG. 25 (A). Western Blot quantifications, using an antibody that specifically recognizes the STXBP1 long isoform, showed a significant and specific overexpression of the long variant only in the AAV treated group with the STXBP1-long cassette (FIG. 25, (B)). Similarly, using a specific antibody for the STXBP1 short isoform, a significant and specific protein overexpression was observed only for the group transduced with STXBP1-short cassette compared to the HET-PBS injected animals (FIG. 25, (C)). Moreover, the AAV treatment with either short or long variants partially rescued syntaxin-1A (STX1A) protein levels, which increased significantly when compared with HET mice injected with vehicle-PBS, as illustrated in FIG. 25 (D). The increase in STX1A levels in the AAV treated groups further confirms a functional impact of the human STXBP1 transgene product expression.

Overall, HET animals treated with either the short or the long variant showed efficient overexpression of the human STXBP1 transgene product and resulted in a similar rescue of STXBP1 haploinsufficiency in this mouse model.

The distribution of STXBP1 transgene product expression in the mouse brain following PND1 injection of AAV9 vector was investigated by immunohistochemistry (IHC), using an additional animal group injected with a viral cassette encoding an HA-tagged fusion with the STXBP1 long variant (as described in Example 7). Fixed frozen sections (12 μm thickness; sagittal sections) were generated with a cryostat-microtome and stored at −80° C. The staining procedure and detection method were as described in Example 8.

As illustrated in FIG. 26, AAV mediated transgene STXBP1 protein expression was detected after 7 weeks through the HA-tag labelling throughout the whole brain (animal 6023), in sagittal sections, mainly in the striatum, hippocampus, cerebral cortex, hypothalamus, pallidum and septum. No major HA signal was observed across these brain regions in HET animals (animal 6009) that received PBS only. IHC data confirms that AAV mediated transduction of a Mecp2_intron_STXBP1 (Long) cassette leads to a wide brain expression of the STXBP1 protein.

### Example 11: AAV Gene Therapy to Rescue Seizure Phenotype in STXBP1 Heterozygous Disease Model

To evaluate the efficacy of AAV vectors in normal and disease conditions, a transgenic mouse model that recapitulates human STXBP1 haploinsufficiency-mediated epilepsy was generated and that has been described by Kovacevic et al. (2018). This mouse model was acquired through a license from the University of Amsterdam. The heterozygous model was generated with Stxbp1 floxed (Stxbp1fl/fl) mice with loxP sites on either side of exon 2 in the Stxbp1 gene. Stxbp1fl/fl were crossed to Ella-Cre (Jax: 003724) to delete Stxbp1 exon 2 in germ line resulting in Stxbp1fl/−null mutant mice. The floxed allele has been outbred to C57BL/6J generating the Stxbp1+/−KO HET mouse strain. Deletion of exon 2 in one allele leads to a premature stop codon and results in expression of a truncated and non-functional STXBP1 protein. All in vivo experiments were conducted in compliance with guidelines issued by the ethics committee for animal experimentation according to Belgian law. The experiments were performed in accordance with the European Committee Council directive (2010/63/EU). All efforts were made to minimize animal suffering.

Heterozygous (HET) KO and wildtype littermate (WT) male mice were bilaterally injected into lateral ventricle with one of two viral vectors encoding the long or the short STXBP1 variants (see Table 17) at postnatal day 1. Experimental conditions are summarized in Table 18. One additional group of mice from each genotype were injected with vehicle-PBS to be used as control. Clinical signs were monitored once a week over the course of the 3 weeks post-injection and daily from week 3 to 7 post-injection in order to assess the overall health status of the mice. Limited mortality across groups related to methodological procedures and aggressive behavior was observed but it was not treatment or genotype related.

TABLE 18
Summary of in vivo experimental conditions

| | Age at | Delivery | | | Sample | In Life | Seizure | Terminal |
|---|---|---|---|---|---|---|---|---|
| Genotype | treatment | Route | Treatment | Titer | size | Assessment | monitoring | Assessment |
| HET | Postnatal | ICV | AAV9(L)* | 9.43 | N = 15 | Clinical | In vivo | Brain, plasma and |
| | day 1 | bilateral | | E12 | | signs, | wireless | organs collection at |
| | | 2 μl/ | | GC/mL | | adverse | EEG video- | 7 weeks post-injection |
| | | hemisphere | AAV9(S)* | 6.91 | N = 16 | effects, body | telemetry | for Biochemical |
| | | | | E12 | | gain weight | recordings | analysis, Histopathology, |
| | | | | GC/mL | | and mortality | (6-7 weeks | Immunohistochemistry, |
| | | | Vehicle- | — | N = 19 | during 7 | post- | transgene expression |
| | | | PBS | | | weeks post- | injection) | |
| | | | | | | injection | | |
| WT | Postnatal | ICV | Vehicle- | — | N = 10 | | | |
| | day 1 | bilateral | PBS | | | | | |
| | | 2 μl/ | | | | | | |
| | | hemisphere | | | | | | |

*Described in Table 17

Six weeks after injections, in vivo wireless EEG (electroencephalogram) video-telemetry recordings were performed for 1 week to evaluate seizure occurrence. STXBP1+/− mice were surgically implanted with subcutaneous telemetry transmitter and cortical EEG electrodes 5 weeks after injections. Surgery was performed under sterile/aseptic conditions. Anaesthetized mice (isoflurane in oxygen-induction: 5% at 2 l/min, maintenance 2.5-1.5% at 1.5 l/min) were placed in a stereotaxic frame with heating pad, holes were drilled on the skull surface of the prefrontal cortex (over bregma) for the recording electrode and on the skull surface of the cerebellum (behind the lambda) for the reference electrode. Thereafter, an Open Source Instruments (OSI) A3028S2 ECoG transmitter was implanted subcutaneously over the dorsum with the attached wires extending subcutaneously up to the cranium where the recording and reference electrodes were positioned through each hole approximately 0.5 mm into the brain parenchyma. Each electrode was secured in place with a screw (Plastics One). The whole assembly was held in place with cyanoacrylate and dental cement forming a small, circular headpiece and the dorsum was closed with nylon absorbable suture material. Post-operative medication and pain management included a second Carprofen dose (10 mg/kg) 24 hours following the pre-surgery dose. After the surgery, mice were recovering in warm-chamber for 2-3 h. For in vivo wireless EEG video-telemetry recordings, mice were group housed (2-3 mice/cage). Mice cages were placed in Faraday enclosures to facilitate recordings. Welfare monitoring of implanted mice was conducted once per day for 2 weeks. Mice were weighed daily for 4 days, thereafter weekly. All recordings were carried in a purposely designed recording room with temperature and humidity control in order to decrease ambient interference and improve the reception of the transmitting signals. Signals were radio transmitted from the implanted transmitter to the antennas placed inside the Faraday enclosures. EEG signal from one recording channel was digitized at 256 Hz (Band-pass filter: 0.3-80 Hz). Spike wave discharges (SWDs), typical of absence seizures, were analysed with an in-house automated seizure detection software. SWDs detection algorithm was based on event duration analysis (>2 s), band frequency analysis (5-9 Hz) and identification of specific fundamental harmonic frequencies. Each SWD detected by the algorithm was confirmed by at least one experienced observer in a blinded fashion. Consequently, EEG analysis was performed during this period for the different cassette vector and vehicle groups. A total of 4 animals were excluded from the analysis due to the occurrence of technical artefacts in the EEG signal in the vector treated groups: (AAV9-MECP2+INTRON-hSTXBP1 (Long Variant) (2 out of 17), and AAV9-MECP2+INTRON-hSTXBP1 (Short Variant) (2 out of 18).

FIG. 27: Analysis of spike wave discharges (SWD) following AAV treatment in STXBP1 HET mouse brain by EEG (6-7 weeks) (A,B) and 24 weeks (C,D) post injection. (A) Average number of SWDs in WT mice injected with vehicle-PBS (WT, n=10), HET mice injected with vehicle-PBS (HET, n=19), HET mice injected with STXBP1 long variant (HET-AAV9(L), n=15) and HET mice injected with STXBP1 short variant (HET-AAV9(S), n=16). SWDs were analyzed 6-7 weeks after injection over a period of 24 h for 7 consecutive days. (B) Analysis of the number of animals that are “seizure free” (without any SWD detected during recordings) and animals “with seizures” (SWD detected during recordings). (C) Average number of SWDs in WT mice injected with vehicle-PBS (WT, n=5), HET mice injected with vehicle-PBS (HET, n=12), HET mice injected with STXBP1 long variant (HET-AAV9(L), n=9) and HET mice injected with STXBP1 short variant (HET-AAV9(S), n=11). SWDs were analyzed 24 weeks after injection over a period of 24 h for 7 consecutive days. (D) Analysis of the number of animals that are “seizure free” (without any SWD detected during recordings) and animals “with seizures” (SWD detected during recordings) 24 weeks after injection. The difference between groups was analyzed by non-parametric one-way ANOVA (Kruskal-Wallis test) followed by a Dunn's post hoc multiple comparisons test (****p<0.0001), (***p<0.001) and for Seizure free analysis a chi-square contingency test was used.

As illustrated in FIG. 27 (A), the average number of SWDs per day recorded over 7 consecutive days for 24 hours was significantly reduced by 70% and 65% in HET mice treated with either the long variant (HET-AAV9(L)) and short variant (HET-AAV9(S)), compared to the vehicle group. HET mice treated with the long variant displayed 26% of seizure free animals (FIG. 27 (B), significant differences compared to Short Variant). Detailed EEG analysis did not detect occurrence of convulsive seizures in the treated animals during recordings (1-week 24/7).

The difference between groups for SWD frequency was analyzed by non-parametric one-way ANOVA followed by a post hoc multiple comparisons test (****p<0.0001) and for Seizure free analysis a chi-square contingency test was used.

Furthermore, biochemical and histopathological analysis was performed on the brain and organs tissues from the animals injected with the different groups. For transgene expression evaluation, mice were sacrificed 7 weeks post injection following the same methodology as described in Example 7. Caudal cortex was collected and subjected to DNA/RNA extraction and matching half medial frontal cortex was used for protein extraction using the same methodology described in Example 7.

A longitudinal 6 months study was performed in a separate group of animals to measure the persistence of effects of SXTBP1 gene therapy treatment using the same experimental design. Twenty four weeks after injections, in vivo wireless EEG (electroencephalogram) video-telemetry recordings were performed for 1 week to evaluate seizure occurrence. As illustrated in FIG. 27 (C), the average number of SWDs per day recorded over 7 consecutive days for 24 hours was significantly reduced by 95% and 92% in HET mice treated with either the long variant (HET-AAV9(L)) or short variant (HET-AAV9(S)) respectively, compared to the vehicle group. HET mice treated with the long or the short variants displayed respectively 78% and 64% of seizure free animals (FIG. 27 (D)). Detailed EEG analysis did not detect occurrence of convulsive seizures in the treated animals during recordings (1-week 24/7).

### Example 12: AAV Gene Therapy to Rescue Behavioral Phenotypes in STXBP1 Heterozygous Disease Model

To evaluate the efficacy of AAV mediated gene therapy on different behavioral disease phenotypes in heterozygous STXBP1 KO (HET) male mice and their sex- and age-matched wildtype (WT) littermates, viral vectors encoding the long or short variant of human STXBP1 under the control of the Mecp2_intron promoter (see Table 17) were bilaterally injected into lateral ventricle. These groups of animals were separate to the ones used in Example 11.

Treated animals were subjected to a battery of behavioral tests from 4 weeks to 22 weeks of age. One additional group of mice from each genotype was injected with vehicle-PBS to be used as control. All behavioral experiments were conducted in compliance with guidelines issued by the ethics committee for animal experimentation according to Belgian law. The experiments were performed in accordance with the European Committee Council directive (2010/63/EU). All efforts were made to minimize animal suffering.

FIG. 28: Analysis of body weight following AAV treatment in STXBP1 HET mice (1-22 weeks post injection) (A) Average body weights as a function of age in WT (n=17) and HET mice (n=16) injected with vehicle-PBS. The difference between groups was analyzed by two-way repeated measures ANOVA followed by an uncorrected Fisher's LSD post hoc multiple comparisons test (*<p.0.05; **p<0.01; ***p<0.001; ****p<0.0001). (B) Average body weight measured at 22 weeks old in WT mice injected with vehicle-PBS (WT, n=17) and HET mice injected with vehicle-PBS (HET, n=16), HET mice injected with STXBP1 long variant (HET-AAV9(L), n=10) and HET mice injected with STXBP1 short variant (HET-AAV9(S), n=13). The difference between groups was analyzed by parametric one-way ANOVA followed by an uncorrected Fisher's LSD post hoc multiple comparisons test **p<0.01; ****p<0.0001; ns, nonsignificant). Bar graphs are mean±SEM.

FIG. 29: Analysis of hindlimb clasping following AAV treatment in STXBP1 HET mice (4-22 weeks post injection) (A) Average hindlimb clasping score as function of age in WT mice injected with vehicle-PBS (WT, n=17) and HET mice injected with vehicle-PBS (HET, n=16), HET mice injected with STXBP1 long variant (HET-AAV9(L), n=10) and HET mice injected with STXBP1 short variant (HET-AAV9(S), n=13). (B) Average hindlimb clasping score recorded at 22 weeks old in WT mice injected with vehicle-PBS (n=17) and HET mice injected with vehicle-PBS (n=16), AAV9/MECP2-int-STXBP1-L (n=10) and AAV9/MECP2-int-STXBP1-S(n=13). The difference between groups was analyzed by non-parametric one-way ANOVA (Kruskal-Wallis test) followed by an uncorrected Dunn's post hoc multiple comparisons test (*<p.0.05; **p<0.01; ****p<0.0001; ns, nonsignificant). Bar graphs are mean±SEM.

FIG. 30: Analysis of STXBP1 HET mice in the wire hanging test following AAV treatment (8 weeks post injection). Latency to fall measured in the four limbs wire hanging test at 8 weeks old in WT mice injected with vehicle-PBS (WT, n=17) and HET mice injected with vehicle-PBS (HET, n=16), HET mice injected with STXBP1 long variant (HET-AAV9(L), n=10) and HET mice injected with STXBP1 short variant (HET-AAV9(S), n=13). The difference between groups was analyzed by parametric one-way ANOVA followed by an uncorrected Fisher's LSD post hoc multiple comparisons test (****p<0.0001; ns, nonsignificant). Bar graphs are mean±SEM.

FIG. 31: Analysis of STXBP1 HET mice in the fear conditioning test following AAV treatment (10 weeks post injection) (A) Average freezing behavior during contextual fear memory test performed at 10 weeks old 24 h after the fear conditioning training phase in WT mice injected with vehicle-PBS (WT, n=17) and HET mice injected with vehicle-PBS (HET, n=16), HET mice injected with STXBP1 long variant (HET-AAV9(L), n=10) and HET mice injected with STXBP1 short variant (HET-AAV9(S), n=13). The difference between groups was analyzed by parametric one-way ANOVA followed by an uncorrected Fisher's LSD post hoc multiple comparisons test (*<p.0.05; ****p<0.0001). (B) Average freezing behavior during cued fear memory test performed in the same animals as in (A) 1 h after the contextual fear memory test. The difference between groups was analyzed by parametric one-way ANOVA followed by an uncorrected Fisher's LSD post hoc multiple comparisons test (*p<0.05; ***p<0.001; ****p<0.0001). Bar graphs are mean±SEM.

### Body Weight

Animal body weight was followed weekly from 1 to 22 weeks after injections. As indicated in FIG. 28 (A), HET mice injected with vehicle-PBS showed a consistent and significant decrease in body weight from 1 to 22 weeks old when compared to their WT littermates injected with vehicle-PBS. This deficit in weight could be rescued by the AAV treatment with the STXBP1 long variant (HET-AAV9(L)) showing a significant difference to the HET vehicle-PBS group at 22 weeks (FIG. 28 (B)). The STXBP1 short variant group showed a trend of increased body weight at 22 weeks of age.

### Hindlimb Clasping

Hindlimb clasping (Guyenet et al., 2010) was recorded once a week from 4 to 10 weeks old and once every 3 weeks from 10 to 22 weeks old. Mice were suspended by their tail and the position of the hindlimbs was observed for 10 s. If the hindlimbs were consistently splayed outward, away from the abdomen, it was assigned a score of 0. If one hindlimb was retracted toward the abdomen for more than 50% of the time suspended, it received a score of 1. If both hindlimbs were partially retracted toward the abdomen for more than 50% of the time suspended, it received a score of 2. If its hindlimbs were entirely retracted and touching the abdomen for more than 50% of the time suspended, it received a score of 3. Each mouse was observed three times and the average score value was used for statistical analysis.

FIG. 29 (A) shows the progression of hindlimb clasping score in the four animal groups between week 4 and 22. At 5 weeks of age, the vehicle treated HET mice (HET-veh) started to display hindlimb clasping that stabilized at 7 weeks old when compared to the control WT littermates (WT-veh), indicating the development of dystonia in the STXBP1 haploinsufficiency model. AAV treatment with either the long or short STXBP1 variants in HET mice attenuated the progression of the hindlimb clasping compared to HET vehicle group over the period of 5 to 22 weeks. At 22 weeks of age, the extent of the hindlimb clasping recorded in the HET mice treated with the STXBP1 long variant was similar to the WT control group (non-significant difference, ns), indicating rescue of the dystonia phenotype (FIG. 29 (B)). The STXBP1 short variant significantly decreased the hindlimb clasping severity score in the HET mice at 22 weeks of age but without restoring the dystonia phenotype to WT level (FIG. 29 (B)).

### Wire Hanging Test

Eight weeks after AAV treatment mice were subjected to the four-limb wire hanging test (Klein et al., 2012) to evaluate the muscle strength. Mice were placed on a wire mesh, which was then inverted and waved gently, so that the mouse gripped the wire. Latency to fall was recorded, with a 90 s cut-off time. As shown in the FIG. 30, HET mice injected with vehicle-PBS displayed a significant increase in latency to fall at 8 weeks old when compared to their WT littermates treated with vehicle-PBS. The increase in latency to fall was abolished in HET mice treated with AAV encoding either the long or the short human STXBP1 variants, suggesting a full rescue of the phenotype in the haploinsufficiency model (FIG. 30).

### Fear Conditioning Test

Ten weeks following AVV treatment a Pavlovian fear conditioning paradigm (Curzon et al., 2009) was used to evaluate associative learning and memory, in which a mouse learns to associate a specific environment (i.e. the context) and a sound (i.e. the cue) with electric foot shocks. The fear memory is manifested by the mouse freezing, then it is subsequently exposed to this specific context or cue without electric shocks. The fear conditioning test was conducted in a chamber that has a grid floor for delivering electrical shocks (Ugo Basile). A camera above the chamber was used to monitor the mouse. During a 6 min training phase, a mouse was placed in the chamber (114-116 lux light intensity, one grey wall, grid floor visible) for 2 min habituation period to evaluate baseline freezing, and then a sound (78-80 dB, 4 kHz) was turned on for 30 s immediately followed by a mild foot shock (2 s, 0.5 mA). The same sound-foot shock association were repeated two more times after the first one with an interval time of 1 min. After the training phase, the mouse returned to its home cage. After 24 h, the mouse was tested for the contextual fear memory. For that, the mouse was placed in the same training chamber and its freezing behavior was monitored for 5 min without any sound or foot shock stimuli. The mouse was then returned to its home cage. One hour later, the mouse was transferred to the chamber after it had been altered with 3 checkered walls, no metal grid visible, white ground floor and 14-16 lux light intensity to create a new context for the cued fear memory test. After 2 min habituation period in the chamber to measure baseline freezing, the same sound cue that was used in the training phase was turned on four times for 30 s without foot shocks while the freezing behavior was monitored during a trial time of 7.5 min. The freezing time was determined using an automated video-based system using Ethovision software (Noldus).

FIG. 31 shows the results for the contextual test (FIG. 31 (A)) and the cued test (FIG. 31 (B)) for the four animal groups. STXBP1 HET mice treated with vehicle-PBS exhibited a profound reduction in both context- and cue-induced freezing behaviors when compared to their WT littermates, indicating a deficit in associative learning and memory in the STXBP1 haploinsufficiency model (FIG. 31 (A,B)). AAV treatment with the long and short STXBP1 variants led to an increase in freezing behavior in the contextual and cued test (FIG. 31 (A,B). The effects of the long variant treatment were significantly different from the HET vehicle treated groups indicating a rescue of the context- and cue-induced freezing behaviors. The short STXBP1 variant treatment led to a significant increase in the cued test when compared to the HET vehicle treated animals (FIG. 31 (B)) and showed a trend to increase the freezing in the contextual test (FIG. 31 (A)). Overall, AAV treatment with the STXBP1 variants had the potential to rescue the associative learning and memory deficits observed in the STXBP1 haploinsufficiency mouse model.

TABLE 19
Overview of behavioral disease symptoms in the STXBP1
haploinsufficiency mouse model. Observations in the HET
STXBP1 mice were compared to the WT littermates and were
classified as decreased (↓) or increased (↑). The effects of AAV
treatment including either the STXBP1 long or short
variant overexpression were labelled as “recovery” (statistically
significant change to HET vehicle treated mice) or “trend”
(change observed but not statistically significant).

| | | Observations | AAV treatment groups | |
|---|---|---|---|---|
| | | in HET | Long | Short |
| Symptoms | Test | (vs WT) | variant | variant |
| Growth deficit | body weight | [symbol] | recovery | trend |
| Epilepsy | Seizures (SWD) | [symbol] | recovery | recovery |
| Motor | Hindlimb clasping (dystonia) | [symbol] | recovery | recovery |
| dysfunction | Wire hanging (muscle strength) | [symbol] | recovery | recovery |
| Psychiatric | Fear conditioning (associative | [symbol] | recovery | trend |
| disorders | learning memory) | | | |

### REFERENCES

Abramov D, Guiberson N G L, Burre J (2020). STXBP1 encephalopathies: Clinical spectrum, disease mechanisms, and therapeutic strategies. Journal of neurochemistry.
Bertrand-Mathon et al. (2015). Increasing the effectiveness of intracerebral injections in adult and neonatal mice: a neurosurgical point of view. Neuroscience Bulletin, Springer Verlag, 2015, 31 (6), pp. 685-696. hal-01196282.
Chen W, Cai Z L, Chao E S, Chen H, Longley C M, Hao S, et al. (2020). Stxbp1/Munc18-1 haploinsufficiency impairs inhibition and mediates key neurological features of STXBP1 encephalopathy. eLife 9.
Curzon et al. (2009) Methods of Behavior Analysis in Neuroscience. 2nd edition. Boca Raton (FL): CRC Press/Taylor & Francis, Chapter 2.
Dulubova I et al. (2007) Munc18-1 binds directly to the neuronal SNARE complex, Proc. Natl. Acad. Sci. USA 104: 2697-2702.
Guyenet et al. (2010) A simple composite phenotype scoring system for evaluating mouse models of cerebellar ataxia, J Vis Exp. 21:1787.
Hamada N, Iwamoto I, Tabata H, Nagata K I (2017). MUNC18-1 gene abnormalities are involved in neurodevelopmental disorders through defective cortical architecture during brain development. Acta Neuropathol Commun 5: 92.
Hamodi, A. et al. (2020). Transverse sinus injections drive robust whole-brain expression of transgenes. eLIFE. DOI: 10.7554/eLife.53639.
Kalidas S, Santosh V, Shareef M M, Shankar S K, Christopher R, Shetty K T (2000). Expression of p67 (Munc-18) in adult human brain and neuroectodermal tumors of human central nervous system. Acta Neuropathol 99: 191-198.
Kim, J. Y et al. (2014). Intracerebroventricular Viral Injection of the Neonatal Mouse Brain for Persistent and Widespread Neuronal Transduction. J. Vis. Exp. (91), e51863, doi:10.3791/51863 (2014).
Klein et al. (2012) Noninvasive in vivo assessment of muscle impairment in the mdx mouse model—a comparison of two common wire hanging methods with two different results, J Neurosci Methods 203:292-7.
Kovacevic J, Maroteaux G, Schut D, Loos M, Dubey M, Pitsch J, et al. (2018). Protein instability, haploinsufficiency, and cortical hyper-excitability underlie STXBP1 encephalopathy. Brain: a journal of neurology 141(5): 1350-1374.
MacLean B, et al. (2010). Skyline: an open source document editor for creating and analyzing targeted proteomics experiments. Bioinformatics 26(7) 966-968.
Meijer M, Cijsouw T, Toonen R F, Verhage M (2015). Synaptic Effects of Munc18-1 Alternative Splicing in Excitatory Hippocampal Neurons. PloS one 10: e0138950.
Orock A, Logan S, Deak F (2018). Munc18-1 haploinsufficiency impairs learning and memory by reduced synaptic vesicular release in a model of Ohtahara syndrome. Molecular and cellular neurosciences 88: 33-42.
Patzke C, Han Y, Covy J, Yi F, Maxeiner S, Wernig M, et al. (2015). Analysis of conditional heterozygous STXBP1 mutations in human neurons. The Journal of clinical investigation 125: 3560-3571.
Ramos-Miguel A, Hercher C, Beasley C L, Barr A M, Bayer T A, Falkai P, et al. (2015). Loss of Munc18-1 long splice variant in GABAergic terminals is associated with cognitive decline and increased risk of dementia in a community sample. Molecular neurodegeneration 10: 65.
Saitsu H, Kato M, Mizuguchi T, Hamada K, Osaka H, Tohyama J, et al. (2008). De novo mutations in the gene encoding STXBP1 (MUNC18-1) cause early infantile epileptic encephalopathy. Nature Genetics 40(6): 782-788.
Stamberger H, Nikanorova M, Willemsen M H, Accorsi P, Angriman M, Baier H, et al. (2016). STXBP1 encephalopathy: A neurodevelopmental disorder including epilepsy. Neurology 86: 954-962.
Swanson D A, Steel J M, Valle D (1998). Identification and characterization of the human ortholog of rat STXBP1, a protein implicated in vesicle trafficking and neurotransmitter release. Genomics 48: 373-376.
Toonen R F, Wierda K, Sons M S, de Wit H, Cornelisse L N, Brussaard A, et al. (2006). Munc18-1 expression levels control synapse recovery by regulating readily releasable pool size. Proceedings of the National Academy of Sciences of the United States of America 103: 18332-18337.
Tremblay, R. G. et al. (2010). Differentiation of mouse Neuro 2A cells into dopamine neurons. J Neurosci Methods 186, 60-67, doi:10.1016/j.jneumeth.2009.11.004.
Uriguen L, Gil-Pisa I, Munarriz-Cuezva E, Berrocoso E, Pascau J, Soto-Montenegro M L, et al. (2013). Behavioral, neurochemical and morphological changes induced by the overexpression of munc18-1a in brain of mice: relevance to schizophrenia. Translational psychiatry 3: e221.
Verhage M, Maia A S, Plomp J J, Brussaard A B, Heeroma J H, Vermeer H, et al. (2000). Synaptic assembly of the brain in the absence of neurotransmitter secretion. Science 287: 864-869.

1. A nucleic acid construct comprising a transgene encoding:
i. a syntaxin binding protein 1 (STXBP1) comprising isoform a, b, c, d, e, f, g or h, having the sequence given in SEQ ID NO: 9, 10, 11, 12, 13, 14, 15, or 16 respectively; or
ii. a sequence having at least 95% sequence identity to SEQ ID NO:9, 10, 11, 12, 13, 14, 15 or 16 and retaining functionality as STXBP1; or
iii. a naturally-occurring variant comprising, with reference to SEQ ID NO:9, one or more mutations as shown in Table 7.

2. The nucleic acid construct according to claim 1, wherein the transgene encodes:
i. STXBP1 transcript variant 1, 2, 3, 4, 5, 6, 7, 8, 9, 10, 11, or 12, having the sequence given in SEQ ID NO: 22, 23, 24, 25, 26, 27, 28, 29, 30, 31, 32 or 33 respectively; or
ii. a sequence having at least 95% or 96% or 97% or 98% or 99% or 99.5% sequence identity to SEQ ID NO: 22, 23, 24, 25, 26, 27, 28, 29, 30, 31, 32 or 33.

3. The nucleic acid construct according to claim 1, wherein the transgene encodes STXBP1 isoform a and comprises a cDNA sequence of SEQ ID NO: 7; or a sequence having at least 95% or 96% or 97% or 98% or 99% or 99.5% sequence identity to SEQ ID NO: 7.

4. The nucleic acid construct according to any of claims 1 to 3, further comprising a promoter operably-linked to said transgene, wherein said promoter comprises:
i. a CAG 1.6 kb promoter of SEQ ID NO: 1; or
ii. a hSYN promoter of SEQ ID NO: 2; or
iii. a MECP2 promoter of SEQ ID NO: 3; or
iv. a hNSE promoter of SEQ ID NO: 4; or
v. a CamKII promoter of SEQ ID NO: 5; or
vi. an endogenous hSTXBP1 promoter of SEQ ID NO: 6; or
vii. a MECP2 promoter of SEQ ID NO: 3 operably linked in a 5′ to 3′ orientation to a MECP2 intron of SEQ ID NO: 37.

5. The nucleic acid construct according to any one of the preceding claims, wherein the construct comprises a SV40 polyadenylation signal sequence of SEQ ID NO: 8.

6. A viral vector comprising the nucleic acid construct according to any one of the preceding claims, wherein the viral vector further comprises an inverted terminal repeat (ITR) at the 5′ and/or 3′ flank of said nucleic acid construct.

7. The viral vector according to claim 6, wherein the 5′ITR and/or the 3′ITR comprises the ITR of a natural adeno-associated virus (AAV).

8. The viral vector according to claim 6 or claim 7, wherein the 3′ITR comprises SEQ ID NO: 18 and/or the 5′ITR comprises SEQ ID NO: 19.

9. A viral particle comprising a nucleic acid construct according to any one of claims 1 to 5 or a viral vector according to any one of claims 6 to 8.

10. The viral particle according to claim 9, comprising a VP1 capsid protein from an AAV selected from the group consisting of AAV2, AAV5, AAV6, AAV8, AAV9, AAV10, AAVtt, or combinations thereof.

11. The viral particle according to claim 10, wherein the capsid protein is from AAVtt or AAV9 and comprises SEQ ID NO: 20 or 21 respectively, or a sequence having at least 98.5% or 99% or 99.5% sequence identity to SEQ ID NO: 20 or 21.

12. The viral particle according to any one of claims 9 to 11 for use in therapy.

13. The viral particle for use according to claim 12 in the treatment and/or prevention of an STXBP1 genetic disorder associated with severe early onset epileptic encephalopathy.

14. The viral particle for use according to claim 12 or claim 13 in the treatment of Ohtahara syndrome, West syndrome or Dravet syndrome.

15. A method of treating and/or preventing a disease characterised by loss of STXBP1 functional activity, comprising administering to a subject in need thereof a viral particle according to any one of claims 9 to 11.

16. The method according to claim 15, wherein said disease is associated with at least one mutation in a patient which leads to a pathological STXBP1 variant, wherein said pathological STXBP1 variant comprises, with reference to SEQ ID NO:9, a mutation or combination of mutations as shown in Table 5 and/or Table 6.


SEQUENCE LISTING (47 sequences)

SEQ ID NO: 1 - DNA, 582 bp, synthetic construct
  source 1..582: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcgtcgaggtgagccccacgttctgcttcactctccccatctcccccccctccccacccccaattttgtatttatttattttttaattattttatgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 2 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 3 - DNA, 229 bp, synthetic construct
  source 1..229: mol_type = other DNA
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 4 - DNA, 1317 bp, synthetic construct
  source 1..1317: mol_type = other DNA
atgcagctggacctaggagagaagcaggagaggaagatccagcacaaaaaatccgaagctaaaaacaggacacagagatgggggaagaaaagagggcagagtgaggcaaaaagagactgaagagatgagggtggccgccaggcactttagataggggagaggctttatttacctctgtttgtttttttttttttttttttttttttttttttgcgaggtagtcttgcttagtctccaggctggagtgcagtggcacaatctcagctcactgcaacttccacctcctgggttcaagcaattctcctgcctcagcctcccgagtagctgggactacaggcgcatgcaaccgcgcctggctaatttttgtatttttagtagaaacggggtttcaccacgttagccaggatggtctggatctcctgacctcgtgatctgcccgcctccgccttccaaagtgctgggattacaggggtgagccacagcgcctggtccctatttacttctgtcttctacctccaggagatcaaagacgctggccttcagacctgatcagactcccaggggcagccaccacatgtatgacagagaacagaggatgcctgtttttgccccaaagctggaaattcatcacaacctgaggcccaggatctgctctgtgccggtcctctgggcagtgtggggtgcagaatggggtgcctaggcctgagcgttgcctggagcctaggccgggggccgccctcgggcaggcgtgggtgagagccaagaccgcgtgggccgcggggtgctggtaggagtggttggagagacttgcgaaggcggctggggtgttcggatttccaataaagaaacagagtgatgctcctgtgtctgaccgggtttgtgagacattgaggctgtcttgggcttcactggcagtgtgggccttcgtacccgggctacaggggtgcggctctgcctgttactgtcgagtgggtcgggccgtgggtatgagcgcttgtgtgcgctggggccaggtcgtgggtgcccccacccttcccccatcctcctcccttccccactccaccctcgtcggtcccccacccgcgctcgtacgtgcgcctccgccggcagctcctgactcatcgggggctccgggtcacatgcgcccgcgcggccctataggcgcctcctccgcccgccgcccgggagccgcagccgccgccgccactgccactcccgctctctcagcgccgccgtcgccaccgccaccgccaccgccactaccaccgagatctgcgatctaagtaagcttggcattccggtactgttggtaaagcc

SEQ ID NO: 5 - DNA, 1289 bp, synthetic construct
  source 1..1289: mol_type = other DNA
cattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 6 - DNA, 726 bp, synthetic construct
  source 1..726: mol_type = other DNA
taaaaagcaatgcccagtgattggaggatttgatgagatgatgcccgcgaggtgcttggcacggagtttgacacaagaactcagtgttggtgaatgcacgaatgcaggtacccagcgacagggaggtgctgtgggtggattcaccctggcttcgctgcggctgggagtgggcgctgctagtaagagatctcgcctccaagctccttccgtgggcaggaaaaaacggaagctcccagaaagaggaaagacaggacccgagcggggtttcaggcagatggagcgcgtcggtagcctgtggccagggatcccagcaccgacgggaaagaggaggcctgggtaccctgcgccccgggcgcgcgcggcgcgtgaggtgagggggagggcgcggctccgcaccagccagcggccgcctggcgcccagccgcatctcggggggcggggttgagcctccgggccgcagtgcgattggcggaggcgagtgggtgacgccaacggccggcgcgaggccccgcccccggcttgccccgcccccgcgcgcgccggcggcggggcagcctcgctctggctcgcgccgcgcccccgcgcccagtccgcgcgtcagtcggtccctagcgcggctgcggggcggagagctgcggctggcccagcgcgcccacctgaggaggcggcggggtccgcaggcgtcgcgggacgaggagatcggagccgggagactcgcgcagcgcc

SEQ ID NO: 7 - DNA, 1812 bp, synthetic construct
  source 1..1812: mol_type = other DNA
atggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggttctactcacattcttactcccaccaaatttctcatggacctgagacaccccgacttcagggagtcctctagggtatcttttgaggatcaggctccaacaatggagtga

SEQ ID NO: 8 - DNA, 135 bp, synthetic construct
  source 1..135: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 9 - protein, 603 aa, Homo sapiens [sequence omitted]

SEQ ID NO: 10 - protein, 594 aa, Homo sapiens
MAPIGLKAVVGEKIMHDVIKKVKKKGEWKVLVVDQLSMRMLSSCCKMTDIMTEGITIVEDINKRREPLPSLEAVYLITPSEKSVHSLISDFKDPPTAKYRAAHVFFTDSCPDALFNELVKSRAAKVIKTLTEINIAFLPYESQVYSLDSADSFQSFYSPHKAQMKNPILERLAEQIATLCATLKEYPAVRYRGEYKDNALLAQLIQDKLDAYKADDPTMGEGPDKARSQLLILDRGFDPSSPVLHELTFQAMSYDLLPIENDVYKYETSGIGEARVKEVLLDEDDDLWIALRHKHIAEVSQEVTRSLKDFSSSKRMNTGEKTTMRDLSQMLKKMPQYQKELSKYSTHLHLAEDCMKHYQGTVDKLCRVEQDLAMGTDAEGEKIKDPMRAIVPILLDANVSTYDKIRIILLYIFLKNGITEENLNKLIQHAQIPPEDSEIITNMAHLGVPIVTDSTLRRRSKPERKERISEQTYQLSRWTPIIKDIMEDTIEDKLDTKHYPYISTRSSASFSTTAVSARYGHWHKNKAPGEYRSGPRLIIFILGGVSLNEMRCAYEVTQANGKWEVLIGSTHILTPQKLLDTLKKLNKTDEEISS

SEQ ID NO: 11 - protein, 591 aa, Homo sapiens
MAPIGLKAVVGEKIMHDVIKKVKKKGEWKVLVVDQLSMRMLSSCCKMTDIMTEGITIVEDINKRREPLPSLEAVYLITPSEKSVHSLISDFKDPPTAKYRAAHVFFTDYALFNELVKSRAAKVIKTLTEINIAFLPYESQVYSLDSADSFQSFYSPHKAQMKNPILERLAEQIATLCATLKEYPAVRYRGEYKDNALLAQLIQDKLDAYKADDPTMGEGPDKARSQLLILDRGFDPSSPVLHELTFQAMSYDLLPIENDVYKYETSGIGEARVKEVLLDEDDDLWIALRHKHIAEVSQEVTRSLKDFSSSKRMNTGEKTTMRDLSQMLKKMPQYQKELSKYSTHLHLAEDCMKHYQGTVDKLCRVEQDLAMGTDAEGEKIKDPMRAIVPILLDANVSTYDKIRIILLYIFLKNGITEENLNKLIQHAQIPPEDSEIITNMAHLGVPIVTDSTLRRRSKPERKERISEQTYQLSRWTPIIKDIMEDTIEDKLDTKHYPYISTRSSASFSTTAVSARYGHWHKNKAPGEYRSGPRLIIFILGGVSLNEMRCAYEVTQANGKWEVLIGSTHILTPQKLLDTLKKLNKTDEEISS

SEQ ID NO: 12 - protein, 589 aa, Homo sapiens
MHDVIKKVKKKGEWKVLVVDQLSMRMLSSCCKMTDIMTEGITIVEDINKRREPLPSLEAVYLITPSEKSVHSLISDFKDPPTAKYRAAHVFFTDSCPDALFNELVKSRAAKVIKTLTEINIAFLPYESQVYSLDSADSFQSFYSPHKAQMKNPILERLAEQIATLCATLKEYPAVRYRGEYKDNALLAQLIQDKLDAYKADDPTMGEGPDKARSQLLILDRGFDPSSPVLHELTFQAMSYDLLPIENDVYKYETSGIGEARVKEVLLDEDDDLWIALRHKHIAEVSQEVTRSLKDFSSSKRMNTGEKTTMRDLSQMLKKMPQYQKELSKYSTHLHLAEDCMKHYQGTVDKLCRVEQDLAMGTDAEGEKIKDPMRAIVPILLDANVSTYDKIRIILLYIFLKNGITEENLNKLIQHAQIPPEDSEIITNMAHLGVPIVTDSTLRRRSKPERKERISEQTYQLSRWTPIIKDIMEDTIEDKLDTKHYPYISTRSSASFSTTAVSARYGHWHKNKAPGEYRSGPRLIIFILGGVSLNEMRCAYEVTQANGKWEVLIGSTHILTPTKFLMDLRHPDFRESSRVSFEDQAPTME

SEQ ID NO: 13 - protein, 580 aa, Homo sapiens
MHDVIKKVKKKGEWKVLVVDQLSMRMLSSCCKMTDIMTEGITIVEDINKRREPLPSLEAVYLITPSEKSVHSLISDFKDPPTAKYRAAHVFFTDSCPDALFNELVKSRAAKVIKTLTEINIAFLPYESQVYSLDSADSFQSFYSPHKAQMKNPILERLAEQIATLCATLKEYPAVRYRGEYKDNALLAQLIQDKLDAYKADDPTMGEGPDKARSQLLILDRGFDPSSPVLHELTFQAMSYDLLPIENDVYKYETSGIGEARVKEVLLDEDDDLWIALRHKHIAEVSQEVTRSLKDFSSSKRMNTGEKTTMRDLSQMLKKMPQYQKELSKYSTHLHLAEDCMKHYQGTVDKLCRVEQDLAMGTDAEGEKIKDPMRAIVPILLDANVSTYDKIRIILLYIFLKNGITEENLNKLIQHAQIPPEDSEIITNMAHLGVPIVTDSTLRRRSKPERKERISEQTYQLSRWTPIIKDIMEDTIEDKLDTKHYPYISTRSSASFSTTAVSARYGHWHKNKAPGEYRSGPRLIIFILGGVSLNEMRCAYEVTQANGKWEVLIGSTHILTPQKLLDTLKKLNKTDEEISS

SEQ ID NO: 14 - protein, 577 aa, Homo sapiens
MAPIGLKAVVGEKIMHDVIKKVKKKGEWKVLVVDQLSMRMLSSCCKMTDIMTEGITIVEDINKRREPLPSLEAVYLITPSEKSVHSLISDFKDPPTAKYRAAHVFFTDSCPDALFNELVKSRAAKVIKTLTEINIAFLPYESQVYSLDSADSFQSFYSPHKAQMKNPILERLAEQIATLCATLKEYPAVRYRGEYKDNALLAQLIQDKLDAYKADDPTMGEGPDKARSQLLILDRGFDPSSPVLHELTFQAMSYDLLPIENDVYKYETSGIGEARVKEVLLDEDDDLWIALRHKHIAEVSQEVTRSLKDFSSSKRMNTGEKTTMRDLSQMLKKMPQYQKELSKYSTHLHLAEDCMKHYQGTVDKLCRVEQDLAMGTDAEGEKIKDPMRAIVPILLDANVSTYDKIRIILLYIFLKNGITEENLNKLIQHAQIPPEDSEIITNMAHLGVPIVTDSTLRRRSKPERKERISEQTYQLSRWTPIIKDIMEDTIEDKLDTKHYPYISTRSSASFSTTAVSARYGHWHKNKAPGEYRSGPRLIIFILGGVSLNEMRCAYEVTQANGKWEVLIGEWPFRTLAR

SEQ ID NO: 15 - protein, 574 aa, Homo sapiens
MAPIGLKAVVGEKIMHDVIKKVKKKGEWKVLVVDQLSMRMLSSCCKMTDIMTEGITIVEDINKRREPLPSLEAVYLITPSEKSVHSLISDFKDPPTAKYRAAHVFFTDSCPDALFNELVKSRAAKVIKTLTEINIAFLPYESQVYSLDSADSFQSFYSPHKAQMKNPILERLAEQIATLCATLKEYPAVRYRGEYKDNALLAQLIQDKLDAYKADDPTMGEGPDKARSQLLILDRGFDPSSPVLHELTFQAMSYDLLPIENDVYKYETSGIGEARVKEVLLDEDDDLWIALRHKHIAEVSQEVTRSLKDFSSSKRMNTGEKTTMRDLSQMLKKMPQYQKELSKYSTHLHLAEDCMKHYQGTVDKLCRVEQDLAMGTDAEGEKIKDPMRAIVPILLDANVSTYDKIRIILLYIFLKNGITEENLNKLIQHAQIPPEDSEIITNMAHLGVPIVTDSTLRRRSKPERKERISEQTYQLSRWTPIIKDIMEDTIEDKLDTKHYPYISTRSSASFSTTAVSARYGHWHKNKAPGEYRSGPRLIIFILGGVSLNEMRCAYEVTQANGKWEVLIVPVEAGS

SEQ ID NO: 16 - protein, 567 aa, Homo sapiens
MAPIGLKAVVGEKIMHDVIKKVKKKGEWKVLVVDQLSMRMLSSCCKMTDIMTEGITIVEDINKRREPLPSLEAVYLITPSEKSVHSLISDFKDPPTAKYRAAHVFFTDSCPDALFNELVKSRAAKVIKTLTEINIAFLPYESQVYSLDSADSFQSFYSPHKAQMKNPILERLAEQIATLCATLKEYPAVRYRGEYKDNALLAQLIQDKLDAYKADDPTMGEGPDKARSQLLILDRGFDPSSPVLHELTFQAMSYDLLPIENDVYKEVTRSLKDFSSSKRMNTGEKTTMRDLSQMLKKMPQYQKELSKYSTHLHLAEDCMKHYQGTVDKLCRVEQDLAMGTDAEGEKIKDPMRAIVPILLDANVSTYDKIRIILLYIFLKNGITEENLNKLIQHAQIPPEDSEIITNMAHLGVPIVTDSTLRRRSKPERKERISEQTYQLSRWTPIIKDIMEDTIEDKLDTKHYPYISTRSSASFSTTAVSARYGHWHKNKAPGEYRSGPRLIIFILGGVSLNEMRCAYEVTQANGKWEVLIGSTHILTPTKFLMDLRHPDFRESSRVSFEDQAPTME

SEQ ID NO: 17 - DNA, 6921 bp, synthetic construct
  source 1..6921: mol_type = other DNA
agaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttcgctgaagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcaggacagtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgcgtcaaaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 18 - DNA, 128 bp, synthetic construct
  source 1..128: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 19 - DNA, 128 bp, synthetic construct
  source 1..128: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 20 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKSGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 21 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - DNA, 3880 bp, synthetic construct
  source 1..3880: mol_type = other DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggttctactcacattcttactcccaccaaatttctcatggacctgagacaccccgacttcagggagtcctctagggtatcttttgaggatcaggctccaacaatggagtgagagccaaagaaacaaagatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 23 - DNA, 3754 bp, synthetic construct
  source 1..3754: mol_type = other DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 24 - DNA, 3745 bp, synthetic construct
  source 1..3745: mol_type = other DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 25 - DNA, 3975 bp, synthetic construct
  source 1..3975: mol_type = other DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagagaaagaggaagactacctagacatgttgctccagagcagaatttccagcagtagattgaaccttcggaggccgagtcccagcccagtgtaaactgagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggttctactcacattcttactcccaccaaatttctcatggacctgagacaccccgacttcagggagtcctctagggtatcttttgaggatcaggctccaacaatggagtgagagccaaagaaacaaagatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 26 - DNA, 4070 bp, synthetic construct
  source 1..4070: mol_type = other DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagagatatatacctaggaatggagttgctggatctctatgtttggttatttgaggagctgccggcctgttttccacatcagctgtaccattttacattctcaccagcagtgcatgacggtccagtgcctttttatcttactgcgcttaaggtgtaattccttctttctcagactggactgtaaattctttgagagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggttctactcacattcttactcccaccaaatttctcatggacctgagacaccccgacttcagggagtcctctagggtatcttttgaggatcaggctccaacaatggagtgagagccaaagaaacaaagatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 27 - DNA, 3726 bp, synthetic construct
  source 1..3726: mol_type = other DNA
agaaagaggaaagacaggacccgagcggggtttcaggcagatggagcgcgtcggtagcctgtggccagggatcccagcaccgacgggaaagaggaggcctgggtaccctgcgccccgggcgcgcgcggcgcgtgagagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 28 - DNA, 3944 bp, synthetic construct
  source 1..3944: mol_type = other DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagagatatatacctaggaatggagttgctggatctctatgtttggttatttgaggagctgccggcctgttttccacatcagctgtaccattttacattctcaccagcagtgcatgacggtccagtgcctttttatcttactgcgcttaaggtgtaattccttctttctcagactggactgtaaattctttgagagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 29 - DNA, 3849 bp, synthetic construct
  source 1..3849: mol_type = other DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagagaaagaggaagactacctagacatgttgctccagagcagaatttccagcagtagattgaaccttcggaggccgagtcccagcccagtgtaaactgagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 30 - DNA, 3856 bp, synthetic construct
  source 1..3856: mol_type = other DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagagaaagaggaagactacctagacatgttgctccagagcagaatttccagcagtagattgaaccttcggaggccgagtcccagcccagtgtaaactggtaaagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 31 - DNA, 3834 bp, synthetic construct
  source 1..3834: mol_type = other DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggggaatggcccttcagaaccttggctaggtgacatcagaagctgcttgtgtatgtaaggaaaatggggcttcctcctaaggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 32 - DNA, 2865 bp, synthetic construct
  source 1..2865: mol_type = other DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgatagtgcccgtggaggcgggaagctgagcccggctcccaggaagcacagtgagggctcagccattgatgatgatgatgatgatgatgatgatgatgatgatgatggtgatgatgatgatgctgctgctgttgacgatgagtccaagccaagcctggggagaaaagaataaatagtctcataaatttaaaaaagcccaatgctcatcaagaagagaacagataaactgtgtattcacccaatggtgagtttaaagcagttaaaaatgcatgacacacagttgcatatgtcaacatgaatagatctcagacacttaacaatgagtgaaaagagcacattatgggaggacacagacaggatagtgcatttatataaagcttcaatgttgcaggctgaaagagtgagggtcgtgatcaactcagtatcctggaggctacatgggtaaacagcaaactgttctcataaatgcagaatgttggcaaactgacaaactgcatctgccgcccagaaggaatgcggagggcagccactccctaagcgcagttttcttgtgattaggtacatctgaagcctgttagcaataatgtgaacctgtgatcaattaagcagctgaccaatcattacctcctcttccctgctctttctacccagtaaatacaaagggctgtagaagctcagagctgctgcttttgctcagtagaagcagggagtcctcttcttcttcccccgaccccttcttttaaaacagtttcttttaagttttcatttctgcgttcatcctccttcattcagtcccgtagtaaccgtggcaaaccacggcacttcaaaacctcataaaaagcaccacatattgcttatttggatacatatgttgtaaatgtgtgaaaacatgcatgggaatgacaaataccaaagtcaggatagtgactggccatggggaagggatggttattattgtcgtcttatatcttcaattccaaatgagccacgggcttaaaagtacttcattgaataaaaaaaaaccaacaatgtattaatgcaa

SEQ ID NO: 33 - DNA, 3772 bp, synthetic construct
  source 1..3772: mol_type = other DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggttctactcacattcttactcccaccaaatttctcatggacctgagacaccccgacttcagggagtcctctagggtatcttttgaggatcaggctccaacaatggagtgagagccaaagaaacaaagatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 34 - DNA, 6946 bp, synthetic construct
  source 1..6946: mol_type = other DNA
tttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttcgctgaagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagaggatccttgaacctctgggcctggttgaggaacctgttaagacggctccgggaaaaaagaggccggtagagcactctcctgccgagccagactcctcctcgggaaccggaaagagcggccagcagcctgcaagaaaaagattgaattttggtcagactggagacgcagactcagtacctgacccccagcctctcggacagccaccagcagccccctctggtctgggaactaatacgatggctagcggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcagcttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgatgtcaaggcttcagttttctcaggccggagcgagtgacattcgggaccagtctaggaactggcttcctggaccctgttaccgccagcagcgagtatcaaagacagccgcggataacaacaacagtgactactcgtggactggagctaccaagtaccacctcaatggcagagactctctggtgaatccgggcccggccatggcaagccacaaggacgatgaagaaaagtactttcctcagagcggggttctcatctttgggaagcaagactcaggcaaaacaaatgtggacattgaaaaggtcatgattacagacgaagaggaaatcaggacaaccaatcccgtggctacggagcagtatggttctgtatctaccaacctccagagcggcaacacccaagcagctaccagcgatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgcgtcaaaagggcgacacaaaatttattctaaatgcataataaatactgataacatcttatagtttgtattatattttgtattatcgttgacatgtataatttttctagagcggccgcagatctcagctggatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctt

SEQ ID NO: 35 - DNA, 30 bp, synthetic construct
  source 1..30: mol_type = other DNA
gagcagaaactcatctcagaagaggatctg

SEQ ID NO: 36 - DNA, 27 bp, synthetic construct
  source 1..27: mol_type = other DNA
tacccttacgatgtaccggattacgca

SEQ ID NO: 37 - DNA, 2000 bp, synthetic construct
  source 1..2000: mol_type = other DNA
gcgctccctcctctcggagagagggctgtggtaaaacccgtccggaaattggccgccgctgccgccaccgccgccgccgccgccgcgccgagcggaggaggaggaggaggcgaggaggagagactgtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcgttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcagggaggaaaagtcagaagaccaggatctccagggcctca

SEQ ID NO: 38 - protein, 36 aa, Homo sapiens
GSTHILTPTKFLMDLRHPDFRESSRVSFEDQAPTME

SEQ ID NO: 39 - protein, 27 aa, Homo sapiens
GSTHILTPQKLLDTLKKLNKTDEEISS

SEQ ID NO: 40 - DNA, 25 bp, synthetic construct
  source 1..25: mol_type = other DNA
gatccagacatgataagatacattg

SEQ ID NO: 41 - DNA, 22 bp, synthetic construct
  source 1..22: mol_type = other DNA
gcaatagcatcacaaatttcac

SEQ ID NO: 42 - DNA, 24 bp, synthetic construct
  source 1..24: mol_type = other DNA
tggacaaaccacaactagaatgca

SEQ ID NO: 43 - protein, 12 aa, Homo sapiens
DNALLAQLIQDK

SEQ ID NO: 44 - protein, 10 aa, Homo sapiens
YETSGIGEAR

SEQ ID NO: 45 - protein, 10 aa, Homo sapiens
ISEQTYQLSR

SEQ ID NO: 46 - protein, 15 aa, Homo sapiens
WEVLIGSTHILTPTK

SEQ ID NO: 47 - protein, 15 aa, Homo sapiens
WEVLIGSTHILTPQK